SEQUENCE LISTING (227 sequences)

SEQ ID NO: 1 - protein, 1231 aa, synthetic construct
  REGION 1..1231: note = Human Complement Factor H (FH)
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 449 aa, synthetic construct
  REGION 1..449: note = Human FH-like protein 1 (FHL-1)
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 3 - protein, 330 aa, synthetic construct
  REGION 1..330: note = Complement Factor H-related 1 (FHR1)
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 4 - protein, 270 aa, synthetic construct
  REGION 1..270: note = Complement Factor H-related 2 (FHR2), isoform 1
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 5 - protein, 243 aa, synthetic construct
  REGION 1..243: note = Complement Factor H-related 2 (FHR2), isoform 2
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTSSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 6 - protein, 330 aa, synthetic construct
  REGION 1..330: note = Complement Factor H-related 3 (FHR3), isoform 1
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 7 - protein, 269 aa, synthetic construct
  REGION 1..269: note = Complement Factor H-related 3 (FHR3), isoform 2
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVNSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 8 - protein, 578 aa, synthetic construct
  REGION 1..578: note = Complement Factor H-related 4 (FHR4A)
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 9 - protein, 331 aa, synthetic construct
  REGION 1..331: note = Complement Factor H-related 4 (FHR4B)
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 10 - protein, 569 aa, synthetic construct
  REGION 1..569: note = Complement Factor H-related 5 (FHR5)
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 11 - protein, 583 aa, synthetic construct
  REGION 1..583: note = Complement Factor I (FI)
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 12 - protein, 1663 aa, synthetic construct
  REGION 1..1663: note = Complement factor C3
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 13 - protein, 645 aa, synthetic construct
  REGION 1..645: note = Human C3 beta chain
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAA

SEQ ID NO: 14 - protein, 915 aa, synthetic construct
  REGION 1..915: note = Human C3 alpha' chain
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 15 - protein, 77 aa, synthetic construct
  REGION 1..77: note = Human C3a
SVQLTEKRMDKVGKYPKELRKCCEDGMRENPMRFSCQRRTRFISLGEACKKVFLDCCNYITELRRQHARASHLGLAR

SEQ ID NO: 16 - protein, 915 aa, synthetic construct; identical to SEQ ID NO: 14
  REGION 1..915: note = Human C3b alpha' chain fragment 1

SEQ ID NO: 17 - protein, 343 aa, synthetic construct
  REGION 1..343: note = Human C3 alpha' chain fragment 2
SEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 18 - protein, 17 aa, synthetic construct
  REGION 1..17: note = C3f
SSKITHRIHWESASLLR

SEQ ID NO: 19 - protein, 206 aa, synthetic construct
  REGION 1..206: note = Human C3c alpha' chain fragment 1
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGR

SEQ ID NO: 20 - protein, 7 aa, synthetic construct
  REGION 1..7: note = FH MS peptide
VTYKCFE

SEQ ID NO: 21 - protein, 16 aa, synthetic construct
  REGION 1..16: note = FHL-1 MS peptide
NGWSPTPRCIRVSFTL

SEQ ID NO: 22 - protein, 18 aa, synthetic construct
  REGION 1..18: note = FHR1 MS peptide
ATFCDFPKINHGILYDEE

SEQ ID NO: 23 - protein, 16 aa, synthetic construct
  REGION 1..16: note = FHR2 MS peptide 1
RGWSTPPKCRSTISAE

SEQ ID NO: 24 - protein, 18 aa, synthetic construct
  REGION 1..18: note = FHR2 MS peptide 2
AMFCDFPKINHGILYDEE

SEQ ID NO: 25 - protein, 20 aa, synthetic construct
  REGION 1..20: note = FHR3 MS peptide
VACHPGYGLPKAQTTVTCTE

SEQ ID NO: 26 - protein, 8 aa, synthetic construct
  REGION 1..8: note = FHR4 MS peptide
YQCQSYYE

SEQ ID NO: 27 - protein, 15 aa, synthetic construct
  REGION 1..15: note = FHR5 MS peptide
RGWSTPPICSFTKGE

SEQ ID NO: 28 - protein, 13 aa, synthetic construct
  REGION 1..13: note = C3.1 peptide
GTAFVIFGIQDGE

SEQ ID NO: 29 - protein, 20 aa, synthetic construct
  REGION 1..20: note = C3.2 peptide
LRRQHARASHLGLARSNLDE

SEQ ID NO: 30 - protein, 15 aa, synthetic construct
  REGION 1..15: note = C3.3 peptide
LRRQHARASHLGLAR

SEQ ID NO: 31 - protein, 23 aa, synthetic construct
  REGION 1..23: note = C3.4 peptide
LNLDVSLQLPSRSSKITHRIHWE

SEQ ID NO: 32 - protein, 12 aa, synthetic construct
  REGION 1..12: note = C3.5 peptide
LNLDVSLQLPSR

SEQ ID NO: 33 - protein, 5 aa, synthetic construct
  REGION 1..5: note = C3.6 peptide
RLGRE

SEQ ID NO: 34 - protein, 11 aa, synthetic construct
  REGION 1..11: note = C3.7 peptide
SSKITHRIHWE

SEQ ID NO: 35 - protein, 6 aa, synthetic construct
  REGION 1..6: note = C3.8 peptide
SASLLR

SEQ ID NO: 36 - protein, 4 aa, synthetic construct
  REGION 1..4: note = C3.9 peptide
RLGR

SEQ ID NO: 37 - protein, 11 aa, synthetic construct
  REGION 1..11: note = C3.10 peptide
HLIVTPSGCGE

SEQ ID NO: 38 - protein, 39 aa, synthetic construct
  REGION 1..39: note = FI peptide 1
CAGTYDGSIDACKGDSGGPLVCMDANNVTYVWGVVSWGE

SEQ ID NO: 39 - protein, 36 aa, synthetic construct
  REGION 1..36: note = FI peptide 2
GTCVCKLPYQCPKNGTAVCATNRRSFPTYCQQKSLE

SEQ ID NO: 40 - protein, 29 aa, synthetic construct
  REGION 1..29: note = FI peptide 3
FPGVYTKVANYFDWISYHVGRPFISQYNV

SEQ ID NO: 41 - protein, 31 aa, synthetic construct
  REGION 1..31: note = FI peptide 4
ANVACLDLGFQQGADTQRRFKLSDLSINSTE

SEQ ID NO: 42 - protein, 30 aa, synthetic construct
  REGION 1..30: note = FI peptide 5
LPRSIPACVPWSPYLFQPNDTCIVSGWGRE

SEQ ID NO: 43 - protein, 26 aa, synthetic construct
  REGION 1..26: note = FI peptide 6
KKCLAKKYTHLSCDKVFCQPWQRCIE

SEQ ID NO: 44 - protein, 28 aa, synthetic construct
  REGION 1..28: note = FI peptide 7
LCCKACQGKGFHCKSGVCIPSQYQCNGE

SEQ ID NO: 45 - protein, 21 aa, synthetic construct
  REGION 1..21: note = FI peptide 8
VKLVDQDKTMFICKSSWSMRE

SEQ ID NO: 46 - protein, 17 aa, synthetic construct
  REGION 1..17: note = FI peptide 9
VKLISNCSKFYGNRFYE

SEQ ID NO: 47 - protein, 17 aa, synthetic construct
  REGION 1..17: note = FI peptide 10
CLHPGTKFLNNGTCTAE

SEQ ID NO: 48 - protein, 15 aa, synthetic construct
  REGION 1..15: note = FI peptide 11
NYNAGTYQNDIALIE

SEQ ID NO: 49 - protein, 15 aa, synthetic construct
  REGION 1..15: note = FI peptide 12
GKFSVSLKHGNTDSE

SEQ ID NO: 50 - protein, 13 aa, synthetic construct
  REGION 1..13: note = FI peptide 13
VGCAGFASVTQEE

SEQ ID NO: 51 - protein, 11 aa, synthetic construct
  REGION 1..11: note = FI peptide 14
MKKDGNKKDCE

SEQ ID NO: 52 - protein, 9 aa, synthetic construct
  REGION 1..9: note = FI peptide 15
YVDRIIFHE

SEQ ID NO: 53 - protein, 10 aa, synthetic construct
  REGION 1..10: note = FI peptide 16
CLHVHCRGLE

SEQ ID NO: 54 - protein, 9 aa, synthetic construct
  REGION 1..9: note = FI peptide 17
RVFSLQWGE

SEQ ID NO: 55 - protein, 9 aa, synthetic construct
  REGION 1..9: note = FI peptide 18
ILTADMDAE

SEQ ID NO: 56 - protein, 8 aa, synthetic construct
  REGION 1..8: note = FI peptide 19
KVTYTSQE

SEQ ID NO: 57 - protein, 7 aa, synthetic construct
  REGION 1..7: note = FI peptide 20
VDCITGE

SEQ ID NO: 58 - protein, 6 aa, synthetic construct
  REGION 1..6: note = FI peptide 21
NCGKPE

SEQ ID NO: 59 - protein, 5 aa, synthetic construct
  REGION 1..5: note = FI peptide 22
TSLAE

SEQ ID NO: 60 - protein, 4 aa, synthetic construct
  REGION 1..4: note = FI peptide 23
KDNE

SEQ ID NO: 61 - protein, 61 aa, synthetic construct
  REGION 1..61: note = C3.1.1
AKIRYYTYLIMNKGRLLKAGRQVREPGQDLVVLPLSITTDFIPSFRLVAYYTLIGASGQRE

SEQ ID NO: 62 - protein, 52 aa, synthetic construct
  REGION 1..52: note = C3.1.2
RSGIPIVTSPYQIHFTKTPKYFKPGMPFDLMVFVTNPDGSPAYRVPVAVQGE

SEQ ID NO: 63 - protein, 50 aa, synthetic construct
  REGION 1..50: note = C3.1.3
KVVLVSLQSGYLFIQTDKTIYTPGSTVLYRIFTVNHKLLPVGRTVMVNIE

SEQ ID NO: 64 - protein, 36 aa, synthetic construct
  REGION 1..36: note = C3.1.4
DTVQSLTQGDGVAKLSINTHPSQKPLSITVRTKKQE

SEQ ID NO: 65 - protein, 35 aa, synthetic construct
  REGION 1..35: note = C3.1.5
GDHGARVVLVAVDKGVFVLNKKNKLTQSKIWDVVE

SEQ ID NO: 66 - protein, 38 aa, synthetic construct
  REGION 1..38: note = C3.1.6
KADIGCTPGSGKDYAGVFSDAGLTFTSSSGQQTAQRAE

SEQ ID NO: 67 - protein, 29 aa, synthetic construct
  REGION 1..29: note = C3.1.7
QATRTMQALPYSTVGNSNNYLHLSVLRTE

SEQ ID NO: 68 - protein, 27 aa, synthetic construct
  REGION 1..27: note = C3.1.8
AHDAQGDVPVTVTVHDFPGKKLVLSSE

SEQ ID NO: 69 - protein, 26 aa, synthetic construct
  REGION 1..26: note = C3.1.9
GIPVKQDSLSSQNQLGVLPLSWDIPE

SEQ ID NO: 70 - protein, 26 aa, synthetic construct
  REGION 1..26: note = C3.1.10
VVADSVWVDVKDSCVGSLVVKSGQSE

SEQ ID NO: 71 - protein, 25 aa, synthetic construct
  REGION 1..25: note = C3.1.11
DLVGKSLYVSATVILHSGSDMVQAE

SEQ ID NO: 72 - protein, 23 aa, synthetic construct
  REGION 1..23: note = C3.1.12
KTVLTPATNHMGNVTFTIPANRE

SEQ ID NO: 73 - protein, 19 aa, synthetic construct
  REGION 1..19: note = C3.1.13
KGRNKFVTVQATFGTQVVE

SEQ ID NO: 74 - protein, 18 aa, synthetic construct
  REGION 1..18: note = C3.1.14
VVLSRKVLLDGVQNPRAE

SEQ ID NO: 75 - protein, 15 aa, synthetic construct
  REGION 1..15: note = C3.1.15
TLNVNFLLRMDRAHE

SEQ ID NO: 76 - protein, 14 aa, synthetic construct
  REGION 1..14: note = C3.1.16
LVNMGQWKIRAYYE

SEQ ID NO: 77 - protein, 15 aa, synthetic construct
  REGION 1..15: note = C3.1.17
SPMYSIITPNILRLE

SEQ ID NO: 78 - protein, 15 aa, synthetic construct
  REGION 1..15: note = C3.1.18
DRQPVPGQQMTLKIE

SEQ ID NO: 79 - protein, 14 aa, synthetic construct
  REGION 1..14: note = C3.1.19
VTITARFLYGKKVE

SEQ ID NO: 80 - protein, 13 aa, synthetic construct
  REGION 1..13: note = C3.1.20
GTAFVIFGIQDGE

SEQ ID NO: 81 - protein, 8 aa, synthetic construct
  REGION 1..8: note = C3.1.21
KFYYIYNE

SEQ ID NO: 82 - protein, 10 aa, synthetic construct
  REGION 1..10: note = C3.1.22
NSPQQVFSTE

SEQ ID NO: 83 - protein, 8 aa, synthetic construct
  REGION 1..8: note = C3.1.23
SLKRIPIE

SEQ ID NO: 84 - protein, 7 aa, synthetic construct
  REGION 1..7: note = C3.1.24
YVLPSFE

SEQ ID NO: 85 - protein, 7 aa, synthetic construct
  REGION 1..7: note = C3.1.25
QRISLPE

SEQ ID NO: 86 - protein, 8 aa, synthetic construct
  REGION 1..8: note = C3.1.26
LQCPQPAA

SEQ ID NO: 87 - protein, 7 aa, synthetic construct
  REGION 1..7: note = C3.1.27
VIVEPTE

SEQ ID NO: 88 - protein, 5 aa, synthetic construct
  REGION 1..5: note = C3.1.28
TMVLE

SEQ ID NO: 89 - protein, 5 aa, synthetic construct
  REGION 1..5: note = C3.1.29
LRPGE

SEQ ID NO: 90 - protein, 4 aa, synthetic construct
  REGION 1..4: note = C3.1.30
FKSE

SEQ ID NO: 91 - protein, 20 aa, synthetic construct
  REGION 1..20: note = FHR1 peptide 2
NYNIALRWTAKQKLYLRTGE

SEQ ID NO: 92 - protein, 19 aa, synthetic construct
  REGION 1..19: note = FHR2 peptide 3
YNFVSPSKSFWTRITCAEE

SEQ ID NO: 93 - protein, 21 aa, synthetic construct
  REGION 1..21: note = FHR3 peptide 2
KGWSPTPRCIRVRTCSKSDIE

SEQ ID NO: 94 - protein, 18 aa, synthetic construct
  REGION 1..18: note = FHR3 peptide 3
NGYNQNYGRKFVQGNSTE

SEQ ID NO: 95 - protein, 18 aa, synthetic construct
  REGION 1..18: note = FHR3 peptide 4
QVKPCDFPDIKHGGLFHE

SEQ ID NO: 96 - protein, 22 aa, synthetic construct
  REGION 1..22: note = FHR3 peptide 5
FMCKLGYNANTSILSFQAVCRE

SEQ ID NO: 97 - protein, 8 aa, synthetic construct
  REGION 1..8: note = FHR3 peptide 6
YQCQPYYE

SEQ ID NO: 98 - protein, 20 aa, synthetic construct
  REGION 1..20: note = FHR4 peptide 2
NSRAKSNGMRFKLHDTLDYE

SEQ ID NO: 99 - protein, 14 aa, synthetic construct
  REGION 1..14: note = FHR4 peptide 3
DGWSHFPTCYNSSE

SEQ ID NO: 100 - protein, 14 aa, synthetic construct
  REGION 1..14: note = FHR4 peptide 4
ISYGNTTGSIVCGE

SEQ ID NO: 101 - protein, 22 aa, synthetic construct
  REGION 1..22: note = FHR4 peptide 5
FMCKLGYNANTSVLSFQAVCRE

SEQ ID NO: 102 - protein, 18 aa, synthetic construct
  REGION 1..18: note = FHR5 peptide 2
GTLCDFPKIHHGFLYDEE

SEQ ID NO: 103 - protein, 18 aa, synthetic construct
  REGION 1..18: note = FHR5 peptide 3
YAMIGNNMITCINGIWTE

SEQ ID NO: 104 - protein, 18 aa, synthetic construct
  REGION 1..18: note = FHR5 peptide 4
YGYVQPSVPPYQHGVSVE

SEQ ID NO: 105 - protein, 18 aa, synthetic construct
  REGION 1..18: note = FHR5 peptide 5
GDTVQIICNTGYSLQNNE

SEQ ID NO: 106 - protein, 16 aa, synthetic construct
  REGION 1..16: note = FHR5 peptide 6
IVCKDGRWQSLPRCVE

SEQ ID NO: 107 - protein, 12 aa, synthetic construct
  REGION 1..12: note = FHR5 peptide 7
DYNPFSQVPTGE

SEQ ID NO: 108 - protein, 10 aa, synthetic construct
  REGION 1..10: note = FHR5 peptide 8
QVKTCGYIPE

SEQ ID NO: 109 - protein, 10 aa, synthetic construct
  REGION 1..10: note = FHR5 peptide 9
ANVDAQPKKE

SEQ ID NO: 110 - protein, 9 aa, synthetic construct
  REGION 1..9: note = FHR5 peptide 10
WTTLPTCVE

SEQ ID NO: 111 - protein, 8 aa, synthetic construct
  REGION 1..8: note = FHR5 peptide 11
KVAVLCKE

SEQ ID NO: 112 - protein, 25 aa, synthetic construct
  REGION 1..25: note = FH peptide 2
SNTGSTTGSIVCGYNGWSDLPICYE

SEQ ID NO: 113 - protein, 23 aa, synthetic construct
  REGION 1..23: note = FH peptide 3
NGWSPTPRCIRVKTCSKSSIDIE

SEQ ID NO: 114 - protein, 21 aa, synthetic construct
  REGION 1..21: note = FH peptide 4
LPKIDVHLVPDRKKDQYKVGE

SEQ ID NO: 115 - protein, 21 aa, synthetic construct
  REGION 1..21: note = FH peptide 5
YYCNPRFLMKGPNKIQCVDGE

SEQ ID NO: 116 - protein, 20 aa, synthetic construct
  REGION 1..20: note = FH peptide 6
NYNIALRWTAKQKLYSRTGE

SEQ ID NO: 117 - protein, 20 aa, synthetic construct
  REGION 1..20: note = FH peptide 7
KWSHPPSCIKTDCLSLPSFE

SEQ ID NO: 118 - protein, 18 aa, synthetic construct
  REGION 1..18: note = FH peptide 8
HGWAQLSSPPYYYGDSVE

SEQ ID NO: 119 - protein, 18 aa, synthetic construct
  REGION 1..18: note = FH peptide 9
ISHGVVAHMSDSYQYGEE

SEQ ID NO: 120 - protein, 15 aa, synthetic construct
  REGION 1..15: note = FH peptide 10
FDHNSNIRYRCRGKE

SEQ ID NO: 121 - protein, 16 aa, synthetic construct
  REGION 1..16: note = FH peptide 11
ITCKDGRWQSIPLCVE

SEQ ID NO: 122 - protein, 15 aa, synthetic construct
  REGION 1..15: note = FH peptide 12
GWIHTVCINGRWDPE

SEQ ID NO: 123 - protein, 16 aa, synthetic construct
  REGION 1..16: note = FH peptide 13
KAKYQCKLGYVTADGE

SEQ ID NO: 124 - protein, 15 aa, synthetic construct
  REGION 1..15: note = FH peptide 14
TTCYMGKWSSPPQCE

SEQ ID NO: 125 - protein, 13 aa, synthetic construct
  REGION 1..13: note = FH peptide 15
SYAHGTKLSYTCE

SEQ ID NO: 126 - protein, 11 aa, synthetic construct
  REGION 1..11: note = FH peptide 16
RVRYQCRSPYE

SEQ ID NO: 127 - protein, 15 aa, synthetic construct
  REGION 1..15: note = FH peptide 17
GFGIDGPAIAKCLGE

SEQ ID NO: 128 - protein, 12 aa, synthetic construct
  REGION 1..12: note = FH peptide 18
HGTINSSRSSQE

SEQ ID NO: 129 - protein, 10 aa, synthetic construct
  REGION 1..10: note = FH peptide 19
YQCQNLYQLE

SEQ ID NO: 130 - protein, 11 aa, synthetic construct
  REGION 1..11: note = FH peptide 20
WTTLPVCIVEE

SEQ ID NO: 131 - protein, 11 aa, synthetic construct
  REGION 1..11: note = FH peptide 21
KIPCSQPPQIE

SEQ ID NO: 132 - protein, 9 aa, synthetic construct
  REGION 1..9: note = FH peptide 22
SQYTYALKE

SEQ ID NO: 133 - protein, 10 aa, synthetic construct
  REGION 1..10: note = FH peptide 23
QVQSCGPPPE

SEQ ID NO: 134 - protein, 9 aa, synthetic construct
  REGION 1..9: note = FH peptide 24
KKDVYKAGE

SEQ ID NO: 135 - protein, 9 aa, synthetic construct
  REGION 1..9: note = FH peptide 25
GLPCKSPPE

SEQ ID NO: 136 - protein, 8 aa, synthetic construct
  REGION 1..8: note = FH peptide 26
KVSVLCQE

SEQ ID NO: 137 - protein, 7 aa, synthetic construct
  REGION 1..7: note = FH peptide 27
HLKNKKE

SEQ ID NO: 138 - protein, 8 aa, synthetic construct
  REGION 1..8: note = FH peptide 28
GGFRISEE

SEQ ID NO: 139 - protein, 8 aa, synthetic construct
  REGION 1..8: note = FH peptide 29
LLNGNVKE

SEQ ID NO: 140 - protein, 7 aa, synthetic construct
  REGION 1..7: note = FH peptide 30
YPTCAKR

SEQ ID NO: 141 - protein, 8 aa, synthetic construct
  REGION 1..8: note = FH peptide 31
STCGDIPE

SEQ ID NO: 142 - protein, 349 aa, synthetic construct
  REGION 1..349: note = C3dg
EGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 143 - protein, 47 aa, synthetic construct
  REGION 1..47: note = C3g
EGVQKEDIPPADLSDQVPDTESETRILLQGTPVAQMTEDAVDAERLK

SEQ ID NO: 144 - protein, 302 aa, synthetic construct
  REGION 1..302: note = C3d
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 145 - protein, 194 aa, synthetic construct
  REGION 1..194: note = Human Complement Receptor 1
  SITE 111: note = misc_feature - Xaa can be any naturally occurring amino acid
  SITE 157: note = misc_feature - Xaa can be any naturally occurring amino acid
  SITE 160: note = misc_feature - Xaa can be any naturally occurring amino acid
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNXAHWSTKPPICQRIPCGLPPTIANGDFISTNRENFHYGSVVTYRCNXGSXGRKVFELVGEPSIYCTSNDDQVGIWSGPAPQCII

SEQ ID NO: 146 - protein, 194 aa, synthetic construct
  REGION 1..194: note = Human Complement Receptor 1 CCPs 8-10
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 147 - protein, 194 aa, synthetic construct
  REGION 1..194: note = Human Complement Receptor 1 CCPs 15-17
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 148 - protein, 388 aa, synthetic construct
  REGION 1..388: note = Human Complement Receptor 1 CCPs 8-10 and 15-17
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 149 - protein, 235 aa, synthetic construct
  REGION 1..235: note = Human Complement Factor I proteolytic domain
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVG

SEQ ID NO: 150 - protein, 246 aa, synthetic construct
  REGION 1..246: note = Human Complement Factor H co-factor region
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 151 - protein, 194 aa, synthetic construct; identical to SEQ ID NO: 146
  REGION 1..194: note = Human Complement Receptor 1 CCPs 8-10

SEQ ID NO: 152 - protein, 194 aa, synthetic construct; identical to SEQ ID NO: 147
  REGION 1..194: note = Human Complement Receptor 1 CCPs 15-17

SEQ ID NO: 153 - protein, 342 aa, synthetic construct
  REGION 1..342: note = GluC
MKGKFLKVSSLFVATLTTATLVSSPAANALSSKAMDNHPQQTQSSKQQTPKIKKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 154 - protein, 274 aa, synthetic construct
  REGION 1..274: note = GluC
VILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 155 - protein, 12 aa, synthetic construct
  REGION 1..12: note = FI peptide 24
VGCAGFASVTQE

SEQ ID NO: 156 - protein, 14 aa, synthetic construct
  REGION 1..14: note = C3a-desArg
LRRQHARASHLGLA

SEQ ID NO: 157 - protein, 5 aa, synthetic construct
  REGION 1..5: note = C3f-desArg
SASLL

SEQ ID NO: 158 - RNA, 21 nt, synthetic construct
  misc_feature 1..21: note = siRNA target 1 (CFHR1, CFHR2)
  source 1..21: mol_type = other RNA
gctcctggtcagtgtaattct

SEQ ID NO: 159 - RNA, 21 nt, synthetic construct
  misc_feature 1..21: note = siRNA target 2 (CFHR1)
  source 1..21: mol_type = other RNA
ggtgagagagtacgttatgaa

SEQ ID NO: 160 - RNA, 21 nt, synthetic construct
  misc_feature 1..21: note = siRNA target 3 (CFHR1)
  source 1..21: mol_type = other RNA
gaatgtaggagcccttatgaa

SEQ ID NO: 161 - RNA, 21 nt, synthetic construct
  misc_feature 1..21: note = siRNA target 4 (CFHR3)
  source 1..21: mol_type = other RNA
gtaccaatgccagccctacta

SEQ ID NO: 162 - RNA, 21 nt, synthetic construct
  misc_feature 1..21: note = siRNA target 5 (CFHR3, CFHR4)
  source 1..21: mol_type = other RNA
ggaaccaccaagatgcataca

SEQ ID NO: 163 - RNA, 21 nt, synthetic construct
  misc_feature 1..21: note = siRNA target 6 (CFHR3)
  source 1..21: mol_type = other RNA
gcatatggttagtgagaagtt

SEQ ID NO: 164 - RNA, 21 nt, synthetic construct
  misc_feature 1..21: note = siRNA target 7 (CFHR4)
  source 1..21: mol_type = other RNA
gaatcacacttggtaactaat

SEQ ID NO: 165 - RNA, 21 nt, synthetic construct
  misc_feature 1..21: note = siRNA target 8 (CFHR4)
  source 1..21: mol_type = other RNA
ggtcaagagtcgagtaccagt

SEQ ID NO: 166 - RNA, 21 nt, synthetic construct
  misc_feature 1..21: note = siRNA target 9 (CFHR3, CFHR4)
  source 1..21: mol_type = other RNA
gaatgctacgatggatatgaa

SEQ ID NO: 167 - RNA, 21 nt, synthetic construct
  misc_feature 1..21: note = siRNA target 10 (CFHR5)
  source 1..21: mol_type = other RNA
gaatcttcaggactaatacat

SEQ ID NO: 168 - RNA, 21 nt, synthetic construct
  misc_feature 1..21: note = siRNA target 11 (CFHR5)
  source 1..21: mol_type = other RNA
gtaaatgttctcgcttcagta

SEQ ID NO: 169 - RNA, 21 nt, synthetic construct
  misc_feature 1..21: note = siRNA target 12 (CFH, CFHR1, CFHR2)
  source 1..21: mol_type = other RNA
gtaccaatgccagaacttgta

SEQ ID NO: 170 - RNA, 21 nt, synthetic construct
  misc_feature 1..21: note = siRNA target 13 (CFH, CFHR1)
  source 1..21: mol_type = other RNA
gagggtaacaagcgaataaca

SEQ ID NO: 171 - RNA, 21 nt, synthetic construct
  misc_feature 1..21: note = siRNA target 14 (CFH, CFHR1)
  source 1..21: mol_type = other RNA
ggtaacaagcgaataacatgt

SEQ ID NO: 172 - RNA, 21 nt, synthetic construct
  misc_feature 1..21: note = siRNA target 15 (CFH, CFHR2)
  source 1..21: mol_type = other RNA
gctttattcaagaacaggtga

SEQ ID NO: 173 - RNA, 21 nt, synthetic construct
  misc_feature 1..21: note = siRNA target 16 (CFHR2)
  source 1..21: mol_type = other RNA
gaaatgtcaattcctaacagt

SEQ ID NO: 174 - RNA, 21 nt, synthetic construct
  misc_feature 1..21: note = siRNA target 17 (CFHR3)
  source 1..21: mol_type = other RNA
gtgagaagttgcagggtaaga

SEQ ID NO: 175 - RNA, 21 nt, synthetic construct
  misc_feature 1..21: note = siRNA target 18 (CFHR3)
  source 1..21: mol_type = other RNA
gtttccccataccccactcat

SEQ ID NO: 176 - RNA, 21 nt, synthetic construct
  misc_feature 1..21: note = siRNA target 19 (CFHR3)
  source 1..21: mol_type = other RNA
gccaccatgtaagatgtacct

SEQ ID NO: 177 - RNA, 21 nt, synthetic construct
  misc_feature 1..21: note = siRNA target 20 (CFHR4)
  source 1..21: mol_type = other RNA
ggatataatgcgaatacatca

SEQ ID NO: 178 - RNA, 21 nt, synthetic construct
  misc_feature 1..21: note = Antisense target 1
  source 1..21: mol_type = other RNA
agaattacactgaccaggagc

SEQ ID NO: 179 - RNA, 21 nt, synthetic construct
  misc_feature 1..21: note = Antisense target 2
  source 1..21: mol_type = other RNA
ttcataacgtactctctcacc

SEQ ID NO: 180 - RNA, 21 nt, synthetic construct
  misc_feature 1..21: note = Antisense target 3
  source 1..21: mol_type = other RNA
ttcataagggctcctacattc

SEQ ID NO: 181 - RNA, 21 nt, synthetic construct
  misc_feature 1..21: note = Antisense target 4
  source 1..21: mol_type = other RNA
tagtagggctggcattggtac

SEQ ID NO: 182 - RNA, 21 nt, synthetic construct
  misc_feature 1..21: note = Antisense target 5
  source 1..21: mol_type = other RNA
tgtatgcatcttggtggttcc

SEQ ID NO: 183 - RNA, 21 nt, synthetic construct
  misc_feature 1..21: note = Antisense target 6
  source 1..21: mol_type = other RNA
aacttctcactaaccatatgc

SEQ ID NO: 184 - RNA, 21 nt, synthetic construct
  misc_feature 1..21: note = Antisense target 7
  source 1..21: mol_type = other RNA
attagttaccaagtgtgattc

SEQ ID NO: 185 - RNA, 21 nt, synthetic construct
  misc_feature 1..21: note = Antisense target 8
  source 1..21: mol_type = other RNA
actggtactcgactcttgacc

SEQ ID NO: 186 - RNA, 21 nt, synthetic construct
  misc_feature 1..21: note = Antisense target 9
  source 1..21: mol_type = other RNA
ttcatatccatcgtagcattc

SEQ ID NO: 187 - RNA, 21 nt, synthetic construct
  misc_feature 1..21: note = Antisense target 10
  source 1..21: mol_type = other RNA
atgtattagtcctgaagattc

SEQ ID NO: 188 - RNA, 21 nt, synthetic construct
  misc_feature 1..21: note = Antisense target 11
  source 1..21: mol_type = other RNA
tactgaagcgagaacatttac

SEQ ID NO: 189 - RNA, 21 nt, synthetic construct
  misc_feature 1..21: note = Antisense target 12
  source 1..21: mol_type = other RNA
tacaagttctggcattggtac

SEQ ID NO: 190 - RNA, 21 nt, synthetic construct
  misc_feature 1..21: note = Antisense target 13
  source 1..21: mol_type = other RNA
tgttattcgcttgttaccctc

SEQ ID NO: 191 - RNA, 21 nt, synthetic construct
  misc_feature 1..21: note = Antisense target 14
  source 1..21: mol_type = other RNA
acatgttattcgcttgttacc

SEQ ID NO: 192 - RNA, 21 nt, synthetic construct
  misc_feature 1..21: note = Antisense target 15
  source 1..21: mol_type = other RNA
tcacctgttcttgaataaagc

SEQ ID NO: 193 - RNA, 21 nt, synthetic construct
  misc_feature 1..21: note = Antisense target 16
  source 1..21: mol_type = other RNA
actgttaggaattgacatttc

SEQ ID NO: 194 - RNA, 21 nt, synthetic construct
  misc_feature 1..21: note = Antisense target 17
  source 1..21: mol_type = other RNA
tcttaccctgcaacttctcac

SEQ ID NO: 195 - RNA, 21 nt, synthetic construct
  misc_feature 1..21: note = Antisense target 18
  source 1..21: mol_type = other RNA
atgagtggggtatggggaaac

SEQ ID NO: 196 - RNA, 21 nt, synthetic construct
  misc_feature 1..21: note = Antisense target 19
  source 1..21: mol_type = other RNA
aggtacatcttacatggtggc

SEQ ID NO: 197 - RNA, 21 nt, synthetic construct
  misc_feature 1..21: note = Antisense target 20
  source 1..21: mol_type = other RNA
tgatgtattcgcattatatcc

SEQ ID NO: 198 - RNA, 21 nt, synthetic construct
  misc_feature 1..21: note = Scramble antisense target 1
  source 1..21: mol_type = other RNA
ggttgcattaggctatcctct

SEQ ID NO: 199 - RNA, 21 nt, synthetic construct
  misc_feature 1..21: note = Scramble antisense target 2
  source 1..21: mol_type = other RNA
gaatagggtcggaagttatga

SEQ ID NO: 200 - RNA, 21 nt, synthetic construct
  misc_feature 1..21: note = Scramble antisense target 3
  source 1..21: mol_type = other RNA
gtgcataacggtagattcaga

SEQ ID NO: 201 - RNA, 21 nt, synthetic construct
  misc_feature 1..21: note = Scramble antisense target 4
  source 1..21: mol_type = other RNA
gagcctcacgctactaaccta

SEQ ID NO: 202 - RNA, 21 nt, synthetic construct
  misc_feature 1..21: note = Scramble antisense target 5
  source 1..21: mol_type = other RNA
gaacaacacggacgctcaata

SEQ ID NO: 203 - RNA, 21 nt, synthetic construct
  misc_feature 1..21: note = Scramble antisense target 6
  source 1..21: mol_type = other RNA
gattgtgagaggttcgtaata

SEQ ID NO: 204 - RNA, 21 nt, synthetic construct
  misc_feature 1..21: note = Scramble antisense target 7
  source 1..21: mol_type = other RNA
ggaattctaatcgaaactact

SEQ ID NO: 205 - RNA, 21 nt, synthetic construct
  misc_feature 1..21: note = Scramble antisense target 8
  source 1..21: mol_type = other RNA
ggtcaaccgtaatggaggtca

SEQ ID NO: 206 - RNA, 21 nt, synthetic construct
  misc_feature 1..21: note = Scramble antisense target 9
  source 1..21: mol_type = other RNA
gagttagagcgcatagaatat

SEQ ID NO: 207 - RNA, 21 nt, synthetic construct
  misc_feature 1..21: note = Scramble antisense target 10
  source 1..21: mol_type = other RNA
gatccaaatgtaccgaattat

SEQ ID NO: 208 - RNA, 21 nt, synthetic construct
  misc_feature 1..21: note = Scramble antisense target 11
  source 1..21: mol_type = other RNA
gaaattcttccgattgagtct

SEQ ID NO: 209 - RNA, 21 nt, synthetic construct
  misc_feature 1..21: note = Scramble antisense target 12
  source 1..21: mol_type = other RNA
gaaagtcacgatatcctgtca

SEQ ID NO: 210 - RNA, 21 nt, synthetic construct
  misc_feature 1..21: note = Scramble antisense target 13
  source 1..21: mol_type = other RNA
gagaaacaagaaggcgtaact

SEQ ID NO: 211 - RNA, 21 nt, synthetic construct
  misc_feature 1..21: note = Scramble antisense target 14
  source 1..21: mol_type = other RNA
ggagtacttagcgaataaaca

SEQ ID NO: 212 - RNA, 21 nt, synthetic construct
  misc_feature 1..21: note = Scramble antisense target 15
  source 1..21: mol_type = other RNA
gcaattagttaggacgatact

SEQ ID NO: 213 - RNA, 21 nt, synthetic construct
  misc_feature 1..21: note = Scramble antisense target 18
  source 1..21: mol_type = other RNA
gtcaacatcgcatagatatta

SEQ ID NO: 214 - RNA, 21 nt, synthetic construct
  misc_feature 1..21: note = Scramble antisense target 17
  source 1..21: mol_type = other RNA
gaagcgaatgggtaggttaga

SEQ ID NO: 215 - RNA, 21 nt, synthetic construct
  misc_feature 1..21: note = Scramble antisense target 18
  source 1..21: mol_type = other RNA
gctccctttcccctaatcaca

SEQ ID NO: 216 - RNA, 21 nt, synthetic construct
  misc_feature 1..21: note = Scramble antisense target 19
  source 1..21: mol_type = other RNA
ggatcacactgcctacgtaat

SEQ ID NO: 217 - RNA, 21 nt, synthetic construct
  misc_feature 1..21: note = Scramble antisense target 20
  source 1..21: mol_type = other RNA
gaaagagacgattattcaact

SEQ ID NO: 218 - DNA, 1297 bp, synthetic construct
  misc_feature 1..1297: note = Homo sapiens complement factor H related 1 (CFHR1), transcript variant 1, mRNA
  source 1..1297: mol_type = other DNA
cagttagtacactgaaattcaaagtcatgctcataactgttaatgaaagcagattcaaagcaacaccaccaccactgaagtatttttagttatataagattggaactaccaagcatgtggctcctggtcagtgtaattctaatctcacggatatcctctgttgggggagaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagatagaatcaatcataaaatgcacacctttattcagaactttagtattaaatcagttcttaatttcatttttaagtattgttttactcctttttattcatacgtaaaattttggattaatttgtgaaaatgtaattataagctgagaccggtggctctcttcttaaaagcaccatattaaaacttggaaaactaa

SEQ ID NO: 219 - DNA, 1498 bp, synthetic construct
  misc_feature 1..1498: note = Homo sapiens complement factor H related 2 (CFHR2), transcript variant 1, mRNA
  source 1..1498: mol_type = other DNA
accacaaaggactttactaaactagcttccagttagtacactgaaattcaaagtcatgctcataactgttaatgaaagcagattcaaagcaacaccaccaccactgaagtatttttagttatataagattggaactaccaagcatgtggctcctggtcagtgtaattctaatctcacggatatcctctgttgggggagaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctcagactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtccactatttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaagaaaaatagaatcaatggcattactattagtaaaatgcacacctttttctgaatttactattatatttgttttcaatttcatttttcaagtactgttttactcatttttattcataaataaagttttgtgttgatttgtgaaaatgcaattacaatctgagatgtgtcacaatggtgaggactatcttcaccaaatctaagtaacaacctaggaattgtctttttttttctttttaaaaaaattgacaataactgtatatattcatggagtacatagtaatgtttccatatatataatgtataatggtcagttagggtaattagtatatccattatctcaaacatttttcatttctttgggttaggagcattaaatattctccttccagctatttggtacttcatagtatattactggtaactgaaggaattatatctagacgttaccccaggtatcttgaaatgtcaattcctaacagtcacagcctgggagctcatgtttgccttctttcagagcttgtaactatgtatatccacataaataatcaaaa

SEQ ID NO: 220 - DNA, 2934 bp, synthetic construct
  misc_feature 1..2934: note = Homo sapiens complement factor H related 3 (CFHR3), transcript variant 1, mRNA
  source 1..2934: mol_type = other DNA
agtgcaactgaaacttttgtattagcatactactgagaatatctaacatgttgttactaatcaatgtcattctgaccttgtgggtttcctgtgctaatggacaagtgaaaccttgtgattttccagacattaaacatggaggtctatttcatgagaatatgcgtagaccatactttccagtagctgtaggaaaatattactcctattactgtgatgaacattttgagactccttcaggaagttactgggattacattcattgcacacaaaatgggtggtcaccagcagtaccatgtctcagaaaatgttattttccttatttggaaaatggatataatcaaaattatggaagaaagtttgtacagggtaactctacagaagttgcctgccatcctggctacggtcttccaaaagcgcagaccacagttacatgtacggagaaaggctggtctcctactcccagatgcatccgtgtcagaacatgctcaaaatcagatatagaaattgaaaatggattcatttccgaatcttcctctatttatattttaaataaagaaatacaatataaatgtaaaccaggatatgcaacagcagatggaaattcttcaggatcaattacatgtttgcaaaatggatggtcagcacaaccaatttgcattaattcttcagaaaagtgtgggcctcctccacctattagcaatggtgataccacctcctttctactaaaagtgtatgtgccacagtcaagagtcgagtaccaatgccagccctactatgaacttcagggttctaattatgtaacatgtagtaatggagagtggtcggaaccaccaagatgcatacatccatgtataataactgaagaaaacatgaataaaaataacataaagttaaaaggaagaagtgacagaaaatattatgcaaaaacaggggataccattgaatttatgtgtaaattgggatataatgcaaatacatcaattctatcatttcaagcagtgtgtcgggaagggatagtggaataccccagatgcgaataaggcagcattgttaccctaaatgtatgtccaacttccacttttccacttctcactcttatggtctcaaagcttgcaaagatagcttctgatattgttgtaatttctactttatttcaaagaaaattaatataatagtttcaatttgcaacttaatatattctcaaaaatatattaaaacaaactaaattattgcttatgcttgtactaaaataataaaaactactcttatattggacttcttatcaatgaattagtaagtatagagacagacagctgaatggctttctgcatattgtatagtatacctagacatagaaacaaaatgactttagattttatttggggaagtaataataccataaaattagatattaaaattgtaagtgaagataaacacactatagtattcccttattgtagccatggtcctctagatgcagttaaccaaatagggtcatttttattaaaagtagtgtttcctggcaaacactgacattacatcattatcatgatttaaaggaaatagtactagagaaggtgaattattatcattttcctgtgaaaaaagaaaagaggttttgctaaccctttcagagcattgggaacacagccagaagtgcattaaatgtatatattaacttgggcaatgttgacactttaggatgctgaagccaggtgcagtggcacacgcctgtaatcctagcactttgagaggccaagctggcaatcatcagaggtcaaaagttcaagaccagcctgttcaacacggtgaaaccctgtctctactaaaaatagaaaaactagctcggcatgatggcgtgcacctgtagtcccagctactcaggaggctgaggtgggagaatcactttaaccagtggggcagaggttgaactgagccaagatagtgccactgcactccagcctgggcaatagagtgagactctgtcttaaaaataaataaataggatgctgaaaattcctatttaaggaaataattctttttctgatttatttaaggctacatttgtattttcttaatacaaatattttgaaagtttcttcatatatggttttttgcatttcttattaagttttgcttcagatttttttttgtcttttctcctgttaattgccttcactctctttctgttctgtacttttctttgtatataaggattttttgaaaagattgtatatccctgttaacaaattgaaatctctattcactttaatagatttttacccccagttagcatatggttagtgagaagttgcagggtaagaagaaaacaatgttccctttcccaacacttttcctgattatagagaaaaaagcatgaacttgtataatgatctagtcctgtacagaatgagaaatattaattgctagactgagaatgttttggtggctaaagctaggaataactttttaagactgaagaatgatataagctatcaaatctaactcagttttcaaataacaggtttgcagagactggagaataaaaagtaaaaaattgttttattaattccagtgactaattctacttcatcaacatacatcataaataaaatttcaaaaataattgctgatatggtttggctgtgtcccacccaattttcaccttgaattatataatcaccatgcatcaaaggtagggccaggtggagataatggaatcatgggagcagtttccccataccccactcatggtagtaaatacatctcatgagatctaatggttttataaatgaaagttcccctggacaagttctcttgcctgccaccatgtaagatgtacctttgctactcattcaccttctgtcatgatggtgaggccttcccagcaatgtggaactgtgagtccattaaacctctttcctttataaatta

SEQ ID NO: 221 - DNA, 2063 bp, synthetic construct
  misc_feature 1..2063: note = Homo sapiens complement factor H related 4 (CFHR4), transcript variant 1, mRNA
  source 1..2063: mol_type = other DNA
aaattcagaatcacacttggtaactaataatgaaagatttcaaaccccaaacagtgcaactgaaacttttgcattactatactactgagaatatctaacatgttgttactaatcaatgtcattctgaccttgtgggtttcctgtgctaatggacaagaagtgaaaccttgtgattttccagaaattcaacatggaggtctatattataagagtttgcgtagactatactttccagcagctgcaggacaatcttattcctattactgtgatcaaaattttgtgactccttcaggaagttactgggattacattcattgcacacaagatggttggtcaccaacggtcccatgcctcagaacatgctcaaaatcagatgtagaaattgaaaatggattcatttctgaatcttcctctatttatattttaaatgaagaaacacaatataattgtaaaccaggatatgcaacagcagagggaaattcttcaggatcaattacatgtttgcaaaatggatggtcaacacaaccaatttgcattaaattttgtgatatgcctgtttttgagaattccagagccaagagtaatggcatgtggtttaagctccatgacacattggactatgaatgctatgatggatatgaaagcagttatggaaacaccacagattccatagtgtgtggtgaagatggctggtcccatttgccaacatgctataattcttcagaaaactgtgggcctcctccacctattagcaatggagataccacgtccttcccgcaaaaagtgtatctgccatggtcaagagtcgagtaccagtgccagtcctactatgaacttcagggttctaaatatgtaacatgtagtaatggagactggtcagaaccaccaagatgcatatcaatgaaaccttgtgagtttccagaaattcaacatggacatctatattatgagaatacgcgtagaccatactttccagtagctacaggacaatcttactcctattactgtgaccaaaattttgtgactccttcaggaagttactgggattacattcactgcacacaagatgggtggttgccaacagtcccatgcctcagaacatgctcaaaatcagatatagaaattgaaaatggattcatttctgaatcttcctctatttatattttaaataaagaaatacaatataaatgtaaaccaggatatgcaacagcagatggaaattcttcaggttcaattacatgtttgcaaaatggatggtcagcacaaccaatttgcattaaattttgtgatatgcctgtttttgagaattccagagccaagagtaatggcatgcggtttaagctccatgacacattggactacgaatgctacgatggatatgaaatcagttatggaaacaccacaggttccatagtgtgtggtgaagatgggtggtcccatttcccaacatgttataattcttcagaaaagtgtgggcctcctccacctattagcaatggtgataccacctcctttctactaaaagtgtatgtgccacagtcaagagtcgagtaccaatgccagtcctactatgaacttcagggttctaattatgtaacatgtagtaatggagagtggtcggaaccaccaagatgcatacatccatgtataataactgaagaaaacatgaataaaaataacatacagttaaaaggaaaaagtgacataaaatattatgcaaaaacaggggataccattgaatttatgtgtaaattgggatataatgcgaatacatcagttctatcatttcaagcagtgtgtagggaaggcatagtggaataccccagatgcgaataaggcagcattgttaccctaaatgtatgtccaacttccacttctcactcttatggtctcaaagcttgcaaagatagcttctgatattgttgtaatttctactttatttcaaagaaaattaatataatagtttcaatttgcaacttaatatgttctcaaaaatatgttaaaacaaactaaattattgcttatgcttgtactaaaataataaaaactacccttatattgga

SEQ ID NO: 222 - DNA, 2814 bp, synthetic construct
  misc_feature 1..2814: note = Homo sapiens complement factor H related 5 (CFHR5), mRNA
  source 1..2814: mol_type = other DNA
agtacattgaaattcaaagtcatgcttgtaactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctatgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggcggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggggaattttattcataaatgtccacagaaacagtaaatgttctcgcttcagtacttaattcatctaatccctcctgtttgtctcaaattataggataactttgaaactttctgaattaacgttatttaaaaggaaatgtagatgttattttagtctctatcttcatgttattatcacttaaaaacctgcgaaagctgtcaacttttgtggttgtagcaagtattaataaatatttataaatcctctaatgtaagtctagctacctatccaatactaaataccccttaaagtattaaatgcactatctgctgtaaaagaa

SEQ ID NO: 223 - DNA, 3962 bp, synthetic construct
  misc_feature 1..3962: note = Homo sapiens complement factor H (CFH), transcript variant 1, mRNA
  source 1..3962: mol_type = other DNA
acaattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctcttcttaaaagcaccatattaaatcctggaaaactaa

SEQ ID NO: 224 - DNA, 23 bp, synthetic construct
  misc_feature 1..23: note = guideRNA 1
  source 1..23: mol_type = other DNA
agattagaattacactgaccagg

SEQ ID NO: 225 - DNA, 23 bp, synthetic construct
  misc_feature 1..23: note = guideRNA 2
  source 1..23: mol_type = other DNA
taatagaaaacttcccctgtagg

SEQ ID NO: 226 - DNA, 23 bp, synthetic construct
  misc_feature 1..23: note = guideRNA 3
  source 1..23: mol_type = other DNA
agacacaaaattatattcacagg

SEQ ID NO: 227 - DNA, 23 bp, synthetic construct
  misc_feature 1..23: note = guideRNA 4
  source 1..23: mol_type = other DNA
aatggtcattctgaatcttcagg

(19) United States
(12) Patent Application Publication
(10) Pub. No.: US 2024/0209367 A1
(43) Pub. Date: Jun. 27, 2024

(54) INHIBITORY NUCLEIC ACIDS FOR FACTOR H FAMILY PROTEINS

(71) Applicant: Complement Therapeutics Limited, London (GB)

(72) Inventors: Simon CLARK, London (GB); Mustafa Munye, London (GB); Sonika Rathi, London (GB)

(21) Appl. No.: 18/519,153

(22) Filed: Nov. 27, 2023

Related U.S. Application Data
(63) Continuation of application No. PCT/EP22/64376, filed on May 26, 2022.

(30) Foreign Application Priority Data
May 27, 2021 (GB) 2107586.6

(51) Int. Cl. C12N 15/113; A61K 38/46; C12N 9/22; C12N 15/11; C12N 15/90; G01N 33/68

(52) U.S. Cl. CPC C12N 15/113; A61K 38/465; C12N 9/22; C12N 15/11; C12N 15/907; G01N 33/6851; G01N 33/6893; C12N 2310/11; C12N 2310/14; C12N 2310/141; C12N 2310/20; C12N 2310/531; C12N 2800/80; G01N 2333/4716

(57) ABSTRACT

Agents for reducing the gene and/or protein expression of FH family proteins are disclosed. Also disclosed are articles encoding the agents and methods of using the agents, including in therapeutic and prophylactic methods.

### RELATED APPLICATIONS

This application is a continuation of International Application No. PCT/EP2022/064376, filed May 26, 2022, which claims priority from United Kingdom Application No. GB 2107586.6, filed May 27, 2021, each of which are herein incorporated by reference in their entireties.

### SEQUENCE LISTING

The instant application contains a sequence listing which has been submitted electronically in XML ST.26 format and is hereby incorporated by reference in its entirety. Said XML copy, created on Nov. 20, 2023, is named 205661_SL and is 314,383 bytes in size.

### TECHNICAL FIELD

The present disclosure relates to the fields of molecular biology, more specifically nucleic acid technology. The present disclosure also relates to methods of medical treatment and prophylaxis.

### BACKGROUND

The complement system contributes to innate host immune defence by assisting in the rapid recognition and elimination of microbial intruders. However, dysregulation of complement can contribute to inflammatory, immune-related, and age-related conditions. As a result inappropriate regulation of the complement system has been implicated in a wide variety of diseases in humans e.g. diseases of the eye and kidney, as well as neurological diseases and cancer (Morgan, B. P., Semin Immunopathol, 2018. 40(1): p. 113-124; Halbgebauer, R., et al., Semin Immunol, 2018. 37: p. 12-20; Ma, Y., et al., Aging Dis, 2019. 10(2): p. 429-462; and Kleczko, E. K., et al., Front Immunol, 2019. 10: p. 954.

Complement pathway activation and control is regulated by a complex interplay between pathway activators and inhibitors. These activators and inhibitors are commonly enzymes which cleave and inactivate complement molecules on biological surfaces and/or in solution to maintain steady regulation of complement activating species. The complement pathways are in a constant state of flux and balance, and disturbances to this balance can lead to inappropriate activation and the consequences above.

One activating molecule is complement component 3 (C3), a member of the alternative complement pathway and amplification loop. C3 comprises a β chain and an α′ chain which associate through interchain disulphide bonds. During complement activation, C3 is cleaved to generate two functional fragments, C3a and C3b. C3a is a potent anaphylatoxin. Deposition of C3b on biological surfaces, e.g. extracellular matrix and cell surfaces, is the central activating mechanism of the alternative pathway. C3b is a potent opsonin, targeting pathogens, antibody-antigen immune complexes and apoptotic cells for phagocytosis by phagocytes and NK cells. Surface-linked C3b also reacts with other complement proteins to form active convertase enzymes that are able to produce further (surface-attachable) C3b molecules, serving to activate and amplify complement responses (Clark, S. J., et al., J Immunol, 2014. 193(10): p. 4962-70). C3b associates with Factor B to form the C3bBb-type C3 convertase and with C3bBb to form the C3bBb3b-type C5 convertase. Proteolytic cleavage of C3 also produces C3a and C3b through the classical complement pathway and the lectin pathway.

Insufficient control of C3 convertases results in massive production of C3b and C3a molecules and a shift of the complement cascade to its terminal lytic pathway. This produces the potent anaphylatoxin, C5a, and the cell lytic protein complex termed the membrane attack complex; both providing strong inflammatory signals (Clark, S. J., et al., supra). This ultimately leads to cell/tissue destruction and a local inflammatory response.

C3b activation of complement is regulated by complement protein factor I (FI). FI prevents complement activation by cleaving C3b to a proteolytically-inactive form, designated iC3b, which is unable to participate in convertase assembly, and further to downstream products iC3dg and C3d. FI requires the presence of a cofactor, examples of which include the blood-borne Factor H (FH) protein and the membrane-bound surface co-factor ‘complement factor 1’ (CR1; CD35). FH and CR1 also help to exert decay-accelerating activity, which can assist in the deconstruction of already formed C3 convertases.

FH is encoded by the CFH gene on human chromosome 1q32 within the RCA (regulators of complement) gene cluster. There is a naturally-occurring truncated form of FH called FH-like protein 1 (FHL-1) which arises from alternative splicing of the CFH gene and has cofactor activity like FH. FH comprises 20 CCP domains. FHL-1 is identical to FH for the first seven CCP domains before terminating with a unique 4-aa C terminus.

Proteins encoded by the CFHR1-5 genes at the RCA locus also exert complement regulatory functions. The CFHR1-5 genes encode a group of five secreted plasma proteins (FHR1 to FHR5) synthesised primarily by hepatocytes. The FHR proteins retain some sequence homology with C3b binding domains of FH and are thought to enhance complement activation (Skerka et al., Mol Immunol. 2013, 56:170-180).

One complement-related disorder is macular degeneration, e.g. age-related macular degeneration (AMD). Macular degeneration is believed to be driven in part by complement-mediated attack on ocular tissues. A major driver of AMD risk is genetic variation at the RCA locus resulting in dysregulation of the complement cascade. AMD is the leading cause of blindness in the developed world: currently responsible for 8.7% of all global blind registrations. It is estimated that 196 million people will be affected by 2020, increasing to 288 million by 2040 (Wong et al. Lancet Glob Heal (2014) 2:e106-16). AMD manifests as the progressive destruction of the macula, the central part of the retina at the back of the eye, leading to loss of central visual acuity. Early stages of the disease see morphological changes in the macula such as the loss of blood vessels in the choriocapillaris (Whitmore et al., Prog Retin Eye Res (2015) 45:1-29); a layer of capillaries found in the choroid (a highly vascularized layer that supplies oxygen and nutrition to the outer retina). The choriocapillaris is separated from the metabolically active retinal pigment epithelium (RPE) by Bruch's membrane (BrM); a thin (2-4 μm), acellular, five-layered sheet of extracellular matrix. The BrM serves two major functions: the substratum of the RPE and a blood vessel wall. The structure and function of BrM is reviewed e.g. in Curcio and Johnson, Structure, Function and Pathology of Bruch's Membrane, In: Ryan et al. (2013), Retina, Vol. 1, Part 2: Basic Science and Translation to Therapy. 5th ed. London: Elsevier, pp 466-481, which is hereby incorporated by reference in its entirety.

The role of complement in AMD is reviewed, for example, by Zipfel et al. Chapter 2, in Lambris and Adamis (eds.), Inflammation and Retinal Disease: Complement Biology and Pathology, Advances in Experimental Medicine and Biology 703, Springer Science+Business Media, LLC (2010), which is hereby incorporated by reference in its entirety. The key characteristics of AMD are indicative of over-active complement, including cell/tissue destruction and a local inflammatory response. Hallmark lesions of early AMD, termed drusen, develop within BrM adjacent to the RPE layer (Bird et al, Surv Ophthalmol 1995, 39(5):367-374). Drusen are formed from the accumulation of lipids, proteins and cellular debris, and include a swathe of complement activation products (Anderson et al., Prog Retin Eye Res 2009, 29:95-112; Whitcup et al., Int J Inflam 2013, 1-10). The presence of drusen within BrM disrupts the flow of nutrients from the choroid across this extracellular matrix to the RPE cells, which leads to cell dysfunction and eventual death, leading to the loss of visual acuity.

‘Dry’ AMD, also known as geographic atrophy, represents around 50% of late-stage AMD cases. In the remaining percentage of late-stage cases, choroidal neovascularisation (CNV) develops, in which the increased synthesis of vascular endothelial growth factor (VEGF) by RPE cells promotes new blood vessel growth from the choroid/choriocapillaris that breaks through BrM into the retina. These new blood vessels leak and eventually form scar tissue; this is referred to as ‘wet’ (neovascular or exudative) AMD. ‘Wet’ AMD is the most virulent form of late-stage AMD and has different disease characteristics to ‘dry’ AMD. There are treatments for wet AMD, where for example the injection of anti-VEGF agents into the vitreous of the eye can slow or reverse the growth of these blood vessels, although it cannot prevent their formation in the first place. Geographic atrophy (‘dry’ AMD) remains untreatable.

FHL-1 predominates at BrM, suggesting an important role for this variant in protection of retinal tissue from complement-mediated attack (Clark, S. J., et al., supra). FH is found in the blood at a higher concentration than FHL-1. Both FH and FHL-1 protect against complement over-activation in the ECM of the choroid (the capillary network underlying BrM). The role of the five FHR proteins are less well understood, although there is some evidence that they may counter the inhibitory effects of FH and FHL-1 (Clark, S. J. and P. N. Bishop, J Clin Med, 2015. 4(1): p. 18-31).

WO2019/215330 describes that FHR4 is a positive regulator of complement activation and prevents FH-mediated C3b breakdown, leading to the formation of C3 convertase and the progression of the complement activation loop. High levels of circulating FHR4, expressed from the liver, indicate an increased risk of developing complement-related disorders.

Defining the exact molecular changes and activation state underpinning the dysregulation of complement processes in human disease tissue remains problematic, largely because it requires measurements at the protein level and an understanding of the relative quantities of the different regulators. It is critical to be able to accurately measure absolute levels of FH and related RCA locus proteins in plasma, as well as levels of FI and C3b itself, for effective diagnosis and treatment of complement-related disorders. Whilst assays have been developed for FH, distinct measurement of FHL-1 and FHR1-5 is difficult due to high sequence homology between all these proteins. This sequence similarity has meant that, with the exception of the full-length FH protein, it has proven difficult to generate antibodies which are specific to only one of these family members in order to obtain useful immunoassays.

In another example, a recent study quantified levels of FH and FHR1-5 using mass spectrometry but the assay was unable to detect biologically important isoform FHL-1 that is found at significant levels in the blood and at key sites of AMD pathogenesis (Zhang, P., et al., Proteomics. 2017; 17(6):10).

### SUMMARY

In a first aspect, the present disclosure provides an agent for reducing gene and/or protein expression of one or more Factor H family proteins.

In some embodiments, the one or more Factor H family proteins are Factor H-related proteins, optionally wherein the Factor H-related proteins are selected from FHR1, FHR2, FHR3, FHR4 and/or FHR5.

In some embodiments the agent is an inhibitory nucleic acid. In some embodiments, the inhibitory nucleic acid comprises or encodes antisense nucleic acid targeting a nucleotide sequence of RNA encoded by one or more genes encoding the one or more Factor H family proteins.

In some embodiments, the inhibitory nucleic acid comprises or encodes antisense nucleic acid targeting a nucleotide sequence comprising, or consisting of, one or more of SEQ ID NO:158, 159, 160, 161, 162, 163, 164, 165, 166, 167, 168, 169, 170, 171, 172, 173, 174, 175, 176 and/or 177. In some embodiments, the inhibitory nucleic acid comprises or encodes antisense nucleic acid comprising or consisting of a sequence having at least 75% sequence identity to one or more of SEQ ID NO:178, 179, 180, 181, 182, 183, 184, 185, 186, 187, 188, 189, 190, 191, 192, 193, 194, 195, 196 and/or 197.

In some embodiments, the inhibitory nucleic acid is an siRNA, shRNA, miRNA or antisense oligonucleotide.

In some embodiments, the agent is a gene-editing tool or gene-editing system. In some embodiments, the agent is selected from: a meganuclease, a chemical nuclease, a zinc finger nuclease (ZFN), a transcription activator-like effector-based nuclease (TALEN), or a CRISPR-Cas system (e.g. a base editing system). The CRISPR-Cas system may comprise a guideRNA (gRNA) and/or a tracrRNA. The CRISPR-Cas system may comprise a single guideRNA (sgRNA). In some embodiments, the CRISPR-Cas system comprises a sequence, e.g. gRNA or sgRNA, having at least 75% sequence identity to SEQ ID NO: 224, 225, 226 or 227.

The present disclosure also provides a nucleic acid, optionally isolated, encoding an agent according to the present disclosure. The present disclosure also provides an expression vector, comprising a nucleic acid according to the present disclosure.

The present disclosure also provides a composition comprising an agent, nucleic acid or expression vector according to the present disclosure, and a pharmaceutically acceptable carrier, diluent, excipient or adjuvant.

The present disclosure also provides a cell comprising an agent, nucleic acid or expression vector according to the present disclosure.

The present disclosure also provides an in vitro or in vivo method for reducing gene and/or protein expression of one or more Factor H family proteins, comprising contacting a cell with an agent, nucleic acid, expression vector or composition according to the present disclosure.

The present disclosure also provides the use of an agent, nucleic acid, expression vector or composition according to the present disclosure, to reduce gene and/or protein expression of one or more Factor H family proteins.

The present disclosure also provides an agent, nucleic acid, expression vector or composition according to the present disclosure, for use in a method of medical treatment or prophylaxis.

The present disclosure also provides an agent, nucleic acid, expression vector or composition according to the present disclosure, for use in a method of treating or preventing a complement-related disorder.

The present disclosure also provides the use of an agent, nucleic acid, expression vector or composition according to the present disclosure, in the manufacture of a medicament for treating or preventing a complement-related disorder.

The present disclosure also provides a method of treating or preventing a complement-related disorder in a subject, comprising administering to a subject a therapeutically- or prophylactically-effective amount of an agent, nucleic acid, expression vector or composition according to the present disclosure.

The present disclosure also provides a method for selecting a subject to be administered an agent, nucleic acid, expression vector or composition according to the present disclosure, the method comprising:
(a) determining the level of a complement protein selected from one or more of FHR1, FHR2, FHR3, FHR4 and/or FHR5, and optionally FHL-1, in a blood sample obtained from the subject;
(b) selecting the subject to be administered an agent, nucleic acid, expression vector or composition according to the present disclosure, if the level of the complement protein determined in (a) is elevated as compared to the level of that complement protein in blood in a control subject that does not have a complement-related disorder.

The present disclosure also provides an agent, nucleic acid, expression vector or composition according to the present disclosure, for use in a method of treating or preventing a complement-related disorder in a subject, wherein the method comprises:
(a) determining the level of a complement protein selected one or more of FHR1, FHR2, FHR3, FHR4 and/or FHR5, and optionally FHL-1, in a blood sample obtained from the subject;
(b) determining whether the level of the complement protein(s) in (a) is elevated as compared to the level of that complement protein(s) in blood in a control subject that does not have a complement-related disorder; and
(c) administering an agent, nucleic acid, expression vector, or composition according to the present disclosure to the subject.

The present disclosure also provides the use of an agent, nucleic acid, expression vector or composition according to the present disclosure in the manufacture of a medicament for treating or preventing a complement-related disorder in a subject, wherein the method comprises:
(a) determining the level of a complement protein selected from one or more of FHR1, FHR2, FHR3, FHR4 and/or FHR5, and optionally FHL-1, in a blood sample obtained from the subject;
(b) determining whether the level of the complement protein(s) in (a) is elevated as compared to the level of that complement protein(s) in blood in a control subject that does not have a complement-related disorder; and
(c) administering an agent, nucleic acid, expression vector or composition according to the present disclosure to the subject.

The present disclosure also provides a method of treating or preventing a complement-related disorder in a subject, comprising:
(a) determining the level of a complement protein selected from one or more of FHR1, FHR2, FHR3, FHR4 and/or FHR5, and optionally FHL-1, in a blood sample obtained from the subject;
(b) determining whether the level of the complement protein is elevated as compared to the level of that complement protein in blood in a control subject that does not have a complement-related disorder; and
(c) administering an agent, nucleic acid, expression vector or composition according to the present disclosure to the subject.

In some embodiments in accordance with the various different aspects of the present disclosure, the complement-related disorder is selected from: macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, kidney injury/damage/dysfunction, glomerular diseases, Membranoproliferative Glomerulonephritis Type II (MPGN II), sepsis, Henoch-Schonlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, anti-neutrophilic cytoplasmic autoantibodies (ANCA) vasculitis, neurodegeneration/neurodegenerative disease, dementia, multiple sclerosis (MS), Lewy body disease, Amyotrophic lateral sclerosis (ALS), Huntington's disease, prion diseases, cancer, lung cancer, glioblastoma e.g. glioblastoma multiforme (GBM), stroke, insulin resistance, diabetes, an infectious disease, Parkinson's disease, and/or Alzheimer's disease. The subject to be treated may be determined to have, or have been determined to have, a complement-related disorder.

### DESCRIPTION

The present disclosure provides agents, such as inhibitory nucleic acids and gene editing systems, for reducing gene and/or protein expression of one or more Factor H family proteins.

Aspects of the present disclosure arise from the measured observation that circulating levels of all five Factor H-related (FHR) proteins in human blood are elevated in individuals suffering from a number of complement-related disorders, such as eye disorders like macular degeneration, kidney disorders, cancers and disorders of the CNS.

Circulating levels of these FHR proteins derive exclusively from the liver as their only known source of expression in the human body. Expression of said proteins is to a large extent genetically driven. The ability of FHR proteins to out-compete the negative regulators of complement activation (i.e. FH and FHL-1) means their increased concentration can pre-dispose a patient to be more complement-active. The detection of overexpression of one or more FHR proteins, e.g. as described herein, is therefore predictive of an individual's likelihood of developing a complement-related disorder.

By targeting expression levels of the FHR proteins in the liver, or by intervening directly in blood, genetically-driven excess FHR proteins can be preventing from entering or removed from the circulation, thus preventing their accumulation in tissues/organs and the subsequent driving of damaging complement activation, inflammation, immune cell recruitment and ECM modelling. In this way, complement-related disorders can be treated or prevented. Currently, no therapeutic designed to reduce circulating levels of FHR proteins exists. Thus, described herein are agents that target each individual CFHR gene, as well as agents e.g. inhibitory nucleic acids that are capable of reducing the expression of multiple CFHR genes simultaneously. Advantageously, the present invention has also identified agents that do not have a simultaneous effect on the level of FH and/or FHL-1. Some agents even increase levels of FH and/or FHL-1 whilst reducing expression of one or more FHR proteins.

Methods disclosed herein may relate in part to the detection and quantification of complement related proteins, particularly one or more FHR proteins and optionally FHL-1 and/or FH. Such methods are useful to identity and stratify patients with disorders related to over-activity of the complement system due to increased circulating levels of FHR proteins. Such methods may be used to stratify patients based on their risk of developing or having complement-related disorders. In some cases, the methods of the present disclosure are used to identify appropriate treatments, such as treatments targeted to the specific complement proteins that are overexpressed in the patient, e.g. agents described herein.

Detection, differentiation and quantitation of highly similar proteins can be achieved using mass spectrometry (MS). In order to achieve good sensitivity by MS, proteins e.g. in a sample are routinely digested into peptides using a specific protease. The industry standard protease for this purpose is trypsin. Other enzymes that are commonly used to digest proteins for MS analysis include elastase, chymotrypsin or LysN.

Trypsin cleaves C-terminal to all K and R residues, provided they are not followed by a proline residue, and yields peptides which retain a basic group at their C-terminus which subsequently helps ionisation and transmission of peptides into the gas phase in a mass spectrometer. Peptides digested by trypsin tend to be ionised more efficiently during MS and thus produce a larger signal than peptides digested by non-trypsin enzymes. Using MS, individual peptides in the sample digest can be detected with a signal proportional to its abundance. The concentration of the parent protein can be derived from the relative abundance (signal) of endogenous peptide compared to an exogenous ‘standard’ peptide e.g. containing a stable isotope.

Trypsin digestion of complement proteins FH and FHL-1 does not produce peptides that can be detected individually using MS alone. The only FHL-1 specific tryptic peptide is a 4-amino acid C-terminal sequence which is too small to be detected reliably by MS techniques. The FHR proteins also share substantial sequence identity, meaning that it is hard to distinguish between them and measure them specifically using e.g. antibody-based assays.

Also described herein is a unique targeted mass spectrometry assay using a non-standard proteolytic enzyme, GluC (V8 protease), to produce distinct proteotypic peptides for all the FHR proteins, as well as proteotypic peptides that can be used to distinguish between FHL-1 and FH, which can be used for the simultaneous detection and accurate measurement in plasma of all seven key regulatory proteins encoded from the CFH gene cluster using a single MS assay: FH, FHL-1, and FHR1, FHR2, FHR3, FHR4 and FHR5.

FHL-1 is a distinct biological entity from FH. The proteins have a similar action but the size of FHL-1 means that its distribution in the body is likely to be distinct from FH. This is apparent in the eye where FHL-1 can cross to the retinal side of Bruch's membrane, e.g. where drusen form, but the larger FH protein cannot, see e.g. Clark et al., J Immunol 2014, 193(10) 4962-4970 and Clark et al., Frontiers in Immunology 2017 8:1778, which are hereby incorporated by reference in their entirety. In this respect, there is evidence that FHL-1 is the prime driver of complement C3b turnover in the eye, meaning that levels of FHL-1 are likely to better inform disease risk than levels of FH.

GluC is also able to produce proteotypic peptides for C3b and FI, enabling direct measurement of C3b itself as well as levels of its proteolytic enzyme and required fluid-phase cofactors. Thus, the methods described herein mean that all these complement proteins can be measured using a single assay.

Furthermore, breakdown of C3b occurs via trypsin-like cleavages at basic residues (K and R) so trypsin digestion of C3b breakdown products is unable to produce useful peptides for analysis. In contrast, C3 turnover can be measured using the MS approach of the present disclosure because GluC digestion also produces proteotypic neopeptides from many C3 inactivation and breakdown products generated during inactivating cleavages. The inventors demonstrate herein that a series of products produced as a result of C3/C3b cleavage can be detected and quantified using the same single GluC/MS assay. This allows the concentrations of all known C3 fragments e.g. iC3b, C3c, C3dg and C3d to be determined accurately.

Thus, the methods described herein can not only measure absolute levels of regulatory complement proteins, but can also track protein products resulting from C3 inactivation and thus assess complement activation and the progression of the amplification loop.

This is advantageous because the measurement of C3 breakdown products is analytically challenging. The pattern by which C3 is broken down is complex: first into C3a and C3b, followed by cleavage of C3b into iC3b (which cannot drive formation of the membrane attack complex (MAC) but can still act as an opsonin), and then subsequent inactivating cleavage of iC3b into C3c via the release of a C3dg fragment. To approach detection of these products with antibodies is problematic. While each sequential cleavage step in this cascade generates a new proteoform (a distinct form of a protein encoded from the same gene, including cleaved forms and splice variants), they share sequence homology and likely only undergo minor structural changes. Directing antibodies at each form is likely to be unsuccessful and while there are methods which can measure single components following some form of separation, e.g. C3dg following polyethylene glycol-based enrichment, simultaneous measurement of all fragments in the same sample is not currently possible.

Thus, described herein is a single methodology for concurrent determination of the presence, absolute levels and relative molar ratios of up to seven individual complement-related proteins from the CFH family plus C3b-inactivating enzyme FI, central complement component C3, and seven proteins derived from C3 breakdown, which may be referred to herein as the “complementome”. The ability to detect absolute levels of so many complement-related proteins in one assay is critical for the successful detection, diagnosis and treatment of complement-related diseases, e.g. using the agents and systems described herein.

### Complement Proteins

Complement is a central part of the innate immunity that serves as a first line of defence against foreign and altered host cells. Complement is activated upon infection with microorganisms to induce inflammation and promote elimination of the pathogens. The complement system is composed of plasma proteins produced mainly by the liver or membrane proteins expressed on cell surface. Complement operates in plasma, in tissues, or within cells. For a review of the complement system, see e.g. Merle N S et al., Front Immunol. 2015 Jun. 2; 6:262, which is hereby incorporated by reference in its entirety.

The complement system can be activated via three distinct pathways: the classical pathway (CP), alternative pathway (AP) and lectin binding pathway (LP). In a healthy individual, the AP is permanently active at low levels to survey for presence of pathogens but host cells are protected against complement attack and are resistant to persistent low-level activation. C3b molecules bound to host cells are inactivated rapidly by a group of membrane-bound or plasma complement regulators.

In response to the recognition of molecular components of microorganisms, complement proteins become sequentially activated in an enzyme cascade: the activation of one protein enzymatically cleaves and activates the next protein in the cascade.

The three pathways converge into the generation of a C3 convertase, which cleaves the central complement component C3 into activation products C3b, a large fragment that acts as an opsonin (binds to foreign microorganisms to increase their susceptibility to phagocytosis), and C3a, an anaphylatoxin that promotes inflammation. Along with factor B (FB), C3b forms the C3 convertase (C3bBb) which cleaves further C3 molecules, generates more C3b and C3a, and amplifies C3b deposition on cell surfaces. This is the complement amplification loop. C3b deposition and activation of complement may occur on acellular structures (i.e. on extracellular matrix), such as Bruch's membrane (BrM) and the intercapillary septa of the choriocapillaris in the eye.

Activated C3 can trigger the lytic pathway, which can damage the plasma membranes of cells and some bacteria. C5a, another anaphylatoxin produced by this process, attracts macrophages and neutrophils and also activates mast cells.

Once activated, the complement system needs tight control, as newly generated complement activation products, e.g. C3b, can induce severe inflammation and cell damage to the host. A number of soluble as well as membrane bound complement regulators ensure regulation of complement activation at the surface of host cells and control different activation phases and sites of action (Skerka et al., Mol Immunol 2013, 56:170-180). Complement regulators are described further herein.

“Complement protein” may be used interchangeably herein with “complement regulator”, “a regulator of complement”, or “protein of the complement system” and refers to a protein component of the complement system or complement cascade, e.g. as described in Merle et al., Front. Immunol., 2015, 6:262 and Merle et al., Front. Immunol., 2015, 6:257, which are hereby incorporated by reference in their entirety. A “complement protein” referred to herein may be involved in any of the three complement pathways and/or in the amplification loop.

In some embodiments a “complement protein” referred to herein is involved in the alternative pathway and/or the complement activation loop. In some embodiments, a “complement protein” referred to herein is involved in the breakdown, turnover and/or inactivation of C3 or C3b, or is a product of said breakdown, turnover and/or inactivation.

In some embodiments herein, a “complement protein” as used herein may refer to one or more of FH, FHL-1, FHR1, FHR2, FHR3, FHR4, FHR5, FI, C3, C3b, C3a, iC3b, C3f, C3c, C3dg, and/or C3d.

### Complement Factor H (FH) Family Proteins

Factor H (FH) regulates the alternative complement pathway and the amplification loop. It inhibits C3 convertase formation by competing with FB binding to C3b and also acts as a cofactor for C3b inactivation to iC3b by Factor I (FI), thus preventing inappropriate complement activation and inflammation. FH also exerts decay-accelerating activity, which can assist in the deconstruction of already formed C3 convertases, see e.g. Clark et al., J Immunol 2014, 193(10) 4962-4970, which is hereby incorporated by reference in its entirety.

The sequence of human FH (Uniprot P08603-1) is provided herein as SEQ ID NO:1. For a review of FH structure and function see e.g. Merle N S et al., Front Immunol. 2015 Jun. 2; 6:262, which is hereby incorporated by reference in its entirety.

Human FH comprises 20 CCP domains. The CFH gene also produces a truncated form of FH, called FHL-1, comprising only the first seven CCP domains before terminating with a unique 4-amino acid C terminus (Clark et al, 2014 supra). The sequence of human FHL-1 (Uniprot: P08603-2) is provided herein as SEQ ID NO:2.

In the eye, full-length FH protein is found on the choroidal side of Bruch's membrane (BrM), with particular accumulation in the choriocapillaris (capillary layer in the choroid). Small amounts have also been found in patches on the RPE side of the BrM, but no FH was observed in the BrM itself. FHL-1 on the other hand has been observed throughout BrM and other ECM structures e.g. drusen (Clark et al, 2014 supra). It is likely that FHL-1 confers greater complement protection to BrM than does FH, whereas FH provides the main protection for the ECM of the choroid. It is thought that FHL-1 is therefore a major regulator of complement in the BrM (a key site in AMD pathogenesis). The methods described herein allow for the individual detection and quantitation of FH and FHL-1.

FH, FHL-1 and FHR1-FHR5, are described in e.g. Clark et al., J Clin Med, 2015. 4(1): 18-31, which is hereby incorporated by reference in its entirety. These proteins may be referred to herein as “Factor H family proteins”. “FHR” stands for “Factor H-related”.

FHR1, FHR2, FHR3, FHR4 and FHR5, encoded by the CFHR genes, are also described in e.g. Skerka et al., Mol Immunol 2013, 56:170-180, which is hereby incorporated by reference in its entirety. These proteins are highly related and share a high degree of sequence identity. The N termini share 36-94% sequence identity, whilst the C-terminal domains are very similar to the FH C-terminus (36-100%). The high amino acid identity among family members is demonstrated by the fact that antibodies raised against FH can detect multiple FHR proteins in plasma and that antibodies generated against FHR proteins cross-react with the other FHRs. This cross-reactivity presents a challenge for purification of FHR proteins from plasma, as well as determining their concentration.

FHR proteins are divided into two groups depending on their conserved domains. FHR1 (SEQ ID NO:3), FHR2 (SEQ ID NO:3, 4), and FHR5 (SEQ ID NO:10) form Group I and are characterised by their conserved N-termini. They exist in plasma as homo- and heterodimers, mediated by the conserved N-terminal domains. Group II contains FHR3 (SEQ ID NO:6, 7) and FHR4 (SEQ ID NO:8, 9) which lack the N-terminal dimerisation domains, but which show a high degree of sequence similarity to portions of FH. All five FHR proteins comprise C-termini sequences that act to recognise and bind C3b, and which are very similar to the C-terminus of FH.

FHR1 is known to compete with FH and FHL-1 for binding to C3b. It is also reported to bind to C3b components of the C5 convertase and interfere with the assembly of the MAC (see e.g. Heinen S et al., Blood (2009) 114 (12): 2439-2447 and Hannan J P et al., PLoS One. 2016; 11(11):e0166200, which are hereby incorporated by reference in their entirety). As used herein, the term “FHR1” includes at least one of FHR1 (SEQ ID NO:3; FHRA) and a second FHR1 isoform (FHRB) with 3 point mutations, and preferably includes both FHR1 isoforms. “FHR1” refers to FHR1 from any species and includes isoforms, fragments, variants or homologues of FHR1 from any species. In preferred embodiments, “FHR1” refers to human FHR1.

FHR2 may inhibit C3 convertase activity, acting to inhibit the amplification loop, but may also activate the amplification loop. There are two FHR2 isoforms (SEQ ID NO:4 and 5). The protein has two glycosylated forms, a single glycosylated form (24 kDa) and a double glycosylated form (28 kDa). As used herein, the term “FHR2” includes at least one of the two isoforms or at least one of the glycosylated forms, and preferably includes both isoforms and any glycosylated forms. “FHR2” refers to FHR2 from any species and includes isoforms, fragments, variants or homologues of FHR2 from any species. In preferred embodiments, “FHR2” refers to human FHR2.

FHR3 binds to C3b and C3d and may have low cofactor activity for FI-mediated cleavage of C3b. FHR3 may also upregulate complement. There are two FHR3 isoforms (SEQ ID NO:6 and 7). FHR3 is detected in plasma in multiple variants (ranging from 35 to 56 kDa), reflecting the existence of four different glycosylated variants of FHR3. As used herein, the term “FHR3” includes at least one of the two isoforms or at least one of the glycosylated variants of FHR3, and preferably includes both isoforms and any glycosylated forms. “FHR3” refers to FHR3 from any species and includes isoforms, fragments, variants or homologues of FHR3 from any species. In preferred embodiments, “FHR3” refers to human FHR3.

The human CFHR4 gene encodes two proteins: FHR4A (SEQ ID NO:8) and FHR4B (SEQ ID NO:9), an alternative splice variant. WO 2019/215330 A1, hereby incorporated by reference in its entirety, describes that FHR4 is a positive regulator of complement activation and prevents FH-mediated C3b breakdown. High levels of FHR4 in tissues are likely to promote local inflammatory responses and cell lysis, leading to disorders associated with complement activation, and circulating FHR4 levels can be used as an indicator of risk of developing complement-related disorders, see e.g. Cipriani et al., Nat Commun 11, 778 (2020), hereby incorporated by reference in its entirety. As used herein, the term “FHR4” includes at least one of FHR4A isoform 1, FHR4A isoform 2 (G20 point deletion from isoform 1) or FHR4B, and preferably includes FHR4A isoforms 1 and 2 as well as FHR4B. “FHR4” refers to FHR4 from any species and includes isoforms, fragments, variants or homologues of FHR4 from any species. In preferred embodiments, “FHR4” refers to human FHR4.

FHR5 also recognises and binds to C3b on self surfaces. FHR5 appears as a glycosylated protein of 62 kDa. As used herein, the term “FHR5” includes any glycosylated variants of FHR5, and preferably includes all isoforms and any glycosylated forms. As used herein, “FHR5” refers to FHR5 from any species and includes isoforms, fragments, variants or homologues of FHR5 from any species. In preferred embodiments, “FHR5” refers to human FHR5.

Given the different roles of the different members of the CFH family in activation and amplification of complement and pathogenesis of complement-related disorders, it is important to be able to distinguish between the presence and levels of proteins encoded by all seven CFH family members. CFH family members, particularly FHR1-5, can also be used as biomarkers for diagnosing or predicting disorders in which dysregulation of complement is pathologically implicated.

### C3, C3b and Breakdown Products

C3 is the central complement component. The pathways by which C3 is processed into various downstream products can lead to activation of complement, e.g. including inflammation and immune responses, or to the inactivation and regulation of complement. It is therefore important in terms of complement pathogenesis and treatment of complement-related disorders to be able to detect and measure the levels, including relative levels, of C3, C3b and their downstream components/processing products.

Processing of C3 is described, for example, in Foley et al. J Thromb Haemostasis (2015) 13: 610-618, which is hereby incorporated by reference in its entirety. Human C3 (UniProt: P01024; SEQ ID NO:12) comprises a 1,663 amino acid sequence (including an N-terminal, 22 amino acid signal peptide). Amino acids 23 to 667 encode C3 β chain (SEQ ID NO:13), and amino acids 749 to 1,663 encode C3b α′ chain (SEQ ID NO:14). C3 β chain and C3 α′ chain associate through interchain disulphide bonds (formed between cysteine 559 of C3 β chain, and cysteine 816 of the C3 α′ chain) to form C3b. C3a is a 77 amino acid fragment corresponding to amino acid positions 672 to 748 of C3 (SEQ ID NO:15), generated by proteolytic cleavage of C3 to form C3b.

Processing of C3b to the inactive form iC3b, which cannot itself promote further complement amplification, involves proteolytic cleavage of the C3b α′ chain at amino acid positions 1303 and 1320 to form an α′ chain fragment 1 (corresponding to amino acid positions 749-1663 of C3; SEQ ID NO:16), and an α′ chain fragment 2 (corresponding to amino acid positions 1321 to 1,663 of C3; SEQ ID NO:17). Thus, iC3b comprises the C3 β chain, C3 α′ chain fragment 1 and C3 α′ chain fragment 2 (associated via disulphide bonds). Cleavage of the α′ chain also liberates C3f, which corresponds to amino acid positions 1304 to 1320 of C3 (SEQ ID NO:18).

iC3b is processed further to C3c comprising the C3 β chain, C3 α′ chain fragment 2 and C3c α′ chain fragment 1 (corresponding to amino acid positions 749-954 of C3; SEQ ID NO:19). This cleavage event produces fragment C3dg (corresponding to amino acid positions 955-1303 of C3; SEQ ID NO:142), which is itself broken down into fragments C3g (corresponding to amino acid positions 955-1001 of C3; SEQ ID NO:143) and C3d (corresponding to amino acid positions 1002-1303 of C3; SEQ ID NO:144).

Processing of C3b to iC3b is performed by Complement Factor I (FI; encoded in humans by the gene CFI). Human Complement Factor I (UniProt: P05156; SEQ ID NO:11) has a 583 amino acid sequence (including an N-terminal, 18 amino acid signal peptide). Amino acids 340 to 574 of the light chain encode the proteolytic domain of FI, which is a serine protease containing the catalytic triad responsible for cleaving C3b to produce iC3b (Ekdahl et al., J Immunol (1990) 144 (11): 4269-74). Proteolytic cleavage of C3b by FI to yield iC3b is facilitated by co-factors, including FH, CR1 and possibly some of the FHR proteins. Co-factors for FI typically bind to C3b and/or FI, and potentiate processing of C3b to iC3b by FI.

As used herein, any reference to a complement protein, e.g. C3, C3b, C3a, FH, FI etc, refers to said protein from any species and include isoforms, fragments, variants or homologues of said protein from any species. In some embodiments, the protein is a mammalian protein (e.g. cynomolgous, human and/or rodent (e.g. rat and/or murine) protein). Isoforms, fragments, variants or homologues of the complement proteins described herein may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the immature or mature protein from a given species, e.g. human protein sequences provided herein. Isoforms, fragments, variants or homologues of complement proteins described herein may optionally be functional isoforms, fragments, variants or homologues, e.g. having a functional property/activity of the reference protein, as determined by analysis by a suitable assay for the functional property/activity.

Agents Capable of Reducing Gene and/or Protein Expression

Aspects and embodiments of the present disclosure relate to agents capable of reducing gene and/or protein expression. As used herein, such agents are capable of reducing or preventing the gene and/or protein expression of one or more given target gene(s)/protein(s). In some embodiments such agents are capable of reducing or preventing the gene and/or protein expression of one or more complement proteins as described herein (e.g. one or more of FH, FHL-1, FHR1, FHR2, FHR3, FHR4, FHR5, FI, C3, C3b, C3a, iC3b, C3f, C3c, C3dg, and/or C3d).

In some embodiments, an agent reduces gene and/or protein expression of one or more Factor H family proteins. In some embodiments, an agent reduces gene and/or protein expression of one or more Factor H family proteins selected from FH, FHL-1, FHR1, FHR2, FHR3, FHR4 and/or FHR5. In some embodiments, an agent reduces gene and/or protein expression of one or more Factor H family proteins selected from FHR1, FHR2, FHR3, FHR4, FHR5, FH and/or FHL-1.

In some cases, such agents are capable of reducing or preventing the gene and/or protein expression of one or more FHR proteins (e.g. one or more of FHR1, FHR2, FHR3, FHR4, and/or FHR5). In some cases, such agents do not reduce or prevent (e.g. significantly reduce or prevent) gene and/or protein expression of FH and/or FHL-1. For example, such agents are capable of reducing or preventing the gene and/or protein expression of one or more FHR proteins whilst not reducing or preventing the gene and/or protein expression of FH and/or FHL-1. In some cases, such agents are capable of reducing or preventing the gene and/or protein expression of one or more FHR proteins whilst increasing the gene and/or protein expression of FH and/or FHL-1. In some cases, an agent described herein inhibits gene and/or protein expression of each of FHR1, FHR2, FHR3, FHR4, and FHR5, but does not inhibit gene and/or protein expression of FH and/or FHL-1 (e.g. it may increase gene and/or protein expression of FH and/or FHL-1).

An ‘agent capable of reducing gene and/or protein expression’ or an ‘agent’ that ‘reduces gene and/or protein expression’ as used herein may be any suitable agent that achieves said effect. The agent may be a nucleic acid, such as an inhibitory nucleic acid described hereinbelow. The agent may be a nuclease-based nucleic acid editing tool, such as a meganuclease, chemical nuclease, zinc finger nuclease (ZFN), a transcription activator-like effector-based nuclease (TALEN), or a Cas9-based system, e.g. as described hereinbelow.

Provided herein is a method for reducing gene and/or protein expression of one or more Factor H family proteins, comprising contacting a cell with an agent according to the present disclosure. The method may be performed in vitro, ex vivo, or in vivo. The cell may be a liver cell, e.g. a hepatocyte.

It will be appreciated that where an agent, e.g. an inhibitory nucleic acid or nucleic acid editing tool, is described as reducing expression of a protein(s), the present disclosure also contemplates reducing gene expression of a gene encoding the relevant protein(s). That is, reference herein to inhibition of gene expression of FH and/or FHL-1 contemplates inhibition of expression of CFH; reference herein to inhibition of gene expression of FHR1 contemplates inhibition of expression of CFHR1; reference herein to inhibition of gene expression of FHR2 contemplates inhibition of expression of CFHR2; reference herein to inhibition of gene expression of FHR3 contemplates inhibition of expression of CFHR3; reference herein to inhibition of gene expression of FHR4 contemplates inhibition of expression of CFHR4; and reference herein to inhibition of gene expression of FHR5 contemplates inhibition of expression of CFHR5.

In some embodiments, an agent, e.g. an inhibitory nucleic acid or nucleic acid editing tool, according to the present disclosure may:
reduce/prevent/inhibit expression of a gene or genes encoding one or more Factor H family proteins (e.g. one or more of FHR1, FHR2, FHR3, FHR4, FHR5, FH and/or FHL-1);
reduce the level of RNA encoding one or more Factor H family proteins (e.g. one or more of FHR1, FHR2, FHR3, FHR4, FHR5, FH and/or FHL-1);
reduce/prevent/inhibit transcription of nucleic acid encoding one or more Factor H family proteins (e.g. one or more of FHR1, FHR2, FHR3, FHR4, FHR5, FH and/or FHL-1) (e.g. from DNA encoding one or more Factor H family proteins to RNA encoding one or more Factor H family proteins);
increase degradation of RNA encoding one or more Factor H family proteins (e.g. one or more of FHR1, FHR2, FHR3, FHR4, FHR5, FH and/or FHL-1);
reduce/prevent/inhibit expression of one or more Factor H family proteins (e.g. one or more of FHR1, FHR2, FHR3, FHR4, FHR5, FH and/or FHL-1) at the protein level;
reduce the level of one or more Factor H family proteins (e.g. one or more of FHR1, FHR2, FHR3, FHR4, FHR5, FH and/or FHL-1);
reduce/prevent/inhibit normal post-transcriptional processing (e.g. splicing, translation and/or post-translational processing) of RNA encoding one or more Factor H family proteins (e.g. one or more of FHR1, FHR2, FHR3, FHR4, FHR5, FH and/or FHL-1); and/or
reduce/prevent/inhibit translation of mRNA encoding one or more Factor H family proteins (e.g. one or more of FHR1, FHR2, FHR3, FHR4, FHR5, FH and/or FHL-1).

In combination with any one or more of the properties above, an agent according to the present disclosure may not reduce/prevent/inhibit expression of a gene or genes encoding FH and/or FHL-1, not reduce the level of RNA encoding FH and/or FHL-1, not reduce/prevent/inhibit transcription of nucleic acid encoding FH and/or FHL-1, not increase degradation of RNA encoding FH and/or FHL-1, not reduce/prevent/inhibit expression of FH and/or FHL-1 at the protein level, not reduce the level of FH and/or FHL-1 proteins, not reduce/prevent/inhibit normal post-transcriptional processing of RNA encoding FH and/or FHL-1, and/or not reduce/prevent/inhibit translation of mRNA encoding FH and/or FHL-1.

It will be appreciated that a given agent may display more than one of the properties recited in the preceding paragraphs. A given agent may be evaluated for the properties recited in the preceding paragraph using suitable assays. The assays may be e.g. in vitro assays, optionally cell-based assays or cell-free assays. The assays may be e.g. ex vivo assays, i.e. performed using cells/tissue/an organ obtains from a subject. The assays may be e.g. in vivo assays, i.e. performed in non-human animals.

Where assays are cell-based assays, they may comprise treating cells with an agent in order to determine whether the agent displays one or more of the recited properties. Assays may employ species labelled with detectable entities in order to facilitate their detection. Assays may comprise evaluating the recited properties following treatment of cells separately with a range of quantities/concentrations of a given agent (e.g. a dilution series). It will be appreciated that the cells employed in such are preferably cells that express Factor H family proteins, e.g. hepatocytes. The assays may comprise treating cells, e.g. with an agent described herein, to reduce or prevent expression and/or activity of Factor H family genes and/or proteins.

Analysis of the results of such assays may comprise determining the concentration at which 50% of the maximal level of the relevant activity is attained. The concentration of agent at which 50% of the maximal level of the relevant activity is attained may be referred to as the ‘half-maximal effective concentration’ of the agent in relation to the relevant activity, which may also be referred to as the ‘EC50’. By way of illustration, the EC50 of a given inhibitory agent for increasing degradation of RNA encoding one or more Factor H family proteins (e.g. one or more of FHR1, FHR2, FHR3, FHR4, FHR5, FH and/or FHL-1) may be the concentration at which 50% of the maximal level of degradation of RNA encoding one or more Factor H family proteins (e.g. one or more of FHR1, FHR2, FHR3, FHR4, FHR5, FH and/or FHL-1) is achieved.

Depending on the property, the EC50 may also be referred to as the ‘half-maximal inhibitory concentration’ or ‘IC50’, this being the concentration of agent at which 50% of the maximal level of inhibition of a given property is observed. By way of illustration, the IC50 of a given inhibitory agent for reducing expression of a gene encoding one or more Factor H family proteins (e.g. one or more of FHR1, FHR2, FHR3, FHR4, FHR5, FH and/or FHL-1) may be the concentration at which 50% of the maximal level of inhibition of expression of the gene is achieved.

Agents capable of reducing/preventing gene expression of one or more Factor H family proteins and/or reducing/preventing transcription of nucleic acid encoding one or more Factor H family proteins and/or reducing the level of RNA encoding one or more Factor H family proteins and/or increasing degradation of RNA encoding one or more Factor H family proteins may be identified using assays comprising detecting and/or quantifying the level of RNA encoding one or more Factor H family proteins (e.g. one or more of FHR1, FHR2, FHR3, FHR4, FHR5, FH and/or FHL-1).

Such assays may comprise quantifying RNA encoding one or more Factor H family proteins by RT-qPCR (a technique well known to the skilled person). The methods may employ primers and/or probes for the detection and/or quantification of RNA encoding one or more Factor H family proteins. Such assays may comprise introducing (e.g. by transfection) into cells that express one or more Factor H family proteins in in vitro culture (i) a putative agent e.g. an inhibitory nucleic acid, or (ii) a control agent (e.g. a nucleic acid known not to influence the level of RNA encoding one or more Factor H family proteins), and subsequently (e.g. after an appropriate period of time, i.e. a period of time sufficient for a reduction in the level of gene expression of one or more Factor H family proteins/transcription of nucleic acid encoding one or more Factor H family proteins/level of RNA encoding one or more Factor H family proteins or an increase in the level of degradation of RNA encoding one or more Factor H family proteins to be observed) measuring the level of RNA encoding one or more Factor H family proteins in cells according to (i) and (ii), and (iii) comparing the level of RNA encoding one or more Factor H family proteins detected to determine whether the putative agent reduces/prevents gene expression of one or more Factor H family proteins/transcription of nucleic acid encoding one or more Factor H family proteins, and/or reduces the level of RNA encoding one or more Factor H family proteins, and/or increases degradation of RNA encoding one or more Factor H family proteins.

Agents, e.g. inhibitory nucleic acids or nucleic acid editing tools, capable of reducing/preventing normal splicing of pre-mRNA encoding one or more Factor H family proteins may be identified using assays comprising detecting and/or quantifying the level of RNA (e.g. mature mRNA) encoding one or more isoforms of one or more Factor H family proteins. Such assays may comprise quantifying RNA (e.g. mature mRNA) encoding one or more isoforms of one or more Factor H family proteins by RT-qPCR.

The methods may employ primers and/or probes for the detection and/or quantification of mature mRNA produced by canonical splicing of pre-mRNA transcribed from a gene encoding one or more Factor H family proteins, and/or primers and/or probes for the detection and/or quantification of mature mRNA produced by alternative splicing of pre-mRNA transcribed from a gene encoding one or more Factor H family proteins.

Mature mRNA produced by canonical splicing of pre-mRNA transcribed from a gene encoding one or more Factor H family proteins may be mature mRNA encoding the major isoform produced by expression of the gene encoding one or more Factor H family proteins. The major isoform may be the most commonly produced/detected isoform. For example, mature mRNA produced by canonical splicing of pre-mRNA transcribed from human FHR2 may be mature mRNA encoding human FHR2 isoform 1 (i.e. having the amino acid sequence shown in SEQ ID NO:4).

Mature mRNA produced by alternative splicing of pre-mRNA transcribed from a gene encoding one or more Factor H family proteins may be mature mRNA encoding an isoform other than the major isoform produced by expression of the gene encoding one or more Factor H family proteins. For example, mature mRNA produced by alternative splicing of pre-mRNA transcribed from human FHR2 may be mature mRNA encoding an isoform of human FHR2 other than isoform 1 (i.e. having an amino acid sequence non-identical to SEQ ID NO:4); e.g. mature mRNA encoding human FHR2 isoform 2 (i.e. having an amino acid sequence non-identical to SEQ ID NO:5).

Such assays may comprise introducing (e.g. by transfection) into cells that express one or more Factor H family proteins in in vitro culture (i) a putative agent, e.g. inhibitory nucleic acid, or (ii) a control agent (e.g. a nucleic acid known not to influence splicing of pre-mRNA encoding one or more Factor H family proteins), and subsequently (e.g. after an appropriate period of time, i.e. a period of time sufficient for an effect on splicing of pre-mRNA encoding one or more Factor H family proteins to be observed) measuring the level of mature mRNA encoding one or more isoforms of one or more Factor H family proteins in cells according to (i) and (ii), and (iii) comparing the level of mature mRNA encoding the isoform(s) to determine whether the putative agent reduces/prevents reduces/prevents normal splicing of pre-mRNA encoding one or more Factor H family proteins.

Agents capable of reducing the level of one or more Factor H family proteins and/or reducing/preventing translation of mRNA encoding one or more Factor H family proteins (e.g. one or more of FHR1, FHR2, FHR3, FHR4, FHR5, FH and/or FHL-1) may be identified using assays comprising detecting the level of the relevant protein(s), e.g. using techniques well known to the skilled person such as antibody/reporter-based methods (western blot, ELISA, immunohisto/cytochemistry, etc.). The methods may employ antibodies specific for one or more Factor H family proteins. Such assays may comprise introducing (e.g. by transfection) into cells that express one or more Factor H family proteins in in vitro culture (i) a putative agent, e.g. inhibitory nucleic acid, or (ii) a control agent (e.g. a nucleic acid known not to influence the level of RNA encoding one or more Factor H family proteins), and subsequently (e.g. after an appropriate period of time, i.e. a period of time sufficient for a reduction in the level of one or more Factor H family proteins to be observed) measuring the level of one or more Factor H family proteins in cells according to (i) and (ii), and (iii) comparing the level of one or more Factor H family proteins detected to determine whether the putative agent reduces the level of the relevant proteins and/or reduces/prevents translation of mRNA encoding one or more Factor H family proteins.

In some embodiments, an agent according to the present disclosure may be capable of reducing the expression of a gene or genes encoding one or more Factor H family proteins (e.g. one or more of FHR1, FHR2, FHR3, FHR4, FHR5, FH and/or FHL-1) in a given assay to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of expression observed in the absence of the agent, or in the presence of the same quantity of a control agent known not to be an inhibitor of the relevant gene(s). In some embodiments, the gene expression of FH and/or FHL-1 may be unaffected by said agent.

In some embodiments, an agent according to the present disclosure may be capable of reducing the expression of a gene or genes encoding one or more Factor H family proteins (e.g. one or more of FHR1, FHR2, FHR3, FHR4, FHR5, FH and/or FHL-1) in a given assay to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level of expression observed in the absence of the agent, or in the presence of the same quantity of a control agent known not to be an inhibitor of the relevant gene(s). In some embodiments, the gene expression of FH and/or FHL-1 may be unaffected by said agent.

In some embodiments, an agent according to the present disclosure may be capable of reducing the level of expression of a gene encoding one or more Factor H family proteins (e.g. one or more of FHR1, FHR2, FHR3, FHR4, FHR5, FH and/or FHL-1) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the agent, or in the presence of the same quantity of a control agent known not to reduce expression of the gene encoding one or more Factor H family proteins, in a given assay. In some embodiments, the level of gene expression of FH and/or FHL-1 may be unaffected by said agent.

In some embodiments, an agent according to the present disclosure may be capable of reducing the level of expression of a gene encoding one or more Factor H family proteins (e.g. one or more of FHR1, FHR2, FHR3, FHR4, FHR5, FH and/or FHL-1) to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the agent, or in the presence of the same quantity of a control agent known not to reduce expression of the gene encoding one or more Factor H family proteins, in a given assay. In some embodiments, the level of gene expression of FH and/or FHL-1 may be unaffected by said agent.

In some embodiments, an agent according to the present disclosure may be capable of reducing the level of RNA encoding one or more Factor H family proteins (e.g. one or more of FHR1, FHR2, FHR3, FHR4, FHR5, FH and/or FHL-1) in a given assay to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the agent, or in the presence of the same quantity of a control agent known not to reduce the level of RNA encoding one or more Factor H family proteins. In some embodiments, the level of RNA encoding FH and/or FHL-1 may be unaffected by said agent.

In some embodiments, an agent according to the present disclosure may be capable of reducing the level of RNA encoding one or more Factor H family proteins (e.g. one or more of FHR1, FHR2, FHR3, FHR4, FHR5, FH and/or FHL-1) in a given assay to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the agent, or in the presence of the same quantity of a control agent known not to reduce the level of RNA encoding one or more Factor H family proteins. In some embodiments, the level of RNA encoding FH and/or FHL-1 may be unaffected by said agent.

In some embodiments, an agent according to the present disclosure may be capable of reducing the level of transcription of nucleic acid encoding one or more Factor H family proteins (e.g. one or more of FHR1, FHR2, FHR3, FHR4, FHR5, FH and/or FHL-1) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the agent, or in the presence of the same quantity of a control agent known not to reduce transcription of nucleic acid encoding one or more Factor H family proteins, in a given assay. In some embodiments, the level of transcription of nucleic acid encoding FH and/or FHL-1 may be unaffected by said agent.

In some embodiments, an agent according to the present disclosure may be capable of reducing the level of transcription of nucleic acid encoding one or more Factor H family proteins (e.g. one or more of FHR1, FHR2, FHR3, FHR4, FHR5, FH and/or FHL-1) to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the agent, or in the presence of the same quantity of a control agent known not to reduce transcription of nucleic acid encoding one or more Factor H family proteins, in a given assay. In some embodiments, the level of transcription of nucleic acid encoding FH and/or FHL-1 may be unaffected by said agent.

In some embodiments, an agent according to the present disclosure may be capable of reducing the level of one or more Factor H family proteins (e.g. one or more of FHR1, FHR2, FHR3, FHR4, FHR5, FH and/or FHL-1) in a given assay to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the agent, or in the presence of the same quantity of a control agent known not to be an inhibitor of the relevant protein(s). In some embodiments, the level of FH and/or FHL-1 protein(s) may be unaffected by said agent.

In some embodiments, an agent according to the present disclosure may be capable of reducing the level of one or more Factor H family proteins (e.g. one or more of FHR1, FHR2, FHR3, FHR4, FHR5, FH and/or FHL-1) in a given assay to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the agent, or in the presence of the same quantity of a control agent known not to be an inhibitor of the relevant protein(s). In some embodiments, the level of FH and/or FHL-1 protein(s) may be unaffected by said agent.

In some embodiments, an agent according to the present disclosure may be capable of reducing normal splicing of pre-mRNA encoding one or more Factor H family proteins (e.g. one or more of FHR1, FHR2, FHR3, FHR4, FHR5, FH and/or FHL-1) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the agent, or in the presence of the same quantity of a control agent known not to reduce normal splicing of pre-mRNA encoding one or more Factor H family proteins, in a given assay. In some embodiments, normal splicing of pre-mRNA encoding FH and/or FHL-1 may be unaffected by said agent.

In some embodiments, an agent according to the present disclosure may be capable of reducing the level of normal splicing of pre-mRNA encoding one or more Factor H family proteins (e.g. one or more of FHR1, FHR2, FHR3, FHR4, FHR5, FH and/or FHL-1) to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the agent, or in the presence of the same quantity of a control agent known not to reduce normal splicing of pre-mRNA encoding one or more Factor H family proteins, in a given assay. In some embodiments, normal splicing of pre-mRNA encoding FH and/or FHL-1 may be unaffected by said agent.

In some embodiments, an agent according to the present disclosure may be capable of reducing translation of mRNA encoding one or more Factor H family proteins (e.g. one or more of FHR1, FHR2, FHR3, FHR4, FHR5, FH and/or FHL-1) to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the agent, or in the presence of the same quantity of a control agent known not to reduce translation of mRNA encoding one or more Factor H family proteins, in a given assay. In some embodiments, translation of mRNA encoding FH and/or FHL-1 may be unaffected by said agent.

In some embodiments, an agent according to the present disclosure may be capable of reducing translation of mRNA encoding one or more Factor H family proteins (e.g. one or more of FHR1, FHR2, FHR3, FHR4, FHR5, FH and/or FHL-1) to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the agent, or in the presence of the same quantity of a control agent known not to reduce translation of mRNA encoding one or more Factor H family proteins, in a given assay. In some embodiments, translation of mRNA encoding FH and/or FHL-1 may be unaffected by said agent.

Preferred levels of reduction in accordance with the preceding paragraphs are reduction to less than 0.5 times/≤50%, e.g. one of less than 0.4 times/≤40%, less than 0.3 times/≤30%, less than 0.2 times/≤20%, less than 0.15 times/≤15%, or less than 0.1 times/≤10%.

In some embodiments, an agent according to the present disclosure may be capable of increasing degradation of RNA encoding one or more Factor H family proteins (e.g. one or more of FHR1, FHR2, FHR3, FHR4, FHR5, FH and/or FHL-1) to more than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 10 times the level observed in the absence of the agent, or in the presence of the same quantity of a control agent known not increase degradation of RNA encoding one or more Factor H family proteins, in a given assay. In some embodiments, RNA encoding FH and/or FHL-1 may be unaffected by said agent, e.g. not degraded.

In some embodiments, an agent according to the present disclosure prevents or silences expression of a gene or genes encoding one or more Factor H family proteins (e.g. one or more of FHR1, FHR2, FHR3, FHR4, FHR5, FH and/or FHL-1). In some embodiments, an agent according to the present disclosure prevents or silences expression of one or more Factor H family proteins (e.g. one or more of FHR1, FHR2, FHR3, FHR4, FHR5, FH and/or FHL-1) at the protein level. As used herein, expression of a given gene(s)/protein(s) may be considered to be ‘prevented’ or ‘silenced’ where the level of expression is reduced to less than 0.1 times/<10% of the level observed in the absence of the agent, or in the presence of the same quantity of a control agent known not to be an inhibitor of the relevant gene(s)/protein(s). In some embodiments, expression of FH and/or FHL-1 may be unaffected, i.e. not silenced, by said agent.

In any embodiment above, the gene and/or protein expression (e.g. via increased transcription and/or translation, or decreased degradation) of one or both of FH and/or FHL-1 may be increased by said agent, e.g. in conjunction with the effect of the agent on the gene and/or protein expression of FHR1, FHR2, FHR3, FHR4, and/or FHR5.

In any embodiment above, an agent described herein may exert the disclosed effects on the gene and/or protein expression of:
a) FHR1;
b) FHR2;
c) FHR3;
d) FHR4;
e) FHR5;
f) FHR1 and FHR2;
g) FHR1 and FHR3;
h) FHR1 and FHR4;
i) FHR1 and FHR5;
j) FHR2 and FHR3;
k) FHR2 and FHR4;
l) FHR2 and FHR5;
m) FHR3 and FHR4;
n) FHR3 and FHR5;
o) FHR4 and FHR5;
p) FHR1, FHR2 and FHR3;
q) FHR1, FHR2 and FHR4;
r) FHR1, FHR2 and FHR5;
s) FHR1, FHR3 and FHR4;
t) FHR1, FHR3 and FHR5;
u) FHR1, FHR4 and FHR5;
v) FHR2, FHR3 and FHR4;
w) FHR2, FHR3 and FHR5;
x) FHR2, FHR4 and FHR5;
y) FHR3, FHR4 and FHR5;
z) FHR1, FHR2, FHR3 and FHR4;
aa) FHR1, FHR2, FHR3 and FHR5;
bb) FHR1, FHR2, FHR4 and FHR5;
cc) FHR2, FHR3, FHR4 and FHR5;
dd) FHR1, FHR3, FHR4 and FHR5; or ee) FHR1, FHR2, FHR3, FHR4 and FHR5.

In some embodiments, an agent according to the present disclosure may have an IC₅₀ value for inhibiting the gene and/or protein expression of one or more Factor H family proteins (e.g. one or more of FHR1, FHR2, FHR3, FHR4, FHR5, FH and/or FHL-1) of ≤1 μM, e.g. one of ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤3 nM, ≤2 nM, ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

In preferred embodiments, an agent according to the present disclosure (e.g. an siRNA) may have an IC₅₀ value for inhibiting the gene and/or protein expression of one or more Factor H family proteins (e.g. one or more of FHR1, FHR2, FHR3, FHR4, FHR5, FH and/or FHL-1) of ≤1 nM, 900 pM, 800 pM, 700 pM, ≤600 pM, ≤500 pM, ≤400 pM, ≤300 pM, ≤200 pM, ≤100 pM, ≤50 pM, ≤40 pM, ≤30 pM, ≤20 pM, 10 pM or ≤1 pM.

Inhibitory nucleic acids and related articles Aspects and embodiments of the present disclosure relate to agents that are, or comprise, inhibitory nucleic acids. As used herein, an ‘inhibitory nucleic acid’ refers to a nucleic acid capable of reducing or preventing the gene and/or protein expression of one or more given target gene(s)/protein(s). The term “agent” as used herein may refer to one or more inhibitory nucleic acids.

Inhibitory nucleic acids according to the present disclosure are suitable for reducing gene and/or protein expression of complement proteins as described herein (e.g. one or more Factor H family proteins). More particularly, aspects and embodiments of the present disclosure relate to inhibitory nucleic acids targeting one or more complement proteins as described herein (e.g. one or more of FH, FHL-1, FHR1, FHR2, FHR3, FHR4, FHR5, FI, C3, C3b, C3a, iC3b, C3f, C3c, C3dg, and/or C3d). In some embodiments, the present disclosure relates to inhibitory nucleic acids targeting one or more of FHR1, FHR2, FHR3, FHR4, FHR5, FH and/or FHL-1. In some embodiments, the present disclosure relates to inhibitory nucleic acids targeting one or more of FHR1, FHR2, FHR3, FHR4, and/or FHR5.

Inhibitory nucleic acids according to the present disclosure may comprise or consist of DNA and/or RNA. Inhibitory nucleic acids may be single-stranded (e.g. in the case of antisense oligonucleotides) or may be double-stranded or comprise double-stranded regions (e.g. in the case of siRNA, miRNA, shRNA, etc.). Inhibitory nucleic acids may comprise both double-stranded and single-stranded regions (e.g. in the case of shRNA and pre-miRNA molecules, which are double-stranded in the stem region of the hairpin structure, and single-stranded in the loop region of the hairpin structure).

In some embodiments, an inhibitory nucleic acid according to the present disclosure may be an antisense nucleic acid as described herein. In some embodiments, an inhibitory nucleic acid may comprise an antisense nucleic acid as described herein. In some embodiments, an inhibitory nucleic acid may encode an antisense nucleic acid as described herein.

As used herein, an ‘antisense nucleic acid’ refers to a nucleic acid (e.g. DNA or RNA) that is complementary to at least a portion of a target nucleotide sequence (e.g. of an RNA encoding a Factor H family protein). Antisense nucleic acids according to the present disclosure are preferably single-stranded nucleic acids, and bind via complementary Watson-Crick base-pairing to a target nucleotide sequence. Complementary base-pairing may involve hydrogen bonding between complementary base pairs. Antisense nucleic acids may be provided in the form of single-stranded molecules, as for example in the case of antisense oligonucleotides, or may be comprised in double-stranded molecular species, as for example in the case of siRNA, miRNA and shRNA molecules.

Complementary base-pairing between the antisense nucleic acid and its target nucleotide sequence may be complete. In such embodiments the antisense nucleic acid comprises, or consists of, the reverse complement of its target nucleotide sequence, and complementary base-pairing occurs between each nucleotide of the target nucleotide sequence and complementary nucleotides in the antisense nucleic acid. Alternatively, complementary base-pairing between the antisense nucleic acid and its target nucleotide sequence may be incomplete/partial. In such embodiments complementary base-pairing occurs between some, but not all, nucleotides of the target nucleotide sequence and complementary nucleotides in the antisense nucleic acid.

Such binding between nucleic acids trough complementary base pairing may be referred to as ‘hybridisation’. Through binding to its target nucleotide sequence, an antisense nucleic acid may form a nucleic acid complex comprising (i) the antisense nucleic acid and (ii) a target nucleic acid comprising the target nucleotide sequence.

The nucleotide sequence of an antisense nucleic acid is sufficiently complementary to its target nucleotide sequence such that it binds or hybridises to the target nucleotide sequence. It will be appreciated that antisense nucleic acids preferably have a high degree of sequence identity to the reverse complement of its target nucleotide sequence. In some embodiments, the antisense nucleic acid comprises or consists of a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of its target nucleotide sequence.

In some embodiments, an antisense nucleic acid according to the present disclosure comprises: a nucleotide sequence which is the reverse complement of its target nucleotide sequence, or a nucleotide sequence comprising 1 to 10 (e.g. one of 1, 2, 3, 4, 5, 6, 7, 8, 9 or 10) substitutions relative to the reverse complement of its target nucleotide sequence.

In some embodiments, the target nucleotide sequence for an antisense nucleic acid according to the present disclosure comprises, or consists of, 5 to 100 nucleotides, e.g. one of 10 to 80, 12 to 50, or 15 to 30 nucleotides.

In some embodiments, the antisense nucleic acid reduces/prevents transcription of nucleic acid comprising its target nucleotide sequence. In some embodiments, the antisense nucleic acid reduces/prevents association of factors required for normal transcription (e.g. enhancers, RNA polymerase) with nucleic acid comprising its target nucleotide sequence.

In some embodiments, the antisense nucleic acid increases/potentiates degradation of nucleic acid comprising its target nucleotide sequence, e.g. through RNA interference. In some embodiments, the antisense nucleic acid reduces/prevents translation of nucleic acid comprising its target nucleotide sequence, e.g. through RNA interference or antisense degradation via RNase H. RNA interference is described e.g. in Agrawal et al., Microbiol. Mol. Bio. Rev. (2003) 67(4): 657-685 and Hu et al., Sig. Transduc. Tar. Ther. (2020) 5(101), both of which are hereby incorporated by reference in their entirety. Briefly, double-stranded RNA molecules are recognised by the argonaute component of the RNA-induced silencing complex (RISC). The double-stranded RNAs are separated into single strands and integrated into an active RISC, by the RISC-Loading Complex (RLC). The RISC-integrated strands bind to their target RNA through complementary base pairing, and depending on the identity of the RISC-integrated RNA and degree of complementarity to the target RNA, the RISC then either cleaves the target RNA resulting in its degradation, or otherwise blocks access of ribosomes preventing its translation. RNAi based therapeutics have been approved for a number of indications (Kim, Chonnam Med J. (2020) 56(2): 87-93).

In some embodiments, the antisense nucleic acid reduces/prevents normal post-transcriptional processing (e.g. splicing and/or translation) of nucleic acid comprising its target nucleotide sequence. In some embodiments, the antisense nucleic acid reduces or alters splicing of pre-mRNA comprising its target nucleotide sequence to mature mRNA. In some embodiments, the antisense nucleic acid reduces translation of mRNA comprising its target nucleotide sequence to protein.

In some embodiments, the antisense nucleic acid reduces/prevents association of factors required for normal post-transcriptional processing (e.g. components of the spliceosome) with nucleic acid comprising its target nucleotide sequence. In such instances, the antisense nucleic may be referred to as a ‘splice-switching’ nucleic acid.

Splice-switching nucleic acids are reviewed e.g. in Haves and Hastings, Nucleic Acids Res. (2016) 44(14): 6549-6563, which is hereby incorporated by reference in its entirety. Splice-switching nucleic acids include e.g. splice-switching oligonucleotides (SSOs). They disrupt the normal splicing of target RNA transcripts by blocking the RNA:RNA base-pairing and/or protein:RNA binding interactions that occur between components of the splicing machinery and pre-mRNA. Splice-switching nucleic acids may be employed to alter the number/proportion of mature mRNA transcripts encoding one or more Factor H family proteins (e.g. one or more of FHR1, FHR2, FHR3, FHR4, FHR5, FH and/or FHL-1). Splice-switching nucleic acids may be designed to target a specific region of the target transcript, e.g. to effect skipping of exon(s) of interest, e.g. exons encoding domains/regions of interest. SSOs often comprise alterations to oligonucleotide sugar-phosphate backbones in order to reduce/prevent RNAse H degradation, such as e.g. phosphorothioate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′O-methyl (2′OMe) and 2′-O-methoxyethyl (MOE) ribose modifications and/or 5′-methylcytosine modifications.

In some embodiments, the antisense nucleic acid inhibits/reduces translation of nucleic acid comprising its target nucleotide sequence. In some embodiments, the antisense nucleic acid reduces/prevents association of factors required for translation (e.g. ribosomes) with nucleic acid comprising its target nucleotide sequence.

It will be appreciated that the target nucleotide sequence to which an antisense nucleic acid binds is a nucleotide sequence encoding a protein of which it is desired to inhibit expression. Accordingly, in aspects and embodiments of the present disclosure, the target nucleotide sequence for an antisense nucleic acid is a nucleotide sequence of a gene(s) encoding one or more Factor H family proteins (e.g. one or more of FHR1, FHR2, FHR3, FHR4, FHR5, FH and/or FHL-1). For example, the target nucleotide sequence may be a nucleotide sequence comprised within one or more of SEQ ID NO: 218 to 223.

The following paragraphs describe target nucleotide sequences that may be targeted by an agent according to the present disclosure, e.g. an inhibitory nucleic acid and/or a gene editing system such as a CRISPR/Cas system comprising a guideRNA.

In some embodiments, the target nucleotide sequence is a nucleotide sequence of DNA encoding one or more Factor H family proteins (e.g. one or more of CFHR1, CFHR2, CFHR3, CFHR4, CFHR5, and/or CFH). In some embodiments, the target nucleotide sequence is a nucleotide sequence of RNA encoded by one or more genes encoding one or more Factor H family proteins (e.g. one or more of CFHR1, CFHR2, CFHR3, CFHR4, CFHR5, and/or CFH). In some embodiments, the target nucleotide sequence is a nucleotide sequence of RNA encoding one or more Factor H family proteins (e.g. one or more of FHR1, FHR2, FHR3, FHR4, FHR5, FH and/or FHL-1). In some embodiments, the target nucleotide sequence comprises one or more nucleotides of an exon of RNA encoding one or more Factor H family proteins (e.g. one or more of FHR1, FHR2, FHR3, FHR4, FHR5, FH and/or FHL-1). In some embodiments, the target nucleotide sequence is a nucleotide sequence of an exon of RNA encoding one or more Factor H family proteins (e.g. one or more of FHR1, FHR2, FHR3, FHR4, FHR5, FH and/or FHL-1).

In some embodiments, the target nucleotide sequence is a nucleotide sequence of RNA (e.g. mRNA) encoded by one or more genes encoding one or more Factor H related proteins, or RNA encoding one or more Factor H related proteins (e.g. one or more of FHR1, FHR2, FHR3, FHR4, and/or FHR5).

In some embodiments, the target nucleotide sequence is a nucleotide sequence of RNA encoded by a gene encoding FHR1. In some embodiments, the target nucleotide sequence is a nucleotide sequence of RNA encoding FHR1. In some embodiments, the target nucleotide sequence comprises one or more nucleotides of an exon of RNA encoding FHR1. In some embodiments, the target nucleotide sequence is a nucleotide sequence of an exon of RNA encoding FHR1. In some embodiments, the target nucleotide sequence is a nucleotide sequence of RNA encoded by a gene encoding FHR2. In some embodiments, the target nucleotide sequence is a nucleotide sequence of RNA encoding FHR2. In some embodiments, the target nucleotide sequence comprises one or more nucleotides of an exon of RNA encoding FHR2. In some embodiments, the target nucleotide sequence is a nucleotide sequence of an exon of RNA encoding FHR2. In some embodiments, the target nucleotide sequence is a nucleotide sequence of RNA encoded by a gene encoding FHR3. In some embodiments, the target nucleotide sequence is a nucleotide sequence of RNA encoding FHR3. In some embodiments, the target nucleotide sequence comprises one or more nucleotides of an exon of RNA encoding FHR3. In some embodiments, the target nucleotide sequence is a nucleotide sequence of an exon of RNA encoding FHR3. In some embodiments, the target nucleotide sequence is a nucleotide sequence of RNA encoded by a gene encoding FHR4. In some embodiments, the target nucleotide sequence is a nucleotide sequence of RNA encoding FHR4. In some embodiments, the target nucleotide sequence comprises one or more nucleotides of an exon of RNA encoding FHR4. In some embodiments, the target nucleotide sequence is a nucleotide sequence of an exon of RNA encoding FHR4. In some embodiments, the target nucleotide sequence is a nucleotide sequence of RNA encoded by a gene encoding FHR5. In some embodiments, the target nucleotide sequence is a nucleotide sequence of RNA encoding FHR5. In some embodiments, the target nucleotide sequence comprises one or more nucleotides of an exon of RNA encoding FHR5. In some embodiments, the target nucleotide sequence is a nucleotide sequence of an exon of RNA encoding FHR5. In some embodiments, the target nucleotide sequence is a nucleotide sequence of RNA encoded by a gene encoding FH. In some embodiments, the target nucleotide sequence is a nucleotide sequence of RNA encoding FH. In some embodiments, the target nucleotide sequence comprises one or more nucleotides of an exon of RNA encoding FH. In some embodiments, the target nucleotide sequence is a nucleotide sequence of an exon of RNA encoding FH.

In some embodiments, the target nucleotide sequence is a nucleotide sequence found in RNA encoding more than one Factor H family protein (e.g. selected from FHR1, FHR1, FHR3, FHR4, FHR5, FH and/or FHL-1, e.g. selected from FHR1, FHR1, FHR3, FHR4, and/or FHR5). Through targeting such nucleotide sequences, a given agent, e.g. inhibitory nucleic acid according to the present disclosure may be able to reduce gene and/or protein expression of the different Factor H family proteins encoded by RNA comprising the relevant target sequence. Any and all combinations of nucleotide sequences found in one or more Factor H family proteins are envisaged.

“Antisense nucleic acid” in the following paragraphs may be present in any agent, for example, in an inhibitory nucleic acid or in a nucleic acid for targeting a gene editing system, e.g. a gRNA or sgRNA.

By way of illustration, SEQ ID NO:158 of the present disclosure is found in the sequence of RNA encoding FHR1 and is also found in the sequence of RNA encoding FHR2. Accordingly, an antisense nucleic acid having a nucleotide sequence with at least 70% sequence identity to SEQ ID NO:178 reduces gene and/or protein expression of at least FHR1 and FHR2. In some cases an antisense nucleic acid having a nucleotide sequence with at least 70% sequence identity to SEQ ID NO:178 reduces gene and/or protein expression of FHR1, FHR2, FHR3, FHR4 and FHR5. In some cases an antisense nucleic acid having a nucleotide sequence with at least 70% sequence identity to SEQ ID NO:178 has no effect on or increases gene and/or protein expression of FH and/or FHL-1.

SEQ ID NO:159 of the present disclosure is found in the sequence of RNA encoding FHR1. Accordingly, an antisense nucleic acid having a nucleotide sequence with at least 70% sequence identity to SEQ ID NO:179 reduces gene and/or protein expression of at least FHR1. In some cases, an antisense nucleic acid having a nucleotide sequence with at least 70% sequence identity to SEQ ID NO:179 reduces gene and/or protein expression of FHR1, FHR3, FHR4 and FHR5. In some cases an antisense nucleic acid having a nucleotide sequence with at least 70% sequence identity to SEQ ID NO:179 has no effect on or increases gene and/or protein expression of FHR2, FH and/or FHL-1.

SEQ ID NO:160 of the present disclosure is found in the sequence of RNA encoding FHR1. Accordingly, an antisense nucleic acid having a nucleotide sequence with at least 70% sequence identity to SEQ ID NO:180 reduces gene and/or protein expression of at least FHR1. In some cases, an antisense nucleic acid having a nucleotide sequence with at least 70% sequence identity to SEQ ID NO:180 reduces gene and/or protein expression of FHR1, FHR3, FHR4 and FHR5. In some cases an antisense nucleic acid having a nucleotide sequence with at least 70% sequence identity to SEQ ID NO:180 has no effect on or increases gene and/or protein expression of FHR2, FH and/or FHL-1.

SEQ ID NO:161 of the present disclosure is found in the sequence of RNA encoding FHR3. Accordingly, an antisense nucleic acid having a nucleotide sequence with at least 70% sequence identity to SEQ ID NO:181 reduces gene and/or protein expression of at least FHR3. In some cases, an antisense nucleic acid having a nucleotide sequence with at least 70% sequence identity to SEQ ID NO:181 reduces gene and/or protein expression of FHR3, FHR4 and optionally FHR5. In some cases an antisense nucleic acid having a nucleotide sequence with at least 70% sequence identity to SEQ ID NO:181 has no effect on or increases gene and/or protein expression of FHR1, FHR2, FHR5, FH and/or FHL-1.

Similarly, SEQ ID NO:162 is found in the sequence of RNA encoding FHR3 and is also found in the sequence of RNA encoding FHR4, and accordingly antisense nucleic acid having a nucleotide sequence with at least 70% sequence identity to SEQ ID NO:182 would be expected to reduce gene and/or protein expression of at least FHR3 and FHR4. In some cases an antisense nucleic acid having a nucleotide sequence with at least 70% sequence identity to SEQ ID NO:181 has no effect on or increases gene and/or protein expression of FHR1, FHR2, FHR3, FHR4, FHR5, FH and/or FHL-1.

SEQ ID NO:163 of the present disclosure is found in the sequence of RNA encoding FHR3. Accordingly, an antisense nucleic acid having a nucleotide sequence with at least 70% sequence identity to SEQ ID NO:183 reduces gene and/or protein expression of at least FHR3. In some cases, an antisense nucleic acid having a nucleotide sequence with at least 70% sequence identity to SEQ ID NO:183 reduces gene and/or protein expression of FHR3, FHR4 and optionally FHR5. In some cases an antisense nucleic acid having a nucleotide sequence with at least 70% sequence identity to SEQ ID NO:183 has no effect on or increases gene and/or protein expression of FHR1, FHR2, FHR5, FH and/or FHL-1.

SEQ ID NO:164 of the present disclosure is found in the sequence of RNA encoding FHR4. Accordingly, an antisense nucleic acid having a nucleotide sequence with at least 70% sequence identity to SEQ ID NO:184 reduces gene and/or protein expression of at least FHR4. In some cases, an antisense nucleic acid having a nucleotide sequence with at least 70% sequence identity to SEQ ID NO:184 reduces gene and/or protein expression of FHR3, FHR4 and optionally FHR5. In some cases an antisense nucleic acid having a nucleotide sequence with at least 70% sequence identity to SEQ ID NO:184 has no effect on or increases gene and/or protein expression of FHR1, FHR2, FHR5, FH and/or FHL-1.

SEQ ID NO:165 of the present disclosure is found in the sequence of RNA encoding FHR4. Accordingly, an antisense nucleic acid having a nucleotide sequence with at least 70% sequence identity to SEQ ID NO:185 reduces gene and/or protein expression of at least FHR4. In some cases, an antisense nucleic acid having a nucleotide sequence with at least 70% sequence identity to SEQ ID NO:185 reduces gene and/or protein expression of FHR2, FHR3 and/or FHR4. In some cases an antisense nucleic acid having a nucleotide sequence with at least 70% sequence identity to SEQ ID NO:184 has no effect on or increases gene and/or protein expression of FHR1, FHR5, FH and/or FHL-1.

SEQ ID NO:166 is found in the sequence of RNA encoding FHR3 and is also found in the sequence of RNA encoding FHR4, and accordingly antisense nucleic acid having a nucleotide sequence with at least 70% sequence identity to SEQ ID NO:186 reduces gene and/or protein expression of at least FHR3 and FHR4. In some cases an antisense nucleic acid having a nucleotide sequence with at least 70% sequence identity to SEQ ID NO:184 has no effect on or increases gene and/or protein expression of FHR1, FHR2, FHR3, FHR4, FHR5, FH and/or FHL-1.

Similarly, SEQ ID NO:169 is found in the sequence of RNA encoding FH and is also found in the sequence of RNA encoding FHR1 and FHR2, and accordingly antisense nucleic acid having a nucleotide sequence with at least 70% sequence identity to SEQ ID NO:189 reduces gene and/or protein expression of at least FH, CFHR1 and CFHR2. Similarly, SEQ ID NO:170 is found in the sequence of RNA encoding FH and is also found in the sequence of RNA encoding FHR1, and accordingly antisense nucleic acid having a nucleotide sequence with at least 70% sequence identity to SEQ ID NO:190 reduces gene and/or protein expression of at least FH and FHR1. Similarly, SEQ ID NO:171 is found in the sequence of RNA encoding FH and is also found in the sequence of RNA encoding FHR1, and accordingly antisense nucleic acid having a nucleotide sequence with at least 70% sequence identity to SEQ ID NO:191 reduces gene and/or protein expression of at least FH and FHR1. Similarly, SEQ ID NO:172 is found in the sequence of RNA encoding FH and is also found in the sequence of RNA encoding FHR2, and accordingly antisense nucleic acid having a nucleotide sequence with at least 70% sequence identity to SEQ ID NO:192 reduces gene and/or protein expression of at least FH and FHR2.

In some embodiments, the target nucleotide sequence is a nucleotide sequence of RNA encoded by a gene encoding FHR1 and/or FHR2. In some embodiments, the target nucleotide sequence is a nucleotide sequence of RNA encoding FHR1 and/or FHR2. In some embodiments, the target nucleotide sequence comprises one or more nucleotides of an exon of RNA encoding FHR1 and/or FHR2. In some embodiments, the target nucleotide sequence is a nucleotide sequence of an exon of RNA encoding FHR1 and/or FHR2. In some embodiments, the target nucleotide sequence is a nucleotide sequence of RNA encoded by a gene encoding FHR3 and/or FHR4. In some embodiments, the target nucleotide sequence is a nucleotide sequence of RNA encoding FHR3 and/or FHR4. In some embodiments, the target nucleotide sequence comprises one or more nucleotides of an exon of RNA encoding FHR3 and/or FHR4. In some embodiments, the target nucleotide sequence is a nucleotide sequence of an exon of RNA encoding FHR3 and/or FHR4. In some embodiments, the target nucleotide sequence is a nucleotide sequence of RNA encoded by a gene encoding FHR1, FHR2 and/or FH. In some embodiments, the target nucleotide sequence is a nucleotide sequence of RNA encoding FHR1, FHR2 and/or FH. In some embodiments, the target nucleotide sequence comprises one or more nucleotides of an exon of RNA encoding FHR1, FHR2 and/or FH. In some embodiments, the target nucleotide sequence is a nucleotide sequence of an exon of RNA encoding FHR1, FHR2 and/or FH. In some embodiments, the target nucleotide sequence is a nucleotide sequence of RNA encoded by a gene encoding FHR1 and/or FH. In some embodiments, the target nucleotide sequence is a nucleotide sequence of RNA encoding FHR1 and/or FH. In some embodiments, the target nucleotide sequence comprises one or more nucleotides of an exon of RNA encoding FHR1 and/or FH. In some embodiments, the target nucleotide sequence is a nucleotide sequence of an exon of RNA encoding FHR1 and/or FH. In some embodiments, the target nucleotide sequence is a nucleotide sequence of RNA encoded by a gene encoding FHR2 and/or FH. In some embodiments, the target nucleotide sequence is a nucleotide sequence of RNA encoding FHR2 and/or FH. In some embodiments, the target nucleotide sequence comprises one or more nucleotides of an exon of RNA encoding FHR2 and/or FH. In some embodiments, the target nucleotide sequence is a nucleotide sequence of an exon of RNA encoding FHR2 and/or FH.

In some embodiments, the target nucleotide sequence is a nucleotide sequence of SEQ ID NO:218, which is a mRNA sequence encoding FHR1 (with thymine (T) replacing uracil (U)). In some embodiments, the target nucleotide sequence is a nucleotide sequence from between positions 1 and 164 (inclusive) of SEQ ID NO:218. In some embodiments, the target nucleotide sequence is a nucleotide sequence from between positions 531 and 721 (inclusive) of SEQ ID NO:218. In some embodiments, the target nucleotide sequence is a nucleotide sequence from between positions 722 and 904 (inclusive) of SEQ ID NO:218.

In some embodiments, the target nucleotide sequence is a nucleotide sequence of SEQ ID NO:219, which is a mRNA sequence encoding FHR2 (with thymine (T) replacing uracil (U)). In some embodiments, the target nucleotide sequence is a nucleotide sequence from between positions 1 and 201 (inclusive) of SEQ ID NO:219. In some embodiments, the target nucleotide sequence is a nucleotide sequence from between positions 757 and 1498 (inclusive) of SEQ ID NO:219.

In some embodiments, the target nucleotide sequence is a nucleotide sequence of SEQ ID NO:220, which is a mRNA sequence encoding FHR3 (with thymine (T) replacing uracil (U)). In some embodiments, the target nucleotide sequence is a nucleotide sequence from between positions 661 and 2934 (inclusive) of SEQ ID NO:220. In some embodiments, the target nucleotide sequence is a nucleotide sequence from between positions 661 and 843 (inclusive) of SEQ ID NO:220. In some embodiments, the target nucleotide sequence is a nucleotide sequence from between positions 844 and 2934 (inclusive) of SEQ ID NO:220.

In some embodiments, the target nucleotide sequence is a nucleotide sequence of SEQ ID NO:221, which is a mRNA sequence encoding FHR4 (with thymine (T) replacing uracil (U)). In some embodiments, the target nucleotide sequence is a nucleotide sequence from between positions 1 and 157 (inclusive) of SEQ ID NO:221. In some embodiments, the target nucleotide sequence is a nucleotide sequence from between positions 716 and 898 (inclusive) of SEQ ID NO:221. In some embodiments, the target nucleotide sequence is a nucleotide sequence from between positions 1280 and 1456 (inclusive) of SEQ ID NO:221. In some embodiments, the target nucleotide sequence is a nucleotide sequence from between positions 1457 and 2063 (inclusive) of SEQ ID NO:221. In some embodiments, the target nucleotide sequence is a nucleotide sequence from between positions 1457 and 1639 (inclusive) of SEQ ID NO:221.

In some embodiments, the target nucleotide sequence is a nucleotide sequence of SEQ ID NO:222, which is a mRNA sequence encoding FHR5 (with thymine (T) replacing uracil (U)). In some embodiments, the target nucleotide sequence is a nucleotide sequence from between positions 363 and 539 (inclusive) of SEQ ID NO:222. In some embodiments, the target nucleotide sequence is a nucleotide sequence from between positions 1623 and 2814 (inclusive) of SEQ ID NO:222.

In some embodiments, the target nucleotide sequence is a nucleotide sequence of SEQ ID NO:223, which is a mRNA sequence encoding FH (with thymine (T) replacing uracil (U)). In some embodiments, the target nucleotide sequence is a nucleotide sequence from between positions 3386 and 3568 (inclusive) of SEQ ID NO:223.

In some embodiments, the target nucleotide sequence is, or comprises, the nucleotide sequence of SEQ ID NO:158, 159, 160, 161, 162, 163, 164, 165, 166, 167, 168, 169, 170, 171, 172, 173, 174, 175, 176 or 177. In some embodiments, the target nucleotide sequence is, or comprises, the nucleotide sequence of SEQ ID NO:158, 159, 160, 161, 162, 163, 164, 165, 166, 167 or 168. In some embodiments, the target nucleotide sequence is or comprises the nucleotide sequence of SEQ ID NO:158. In some embodiments, the target nucleotide sequence is or comprises the nucleotide sequence of SEQ ID NO:159. In some embodiments, the target nucleotide sequence is or comprises the nucleotide sequence of SEQ ID NO:160. In some embodiments, the target nucleotide sequence is or comprises the nucleotide sequence of SEQ ID NO:161. In some embodiments, the target nucleotide sequence is or comprises the nucleotide sequence of SEQ ID NO:162. In some embodiments, the target nucleotide sequence is or comprises the nucleotide sequence of SEQ ID NO:163. In some embodiments, the target nucleotide sequence is or comprises the nucleotide sequence of SEQ ID NO:164. In some embodiments, the target nucleotide sequence is or comprises the nucleotide sequence of SEQ ID NO:165. In some embodiments, the target nucleotide sequence is or comprises the nucleotide sequence of SEQ ID NO:166. In some embodiments, the target nucleotide sequence is or comprises the nucleotide sequence of SEQ ID NO:167. In some embodiments, the target nucleotide sequence is or comprises the nucleotide sequence of SEQ ID NO:168. In some embodiments, the target nucleotide sequence is or comprises the nucleotide sequence of SEQ ID NO:169. In some embodiments, the target nucleotide sequence is or comprises the nucleotide sequence of SEQ ID NO:170. In some embodiments, the target nucleotide sequence is or comprises the nucleotide sequence of SEQ ID NO:171. In some embodiments, the target nucleotide sequence is or comprises the nucleotide sequence of SEQ ID NO:172. In some embodiments, the target nucleotide sequence is or comprises the nucleotide sequence of SEQ ID NO:173. In some embodiments, the target nucleotide sequence is or comprises the nucleotide sequence of SEQ ID NO:174. In some embodiments, the target nucleotide sequence is or comprises the nucleotide sequence of SEQ ID NO:175. In some embodiments, the target nucleotide sequence is or comprises the nucleotide sequence of SEQ ID NO:176. In some embodiments, the target nucleotide sequence is or comprises the nucleotide sequence of SEQ ID NO:177.

In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:158, 159, 160, 161, 162, 163, 164, 165, 166, 167, 168, 169, 170, 171, 172, 173, 174, 175, 176 or 177. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:158, 159, 160, 161, 162, 163, 164, 165, 166, 167 or 168. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:158. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:159. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:160. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:161. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:162. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:163. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO: 164. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:165. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:166. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:167. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:168. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:169. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:170. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO: 171. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:172. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:173. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:174. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:175. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:176. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:177.

In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:178, 179, 180, 181, 182, 183, 184, 185, 186, 187, 188, 189, 190, 191, 192, 193, 194, 195, 196 or 197. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:178, 179, 180, 181, 182, 183, 184, 185, 186, 187 or 188. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:178. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:179. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:180. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:181. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:182. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:183. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:184. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:185. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:186. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:187. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:188. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:189. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:190. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:191. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:192. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:193. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:194. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:195. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:196. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:197.

The skilled person is readily able in view of the present disclosure to select suitable inhibitory nucleic acids for reducing gene and/or protein expression of a given Factor H family protein.

In embodiments wherein it is desirable to inhibit gene and/or protein expression of FHR1, an inhibitory nucleic acid may comprise or encode antisense nucleic acid having a target nucleotide sequence which is a nucleotide sequence encoding FHR1 (e.g. a nucleotide sequence of RNA encoded by a gene encoding FHR1, or a nucleotide sequence of RNA encoding FHR1). In some embodiments, the target nucleotide sequence comprises one or more nucleotides of an exon of RNA encoding FHR1 (e.g. is a nucleotide sequence of an exon of RNA encoding FHR1). In some embodiments, the target nucleotide sequence is a nucleotide sequence of SEQ ID NO:218 (e.g. a nucleotide sequence from between positions 1 and 164 (inclusive) of SEQ ID NO:218, a nucleotide sequence from between positions 531 and 721 (inclusive) of SEQ ID NO:218, or a nucleotide sequence from between positions 722 and 904 (inclusive) of SEQ ID NO:218). In some embodiments, the target nucleotide sequence is or comprises SEQ ID NO:158, 159, 160, 169, 170 or 171. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:158, 159, 160, 169, 170 or 171. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:178, 179, 180, 189, 190 or 191.

In embodiments wherein it is desirable to inhibit gene and/or protein expression of FHR2, an inhibitory nucleic acid may comprise or encode antisense nucleic acid having a target nucleotide sequence which is a nucleotide sequence encoding FHR2 (e.g. a nucleotide sequence of RNA encoded by a gene encoding FHR2, or a nucleotide sequence of RNA encoding FHR2). In some embodiments, the target nucleotide sequence comprises one or more nucleotides of an exon of RNA encoding FHR2 (e.g. is a nucleotide sequence of an exon of RNA encoding FHR2). In some embodiments, the target nucleotide sequence is a nucleotide sequence of SEQ ID NO:219 (e.g. a nucleotide sequence from between positions 1 and 201 (inclusive) of SEQ ID NO:219 or a nucleotide sequence from between positions 757 and 1498 (inclusive) of SEQ ID NO:219). In some embodiments, the target nucleotide sequence is or comprises SEQ ID NO:158, 169, 172 or 173. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:158, 169, 172 or 173. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:178, 189, 192 or 193.

In embodiments wherein it is desirable to inhibit gene and/or protein expression of FHR3, an inhibitory nucleic acid may comprise or encode antisense nucleic acid having a target nucleotide sequence which is a nucleotide sequence encoding FHR3 (e.g. a nucleotide sequence of RNA encoded by a gene encoding FHR3, or a nucleotide sequence of RNA encoding FHR3). In some embodiments, the target nucleotide sequence comprises one or more nucleotides of an exon of RNA encoding FHR3 (e.g. is a nucleotide sequence of an exon of RNA encoding FHR3). In some embodiments, the target nucleotide sequence is a nucleotide sequence of SEQ ID NO:220 (e.g. a nucleotide sequence from between positions 661 and 2934 (inclusive) of SEQ ID NO:220, a nucleotide sequence from between positions 661 and 843 (inclusive) of SEQ ID NO:220, or a nucleotide sequence from between positions 844 and 2934 (inclusive) of SEQ ID NO:220). In some embodiments, the target nucleotide sequence is or comprises SEQ ID NO:161, 162, 163, 166, 174, 175 or 176. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:161, 162, 163, 166, 174, 175 or 176. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:181, 182, 183, 186, 194, 195 or 196.

In embodiments wherein it is desirable to inhibit gene and/or protein expression of FHR4, an inhibitory nucleic acid may comprise or encode antisense nucleic acid having a target nucleotide sequence which is a nucleotide sequence encoding FHR4 (e.g. a nucleotide sequence of RNA encoded by a gene encoding FHR4, or a nucleotide sequence of RNA encoding FHR4). In some embodiments, the target nucleotide sequence comprises one or more nucleotides of an exon of RNA encoding FHR4 (e.g. is a nucleotide sequence of an exon of RNA encoding FHR4). In some embodiments, the target nucleotide sequence is a nucleotide sequence of SEQ ID NO:221 (e.g. a nucleotide sequence from between positions 1 and 157 (inclusive) of SEQ ID NO:221, a nucleotide sequence from between positions 716 and 898 (inclusive) of SEQ ID NO:221, a nucleotide sequence from between positions 1280 and 1456 (inclusive) of SEQ ID NO:221, a nucleotide sequence from between positions 1457 and 2063 (inclusive) of SEQ ID NO:221, or a nucleotide sequence from between positions 1457 and 1639 (inclusive) of SEQ ID NO:221). In some embodiments, the target nucleotide sequence is or comprises SEQ ID NO:162, 164, 165, 166 or 177. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:162, 164, 165, 166 or 177. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:182, 184, 185, 186 or 197.

In embodiments wherein it is desirable to inhibit gene and/or protein expression of FHR5, an inhibitory nucleic acid may comprise or encode antisense nucleic acid having a target nucleotide sequence which is a nucleotide sequence encoding FHR5 (e.g. a nucleotide sequence of RNA encoded by a gene encoding FHR5, or a nucleotide sequence of RNA encoding FHR5). In some embodiments, the target nucleotide sequence comprises one or more nucleotides of an exon of RNA encoding FHR5 (e.g. is a nucleotide sequence of an exon of RNA encoding FHR5). In some embodiments, the target nucleotide sequence is a nucleotide sequence of SEQ ID NO:222 (e.g. a nucleotide sequence from between positions 363 and 539 (inclusive) of SEQ ID NO:222, or a nucleotide sequence from between positions 1623 and 2814 (inclusive) of SEQ ID NO:222). In some embodiments, the target nucleotide sequence is or comprises SEQ ID NO:167 or 168. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:167 or 168. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:187 or 188.

In embodiments wherein it is desirable to inhibit gene and/or protein expression of FH and/or FHL-1, an inhibitory nucleic acid may comprise or encode antisense nucleic acid having a target nucleotide sequence which is a nucleotide sequence encoding FH and/or FHL-1 (e.g. a nucleotide sequence of RNA encoded by a gene encoding FH and/or FHL-1, or a nucleotide sequence of RNA encoding FH and/or FHL-1). In some embodiments, the target nucleotide sequence comprises one or more nucleotides of an exon of RNA encoding FH and/or FHL-1 (e.g. is a nucleotide sequence of an exon of RNA encoding FH and/or FHL-1). In some embodiments, the target nucleotide sequence is a nucleotide sequence of SEQ ID NO:223 (e.g. a nucleotide sequence from between positions 3386 and 3568 (inclusive) of SEQ ID NO:223). In some embodiments, the target nucleotide sequence is or comprises SEQ ID NO:169, 170, 171 or 172. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:169, 170, 171 or 172. In some embodiments, an inhibitory nucleic acid may comprise or encode antisense nucleic acid comprising or consisting of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:189, 190, 191 or 192.

In some embodiments, an agent, e.g. inhibitory nucleic acid described herein inhibits gene and/or protein expression of FHR1 only, i.e. it does not inhibit gene and/or protein expression of FHR2, FHR3, FHR4, FHR5, FH or FHL-1. In some embodiments, an agent, e.g. inhibitory nucleic acid described herein inhibits gene and/or protein expression of FHR2 only, i.e. it does not inhibit gene and/or protein expression of FHR1, FHR3, FHR4, FHR5, FH or FHL-1. In some embodiments, an agent, e.g. inhibitory nucleic acid described herein inhibits gene and/or protein expression of FHR3 only, i.e. it does not inhibit gene and/or protein expression of FHR1, FHR2, FHR4, FHR5, FH or FHL-1. In some embodiments, an agent, e.g. inhibitory nucleic acid described herein inhibits gene and/or protein expression of FHR4 only, i.e. it does not inhibit gene and/or protein expression of FHR1, FHR2, FHR3, FHR5, FH or FHL-1. In some embodiments, an agent, e.g. inhibitory nucleic acid described herein inhibits gene and/or protein expression of FHR5 only, i.e. it does not inhibit gene and/or protein expression of FHR1, FHR2, FHR3, FHR4, FH or FHL-1. In some embodiments, an agent, e.g. inhibitory nucleic acid described herein inhibits gene and/or protein expression of FH only, i.e. it does not inhibit gene and/or protein expression of FHR1, FHR2, FHR3, FHR4, FHR5, or FHL-1. In some embodiments, an agent, e.g. inhibitory nucleic acid described herein inhibits gene and/or protein expression of FHL-1 only, i.e. it does not inhibit gene and/or protein expression of FHR1, FHR2, FHR3, FHR4, FHR5, or FH. In some embodiments, an agent, e.g. inhibitory nucleic acid described herein inhibits gene and/or protein expression of FH and FHL-1 only, i.e. it does not inhibit gene and/or protein expression of FHR1, FHR2, FHR3, FHR4, or FHR5.

In some embodiments an agent, e.g. inhibitory nucleic acid described herein inhibits gene and/or protein expression of one or more Factor H family proteins, wherein the inhibited protein is not FH and/or FHL-1. That is, an agent, e.g. inhibitory nucleic acid described herein may inhibit gene and/or protein expression of one or more Factor H family proteins selected from FHR1, FHR2, FHR3, FHR4, and/or FHR5, but does not inhibit gene and/or protein expression of FH and/or FHL-1. In some cases, an agent, e.g. inhibitory nucleic acid described herein inhibits gene and/or protein expression of each of FHR1, FHR2, FHR3, FHR4, and FHR5, but does not inhibit gene and/or protein expression of FH and/or FHL-1.

In some embodiments an agent, e.g. inhibitory nucleic acid described herein inhibits gene and/or protein expression of one or more Factor H family proteins (e.g. FHR2, FHR3, FHR4, FHR5, FH and/or FHL-1) but does not inhibit gene and/or protein expression of FHR1. In some embodiments an agent, e.g. inhibitory nucleic acid described herein inhibits gene and/or protein expression of one or more Factor H family proteins (e.g. FHR1, FHR3, FHR4, FHR5, FH and/or FHL-1) but does not inhibit gene and/or protein expression of FHR2. In some embodiments an agent, e.g. inhibitory nucleic acid described herein inhibits gene and/or protein expression of one or more Factor H family proteins (e.g. FHR1, FHR2, FHR4, FHR5, FH and/or FHL-1) but does not inhibit gene and/or protein expression of FHR3. In some embodiments an agent, e.g. inhibitory nucleic acid described herein inhibits gene and/or protein expression of one or more Factor H family proteins (e.g. FHR1, FHR2, FHR3, FHR5, FH and/or FHL-1) but does not inhibit gene and/or protein expression of FHR4. In some embodiments an agent, e.g. inhibitory nucleic acid described herein inhibits gene and/or protein expression of one or more Factor H family proteins (e.g. FHR1, FHR2, FHR3, FHR4, FH and/or FHL-1) but does not inhibit gene and/or protein expression of FHR5.

In some embodiments an agent, e.g. inhibitory nucleic acid described herein inhibits gene and/or protein expression of one or more Factor H family proteins (e.g. FHR2, FHR3, FHR4, FHR5, FH and/or FHL-1) but does not inhibit gene and/or protein expression of FHR1 or FHR3. In some embodiments an agent, e.g. inhibitory nucleic acid described herein inhibits gene and/or protein expression of one or more Factor H family proteins (e.g. FHR2, FHR3, FHR4, FHR5, FH and/or FHL-1) but does not inhibit gene and/or protein expression of FHR3 or FH.

In some embodiments, the target nucleotide sequence is not a nucleotide sequence of SEQ ID NO:221, which is a mRNA sequence encoding FHR4 (with thymine (T) replacing uracil (U)).

In some embodiments, the target nucleotide sequence is not a nucleotide sequence of SEQ ID NO:218, which is a mRNA sequence encoding FHR1 (with thymine (T) replacing uracil (U)). In some embodiments, the target nucleotide sequence is not a nucleotide sequence of SEQ ID NO:219, which is a mRNA sequence encoding FHR2 (with thymine (T) replacing uracil (U)). In some embodiments, the target nucleotide sequence is not a nucleotide sequence of SEQ ID NO:220, which is a mRNA sequence encoding FHR3 (with thymine (T) replacing uracil (U)). In some embodiments, the target nucleotide sequence is not a nucleotide sequence of SEQ ID NO:222, which is a mRNA sequence encoding FHR5 (with thymine (T) replacing uracil (U)). In some embodiments, the target nucleotide sequence is not a nucleotide sequence of SEQ ID NO:223, which is a mRNA sequence encoding FH (with thymine (T) replacing uracil (U)).

In some embodiments an agent, e.g. inhibitory nucleic acid described herein does not possess substantial sequence identity to a sequence disclosed in any one of WO 2007/144621, WO 2019/051443, WO 2006/088950, or WO 2012/112955, which are hereby incorporated by reference in their entirety.

In some embodiments an agent, e.g. inhibitory nucleic acid described herein comprises or consists of a sequence having less than 97%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, or less than 20% sequence identity to a sequence disclosed in any one of WO 2007/144621, WO 2019/051443, WO 2006/088950, or WO 2012/112955 (e.g. SEQ ID NO:3, 4 or 5 disclosed in WO 2007/144621, or SEQ ID NO: 1, 2, 16, 17 or 18 disclosed in WO 2019/051443).

In some embodiments, an inhibitory nucleic acid is selected from: an siRNA, miRNA, shRNA, pri-miRNA, pre-miRNA, saRNA, snoRNA, or antisense oligonucleotide (e.g. a gapmer), or a nucleic acid encoding the same. In some embodiments, an inhibitory nucleic acid is selected from: an siRNA, miRNA, shRNA. In some embodiments, an inhibitory nucleic acid is an siRNA.

In some embodiments, an inhibitory nucleic acid may comprise an antisense nucleic acid described herein, e.g. as part of a larger nucleic acid species. For example, in some embodiments, an inhibitory nucleic acid may be an siRNA, miRNA, shRNA, pri-miRNA, pre-miRNA, saRNA or snoRNA comprising an antisense nucleic acid described herein.

In some embodiments, an inhibitory nucleic acid is a small interfering RNA (siRNA). As used herein, ‘siRNA’ refers to double-stranded RNA molecule having a length between 17 to 30 (e.g. 20 to 27) base pairs, which is capable of engaging the RNA interference (RNAi) pathway for the targeted degradation of target RNA. Double-stranded siRNA molecules may be formed as a nucleic acid complex of RNA strands having high complementarity. In some embodiments siRNA molecules comprise symmetric 3′ overhangs, e.g. comprising one or two nucleotides (e.g. a ‘UU’ 3′ overhang). The strand of the siRNA molecule having complementarity to a target nucleotide sequence (i.e. the antisense nucleic acid) is referred to as the guide strand, and the other strand is referred to as the passenger strand. The structure and function of siRNAs is described e.g. in Kim and Rossi, Biotechniques. 2008 April; 44(5): 613-616. In some embodiments, an inhibitory nucleic acid comprises the guide and passenger strands of an siRNA.

In some embodiments, an inhibitory nucleic acid of the present disclosure comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to a SEQ ID NO in Column A of Table 12, and a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to a SEQ ID NO in Column B of Table 12. In some cases, the sequence from column A and sequence from column B are provided in the same row of Table 12.

In some embodiments, the guide strand of an siRNA according to the present disclosure may comprise or consist of an antisense nucleic acid according to an embodiment of an antisense nucleic acid described herein (e.g. a sequence having at least 70% sequence identity to one or more of SEQ ID NO: 178 to 197).

In some embodiments, an inhibitory nucleic acid is a microRNA (miRNA), or a precursor thereof (e.g. a pri-miRNA or a pre-miRNA). miRNA molecules have a similar structure, but are encoded endogenously, and derived from processing of short hairpin RNA molecules. They are initially expressed as long primary transcripts (pri-miRNAs), which are processed within the nucleus into 60 to 70 nucleotide hairpins (pre-miRNAs), which are further processed in the cytoplasm into small double stranded nucleic acids that interact with RISC and target mRNA. miRNAs comprise “seed sequences” that are essential for binding to target mRNA. “Seed sequences” usually comprise six nucleotides and are situated at positions 2 to 7 at the miRNA 5′ end.

In some embodiments, an inhibitory nucleic acid is a short hairpin RNA (shRNA). shRNA molecules comprise sequences of nucleotides having a high degree of complementarity that associate with one another through complementary base pairing to form a stem region in the hairpin. The sequences of nucleotides having a high degree of complementarity may be linked by one or more nucleotides that form a loop region in the hairpin. shRNA molecules may be processed (e.g. via catalytic cleavage by DICER) to form siRNA or miRNA molecules. shRNA may have a length of between 35 to 100 (e.g. 40 to 70) nucleotides. The stem region of the hairpin may a length between 17 to 30 (e.g. 20 to 27) base pairs. The stem region may comprise G-U pairings to stabilise the hairpin structure.

In some embodiments, an inhibitory nucleic acid is a dicer small interfering RNA (dsiRNA). As used herein, ‘dsiRNA’ refers to a double-stranded RNA molecule having a length of ˜27 base pairs, which is processed by Dicer to siRNA for RNAi-mediated degradation of target RNA. DsiRNAs are described e.g. in Raja et al., Asian J Pharm Sci. (2019) 14(5): 497-510, which is hereby incorporated by reference in their entirety. DsiRNAs are optimised for Dicer processing and may have increased potency compared with 21-mer siRNAs (see e.g. Kim et al., Nat Biotechnol. (2005) 23(2):222-226), which may be related to the link between Dicer-mediated nuclease activity and RISC loading.

siRNA, dsiRNAs, miRNAs and shRNAs for the targeted inhibition of gene and/or protein expression of one or more given target gene(s)/protein(s) may be identified/designed in accordance with principles and/or using tools well known to the skilled person. Parameters and tools for designing siRNA and shRNA molecules are described e.g. in Fakhr et al., Cancer Gene Therapy (2016) 23:73-82 (hereby incorporated by reference in its entirety). Software that may be used by the skilled person for the design of such molecules is summarised in Table 1 of Fakhr et al., Cancer Gene Therapy (2016) 23:73-82, and includes e.g. siRNA Wizard (InvivoGen). Details for making such molecules can be found in the websites of commercial vendors such as Ambion, Dharmacon, GenScript, Invitrogen and OligoEngine.

In some embodiments, an inhibitory nucleic acid is an antisense oligonucleotide (ASO). ASOs are single-stranded nucleic acid molecules comprising or consisting of an antisense nucleic acid to a target nucleotide sequence. An antisense oligonucleotide according to the present disclosure may comprise or consist of an antisense nucleic acid as described herein. ASOs can modify expression of RNA molecules comprising their target nucleotide sequence by altering splicing, or by recruiting RNase H to degrade the RNA comprising the target nucleotide sequence. RNase H recognises nucleic acid complex molecules formed when the ASO binds to RNA comprising its target nucleotide sequence. ASOs according to the present disclosure may comprise or consist of an antisense nucleic acid according to the present disclosure. ASOs may comprise 17 to 30 nucleotides in length. Many ASOs are designed as chimeras, comprising a mix of bases with different chemistries, or as gapmers, comprising a central DNA portion surrounded by ‘wings’ of modified bases. ASOs are described in e.g. Scoles et al., Neurol Genet. 2019 April; 5(2): e323. ASOs sometimes comprise alterations to the sugar-phosphate backbone in order to reduce/prevent RNAse H degradation, such as e.g. phosphorothioate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′O-methyl (2′OMe) and 2′-O-methoxyethyl (MOE) ribose modifications and/or 5′-methylcytosine modifications.

Inhibitory nucleic acids according to the present disclosure may comprise chemically modified nucleotide acids, e.g. in which the phosphonate and/or ribose and/or base is/are chemically modified. Such modifications may influence the activity, specificity and/or stability of nucleic acid. One or more (e.g. one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or all) nucleotides of an inhibitory nucleic acid may comprise such chemical modification.

Modifications contemplated in accordance with inhibitory nucleic acids according to the present disclosure include those described in Hu et al., Sig. Transduc. Tar. Ther. (2020) 5(101) (incorporated by reference hereinabove), in particular those shown in FIG. 2 of Hu et al., Sig. Transduc. Tar. Ther. (2020) 5(101). Further modifications contemplated in accordance with inhibitory nucleic acids according to the present disclosure include those described in Selvam et al., Chem Biol Drug Des. (2017) 90(5): 665-678, which is hereby incorporated by reference in its entirety).

In some embodiments, an inhibitory nucleic acid according to the present disclosure comprises a phosphonate modification selected from: phosphorothioate (e.g. Rp isomer, Sp isomer), phosphorodithioate, methylphosphonate, methoxypropylphosphonate, 5′-(E)-vinylphosphonate, 5′-methyl phosphonate, (S)-5′-C-methyl with phosphate, 5′-phosphorothioate, and peptide nucleic acid.

In some embodiments, an inhibitory nucleic acid according to the present disclosure comprises a ribose modification selected from: 2′-O-methyl, 2′-O-methoxyethyl, 2′-fluoro, 2′-deoxy-2′-fluoro, 2′-methoxyethyl, 2′-O-alkyl, 2′-O-allyl, 2′-C-allyl, 2′-deoxy, 2′-hydroxyl, 2′-arabino-fluoro, 2′-O-benzyl, 2′-O-methyl-4-pyridine, locked nucleic acid, (S)-cEt-BNA, tricyclo-DNA, PMO, unlocked nucleic acid, hexitol nucleic acid, and glycol nucleic acid.

In some embodiments, an inhibitory nucleic acid according to the present disclosure comprises a base modification selected from: pseudouridine, 2′-thiouridine, N6′-methyladenosine, 5′-methylcytidine, 5′-fluoro-2′-deoxyuridine, N-ethylpiperidine 7′-EAA triazole-modified adenine, N-ethylpiperidine 6′-triazole-modified adenine, 6′-phenylpyrrolo-cytosine, 2′,4′-difluorotoluyl ribonucleoside and 5′-nitroindole.

In some embodiments, an inhibitory nucleic acid according to the present disclosure comprises modification to incorporate a moiety facilitating delivery to, and/or uptake by, a cell type or tissue of interest. Modifications to nucleic acids to facilitate targeted delivery to cell types and/or tissues of interest are described e.g. in Lorenzer et al., J Control Release (2015) 203:1-15, which is hereby incorporated by reference in its entirety.

In some embodiments, an inhibitory nucleic acid according to the present disclosure comprises modification to incorporate a moiety facilitating delivery to, and/or uptake by, a liver cell or hepatic tissue.

In some embodiments, an inhibitory nucleic acid is conjugated to N-acetylgalactosamine (GalNAc). GalNAc interacts with the asialoglycoprotein receptor (ASGPR) expressed by hepatocytes. Nucleic acids conjugated to GalNAc are efficiently internalised by hepatic cells via receptor-mediated endocytosis following binding of GalNAc to ASGPR (see e.g. Nair et al., J. Am. Chem. Soc. (2014) 136(49): 16958-16961). In some embodiments, an inhibitory nucleic acid is conjugated to one or more (e.g. 1, 2, 3, 4 or more) GalNAc moieties. In some embodiments, one or more GalNAc moieties may be covalently associated to the 5′ or 3′ end of a strand of an inhibitory nucleic acid.

In some embodiments, an inhibitory nucleic acid is conjugated to α-tocopherol (i.e. vitamin E). Nucleic acid-α-tocopherol conjugates have been employed for targeted delivery of inhibitory nucleic acid to the liver (see e.g. Nishina et al., Mol Ther. (2008) 16(4):734-740). In some embodiments, an inhibitory nucleic acid is conjugated to one or more (e.g. 1, 2, 3, 4 or more) α-tocopherol moieties. In some embodiments, one or more α-tocopherol moieties may be covalently associated to the 5′ or 3′ end of a strand of an inhibitory nucleic acid.

In embodiments wherein inhibitory nucleic acids comprise nucleotides comprising chemical modification as described herein, the nucleotide sequence is nevertheless evaluated for the purposes of sequence comparison in accordance with the present disclosure as if the equivalent unmodified nucleotide were instead present. Nucleic acids comprising nucleotides comprising modified phosphate groups are evaluated for the purposes of nucleotide sequence comparison as if the nucleic acid only comprises nucleotides comprising unmodified phosphate groups. Nucleic acids comprising nucleotides comprising modified ribose groups are evaluated for the purposes of nucleotide sequence comparison as if the nucleic acid only comprises nucleotides comprising unmodified ribose groups.

Nucleic acids comprising nucleotides comprising modified base groups are evaluated for the purposes of nucleotide sequence comparison as if the respective modified bases were unmodified. By way of illustration, nucleic acids comprising nucleotides comprising pseudouridine, 2-thiouridine and/or 5′-fluoro-2′-deoxyuridine are evaluated for the purposes of nucleotide sequence comparison as if nucleotides comprising uridine were instead present at their respective positions. By way of illustration, nucleic acids comprising nucleotides comprising N6′-methyladenosine, N-ethylpiperidine 7′-EAA triazole-modified adenine and/or N-ethylpiperidine 6′-triazole-modified adenine are evaluated for the purposes of nucleotide sequence comparison as if nucleotides comprising adenine were instead present at their respective positions. By way of illustration, nucleic acids comprising nucleotides comprising 5′-methylcytidine and/or 6′-phenylpyrrolo-cytosine are evaluated for the purposes of nucleotide sequence comparison as if nucleotides comprising cytosine were instead present at their respective positions.

Inhibitory nucleic acids may be made recombinantly by transcription of a nucleic acid sequence, e.g. contained within vector. Transcription may be performed in cell-free transcription reactions, or in a cell comprising nucleic acid encoding the inhibitory nucleic acid. In some embodiments inhibitory nucleic acids are produced within a cell, e.g. by transcription from a vector. Vectors encoding such molecules may be introduced into cells in any of the ways known in the art. Optionally, expression of the nucleic acid can be regulated using a cell- (e.g. a liver cell) specific promoter.

For example, shRNA molecules may be produced within a cell by transcription from a vector. shRNAs may be produced within a cell by transfecting the cell with a vector encoding the shRNA sequence under control of an RNA polymerase promoter.

Inhibitory nucleic acids may also be synthesised using standard solid or solution phase synthesis techniques which are well known in the art. Solid phase synthesis may use phosphoramidite chemistry. Briefly, a solid supported nucleotide may be detritylated, then coupled with a suitably activated nucleoside phosphoramidite to form a phosphite triester linkage. Capping may then occur, followed by oxidation of the phosphite triester with an oxidant, typically iodine. The cycle may then be repeated to yield a polynucleotide.

### Genome Editing Tools and Related Articles

Aspects and embodiments of the present disclosure relate to agents that are, or comprise, well-known gene editing tools or systems. As used herein, an ‘genome/gene editing tool’ or ‘genome/gene editing system’ refers to a tool or system capable of reducing or preventing the gene and/or protein expression of one or more given target gene(s)/protein(s).

A genome editing tool/system may comprise a nuclease, i.e. a polypeptide possessing nuclease activity. Nucleases are reviewed e.g. in Yang, Q Rev Biophys. 2011 February; 44(1):1-93, which is hereby incorporated by reference in its entirety. Nucleases are broadly divided into endonucleases and exonucleases, depending on the region of the target nucleic acid on which they act; endonucleases act on regions within target nucleic acids, whereas exonucleases digest nucleic acids from one or both of the 5′ and 3′ ends of target nucleic acids.

Target sequences for a gene editing tool/system are described hereinabove. Nucleases described herein may act on DNA, RNA, or both DNA and RNA. Nucleases that act on DNA may be said to have deoxyribonuclease (DNase) activity. Such nucleases may be referred to as DNAses. Nucleases that act on RNA may be said to have ribonuclease (RNase) activity. Such nucleases may be referred to as RNAses.

A target sequence for an agent described herein may comprise all or part of SEQ ID NO: 218, 219, 220, 221, 222 and/or 223.

Nucleases described herein may be capable of cleaving a single-stranded or double-stranded nucleic acid substrates, or both. Nucleases that act on single-stranded nucleic acid molecules may be said to have single-stranded nuclease activity. Such nucleases may be referred to as single-stranded nucleases. Nucleases that act on double-stranded nucleic acid molecules may be said to have double-stranded nuclease activity. Such nucleases may be referred to as double-stranded nucleases.

Some nucleases act on their substrate nucleic acid in a non-specific fashion. Some nucleases are site-specific, having their nuclease activity targeted to a particular region or regions of substrate nucleic acid based on the recognition of a given structure(s) (structure-specific) or nucleic acid sequence(s) (sequence-specific). Some nucleases can be targeted to particular structure(s)/sequence(s) of substrate nucleic acid. Such nucleases may be referred to as being ‘targetable’, ‘programmable’, or ‘site-specific nucleases’ (SSNs). Targetable nucleases are reviewed e.g. in Carroll, Annu Rev Biochem. (2014) 83:409-39, which is hereby incorporated by reference in its entirety.

Guided nucleases employ guide nucleic acid molecules to direct the guided nuclease to particular structures formed by, and/or sequences of, substrate nucleic acid. The guide nucleic acid may target the nuclease to a particular region or regions of substrate nucleic acid through complementary base-pairing between nucleotides of the guide nucleic acid and nucleotides of the substrate nucleic acid. Nucleic acid-guided nucleases may use RNA guides (RNA-guided nucleases), DNA (DNA-guided nucleases), or both RNA and DNA guides.

Gene editing using site-specific nucleases (SSNs) is reviewed e.g. in Eid and Mahfouz, Exp Mol Med. 2016 October, 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively, DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a Fokl endonuclease domain). The DNA-binding domain may be identified by screening a Zinc Finger array capable of binding to the target nucleic acid sequence.

TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a Fokl endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C, ‘NI’ binds to A, ‘NG’ binds to T and ‘NN’ or ‘NK’ binds to G (Moscou and Bogdanove, Science (2009) 326(5959):1501.).

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

Other targeted approaches for genome engineering, such as meganucleases, are reviewed in Silva et al., Curr Gene Ther. 2011 February; 11(1): 11-27, which is hereby incorporated by reference in its entirety.

In some embodiments, a genome editing system for reducing expression of one or more Factor H family genes/proteins according to the present disclosure is selected from: a ZFN system, a TALEN system, a CRISPR/Cas system, a CRISPR/Cas9 system, a CRISPR/Cpf1 system, a CRISPR/C2c1 system, a CRISPR/C2c2 system, a CRISPR/C2c3 system, or a meganuclease.

In some embodiments, a CRISPR/Cas system for use as an agent described herein comprises a Cas nuclease, a crispr RNA (crRNA or guide RNA (gRNA)) and a trans-activating crRNA (trRNA or tracrRNA). In this system, the crRNA comprises a sequence complementary to the target DNA and serves to direct the Cas nuclease to the target site in the genome and the tracrRNA serves as a binding scaffold for the Cas nuclease which is required for Cas activity. In some embodiments, the CRISPR/Cas system comprises a guide RNA (gRNA).

In some embodiments, a CRISPR/Cas genome editing system described herein comprises a Cas nuclease and a single-guide RNA (sgRNA) to direct the Cas nuclease to the target site in the target gene. An sgRNA comprises a target-specific crRNA fused to a scaffold tracrRNA in a single nucleic acid.

In some embodiments a crRNA, gRNA, or sgRNA described herein comprises or consists of a sequence according to SEQ ID NO: 224 to SEQ ID NO: 227. In some embodiments a crRNA, gRNA, or sgRNA described herein comprises or consists of a sequence having at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 224 to SEQ ID NO: 227.

In some embodiments, a crRNA, gRNA, or sgRNA described herein comprises or consists of a sequence having at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 224 and reduces or prevents gene expression of one or more of FHR1, FHR2, FHR3, FHR4 and/or FHR5, e.g. when used in a CRISPR/Cas system. In some cases, said CRISPR/Cas system does not reduce or prevent gene expression of FH and/or FHL-1.

In some embodiments, a crRNA, gRNA, or sgRNA described herein comprises or consists of a sequence having at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 225, 226 or 227, and reduces or prevents gene expression of one or more of FHR1, FHR2, FHR3, FHR4 and/or FHR5, e.g. when used in a CRISPR/Cas system. In some cases, said CRISPR/Cas system reduces expression of FHR1, FHR2 and FHR5. In some cases, said CRISPR/Cas system does not reduce or prevent gene expression of FH and/or FHL-1.

Design of suitable guide RNAs for use in a CRISPR/Cas system are well within the routine practice of a skilled person using publicly-available software platforms e.g. from Harvard University (http://chopchop.cbu.uib.no/), Synthego (https://design.synthego.com/#/), or Integrated DNA Technologies (https://eu.idtdna.com/site/order/designtool/index/CRISPR_PREDESIGN).

CRISPR/Cas systems have been adapted for use in gene silencing. For example, in some embodiments, the nucleic acid may encode a fusion protein comprising a Cas protein or peptide fused to a transcriptional repressor. In some embodiments, the Cas protein is catalytically dead. The fusion protein may be directed to a site of interest in the genome by either an sgRNA or a crRNA. On binding of the fusion protein to the site of interest, the transcriptional repressor can reduce the expression of a gene of interest.

The CRISPR/Cas system may also be used to introduce a targeted modification into a target nucleic acid sequence, e.g. genomic DNA, for example using base editing technology or prime editing technology. Base editing and base editors (i.e. polypeptides possessing ‘base editing’ activity) are described e.g. in Rees and Liu Nat Rev Genet (2018) 19(12): 770-788 (revised in accordance with Rees and Liu, Nat Rev Genet (2018) 19:801), which are hereby incorporated by reference in their entirety.

This can be achieved using Cas proteins fused to a base editor, such as an adenine base editor or a cytidine base editor, e.g. cytidine deaminase, as disclosed in, for example, WO2017070633A2 which is hereby incorporated by reference in its entirety. Cytidine deaminases include e.g. CDA, APOBEC1, APOBEC1 (W90Y/R126E), APOBEC1 (W90Y/R132E), APOBEC1 (R126E/R132E), APOBEC1 (W90Y/R126E/R132E), CDA1, APOBEC3A, APOBEC3A (N37G), AID, AID (P182X) and AIDΔ. Adenine base editors include e.g. adenosine deaminases, which catalyse conversion of adenosine to inosine. Adenosine deaminases include e.g. TadA, and variants thereof.

For example, the CRISPR/Cas system may be a Prime editing system. In such a prime editing system, a fusion protein may be used. For example, the fusion protein may comprise a catalytically impaired Cas domain (e.g. a “nickase”) and a reverse transcriptase. The catalytically impaired Cas domain may be capable of cutting a single strand of DNA to produce a nicked DNA duplex. A Prime editing system may include a prime editing guide RNA (pegRNA) which includes an extended sgRNA comprising a primer binding site and a reverse transcriptase template sequence. Upon nicking of the DNA duplex by the catalytically impaired Cas, the primer binding site allows the 3′ end of the nicked DNA strand to hybridize to the pegRNA, while the RT template serves as a template for the synthesis of edited genetic information.

Articles that Encode and/or Deliver Agents of the Invention

The present disclosure provides nucleic acid comprising or encoding an agent according to the present disclosure, e.g. encoding an inhibitory nucleic acid or at least part of a gene editing system/complex. In some embodiments, nucleic acid comprising or encoding an agent comprises, or consists of, DNA and/or RNA.

The nucleic acid may comprise a DNA or an mRNA sequence encoding a Cas protein or peptide, for example a Cas9 protein or peptide. In some embodiments, the nucleic acid comprises an sgRNA (e.g. as described herein). In some embodiments, the nucleic acid comprises a crRNA (gRNA) and/or a tracrRNA. In some embodiments, the nucleic acid comprises a DNA or mRNA encoding a Cas protein or peptide, a crRNA and a tracrRNA. In some embodiments, the nucleic acid comprises a DNA or mRNA encoding a Cas protein or peptide and a crRNA (gRNA) and/or tracrRNA. In some embodiments, the nucleic acid comprises a DNA or mRNA encoding a Cas protein or peptide and a sgRNA.

The present disclosure also provides a vector comprising the nucleic acid comprising or encoding an agent according to the present disclosure.

Nucleic acids and vectors according to the present disclosure may be provided in purified or isolated form, i.e. from other nucleic acid, or naturally-occurring biological material.

The nucleotide sequence of a nucleic acid comprising or encoding an agent according to the present disclosure may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. A vector may include regulatory elements, such as a polyadenylation site. Any suitable vectors, promoters, enhancers and termination codons known in the art may be used to express nucleic acid from a vector according to the present disclosure.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence.

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus (AAV) vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety. In some embodiments, the lentiviral vector may be pELNS, or may be derived from pELNS. In some embodiments, the vector may be a vector encoding CRISPR/Cas9. In some embodiments, the adeno-associated virus (AAV) vector is selected from AAV serotype 1, 2, 3, 4, 5, 6, 7, 8, 9, 10 or 11, or hybrids and/or mutants thereof. In some embodiments, the AAV vector is an AAV serotype 2 (AAV-2) vector, or a hybrid and/or mutant thereof.

Viral and non-viral delivery systems for introducing genetic material into cells are reviewed, for example, in Nayerossadat et al., Adv Biomed Res. 2012; 1: 27; MacLaren et al. Ophthalmology. 2016, 123(10 Suppl): S98-S106; Petit and Punzo, Discov Med. 2016, 22(121): 221-229; Aguirre, Invest Ophthalmol Vis Sci. 2017, 58(12): 5399-5411; Lundstrom, Diseases. 2018, 6(2): 42; which are hereby incorporated by reference in their entirety. Any suitable nucleotide or vector delivery method can be used in the context of the present invention.

In some embodiments, the vector may be a eukaryotic vector, e.g. a vector comprising the elements necessary for expression of nucleic acid from the vector in a eukaryotic cell. In some embodiments, the vector may be a mammalian vector, e.g. comprising a cytomegalovirus (CMV) or SV40 promoter to drive expression.

The present disclosure also provides a plurality of agents, e.g. inhibitory nucleic acids or components of a gene editing system/complex, according to the present disclosure. The present disclosure also provides nucleic acids and vectors comprising or encoding a plurality of such agents according to the present disclosure.

Individual agents of a plurality of agents according to the present disclosure may be identical or non-identical. Similarly, in embodiments wherein a nucleic acid/vector comprising or encoding an agent according to the present disclosure comprises/encodes more than one agent according to the present disclosure, the agent comprised/encoded by the nucleic acid/vector may be identical or non-identical.

In some embodiments, nucleic acids/vectors may encode one of 1, 2, 3, 4, 5, 6, 7, 8, 9, 10, 11, 12, 13, 14, 15, 16, 17, 18, 19 or 20 agents, e.g. inhibitory nucleic acids, according to the present disclosure. In some embodiments, nucleic acids/vectors may encode multiple (e.g. 2, 3, 4, 5, 6, 7, 8, 9, 10, etc.) copies of a given agent according to the present disclosure.

In some embodiments, a plurality of agents according to the present disclosure may be a plurality of non-identical agents. In some embodiments, a plurality of agents may comprise one of 1, 2, 3, 4, 5, 6, 7, 8, 9, 10, 11, 12, 13, 14, 15, 16, 17, 18, 19 or 20 non-identical agents, e.g. inhibitory nucleic acids. In some embodiments, nucleic acids/vectors may comprise/encode a plurality of non-identical agents according to the present disclosure.

The following paragraphs further define pluralities of non-identical agents in accordance with embodiments of plurality of agents according to the present disclosure, and also in accordance with embodiments of nucleic acids/vectors comprising/encoding a plurality of non-identical agents according to the present disclosure.

In some embodiments, the non-identical agents may reduce gene and/or protein expression of non-identical Factor H family proteins (e.g. selected from FHR1, FHR2, FHR3, FHR4, FHR5, FH and/or FHL-1).

In some embodiments, the non-identical agents, e.g. inhibitory nucleic acids, may comprise or encode non-identical antisense nucleic acids. In such embodiments, the non-identical antisense nucleic acids may each independently conform to any embodiment of an antisense nucleic acid as described hereinabove.

In some embodiments, the non-identical agents, e.g. inhibitory nucleic acids, may comprise or encode antisense nucleic acids targeting non-identical target nucleotide sequences. In such embodiments, the non-identical target nucleotide sequences may each independently conform to any embodiment of a target nucleotide sequence for an antisense nucleic acid as described hereinabove.

In some embodiments, the target nucleotide sequences of the antisense nucleic acids comprised/encoded by the non-identical inhibitory nucleic acids may be of RNA encoded by genes encoding non-identical Factor H family proteins (e.g. selected from FHR1, FHR2, FHR3, FHR4, FHR5, FH and/or FHL-1). In some embodiments, the target nucleotide sequences may be of RNA encoding non-identical Factor H family proteins (e.g. selected from FHR1, FHR2, FHR3, FHR4, FHR5, FH and/or FHL-1). In some embodiments, the target nucleotide sequences comprise one or more nucleotides of exons of RNA encoding non-identical Factor H family proteins (e.g. selected from FHR1, FHR2, FHR3, FHR4, FHR5, FH and/or FHL-1). In some embodiments, the target nucleotide sequences are nucleotide sequences of exons of RNA encoding non-identical Factor H family proteins (e.g. selected from FHR1, FHR2, FHR3, FHR4, FHR5, FH and/or FHL-1).

It will be appreciated that in some embodiments it is desirable for the individual inhibitory nucleic acids of a plurality of non-identical inhibitory nucleic acids to be selected in order to provide for reducing gene and/or protein expression of more than one Factor H family protein (e.g. selected from FHR1, FHR2, FHR3, FHR4, FHR5, FH and/or FHL-1).

In some embodiments, a plurality of non-identical inhibitory nucleic acids for reducing gene and/or protein expression of more than one Factor H family protein comprises inhibitory nucleic acids for reducing gene and/or protein expression of one of the following combinations of Factor H family proteins: FH and/or FHL-1, FHR1, FHR2, FHR3, FHR4 and FHR5; FH and/or FHL-1, FHR1, FHR2, FHR3 and FHR4; FH and/or FHL-1, FHR1, FHR2, FHR3 and FHR5; FH and/or FHL-1, FHR1, FHR2, FHR4 and FHR5; FH and/or FHL-1, FHR1, FHR3, FHR4 and FHR5; FH and/or FHL-1, FHR2, FHR3, FHR4 and FHR5; FHR1, FHR2, FHR3, FHR4 and FHR5; FH and/or FHL-1, FHR1, FHR2 and FHR3; FH and/or FHL-1, FHR1, FHR2 and FHR4; FH and/or FHL-1, FHR1, FHR2 and FHR5; FH and/or FHL-1, FHR1, FHR3 and FHR4; FH and/or FHL-1, FHR1, FHR3 and FHR5; FH and/or FHL-1, FHR1, FHR4 and FHR5; FH and/or FHL-1, FHR2, FHR3 and FHR4; FH and/or FHL-1, FHR2, FHR3 and FHR5; FH and/or FHL-1, FHR2, FHR4 and FHR5; FH and/or FHL-1, FHR3, FHR4 and FHR5; FHR1, FHR2, FHR3 and FHR4; FHR1, FHR2, FHR3 and FHR5; FHR1, FHR2, FHR4 and FHR5; FHR1, FHR3, FHR4 and FHR5; FHR2, FHR3, FHR4 and FHR5; FH and/or FHL-1, FHR1 and FHR2; FH and/or FHL-1, FHR1 and FHR3; FH and/or FHL-1, FHR1 and FHR4; FH and/or FHL-1, FHR1 and FHR5; FH and/or FHL-1, FHR2 and FHR3; FH and/or FHL-1, FHR2 and FHR4; FH and/or FHL-1, FHR2 and FHR5; FH and/or FHL-1, FHR3 and FHR4; FH and/or FHL-1, FHR3 and FHR5; FH and/or FHL-1, FHR4 and FHR5; FHR1, FHR2 and FHR3; FHR1, FHR2 and FHR4; FHR1, FHR2 and FHR5; FHR1, FHR3 and FHR4; FHR1, FHR3 and FHR5; FH and/or FHL-1 and FHR1; FH and/or FHL-1 and FHR2; FH and/or FHL-1 and FHR3; FH and/or FHL-1 and FHR4; FH and/or FHL-1 and FHR5; FHR1 and FHR2; FHR1 and FHR3; FHR1 and FHR4; FHR1 and FHR5; FHR2 and FHR3; FHR2 and FHR4; FHR2 and FHR5; FHR3 and FHR4; FHR3 and FHR; or FHR4 and FHR5.

Appropriate combinations of agents, e.g. inhibitory nucleic acids, to be employed to reduce gene and/or protein expression of the combinations of Factor H family proteins described in the preceding paragraph will be immediately apparent to the skilled person in view of embodiments of agents, e.g. inhibitory nucleic acids, disclosed herein.

In some embodiments, an inhibitory nucleic acid according to the present disclosure is non-identical to a nucleic acid disclosed in WO 2019/215330 A1, which is hereby incorporated by reference in its entirety. In some embodiments, the nucleotide sequence of an inhibitory nucleic acid according to the present disclosure is non-identical to the nucleotide sequence of a nucleic acid disclosed in WO 2019/215330 A1.

The present disclosure also provides a cell comprising or expressing (i) an agent according to the present disclosure, (ii) nucleic acid comprising or encoding an agent according to the present disclosure, and/or (iii) a vector comprising nucleic acid comprising or encoding an agent according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell may be a human cell.

The present disclosure also provides a method for producing a cell comprising a nucleic acid or vector according to the present disclosure, comprising introducing a nucleic acid or vector according to the present disclosure into a cell. In some embodiments, introducing a nucleic acid or vector according to the present disclosure into a cell comprises transformation, transfection, electroporation or transduction (e.g. retroviral transduction).

The present disclosure also provides a method for producing an agent, e.g. inhibitory nucleic acid, according to the present disclosure or a nucleic acid comprising or encoding an agent, e.g. inhibitory nucleic acid, according to the present disclosure, comprising culturing a cell comprising nucleic acid comprising or encoding an agent according to the present disclosure or a vector according to the present disclosure under conditions suitable for expression of the nucleic acid or vector by the cell. In some embodiments, the methods are performed in vitro.

The present disclosure also provides compositions comprising agents and articles described herein (including inhibitory nucleic acids, gene editing tools/systems and nucleic acids for use therein, nucleic acids comprising/encoding such agents, expression vectors comprising/encoding such agents) or cells according to the present disclosure.

In therapeutic and prophylactic applications, the compositions of the present disclosure are preferably formulated as a medicament or pharmaceutical composition (suitable for clinical use). Such compositions may comprise the agent or cell together with one or more other pharmaceutically-acceptable ingredients well known to those skilled in the art. Such ingredients include, but are not limited to, pharmaceutically-acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation. Suitable carriers, adjuvants, excipients, etc. can be found in standard pharmaceutical texts, for example, Remington's Pharmaceutical Sciences, 20th Edition, 2000, pub. Lippincott, Williams & Wilkins, and Handbook of Pharmaceutical Excipients, 2nd edition, 1994.

Compositions according to the present disclosure may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The compositions may be prepared for topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal routes of administration which may include injection or infusion, or for administration as an eye drop (i.e. ophthalmic administration). Suitable formulations may comprise the selected agent in a sterile or isotonic medium. The formulation and mode of administration may be selected according to the agent to be administered, and disease to be treated/prevented.

The compositions of the present disclosure may be formulated in fluid, including gel, form. Fluid formulations may be formulated for administration by injection or infusion (e.g. via catheter) to a selected organ or region of the human or animal body. A further aspect of the present disclosure relates to a method of formulating or producing a medicament or pharmaceutical composition according to the present disclosure, the method comprising formulating a pharmaceutical composition or medicament by mixing an agent with a pharmaceutically acceptable carrier, adjuvant, excipient or diluent.

Agents and articles according to the present disclosure (e.g. nucleic acids (including inhibitory nucleic acids, expression vectors), cells and compositions) may be formulated to facilitate delivery to, and/or uptake by, a cell/tissue, e.g. a target cell/tissue of interest. In some embodiments, the inhibitory nucleic acid employs a delivery platform described in Hu et al., Sig. Transduc. Tar. Ther. (2020) 5(101) (incorporated by reference hereinabove).

Agents and articles according to the present disclosure may be linked to a moiety in order to facilitate delivery to, and/or uptake, by a cell/tissue. Strategies for facilitating intracellular delivery of molecular cargo are reviewed e.g. in Li et al., Int. J. Mol. Sci. (2015) 16: 19518-19536 and Fu et al., Bioconjug Chem. (2014) 25(9): 1602-1608, which are hereby incorporated by reference in their entirety.

In some embodiments, agents and articles (e.g. nucleic acids) may be formulated with a cationic polymer. In some embodiments, agents and articles (e.g. nucleic acids) may be encapsulated in a nanoparticle or a liposome.

In some embodiments, agents and articles according to the present disclosure may be linked to a moiety or otherwise formulated to facilitate delivery to, and/or uptake by, a liver cell or hepatic tissue.

In some embodiments, a nucleic acid according to the present disclosure may be conjugated to N-acetylgalactosamine (GalNAc). In some embodiments, a nucleic acid is conjugated to one or more (e.g. 1, 2, 3, 4 or more) GalNAc residues. In some embodiments, a nucleic acid is conjugated to one or more (e.g. 1, 2, 3, 4 or more) GalNAc moieties. In some embodiments, one or more GalNAc moieties may be covalently associated to the 5′ or 3′ end of a strand of a nucleic acid.

In some embodiments, a nucleic acid according to the present disclosure is conjugated to α-tocopherol (i.e. vitamin E). In some embodiments, a nucleic acid is conjugated to one or more (e.g. 1, 2, 3, 4 or more) α-tocopherol moieties. In some embodiments, one or more α-tocopherol moieties may be covalently associated to the 5′ or 3′ end of a strand of a nucleic acid.

In some embodiments, a nanoparticle is a nanoparticle described in Chen et al., Mol Ther Methods Clin Dev. (2016) 3:16023, which is hereby incorporated by reference in its entirety. In some embodiments, a nanoparticle is a PLGA, polypeptide, poly(β-amino ester), DOPE, β-cyclodextrin-containing polycation, linear PEI, PAMAM dendrimer, branched PEI, chitosan or polyphosophoester nanoparticle.

In some embodiments, articles (e.g. nucleic acids) may be (covalently or non-covalently) associated with a cell-penetrating peptide (e.g. a protein transduction domain, trojan peptide, arginine-rich peptide, vectocell peptide), a cationic polymer, a cationic lipid or a viral carrier. In some embodiments, articles (e.g. nucleic acids) may be associated with a peptide/polypeptide (e.g. antibody, peptide aptamer, ligand for a cell surface molecule/fragment thereof) or a nucleic acid (e.g. nucleic acid aptamer) capable of binding to a target cell of interest or an antigen thereof. Pharmaceutical agents, such as those described herein, may be administered using lipid-based drug delivery systems, e.g. as described in Kalepu et al., Acta Pharmaceutica Sinica B, 2013, 3, 6, 361-372 and Barba et al., Pharmaceutics. 2019 Jul. 24, 11(8):360, which are hereby incorporated by reference in their entirety.

Methods for nucleic acid delivery are known in the art and can be found, for example, in Tatiparti K et al. “siRNA Delivery Strategies: A Comprehensive Review of Recent Developments.” Ed. Thomas Nann. Nanomaterials 7.4 (2017): 77, and Lehto T et al., Adv Drug Deliv Rev. 2016, 106(Pt A):172-182, which are hereby incorporated by reference in their entirety. For example, nucleic acid may be delivered naked, or by using nanoparticles, polymers, peptides e.g. cell-penetrating peptides, or by ex vivo transfection. Nanoparticles may be organic, e.g. micelles, liposomes, proteins, solid-lipid particles, solid polymer particles, dendrimers, and polymer therapeutics. Nanoparticles may be inorganic such as nanotubes or metal particles, optionally with organic molecules added. Viruses present another nanoparticle delivery option. Nanoparticles may be optimised to improve rate of endocytosis, avoid renal clearance and filtration, improve thermal stability, improve pH stability, prevent toxic effects, and improve nucleic acid loading efficiency. Further encapsulation methods are described in e.g. US 2015/0157675 A1.

In some cases, nucleic acids, e.g. nanoparticle based formulations thereof, may be formulated for pulmonary administration for subsequent delivery to non-lung tissues, see e.g. US 2015/0157565 A1, which is hereby incorporated in its entirety.

### Therapeutic and Prophylactic Applications

The agents, inhibitory nucleic acids, gene editing tools/systems, nucleic acids, expression vectors and compositions described herein find use in therapeutic and prophylactic methods.

The present disclosure provides an agent, inhibitory nucleic acid, gene editing tool/system, nucleic acid, expression vector, or composition described herein for use as a medicament. The present disclosure provides an agent, inhibitory nucleic acid, gene editing tool/system, nucleic acid, expression vector, or composition described herein for use in a method of medical treatment or prophylaxis. Also provided is the use of an agent, inhibitory nucleic acid, gene editing tool/system, nucleic acid, expression vector, or composition described herein in the manufacture of a medicament for treating or preventing a disease or condition. Also provided is a method of treating or preventing a disease or condition, comprising administering to a subject a therapeutically or prophylactically effective amount of an agent, inhibitory nucleic acid, gene editing tool/system, nucleic acid, expression vector, or composition described herein.

The terms “disorder”, “disease” and “condition” may be used interchangeably and refer to a pathological issue of a body part, organ or system which may be characterised by an identifiable group of signs or symptoms.

“Treatment” may refer to treating, preventing, or reducing the likelihood of a complement-related disorder, such as those described herein. As used herein, ‘treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention/prophylaxis of a disease/condition may refer to prevention of a worsening of the condition or prevention of the development of the disease/condition, e.g. preventing an early stage disease/condition developing to a later, chronic, stage.

Treatment of a complement-related disorder as described herein may involve modifying at least one cell of a subject to express or comprise a nucleic acid provided herein. Treatment of a complement-related disorder as described herein may involve modifying at least one cell of a subject to express or comprise a polypeptide provided herein, e.g. via a nucleic acid provided herein. Treatment of a complement-related disorder as described herein may involve administering to a subject a vector comprising or consisting of a nucleic acid as described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition a later stage (e.g. a chronic stage or metastasis).

The terms “develop”, “developing”, and “development”, e.g. of a disorder, as used herein refer both to the onset of a disease as well as the progression, exacerbation or worsening of a disease state. The term “biomarker(s)” as used herein refers to one or more measurable indicators of a biological state or condition.

It will be appreciated that the articles, e.g. inhibitory nucleic acids, of the present disclosure may be used for the treatment/prevention of any disease/condition that would derive therapeutic or prophylactic benefit from a reduction in the level of gene and/or protein expression of the complement protein(s) targeted by the inhibitory nucleic acid (i.e. the complement protein(s) the inhibitory nucleic acid is suitable for reducing/preventing gene and/or protein expression of).

The disease/condition to be treated/prevented in accordance with the present disclosure may be a disease/condition in which the relevant complement protein(s) are pathologically implicated, e.g. a disease/condition in which an increased level of gene and/or protein expression of the relevant complement protein(s) is positively associated with the onset, development or progression of the disease/condition, and/or severity of one or more symptoms of the disease/condition, or for which an increased level of gene and/or protein expression of the relevant complement protein(s) is a risk factor for the onset, development or progression of the disease/condition.

In some embodiments, the disease/condition to be treated/prevented in accordance with the present disclosure is a disease/condition characterised by an increase in the level of gene and/or protein expression of the relevant complement protein(s), e.g. as compared to the level of gene and/or protein expression of the relevant complement protein(s) in the absence of the disease/condition. Treatment in accordance with the methods of the present disclosure may achieve a reduction in the level of gene and/or protein expression of the relevant complement protein(s) in the subject.

In aspects and embodiments of the present disclosure, the disease/condition to be treated is a complement-related disorder. As used herein, a “complement-related disorder” is a disorder, disease or condition that comprises, or arises from, deficiencies or abnormalities in the complement system. In some embodiments, the complement-related disorder is a disorder driven by complement activation or complement over-activation.

In any embodiment described herein the disorder is one in which the complement system, or activation/over-activation/dysregulation thereof, is pathologically-implicated. The complement related disorder may be any disorder described herein. “Pathologically-implicated” as used herein may refer to a protein level which is raised or lowered in the disorder compared with a reference value, and/or where the protein contributes towards the pathology of the disorder. The selection or combination of complement protein(s) detected/determined may depend on the complement-related disorder of interest and the complement protein(s) that are useful biomarkers for said disorder.

Complement-related disorders may comprise disruption of the classical, alternative and/or lectin complement pathways. In some cases, the disorder may be associated with deficiencies in, abnormalities in, or absence of regulatory components of the complement system. In some embodiments, the disorder may be a disorder associated with the alternative complement pathway, disruption of the alternative complement pathway and/or associated with deficiencies in, abnormalities in, or absence of regulatory components of the alternative complement pathway. In some cases, the disorder is associated with the complement amplification loop. In some cases, the disorder is associated with inappropriate activation, over-activation, or dysregulation of the complement system, in whole or in part, e.g. C3 convertase assembly, C3b production, C3b deposition, and/or the amplification loop.

In some cases, the disorder is associated with any one or more of C3, C3b, iC3b, FI, FH, FHL-1, or FHR1-FHR5. In some cases, the disorder is associated with deficiencies or abnormalities in the activity of any one or more of C3, C3b, iC3b, FI, FH, FHL-1, or FHR1-FHR5. In some cases one or more of these proteins are pathologically-implicated in the disorder, e.g. have raised or lower levels compared with a reference value.

In some cases, the disorder is associated with one or more of CR1, CD46, CD55, C4BP, Factor B (FB), Factor D (FD), SPICE, VCP (or VICE) and/or MOPICE. In some cases, the disorder is associated with deficiencies or abnormalities in the activity of one or more of CR1, CD46, CD55, C4BP, Factor B, Factor D, SPICE, VCP (or VICE) and/or MOPICE, or where one or more of these proteins are pathologically implicated.

In some embodiments, the disorder may be a disorder associated with C3 or a C3-containing complex, an activity/response associated with C3 or a C3-containing complex, or a product of an activity/response associated with C3 or a C3-containing complex. That is, in some embodiments, the disorder is a disorder in which C3, a C3-containing complex, an activity/response associated with C3 or a C3-containing complex, or the product of said activity/response is pathologically implicated. In some embodiments, the disorder may be associated with an increased level of C3 or a C3-containing complex, an increased level of an activity/response associated with C3 or a C3-containing complex, or an increased level of a product of an activity/response associated with C3 or a C3-containing complex as compared to the control state. In some embodiments, the disorder may be associated with a decreased level of C3 or a C3-containing complex, a decreased level of an activity/response associated with C3 or a C3-containing complex, or a decreased level of a product of an activity/response associated with C3 or a C3-containing complex as compared to the control state.

In some embodiments, the disorder may be a disorder associated with C3b or a C3b-containing complex, an activity/response associated with C3b or a C3b-containing complex, or a product of an activity/response associated with C3b or a C3b-containing complex. That is, in some embodiments, the disorder is a disorder in which C3b, a C3b-containing complex, an activity/response associated with C3b or a C3b-containing complex, or the product of said activity/response is pathologically implicated. In some embodiments, the disorder may be associated with an increased level of C3b or a C3b-containing complex, an increased level of an activity/response associated with C3b or a C3b-containing complex, or increased level of a product of an activity/response associated with C3b or a C3b-containing complex as compared to the control state. In some embodiments, the disorder may be associated with a decreased level of C3b or a C3b-containing complex, a decreased level of an activity/response associated with C3b or a C3b-containing complex, or a decreased level of a product of an activity/response associated with C3b or a C3b-containing complex as compared to the control state.

In some embodiments, the disorder may be a disorder associated with any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46 or a product of an activity/response associated with any one or more of FH, FHL-1, FI, FHR1-FHR5, FB, FD, CR1 and/or CD46. In some embodiments, the disorder is a disorder in which any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46, or the product of said activity/response is pathologically implicated. In some embodiments, the disorder may be associated with a decreased level of any one or more of FH, FHL-1, FI, FHR1-FHR5, FB, FD, CR1 and/or CD46, a decreased level of an activity/response associated with any one or more of FH, FHL-1, FI, FHR1-FHR5, FB, FD, CR1 and/or CD46, or a decreased level of a product of an activity/response associated with any one or more of FH, FHL-1, FI, FHR1-FHR5, FB, FD, CR1 and/or CD46 as compared to a control state.

In some embodiments, the disorder may be associated with an increased level of any one or more of FHR1, FHR2, FHR3, FHR4 and/or FHR5, an increased level of an activity/response associated with any one or more of FHR1, FHR2, FHR3, FHR4 and/or FHR5, or an increased level of a product of an activity/response associated with any one or more of FHR1, FHR2, FHR3, FHR4 and/or FHR5 as compared to a control state, see e.g. Zhu et al., Kidney Int. 2018 July; 94(1):150-158; Pouw et al., Front Immunol. 2018 Apr. 24; 9:848; both hereby incorporated by reference in their entirety. The methods may comprise determining the systemic level of any combination of FHR1 to FHR5, e.g. using a technique described herein.

Subjects with elevated levels of FHR1, FHR2, FHR3, FHR4 and/or FHR5, and optionally FH/FHL-1, and/or increased expression of a gene(s) encoding one or more of said proteins, may derive therapeutic or prophylactic benefit from said levels being reduced. The level of expression of a Factor H family gene may be measured using techniques described herein and/or that are described in the art, as reviewed in, for example, Roth CM, Curr. Issues Mol. Biol. 2002 4:93-100 and Kukurba K R and Montgomery S B, Cold Spring Harb Protoc. 2015, (11):951-969, which are hereby incorporated by reference in their entirety. For example, gene expression can be measured using quantitative PCR, real-time PCT, sequencing techniques e.g. RNA-seq, next-generation sequencing, microarrays, Northern blot, and ribonuclease protection assay (RPA). One skilled in the art will be able to appreciate a suitable technique(s) for measuring expression of CFHR1-5, as required. In some cases, the total RNA or cDNA may be extracted and isolated first from a cell sample.

In particular embodiments, the disorder is characterised by elevated levels of any one or more FH family proteins, e.g. any one or more of FHR1, FHR2, FHR3, FHR4 and/or FHR5. The elevated levels may be in a subject. That is, the subject to be assessed or treated may have (or be/have been determined to have) elevated levels of one or more of FHR1, FHR2, FHR3, FHR4 and/or FHR5, e.g. assessed by a method provided herein. The disorder may be characterised by elevated circulating levels of one or more of FHR1, FHR2, FHR3, FHR4 and/or FHR5, i.e. in a blood- or plasma-derived sample as described herein. The disorder may be characterised by elevated expression of one or more of FHR1, FHR2, FHR3, FHR4 and/or FHR5 by hepatocytes. The disorder may be characterised by elevated levels of one or more of FHR1, FHR2, FHR3, FHR4 and/or FHR5 detected in a tissue of interest, e.g. eye, kidney, brain, lung, tumor, vascular tissue. Elevated levels can be determined by comparison to a control value(s)/subject(s) as described herein.

Not all subjects with a complement-related disorder may have elevated levels of one or more FHR proteins. Thus, some subjects with a complement-related disorder may have elevated levels of one or more FHR proteins, and some subjects with the same complement-related disorder may not. In some cases, e.g. as described herein, the presence of elevated levels of one or more FHR proteins can indicate a worse prognosis. Determining the levels of one or more FHR proteins therefore may provide a distinct population of patients who will benefit in particular from treatment with the nucleic acids and proteins described herein, e.g. as compared to patients with normal levels of FHR proteins.

The complement-related disorder may be characterised by altered levels of FH and/or FHL-1, either up or down, e.g. in addition to the elevated levels of one or more FHR proteins.

In some embodiments, the disorder may be associated with an increased level of any one or more of FHR1, FHR2, FHR3 and/or FHR5, an increased level of an activity/response associated with any one or more of FHR1, FHR2, FHR3 and/or FHR5, or an increased level of a product of an activity/response associated with any one or more of FHR1, FHR2, FHR3 and/or FHR5. In some embodiments the disorder may be associated with an increased level of FHR4, an increased level of an activity/response associated with FHR4, or an increased level of a product of an activity/response associated with FHR4 as compared to a control state, see e.g. WO 2019/215330 and Cipriani et al., Nat Commun 11, 778 (2020), both hereby incorporated by reference in their entirety. In some embodiments the disorder may be associated with an increased level of FHL-1. In some embodiments the disorder may not be associated with an increased level of FHR4, an increased level of an activity/response associated with FHR4, or an increased level of a product of an activity/response associated with FHR4 as compared to a control state.

In some embodiments the disorder is associated with increased levels of any one or more of C3, C3b, C3 convertase and/or C3bBb as compared to a control state. In some embodiments the disorder is associated with decreased levels of any one or more of C3, C3b, C3 convertase and/or C3bBb as compared to a control state. In some embodiments, the disorder is associated with increased levels of iC3b as compared to a control state. In some embodiments, the disorder is associated with decreased levels of iC3b as compared to a control state. In some embodiments the disorder is associated with increased levels of any one or more of C3a, C3f, C3c, C3dg, C3d, and/or C3g as compared to a control state. In some embodiments the disorder is associated with decreased levels of any one or more of C3a, C3f, C3c, C3dg, C3d, and/or C3g as compared to a control state.

In some cases, the methods described herein find use in treating or preventing a disorder which would benefit from one or more of: a reduction in the level or activity of one or more of C3bBb-type C3 convertase, C3bBb3b-type C5 convertase and/or C4b2a3b-type C5 convertase; a reduction in the level of one or more of C3, C3b, C3a, iC3b, FHR1, FHR2, FHR3, FHR4, FHR5, C5b and/or C5a; or an increase in the level of one or more of iC3b, C3f, C3c, C3dg, C3d, C3g, FH, FHL-1, FI, FH, FHL-1, FHR1, FHR2, FHR3, FHR4 and/or FHR5 as compared to reference value(s).

In some cases, the methods described herein find use in treating or preventing a disorder which would benefit from a reduction in the level or activity of one or more of FHR1, FHR2, FHR3, FHR4 and/or FHR5, and optionally FHL-1.

The disorder may be an ocular disorder. In some embodiments, a disease or condition to be treated or prevented as described herein is a complement-related ocular disease. In some embodiments, the disorder is macular degeneration. In some embodiments, the disorder may be selected from, i.e. is one or more of, age-related macular degeneration (AMD), choroidal neovascularisation (CNV), macular dystrophy, and diabetic maculopathy. As used herein, the term “AMD” includes early AMD, intermediate AMD, late/advanced AMD, geographic atrophy (‘dry’ (i.e. non-exudative) AMD), and ‘wet’ (i.e. exudative or neovascular) AMD, each of which may be a disorder in its own right that is detected, treated and/or prevented as described herein. In some embodiments the disease or condition to be treated or prevented is a combination of the diseases/conditions above, e.g. ‘dry’ and ‘wet’ AMD. In some embodiments the disease or condition to be treated or prevented is not ‘wet’ AMD or choroidal neovascularisation. AMD is commonly-defined as causing vision loss in subjects age 50 and older. In some embodiments a subject to be treated is age 50 or older, i.e. is at least 50 years old.

As used herein “early AMD” refers to a stage of AMD characterised by the presence of medium-sized drusen, commonly having a diameter of up to ˜200 μm, within Bruch's membrane adjacent to the RPE layer. Subjects with early AMD typically do not present with significant vision loss. As used herein “intermediate AMD” refers to a stage of AMD characterised by large drusen and/or pigment changes in the retina. Intermediate AMD may be accompanied by some vision loss. As used herein “late AMD” refers to a stage of AMD characterised by the presence of drusen and vision loss, e.g. severe central vision loss, due to damage to the macula. In all stages of AMD, ‘reticular pseudodrusen’ (RPD) or ‘reticular drusen’ (also referred to as subretinal drusenoid deposits (SDD)) may be present, referring to the accumulation of extracellular material in the subretinal space between the neurosensory retina and RPE. “Late AMD” encompasses ‘dry’ and ‘wet’ AMD. In ‘dry’ AMD (also known as geographic atrophy), there is a gradual breakdown of the light-sensitive cells in the macula that convey visual information to the brain and of the supporting tissue beneath the macula. In ‘wet’ AMD (also known as choroidal neovascularization, neovascular and exudative AMD), abnormal blood vessels grow underneath and into the retina. These vessels can leak fluid and blood which can lead to swelling and damage of the macula and subsequent scar formation. The damage may be rapid and severe.

In some embodiments the disorder is early-onset macular degeneration (EOMD). As used herein “EOMD” refers to a phenotypically severe sub-type of macular degeneration that demonstrates a much earlier age of onset than classical AMD and results in many more years of substantial visual loss. Sufferers may show an early-onset drusen phenotype comprising uniform small, slightly raised, yellow subretinal nodules randomly scattered in the macular, also known as ‘basal laminar drusen’ or ‘cuticular drusen’. EOMD may also be referred to as “middle-onset macular degeneration”. The EOMD subset is described in e.g. Boon C J et al. Am J Hum Genet 2008; 82(2):516-23; van de Ven J P, et al. Arch Ophthalmol 2012; 130(8):1038-47; and Taylor, R. L. et al., Ophthalmol. 2019, 126, 1410-1421, all of which are hereby incorporated by reference in their entirety. As with other types of macular degeneration, EOMD is related to complement dysregulation and disrupted Factor H activity. In some embodiments a subject to be treated is age 49 or younger. In some embodiments a subject to be treated is between ages 15 and 49, i.e. is between 15 and 49 years old. In some embodiments the disease or condition to be treated is a macular dystrophy. A macular dystrophy can be a genetic condition, usually caused by a mutation in a single gene, that results in degeneration of the macula.

In some embodiments the disorder is one associated with the kidney, e.g. nephropathy/a nephropathic disorder. In some cases, the disorder is a neurological and/or neurodegenerative disorder. In some cases, the disorder is associated with autoimmunity, e.g. an autoimmune disease. In some cases, the disorder is associated with inflammation, e.g. an inflammatory disease. In some cases the disorder is characterised by the deposition of C3, e.g. the glomerular pathologies (see e.g. Skerka et al 2013, supra).

Numerous FHR proteins have been implicated in complement-related kidney disorders. FH, FHL-1, FHR1, FHR2, FHR3 and FHR5 have been implicated in IgA nephropathy (see e.g. Poppelaars et al., J Clin Med. 2021, 10(20):4715; Zhu et al., Kidney Int. 2018 July, 94(1):150-158). Poppelaars et al suggest that FHR1 and FHR5 compete with the regulatory function of Factor H, such that the FHR proteins amplify alternative pathway activation and thereby stimulate development and progression of IgA nephropathy.

FHR5 has been implicated in C3 glomerulopathy and renal impairment (see e.g. Medjeral-Thomas et al., Kidney Int Rep. 2019, 4(10):1387-1400), as well as glomerular damage and kidney injury (e.g. Malik et al., PNAS, 2021, 118(13) e2022722118). Abnormal FHR hybrid proteins have also been reported in C3 glomerulopathy, and are thought to compete with FH for C3b binding and regulation (see e.g. Wong & Kavanagh, Semin Immunopathol. 2018; 40(1): 49-64). Wong & Kavanagh also discuss the involvement of FH, FHR1 and FHR3 in atypical Haemolytic Uremic Syndrome (aHUS) and paroxysmal nocturnal hemoglobinuria (PNH). FHR1 and FHR5 were detected in the glomeruli of patients with Dense Deposit Disease (DDD)/membranoproliferative glomerulonephritis type II, see e.g. Sethi et al., Kidney Int. 2009, 75(9):952-60, and Abrera-Abeleda et al., J Med Genet. 2006, 43(7): 582-589.

Schafer et al., Front Immunol. 2016, 7:542 describes the role of elevated FHR3 in a selection of autoimmune diseases, including kidney diseases and rheumatoid patients. Goicoechea de Jorge et al., PNAS 2013, 110(12):4685-90 discloses that CFH mutations increase susceptibility to aHUS, DDD, and meningococcal sepsis, and that FHR3 is involved in the pathogenesis of systemic lupus erythematosus (SLE). It also reports that dimerization of the FHR proteins, e.g. FHR1, FHR2 and FHR5, enhanced their ability to compete with FH for C3b binding and deregulate complement activation. Legatowicz-Koprowska et al., Reumatologia. 2020, 58(6): 357-366 reports the absence of complement cascade proteins in patients with primary Sjögren's syndrome. Increased levels of FHR proteins have been implicated in anti-neutrophilic cytoplasmic autoantibodies (ANCA) vasculitis (Skerka et al., Br J Pharmacol. 2021 July; 178(14):2823-2831).

Activation of the complement alternative pathway and upregulation of FHR proteins has been suggested as having a role in the development of atherosclerosis (see e.g. Speidl et al., J Thromb Haemost. 2011, 9(3):428-40; Malik et al., Circulation. 2010, 122(19):1948-56; Machalinska et al., Acta Ophthalmol. 2012, 90(8):695-703); and Irmscher et al., Nature Scientific Reports, volume 11, Article number: 22511 (2021).

In some embodiments the disorder may be selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schonlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, and Alzheimer's disease.

In some cases, the disorder is cancer. Complement activation plays a role in the development and progression of cancer. DeCordova et al., Immunobiology. 2019, 224(5):625-631 reports that FHR5 is secreted by primary tumor cells derived from Glioblastoma multiforme (GBM) patients and may be used by the cells to resist complement mediated lysis. Afshar-Kharghan, J Clin Invest. 2017, 127(3):780-789 reports that expression of complement factors is increased in malignant tumors, including the FHR proteins which would outcompete FH and lead to complement dysregulation. Alternatively, if tumors become hypoxic, then this can lead to a downregulation in FH expression and thus an increase in complement inflammatory activity, such that inhibition of complement activation is a therapeutic option (e.g. Pio et al., Semin Immunol. 2013, 25(1):54-64). FH has been reported as a biomarker for lung cancer, squamous lung cancer, bladder cancer, ovarian cancer, liver cancer and SCC (e.g. Revel et al., Antibodies (Basel). 2020, 9(4): 57).

The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine. In some cases the cancer is lung cancer. In some cases the cancer is glioblastoma e.g. glioblastoma multiforme (GBM). In some embodiments the complement-related disorder is an indoleamine 2,3-dioxygenase 1 (IDO)-expressing cancer, e.g. as described in WO2022/058447.

In some cases the disorder is neurodegeneration or neurodegenerative disease. The disorder may comprise progressive atrophy and loss of function of neurons. The disorder may be selected from Parkinson's disease, Alzheimer's disease, dementia, stroke, Lewy body disease, Amyotrophic lateral sclerosis (ALS), multiple sclerosis (MS), Huntington's disease and prion diseases.

The role of complement in various diseases is described in e.g. Morgan, B. P., Complement in the pathogenesis of Alzheimer's disease. Semin Immunopathol, 2018. 40(1): p. 113-124; Halbgebauer, R., et al., Janus face of complement-driven neutrophil activation during sepsis. Semin Immunol, 2018. 37: p. 12-20; Ma, Y., et al., Significance of Complement System in Ischemic Stroke: A Comprehensive Review. Aging Dis, 2019. 10(2): p. 429-462; Bonifati and Kishore, Role of complement in neurodegeneration and neuroinflammation. Mol Immunol. 2007 February; 44(5):999-1010; Kleczko, E. K., et al., Targeting the Complement Pathway as a Therapeutic Strategy in Lung Cancer. Front Immunol, 2019. 10: p. 954; and Schafer N. et al., Complement Regulator FHR-3 Is Elevated either Locally or Systemically in a Selection of Autoimmune Diseases, Front Immunol. 2016; 7: 542, which are all hereby incorporated by reference in their entirety. For example, FHL-1 is expressed more in certain tumour cell lines than FH (Junnikkala et al (2000) J. Immunol. 164: 6075-81) and glioblastoma tumours have been shown to express FHR proteins (DeCordova et al. (2019) Immunobiology 224: 625-631), both references hereby incorporated in their entirety. Being able to measure and differentiate between FH family proteins is advantageous.

Elevated levels of FHR1 and FHR3 have been found in plasma from patients with Alzheimer's disease, see e.g. Chen & Xia, J Alzheimers Dis. 2020, 76(1): 349-368; and Ashton et al., ALzhemers Dement (Amst) 2015, 1(1): 48-60 (see also Clark and Bishop J Clin Med. 2015 January; 4(1): 18-31). Thus, elevated levels of FHR proteins are associated with dementia-related disorders. Increased levels of FHR proteins (FHRs 1, 2 and 5) are associated with multiple sclerosis, see e.g. Loveless et al., Brain Pathol. 2018 July; 28(4): 507-520. Pouw and Ricklin, Semin Immunopathol. 2021, 43(6):757-771 discusses the role of FHR proteins as FH competitors and reviews the adverse effect of complement activation in the central nervous system, such as in the context of Alzheimer's disease, Parkinson's disease, schizophrenia, myasthenia gravis (MG), amyotrophic lateral sclerosis (ALS), and Guillain-Barre syndrome (GBS).

Increased circulating FH levels in subjects with altered glucose tolerance has been linked to insulin resistance and metabolic disturbances (Moreno-Navarrete et al., Diabetes. 2010, 59(1):200-9).

In some embodiments, the complement-related disorder is an infectious disease. Complement is a major component of the innate immune system involved in defending against foreign pathogens, including bacteria, viruses, fungi and parasites. Activation of complement leads to robust and efficient proteolytic cascades, which result in opsonization and lysis of the pathogen as well as in the generation of the classical inflammatory response through the production of potent proinflammatory molecules. The role of complement in innate and adaptive immune responses is reviewed in e.g. Dunkelberger, J., Song, WC. Cell Res 2010; 20, 34-50, and Rus H et al., Immunol Res. 2005; 33(2):103-12, which are hereby incorporated by reference in their entirety.

In some embodiments the complement-related disorder is infection by severe acute respiratory syndrome-related coronavirus (SARSr-CoV). In some embodiments the complement-related disorder is infection with SARS-CoV-2. In some embodiments the complement-related disorder is a disease/condition caused or exacerbated by SARS-CoV-2 infection, e.g. COVID-19 or another disease/condition for which infection with SARS-CoV-2 is a contributing factor. WO2022/058447 describes significantly elevated levels of FHR1, FHR2, FHR3, FHR4, FHR5 and FHL-1 in the blood of COVID-19 patients having severe disease relative to healthy control subjects.

All references in the paragraphs above are hereby incorporated by reference in their entirety.

Thus, in some embodiments the complement-related disorder (e.g. that is characterised by elevated levels of one or more of FHR1, FHR2, FHR3, FHR4 and/or FHR5) is selected from macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, kidney injury/damage/dysfunction, glomerular diseases, Membranoproliferative Glomerulonephritis Type II (MPGN II), sepsis, Henoch-Schonlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, anti-neutrophilic cytoplasmic autoantibodies (ANCA) vasculitis, neurodegeneration/neurodegenerative disease, dementia, multiple sclerosis (MS), Lewy body disease, Amyotrophic lateral sclerosis (ALS), Huntington's disease, prion diseases, cancer, lung cancer, glioblastoma e.g. gliobiastoma multiforme (GBM), stroke, insulin resistance, diabetes, an infectious disease, Parkinson's disease, and/or Alzheimer's disease.

Administration of the articles of the present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration of the articles of the present disclosure may be topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal, or may be as an eye drop (i.e. ophthalmic administration).

In some aspects and embodiments in accordance with the present disclosure, an agent, inhibitory nucleic acid, gene editing tool/system, nucleic acid, expression vector, or composition described herein is administered to the liver, e.g. to one or more hepatocytes. In some cases, an agent, inhibitory nucleic acid, gene editing tool/system, nucleic acid, expression vector, or composition described herein is administered to the blood (i.e. intravenous/intra-arterial administration). In some cases, an agent, inhibitory nucleic acid, gene editing tool/system, nucleic acid, expression vector, or composition described herein is administered subcutaneously.

Some aspects and embodiments in accordance with the present disclosure may involve targeted delivery of articles of the present disclosure, i.e. wherein the concentration of the relevant agent in the subject is increased in some parts of the body relative to other parts and/or wherein the relevant agent is delivered via a controlled-release technique. Examples of methods for nucleic acid delivery are described hereinabove. In some embodiments, the methods comprise intravenous, intra-arterial, intramuscular or subcutaneous administration and wherein the relevant agent is formulated in a targeted agent delivery system. Suitable targeted delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and delivery systems will be apparent to one skilled in the art. In some cases, the relevant agent is formulated for targeted delivery to a specific organ(s) or tissue(s). In some cases, the relevant agent is delivered to the liver. In some cases, the methods comprise intravenous, intra-arterial, intramuscular or subcutaneous administration and wherein the relevant agent is formulated for targeted delivery to the liver.

The particular mode and/or site of administration may be selected in accordance with the location where reduction of gene and/or protein expression of a Factor H family protein (e.g. FHR protein) is required. In some cases, the methods comprise intravenous and/or intra-arterial administration. In some cases, the methods comprise administration to the eye. Should reduction of expression of a gene encoding a Factor H family protein (e.g. FHR protein) be required, administration may be to the liver. In some cases, the relevant agent is delivered to one or more hepatocytes.

Administration of an agent or article of the present disclosure may be alone or in combination with other treatments, either simultaneously or sequentially dependent upon the condition to be treated.

Simultaneous administration refers to administration with another therapeutic agent together, for example as a pharmaceutical composition containing both agents (combined preparation), or immediately after each other and optionally via the same route of administration, e.g. to the same tissue, artery, vein or other blood vessel. Sequential administration refers to administration of one agent followed after a given time interval by separate administration of another agent. It is not required that the two agents are administered by the same route, although this is the case in some embodiments. The time interval may be any time interval.

In some embodiments, therapeutic or prophylactic intervention according to the present disclosure may further comprise administering another agent for the treatment/prevention of a complement-related disorder. Exemplary agents for the treatment/prevention of a complement-related disorder to be employed in such embodiments include C1 inhibitors, C5 inhibitors, C5a inhibitors, C5aR antagonists, C3 inhibitors, C3a inhibitors, C3b inhibitors, C3aR antagonists, classical pathway inhibitors, alternative pathway inhibitors, FH-supplementation therapy and/or MBL pathway inhibitors. Specific complement-targeted therapeutics include without limitation one or more of human C1 esterase inhibitor (C1-INH), eculizumab (Soliris®, Alexion; a humanized monoclonal IgG2/4-antibody targeting C5), APL-2 (Apellis), mubodina (Adienne Pharma and Biotech), ergidina (Adienne Pharma and Biotech), POT-4 (a cyclic peptide inhibitor of C3; Alcon), rituximab (Biogen Idec, Genentech/Roche), ofatumumab (Genmab, GSK), compstatin analogues, soluble and targeted forms of CD59, PMX53 and PMX205, (Cephalon/Teva), JPE-1375 (Jerini), CCX168 (ChemoCentryx), NGD-2000-1 (former Neurogen), Cinryze (Shire), Berinert (CSL Behring), Cetor (Sanquin), Ruconest/Conestat alfa (Pharming), TNT009 (True North), OMS721 (Omeros), CLG561 (Novartis), AMY-101 (Amyndas), APL-1 (Apellis), APL-2 (Apellis), Mirococept (MRC), Lampalizumab (FCD4514S, Genentech/Roche), ACH-4471 (Achillion), ALXN1210 (Alexion), Tesidolumab/LFG316 (Novartis/Morphosys), Coversin (Akari), RA101495 (Ra Pharma), Zimura (ARC1905, Ophthotech), ALN-CC5 (Alnylam), IFX-1 (InflaRx), ALXN1007 (Alexion), Avacopan/CCX168 (Chemocentryx) and/or one or more therapeutic agents as described in e.g. Ricklin and Lambris, Adv Exp Med Biol. 2013, 734: 1-22; Ricklin and Lambris, Semin Immunol. 2016, 28(3):208-22; Melis J P M et al., Mol Immunol. 2015 67(2):117-130; Thurman J M, Nephrol Dial Transplant, 2017 32: i57-i64; Cashman S M et al., PLoS One. 2011, 6(4):e19078; Bora N S et al., J Biol Chem. 2010, 285(44):33826-33; and Clark et al., J Clin Med 2015, 4(1):18-31, WO 2018/224663 and WO 2019/138137, all of which are hereby incorporated by reference in their entirety.

Other therapeutic agents or techniques suitable for use in connection with the present disclosure may comprise nutritional therapy, photodynamic therapy (PDT), laser photocoagulation, anti-VEGF (vascular endothelial growth factor) therapy, and/or additional therapies known in the art, see e.g. AI-Zamil W M and Yassin S A, Clin Interv Aging. 2017 Aug. 22; 12:1313-1330). Anti-VEGF therapy may comprise agents such as ranibizumab (Lucentis, made by Genentech/Novartis), Avastin (Genentech), bevacizumab (off label Avastin), and aflibercept (Eylea®/VEGF Trap-Eye from Regeneron/Bayer). Further agents or techniques suitable for use with the present disclosure include APL-2 (Apellis), AdPEDF (GenVec), encapsulated cell technology (ECT; Neurotech), squalamine lactate (EVIZON™, Genaera), OT-551 (antioxidant eye drops, Othera), anecortave actate (Retaane®, Alcon), bevasiranib (siRNA, Acuity Pharmaceuticals), pegaptanib sodium (Macugen®), and AAVCAGsCD59 (Clinical trial identifier: NCT03144999).

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments the therapeutic or prophylactic intervention comprises a stem cell transplant.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

Multiple doses of the articles of the present disclosure may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or31 days, or 1, 2, 3, 4, 5, or 6 months. By way of example, doses may be given once every 7, 14, 21 or 28 days (plus or minus 3, 2, or 1 days).

Articles of the present disclosure may be formulated in a sustained release delivery system, in order to release the agent(s), inhibitory nucleic acid(s), nucleic acid(s), expression vector(s) and composition(s) at a predetermined rate. Sustained release delivery systems may maintain a constant drug/therapeutic concentration for a specified period of time. In some embodiments, nucleic acid(s), nucleic acid(s), expression vector(s) or composition(s) described herein are formulated in a liposome, gel, implant, device, or drug-polymer conjugate e.g. hydrogel.

### Selection for Therapeutic or Prophylactic Intervention

In some aspects the present disclosure provides methods comprising selecting treatment for and/or treating subjects/patients that have a complement-related disorder or have been identified as having a complement-related disorder, e.g. by determining the level of a complement protein as described herein.

The methods described herein may be diagnostic, prognostic and/or predictive of the risk of onset or progression of a complement-related disorder. Diagnostic methods can be used to determine the diagnosis or severity of a disease, prognostic methods help to predict the likely course of disease in a defined clinical population under standard treatment conditions, and predictive methods predict the likely response to a treatment in terms of efficacy and/or safety, thus supporting clinical decision-making. Such methods may be used prior to administration of an agent, such as an inhibitory nucleic acid or gene editing tool/system described herein. Subjects with elevated levels of FHR1, FHR2, FHR3, FHR4 and/or FHR5 may derive therapeutic or prophylactic benefit from the activity levels of any one or more of said proteins being reduced, e.g. using any agent described herein.

In some aspects, a subject may be selected for therapeutic or prophylactic intervention in accordance with the present disclosure by a method comprising:
(a) determining the level of a complement protein selected from one or more of FHR1, FHR2, FHR3, FHR4 and/or FHR5, and optionally FH and/or FHL-1, in a blood sample obtained from the subject;
(b) selecting the subject for treatment with an agent, e.g. inhibitory nucleic acid, gene editing tool/system, nucleic acid, expression vector or composition described herein if the level of one or more of FHR1, FHR2, FHR3, FHR4 and/or FHR5, and optionally FHL-1, determined in (a) is elevated as compared to the level of that complement protein(s) in blood in a control subject that does not have a complement-related disorder.

The subject may then be treated with an agent as described herein.

Also provided is a method of treating/preventing a complement-related disorder comprising:
(a) determining the level of a complement protein selected from one or more of FHR1, FHR2, FHR3, FHR4 and/or FHR5, and optionally FH and/or FHL-1, in a blood sample obtained from a subject;
(b) determining whether the level of FHR1, FHR2, FHR3, FHR4 and/or FHR5, and optionally FHL-1, is elevated as compared to the level of that complement protein(s) in blood in a control subject that does not have a complement-related disorder; and
(c) administering an agent, e.g. inhibitory nucleic acid, gene editing tool/system, nucleic acid, expression vector or composition described herein to the subject.

Also provided is an agent, gene editing tool/system, inhibitory nucleic acid, nucleic acid, expression vector or composition described herein for use as a medicament. For example the agent may be provided for use in a method of treating/preventing a complement-related disorder, the method comprising:
(a) determining the level of a complement protein selected from one or more of FHR1, FHR2, FHR3, FHR4 and/or FHR5, and optionally FH and/or FHL-1, in a blood sample obtained from a subject;
(b) determining whether the level of FHR1, FHR2, FHR3, FHR4 and/or FHR5, and optionally FHL-1, is elevated as compared to the level of that complement protein in blood in a control subject that does not have a complement-related disorder; and
(c) administering an agent, gene editing tool/system, inhibitory nucleic acid, nucleic acid, expression vector or composition described herein to the subject.

Also provided is the use of an agent, gene editing tool/system, inhibitory nucleic acid, nucleic acid, expression vector or composition described herein in the manufacture of a medicament for treating/preventing a complement-related disorder, comprising:
(a) determining the level of a complement protein selected from one or more of FHR1, FHR2, FHR3, FHR4 and/or FHR5, and optionally FH and/or FHL-1, in a blood sample obtained from a subject;
(b) determining whether the level of one or more of FHR1, FHR2, FHR3, FHR4 and/or FHR5, and optionally FHL-1, is elevated as compared to the level of that complement protein in blood in a control subject that does not have a complement-related disorder; and
(c) administering an agent, gene editing tool/system, inhibitory nucleic acid, nucleic acid, expression vector or composition described herein to the subject.

Any method described herein may comprise one or more of:
(a) determining the level of a complement protein selected from one or more of FHR1, FHR2, FHR3, FHR4 and/or FHR5, and optionally FH and/or FHL-1, in a blood sample obtained from a subject;
(b) determining whether the level of the complement protein (e.g. one or more of FHR1, FHR2, FHR3, FHR4 and/or FHR5, and optionally FHL-1) is elevated as compared to the level of that complement protein in blood in a control subject that does not have a complement-related disorder; and/or
(c) administering an agent, gene editing tool/system, inhibitory nucleic acid, nucleic acid, expression vector or composition described herein to the subject in order to reduce the gene and/or protein expression of one or more complement protein(s) found to be elevated in step (b).

Methods provided herein may be useful for determining the risk of a subject developing a serious complement-related disorder, e.g. the methods are useful for distinguishing between subjects who may develop a mild complement-related disorder and subjects who are at risk of serious disease, and/or identifying subjects who are likely to develop serious disease. Methods described herein may also be useful for assessing whether treatment for a complement-related disorder is/has been effective or successful.

As described herein, the methods may comprise determining whether the level of a complement protein is altered, e.g. increased or decreased, as compared to the level of that complement protein in blood in a control subject that does not have a complement-related disorder, or a subject that has a complement-related disorder that is not associated with an altered level of said protein. In some cases, the methods may comprise determining the relative concentrations of complement proteins compared to each other, e.g. the level of a complement protein may be elevated as compared to the level of a different complement protein, which may be unaltered or decreased, in the same subject or as compared to a control subject.

In some embodiments the level of a complement protein is determined using any suitable technique known in the art and available to a skilled person. In some embodiments the level of a complement protein is determined by mass spectrometry and/or digesting the protein with endoproteinase GluC, e.g. as described herein.

In some embodiments the methods described herein are performed in vitro or ex vivo. For example, a sample may be obtained from a subject of interest, or a control subject, and the steps that involve determining the level of a complement protein, determining whether a subject has or is at risk of developing a complement-related disorder, and digesting at least one complement protein are performed in vitro or ex vivo. Steps of methods that involve treating a subject may be performed in vivo.

The methods described herein may be useful in monitoring the success of therapeutic or prophylactic intervention, including past or ongoing treatment, for complement-related disorders. In some embodiments, the methods described herein may comprise administering an agent, inhibitory nucleic acid, nucleic acid, expression vector, or composition described herein to a subject who has/has been determined to have a complement-related disorder, and then re-determining the level of one or more of FHR1, FHR2, FHR3, FHR4 and/or FHR5, and optionally FH and/or FHL-1, after such therapeutic/prophylactic intervention. Such methods are useful for determining the efficacy of therapeutic/prophylactic intervention and the progression of the disorder.

In some aspects, the present disclosure provides methods of predicting, based on the analysis described herein of a sample from a subject, whether a subject is at risk of developing a complement-related disorder, has a complement-related disorder, is in need of therapeutic/prophylactic intervention for a complement-related disorder, will respond to therapeutic/prophylactic intervention for a complement-related disorder, and/or is responding/has responded to therapeutic/prophylactic intervention for a complement-related disorder. The methods may be used for determining whether a subject is at risk of onset of the disorder, and/or is at risk of progression, exacerbation or worsening of the disorder.

The methods described herein may be used for determining whether a subject is at risk of onset or progression of a complement-related disorder, e.g. as described herein. The complement related disorder may be macular degeneration, e.g. EOMD and/or AMD. In some cases, the disorder is selected from EOMD, AMD, geographic atrophy (‘dry’ (i.e. non-exudative) AMD), early AMD, intermediate AMD, late/advanced AMD, ‘wet’ (neovascular or exudative) AMD, choroidal neovascularisation (CNV) and retinal dystrophy. In some cases, the subject has or is suspected to have a complement-related disorder. In some cases the disorder is AMD. In some cases the disorder is EOMD.

Thus the present disclosure provides a method for determining whether a subject is at risk of developing macular degeneration, e.g. EOMD and/or AMD, the method comprising:
(a) digesting one or more complement proteins in a sample obtained from the subject with endoproteinase GluC to obtain one or more peptides, e.g. wherein the one or more complement proteins is selected from one or more of FHR1, FHR2, FHR3, FHR4 and/or FHR5, and optionally FH and/or FHL-1;
(b) determining the level of the one or more peptides by mass spectrometry;
(c) using the results of (b) to determine the level of the one or more complement proteins; and
(d) determining that the subject has, or is at risk of developing, macular degeneration if the level of the FHR1, FHR2, FHR3, FHR4 and/or FHR5, and optionally FHL-1, is elevated as compared to the level of that complement protein(s) in blood in a control subject that does not have a complement-related disorder.

Also provided herein is a method for assessing the propensity or predisposition of a subject to develop a complement-related disorder, comprising steps (a) to (d) above.

Methods described herein may also be useful for assessing whether therapeutic/prophylactic intervention for a complement-related disorder is/has been effective or successful.

In some aspects, the methods described herein may be useful for determining whether a subject is likely to respond or not respond to a therapeutic intervention, or whether a subject is responding to a therapeutic intervention. The methods should enable patients to receive the most effective therapy for their particular pathological requirements.

In some cases, the subject has or is suspected to have a complement-related disorder. In some cases, the methods provided herein comprise determining if a subject has or is suspected to have a complement-related disorder. In some cases the disorder is AMD. In some cases the disorder is EOMD.

In some aspects, the present disclosure provides a method for treating or preventing a complement-related disorder in a subject, the method comprising administering a therapeutically- or prophylactically-effective amount of an agent, inhibitory nucleic acid, gene editing tool/system, nucleic acid, expression vector, or composition according to the present disclosure, wherein the subject to be treated has been determined to have atypical presence or levels of one or more complement proteins, e.g. detected/determined as described herein, as compared to a control subject and/or reference value(s). In some aspects the subject has been determined to be at risk of developing a complement-related disorder, and/or identified as having a complement-related disorder.

In other aspects, the present disclosure provides an agent, inhibitory nucleic acid, gene editing tool/system, nucleic acid, expression vector, or composition according to the present disclosure for use in a method of treating or preventing a complement-related disorder in a subject, the method comprising administering a therapeutically- or prophylactically-effective amount of the agent, inhibitory nucleic acid, gene editing tool/system, nucleic acid, expression vector, or composition, wherein the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

In some aspects, provided is the use of an agent, inhibitory nucleic acid, gene editing tool/system, nucleic acid, expression vector, or composition according to the present disclosure in the manufacture of a medicament for treating or preventing a complement-related disorder in a subject, wherein the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

Also provided is a method of treating or preventing a complement-related disorder in a subject, or an agent, inhibitory nucleic acid, gene editing tool/system, nucleic acid, expression vector, or composition according to the present disclosure for use in a method of treating or preventing a complement-related disorder in a subject, the method comprising administering a therapeutically- or prophylactically-effective amount of an agent, inhibitory nucleic acid, gene editing tool/system, nucleic acid, expression vector, or composition according to the present disclosure, wherein the subject is selected for treatment if the subject has/has been determined to have atypical presence or levels of one or more complement proteins, e.g. determined as described herein, as compared to a reference value(s). In some aspects the subject has been determined to be at risk of developing a complement-related disorder, and/or identified as having a complement-related disorder.

In various aspects provided herein, the subject to which therapeutic or prophylactic intervention is to be administered has atypical presence or levels of at least one complement protein, preferably one or more of FH, FHL-1, FHR1, FHR2, FHR3, FHR4, FHR5, FI, C3, C3b, C3a, iC3b, C3f, C3c, C3dg, and/or C3d. In some embodiments, the subject has atypical presence of levels of one or more of FHR1, FHR2, and/or FHR3, and optionally FHR4 and/or FHR5, and/or FHL-1. The subject may benefit from therapeutic or prophylactic intervention to reduce the level of any complement proteins that are increased as compared to a reference value(s) and/or from therapeutic or prophylactic intervention to increase the level of any complement proteins that are decreased as compared to a reference value(s).

Methods provided herein for assessing the risk of development, i.e. the onset or risk of progression of, or for identifying subjects having/at risk of, a complement-related disorder may be performed in conjunction with additional diagnostic methods and/or tests for such disorders that will be known to one skilled in the art. In some cases, methods for assessing the risk of development of a complement-related disorder comprise further techniques selected from: CH50 or AH50 measurement via haemolytic assay, measurement of neoantigen formation during MAC complex (C5b, C6, C7, C8, C9) generation, C3 deficiency screening, mannose-binding lectin assays, immunochemical assays to quantify individual complement components, flow cytometry to assess cell-bound regulatory proteins e.g. CD55, CD59 and CD35, and/or renal function tests, see e.g. Shih A R and Murali M R, Am. J. Hematol. 2015, 90(12):1180-1186, Ogedegbe H O, Laboratory Medicine, 2007, 38(5):295-304, and Gowda S et al., N Am J Med Sci. 2010, 2(4): 170-173, which are herein incorporated by reference in their entirety.

In some cases, methods provided herein for assessing the risk of development of AMD and/or EOMD comprise further assessment techniques selected from: dark adaptation testing, contrast sensitivity testing e.g. Pelli Robson, visual acuity testing using e.g. a Snellen chart and/or Amsler grid, Farnsworth-Munsell 100 hue test and Maximum Color Contrast Sensitivity test (MCCS) for assessing colour acuity and colour contrast sensitivity, preferential hyperacuity perimetry (PHP), fundus photography of the back of the eye, fundus examination, fundus autofluorescence, optical coherence tomography, angiography e.g. fluorescence angiography, fundus fluorescein angiography, indocyanine green angiography, optical coherence tomography angiography, adaptive optics retinal imaging, deep learning analysis of fundus images, electroretinogram methods, and/or methods to measure histological changes such as atrophy, retinal pigment changes, exudative changes e.g. hemorrhages in the eye, hard exudates, subretinal/sub-RPE/intraretinal fluid, and/or the presence of drusen.

Methods described herein may take into account lifestyle factors known to contribute to risk of developing complement-related disorders. For example, lifestyle factors that may cause or contribute to AMD include smoking, being overweight, high blood pressure and having a family history of AMD.

The methods provided herein may comprise determining in a subject the presence or absence of a genetic profile characterised by polymorphisms in the subject's genome associated with complement dysregulation. The polymorphisms may be found within or near genes such as CCL28, FBN2, ADAM12, PTPRC, IGLC1, HS3ST4, PRELP, PPID, SPOCK, APOB, SLC2A2, COL4A1, MYOC, ADAM19, FGFR2, C8A, FCN1, IFNAR2, C1NH, C7 and ITGA4. A genetic profile associated with complement dysregulation may comprise one or more, often multiple, single nucleotide polymorphisms, e.g. as set out in Tables I and II of US 2010/0303832, which is hereby incorporated by reference in its entirety.

Genetic factors are thought to play a role in the development of AMD and EOMD. Thus, any of the assessment or therapeutic/prophylactic methods described herein may be performed in conjunction with methods to assess AMD-associated and/or EOMD-associated and/or macular dystrophy-associated genetic variants. In some cases a complement-related disorder described herein may comprise a genetic element and/or a genetic risk factor.

In some cases, a method provided herein may comprise determining in a subject the presence or absence of one or more genetic factors associated with AMD and/or EOMD, e.g. one or more AMD- or EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present disclosure involve determining whether a subject possesses one or more such risk factors.

In some embodiments, the one or more genetic factors may be located on chromosome 1 at or near the RCA locus, e.g. in the CFH/CFHR genes/the CFH locus. In some embodiments, the presence of one or more CFH locus AMD-risk variants increase disease risk via increase of FHR protein levels.

The one or more genetic factors may be located in one or more of: CFH e.g. selected from Y402H (i.e. rs1061170^{c}), rs1410996c, I62V (rs800292), A473A (rs2274700), R53C, D90G, D936E (rs1065489), R1210C, IVS1 (rs529825), IVS2 insTT, IVS6 (rs3766404), A307A (rs1061147), IVS10 (rs203674), rs3753396, R1210C, rs148553336, rs191281603, rs35292876, and rs800292; CFHR4 e.g. selected from rs6685931, and rs1409153; CFI e.g. selected from G119R, and rs141853578; CFB e.g. rs4151667, C2 e.g. rs9332739, C9 e.g. P167S; and/or C3 e.g. K155Q. In some embodiments, a genetic factor is Y402H (i.e. rs1061170c). In some embodiments, a genetic factor is rs3753396. In some embodiments, a genetic factor is rs6685931 and/or rs1409153. In some embodiments, a genetic factor is at intronic KCNT2 rs61820755. In some embodiments, a genetic factor is not rs6685931.

In some embodiments a genetic factor is rs61820755, and may be associated with FHL-1.

In any embodiment herein, the genetic risk factors may be present in combination with elevated levels of one or more FHR proteins. The one or more genetic factors at the CFH locus may be selected from intergenic CFHR1/CFHR4 rs149369377 and/or rs61820755 for FHR-1, CFHR2 rs4085749 for FHR-2, intronic CFH rs70620 for FHR-3, rs12047098 for FHR-4, intronic KCNT2 rs72732232 for FHR-5. The presence of any one or more of these genetic factors indicates that the subject has or is likely to develop a complement-related disorder.

The one or more genetic risk factors may be selected from rs10922109, rs570618, rs121913059 (R1210C), rs148553336, rs187328863, rs61818925, rs35292876, and rs191281603.

The one or more genetic factors may be selected from one or more of rs113721756 on chromosome 10, rs111260777 on chromosome 11, rs117468955 on chromosome 12, rs200404865 on chromosome 13, rs4790395 on chromosome 17 and rs117115124 on chromosome 19. These factors may be present separately, or in addition to, genetic factors at the CFH locus. These factors may be present in combination with elevated FHR-3 levels.

Any and all combinations of genetic factors are envisaged, e.g. those described herein or additional factors, including their detection/assessment as below).

The methods described herein may involve detecting combinations of risk factors to assess the risk of a subject developing a complement related disorder, e.g. if one or both of the risk factors are present in a subject. For example:
rs10922109 and intergenic CFHR1/CFHR4 rs149369377
rs10922109 and CFHR2 rs4085749
rs10922109 and intronic CFH rs70620
rs10922109 and intergenic CFHR1-CFHR4 rs12047098
rs570618 and intergenic CFHR1/CFHR4 rs149369377
rs570618 and CFHR2 rs4085749
rs148553336 and intronic KCNT2 rs72732232
rs61818925 and CFHR2 rs4085749; and/or
rs61818925 and intergenic CFHR1-CFHR4 rs12047098.

Assessment of the presence of any genetic risk factor provided herein may be combined with the detection of any one or more of FHR1, FHR2, FHR3, FHR4 and/or FHR5, and optionally FHL-1 as described herein. For example, the presence of genetic factor rs10922109 may be assessed in combination with the detection of any one or more of FHR-1, FHR-2, FHR-3, and/or FHR-4; rs570618 may be assessed in combination with the detection of FHR-1 and/or FHR-2; rs61818925 may be assessed in combination with the detection of FHR-2 and/or FHR-4; and rs148553336 may be assessed in combination with the detection of FHR-5.

In any embodiment or method herein, the method may comprise a step of determining that the subject has or is likely to develop a complement-related disorder if one or more genetic factors, e.g. those described herein, are present.

Thus provided herein is a method of identifying a subject having a complement-related disorder or at risk of developing a complement-related disorder, the method comprising assessing the subject for one or more genetic risk factors, e.g. any of those described herein or others, and determining that the subject has or is likely to develop a complement-related disorder if the one or more genetic risk factors are present in the subject.

Provided herein is a method of determining whether a subject has, or is at risk of developing, a complement-related disorder, the method comprising assessing the subject for one or more genetic risk factors, e.g. any of those described herein or others, and determining that the subject has or is likely to develop a complement-related disorder if the one or more genetic risk factors are present in the subject.

Also provided is a method for selecting therapeutic/prophylactic intervention for and/or administering therapeutic/prophylactic intervention to subjects/patients that have a complement-related disorder or have been identified as having a complement-related disorder, e.g. using the steps above.

Also provided is a method for selecting a subject for therapeutic/prophylactic intervention with an agent, inhibitory nucleic acid, gene editing tool/system, nucleic acid, expression vector, or composition described herein; a method for selecting an agent, inhibitory nucleic acid, gene editing tool/system, nucleic acid, expression vector, or composition described herein for a subject; methods of treatment; an agent, inhibitory nucleic acid, gene editing tool/system, nucleic acid, expression vector, or composition described herein for use in a method of treatment; and the use of an agent, inhibitory nucleic acid, gene editing tool/system, nucleic acid, expression vector, or composition described herein in the manufacture of a medicament for the treatment of a complement-related disorder, wherein the method uses the steps above to assess genetic risk factors (either alone or in combination with determining the level of a complement protein e.g. an FHR protein as described herein and/or determining whether the level of one or more of FHR1, FHR2, FHR3, FHR4 and/or FHR5, and optionally FHL-1, is elevated as compared to the level of that complement protein in blood in a control subject that does not have a complement-related disorder, as described herein).

Any such method comprising detecting and assessing genetic risk factors may comprise a step of administering an agent, inhibitory nucleic acid, gene editing tool/system, nucleic acid, expression vector, or composition described herein, e.g. to a subject that has been determined to have or be likely to develop a complement-related disorder.

Other suitable genetic risk factors and genetic variants will be known in the art and may be as described in e.g. Edwards A O et al., Science 2005, 308(5720):421-4; Hageman G S et al., Proc Natl Acad Sci USA. 2005, 102(20):7227-7232; Haines J L et al., Science 2005, 308(5720):419-21, Klein R J et al., Science 2005, 308(5720):385-389; Fritsche et al., Nat Genet. 2016, 48(2):134-43; US 2010/0303832; Clark S et al., J Clin Med. 2015, 4(1):18-31, Cipriani, V. et al., Nat Commun. 2020, 11, 778; or Hageman G S et al, Hum Genomics. 2011, 5, 420 (2011), each hereby incorporated by reference in its entirety.

In some cases, the methods provided herein further comprise determining in a subject the presence or absence of one or more genetic factors associated with EOMD, e.g. one or more EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present disclosure involve determining whether a subject possesses one or more such risk factors. In some embodiments the subject may possess one or more risk factors for early-onset macular degeneration (EOMD).

EOMD is thought to be caused by monogenic inheritance of rare variants of the CFH gene (see e.g. Boon C J et al. Am J Hum Genet 2008; 82(2):516-23; van de Ven J P, et al. Arch Ophthalmol 2012; 130(8):1038-47; Yu Y et al. Hum Mol Genet 2014; 23(19):5283-93; Duvvari M R, et al. Mol Vis 2015; 21:285-92; Hughes A E, et al. Acta Ophthalmol 2016; 94(3):e247-8; Wagner et al. Sci Rep 2016; 6:31531; Taylor R L et al, Ophthalmology. 2019 Mar. 21. pii: S0161-6420(18):33171-3). In some embodiments, the subject may possess one or more of EOMD-associated genetic variants. EOMD-associated genetic variants are described in e.g. Servais A et al. Kidney Int, 2012; 82(4):454-64 and Dragon-Durey M A, et al. J Am Soc Nephrol 2004; 15(3):787-95; which are hereby incorporated by reference in their entirety. In some embodiments, the subject may possess one or more of the following EOMD-associated genetic variants: CFH c.1243del, p.(Ala415Profs*39) het; CFH c.350+1G>T het; CFH c.619+1G>A het; CFH c.380G>A, p.(Arg127His); CFH c.694C>T, p.(Arg232Ter); or CFH c.1291T>A, p.(Cys431Ser).

In some cases, the methods provided herein comprise screening for deletions within the RCA locus (a region of DNA sequence located on chromosome one that extends from the CFH gene through to the CD46 (MCP) gene) that are associated with AMD and/or EOMD risk or protection.

Methods for determining the presence or absence of genetic factors include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, PCR, DNA sequencing e.g. Sanger sequencing or Next-Generation sequencing, allele specific oligonucleotide (ASO) probes, and oligonucleotide microarrays or beads. Other suitable methods are described in e.g. Edenberg H J and Liu Y, Cold Spring Harb Protoc; 2009; doi:10.1101/pdb.top62, and Tsuchihashi Z and Dracopoli N C, Pharmacogenomics J., 2002, 2:103-110.

In some embodiments, the subject is selected for therapeutic or prophylactic treatment with an agent, inhibitory nucleic acid, gene editing tool/system, nucleic acid, expression vector, or composition described herein based on their being determined to possess one or more genetic factors for AMD and/or EOMD, e.g. one or more AMD-associated and/or EOMD-associated genetic variants, or for a macular dystrophy. In some embodiments, the subject has been determined to have one or more such genetic factors. In some embodiments, the methods provided herein comprise determining whether a subject possesses one or more such genetic factors. Examples of such methods and genetic factors are described herein. Thus, provided herein is a method of diagnosing, treating or preventing a complement-related disorder in a subject, wherein the subject has/has been determined/is determined to possess one or more genetic factors for AMD and/or EOMD, and wherein the subject has/has been determined/is determined to have atypical presence or levels of one or more complement proteins, e.g. detected/determined as described herein, as compared to a reference value(s); optionally wherein the method comprises administering an agent, inhibitory nucleic acid, gene editing tool/system, nucleic acid, expression vector, or composition described herein.

Provided is a method of treating or preventing a complement-related disorder in a subject, wherein the subject is characterised as having an atypical presence or levels of one or more complement proteins, e.g. detected/determined as described herein.

Also provided is a complement-targeted therapeutic agent for use in a method of treating or preventing a complement-related disease in a subject, wherein the subject is characterised as having an atypical presence or levels of one or more complement proteins, e.g. detected/determined as described herein.

### Methods for Assessing Complement-Related Disorders

The present disclosure also provides methods for assessing the risk of onset, risk of progression, or risk of development of a complement-related disorder. The complement related disorder may be any disorder in which the complement system, or activation/over-activation/dysregulation thereof, is pathologically implicated. The complement related disorder may be any disorder described herein. The methods described herein may be useful in monitoring the success of treatment, including past or ongoing treatment, for complement-related disorders. Such treatment may involve one or more agents as described herein.

In some aspects, provided is a method of identifying a subject having a complement-related disorder or at risk of developing a complement-related disorder, the method comprising:
(a) determining the level of a complement protein selected from one or more of FHR1, FHR2, FHR3, FHR4 and/or FHR5 in a blood sample obtained from the subject;
(b) determining that the subject has or is likely to develop a complement-related disorder if the level of the complement protein determined in (a) is elevated as compared to the level of that complement protein in blood in a control subject that does not have a complement-related disorder.

In some aspects, there is provided a method of determining whether a subject has, or is at risk of developing, a complement-related disorder, the method comprising:
(a) determining the level of a complement protein selected from one or more of FHR1, FHR2, FHR3, FHR4 and/or FHR5 in a blood sample obtained from the subject;
(b) determining that the subject has or is likely to develop a complement-related disorder if the level of the complement protein determined in (a) is elevated as compared to the level of that complement protein in blood in a control subject that does not have a complement-related disorder.

In some embodiments step (a) comprises determining the level of two of the complement proteins selected from FHR1, FHR2 and/or FHR3. In some embodiments step (a) comprises determining the level of three of the complement proteins selected from FHR1, FHR2 and FHR3.

In some embodiments step (a) comprises, or further comprises, determining the level of FHR4 and/or FHR5. The methods described herein may comprise determining that the subject has or is likely to develop a complement-related disorder if the level of FHR4 and/or FHR5 is elevated as compared to the level of that complement protein in blood in a control subject that does not have a complement-related disorder.

In some embodiments step (a) comprises, or further comprises, determining the level of FH and/or FHL-1. The method may comprise determining the level of FHL-1, alone or in combination with other complement protein(s), and determining that the subject has or is likely to develop a complement-related disorder if the level of FHL-1 is altered, e.g. elevated, as compared to the level of FHL-1 in blood in a control subject that does not have a complement-related disorder. The level of FH and/or FHL-1 may be increased or decreased compared to a control subject.

Determining the level of two or more complement proteins may be performed simultaneously, concurrently or sequentially. The complement proteins may be detected in the same assay, or in one or more separate assays. Determining the level of a second or subsequent complement protein may be performed concurrently with, prior to or after determining the level of a first complement protein. In some embodiments, steps (a) and (b) may be repeated one or more times on the same subject at appropriate time intervals in order to assess the progression of a complement-related disorder.

Any aspect or embodiment described herein may comprise determining the level of (e.g. expression of) any one of the following genes/proteins, e.g. in a subject:
a) FHR1;
b) FHR2;
c) FHR3;
d) FHR4;
e) FHR5;
f) FHR1 and FHR2;
g) FHR1 and FHR3;
h) FHR1 and FHR4;
i) FHR1 and FHR5;
j) FHR2 and FHR3;
k) FHR2 and FHR4;
l) FHR2 and FHR5;
m) FHR3 and FHR4;
n) FHR3 and FHR5;
o) FHR4 and FHR5;
p) FHR1, FHR2 and FHR3;
q) FHR1, FHR2 and FHR4;
r) FHR1, FHR2 and FHR5;
s) FHR1, FHR3 and FHR4;
t) FHR1, FHR3 and FHR5;
u) FHR1, FHR4 and FHR5;
v) FHR2, FHR3 and FHR4;
w) FHR2, FHR3 and FHR5;
x) FHR2, FHR4 and FHR5;
y) FHR3, FHR4 and FHR5;
z) FHR1, FHR2, FHR3 and FHR4;
aa) FHR1, FHR2, FHR3 and FHR5;
bb) FHR1, FHR2, FHR4 and FHR5;
cc) FHR2, FHR3, FHR4 and FHR5;
dd) FHR1, FHR3, FHR4 and FHR5; or
ee) FHR1, FHR2, FHR3, FHR4 and FHR5;
   or any of (a) to (ee) in combination with determining the level of FH and/or FHL-1, e.g. FHR1, FHR2, FHR3, plus FH and/or FHL-1; or FHR1, FHR2, FHR3, FHR4, FHR5, plus FH and/or FHL-1.

The selection or combination of complement protein(s) detected may depend on the complement-related disorder of interest and the complement protein(s) that are useful biomarkers for an individual disorder. For example, detecting one or more of FHR1, FHR2, FHR3, FHR4, FHR5, FH and/or FHL-1L-1 is predictive of AMD risk, whereas other particular complement proteins and combinations thereof are predictive for other complement-related disorders, see e.g. the disorders and references described herein. The present disclosure allows the precise detection and distinction of any one or more of the complement proteins described herein, thus allowing the absolute levels of said proteins to inform the likelihood of disorder onset and/or progression according to the variations in protein levels in each disorder. The complement protein(s) may be detected in a sample obtained from a subject. For example, the sample may be obtained to inform appropriate treatment and/or progression of the disorder.

In some cases, any aspect described herein may comprise determining the level of any one or more complement proteins selected from FHR1, FHR2, FHR3, FHR4, FHR5, FH and/or FHL-1, e.g. in a blood sample obtained from a subject, and then determining that the subject has or is likely to develop a complement-related disorder if the level of the complement protein(s) is altered as compared to the level of that complement protein(s) in blood in a control subject that does not have a complement-related disorder. The term “altered” as used herein refers to the level of the complement protein(s) increasing or decreasing, e.g. the level of one or more complement proteins may be higher or lower as compared to the level of those complement proteins in blood in a control subject that does not have a complement-related disorder. In some cases, the level of the complement protein may be decreased as compared to the level of that complement protein in blood in a control subject that does not have a complement-related disorder. In some cases, where the level of two or more complement proteins is determined, the level of one or more complement proteins may be elevated whilst the level of one or more different complement proteins may be decreased as compared to the levels of those complement proteins in blood in a control subject that does not have a complement-related disorder.

In some embodiments the level of a complement protein is determined using any suitable technique known in the art and available to a skilled person. In some embodiments the level of a complement protein is determined by mass spectrometry and/or digesting the protein with endoproteinase GluC, e.g. as described herein. Determining the level of a complement protein(s) may involve detecting any combination of peptides produced by digestion with GluC, as described herein. The level of a complement protein may be determined using, for example, an enzyme-linked immunosorbent assay (ELISA/EIA) e.g. as described in van Beek et al., Front Immunol. 2017; 8: 1328; van Beek et al. Front Immunol. 2018; 9: 1727; and Pouw et al., PLoS One. 2016 Mar. 23; 11(3):e0152164; which are hereby incorporated by reference in their entirety. The level of a complement protein may be determined using, for example, Western blotting or dot blotting with appropriate antibodies, HPLC, protein immunoprecipitation or immunoelectrophoresis.

Any aspect described herein may comprise an initial step of obtaining a sample and/or at least one protein, e.g. complement protein, from the subject. Suitable sources of samples are described herein. The methods described herein may comprise determining the level of circulating FHR1, FHR2, and/or FHR3, circulating FHR4 and/or FHR5, and optionally circulating FH and/or FHL-1. Circulating proteins may be present in e.g. blood or lymph.

Any method described herein may comprise determining the level of one or more of C3, C3b, C3a, iC3b, C3f, C3c, C3dg, and/or C3d, e.g. as described herein.

In some aspects, there is provided a method of determining whether a subject has, or is at risk of developing, a complement-related disorder, the method comprising:
(a) digesting at least one complement protein selected from FHR1, FHR2, FHR3 FHR4 and/or FHR5, in a sample obtained from the subject with endoproteinase GluC to obtain one or more peptides;
(b) determining the level of the one or more peptides by mass spectrometry;
(c) using the results of (b) to determine the level of one or more complement proteins; and
(d) determining that the subject has, or is at risk of developing, a complement-related disorder if the level of one or more complement proteins determined in (c) is elevated as compared to the level of that complement protein(s) in blood in a control subject that does not have a complement-related disorder.

In some embodiments the method further comprises digesting one or both of FH and/or FHL-1 with endoproteinase GluC to obtain one or more peptides, determining the level of the one or more peptides by mass spectrometry and/or using the results of the mass spectrometry to determine the level of FH and/or FHL-1. Exemplary combinations of complement proteins for use in the methods of the present disclosure are described above.

Also described herein is the use of a complement protein selected from one or more of FHR1, FHR2, FHR3, FHR4 and/or FHR5, and optionally FH and/or FHL-1, for identifying a subject having a complement-related disorder or at risk of developing a complement-related disorder, or for determining whether a subject has or is at risk of developing a complement-related disorder, the use comprising:
(a) determining the level of a complement protein selected from one or more of FHR1, FHR2, FHR3, FHR4 and/or FHR5, and optionally FH and/or FHL-1, in a blood sample obtained from the subject; and
(b) determining that the subject has or is likely to develop a complement-related disorder if the level of FHR1, FHR2, FHR3, FHR4 and/or FHR5, and optionally FHL-1, is elevated as compared to the level of that complement protein in blood in a control subject that does not have a complement-related disorder.

Also provided is the use of a complement protein selected from one or more of FHR1, FHR2, FHR3, FHR4 and/or FHR5, and optionally FH and/or FHL-1, as a biomarker e.g. for identifying a subject having a complement-related disorder or at risk of developing a complement-related disorder, or for determining whether a subject has or is at risk of developing a complement-related disorder, the use comprising the steps described hereinabove.

In one aspect, provided is the use of endoproteinase GluC in a method for determining the presence and/or level of a complement protein, e.g. in a sample or a subject, e.g. according to the methods described herein. Also provided is the use of endoproteinase GluC in a method of identifying a subject having a complement-related disorder or at risk of developing a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject has or is likely to develop a complement-related disorder.

Also provided is the use of GluC in a method of selecting a subject for treatment of a complement-related disorder with a complement-targeted therapeutic, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject is in need of a complement-targeted therapeutic.

In some embodiments the methods described herein are performed in vitro or ex vivo. For example, a sample may be obtained from a subject of interest, and/or a control subject, and the determining steps are performed in vitro or ex vivo.

In methods described herein the level of the complement protein(s) is compared to the level of a reference value or level, sometimes called a control. In some cases the level of the complement protein(s) is compared to the level of the same complement protein in a control subject that does not have a complement-related disorder. A reference value may be obtained from a control sample, which itself may be obtained from a control subject. Data or values obtained from the individual to be tested, e.g. from a sample, can be compared to data or values obtained from the control sample. In some cases, the control is a spouse, partner, or friend of the subject.

As used herein the term “reference value” refers to a known measurement value used for comparison during analysis. In some cases, the reference value is one or a set of test values obtained from an individual or group in a defined state of health. The reference value may be one or a set of test values obtained from a control. In some cases, the reference value is/has been obtained from determining the level of complement proteins in subjects known not to have a complement-related disorder. In some cases, the reference value is/has been obtained from determining the level of complement proteins in subjects which have a complement-related disorder that is not associated with elevated levels of FHR protein(s), e.g. a subset of subjects in which FHR proteins are not considered to be a pathological factor. In some cases, the reference value is set by determining the level or amount of a complement protein previously from the individual to be tested e.g. at an earlier stage of disease progression, or prior to onset of the disease. The reference value may be taken from a sample obtained from the same subject, or a different subject or subject(s). The sample may be derived from the same tissue/cells/bodily fluid as the sample used by the present disclosure. The reference value may be a standard value, standard curve or standard data set. Values/levels which deviate significantly from reference values may be described as atypical values/levels.

In some cases the control may be a reference sample or reference dataset, or one or more values from said sample or dataset. The reference value may be derived from a reference sample or reference dataset. The reference value may be derived from one or more samples that have previously been obtained from one or more subjects that are known not to have a complement-related disorder and/or known or expected not to be at risk of developing a complement-related disorder. The reference value may be derived from one or more samples that have previously been obtained from one or more subjects that are known to have a complement-related disorder. The reference value may be derived from one or more samples that have previously been obtained from one or more subjects that are known to be at risk of developing a complement-related disorder. The reference value may be consensus level or an average, or mean, value calculated from a reference dataset, e.g. a mean protein level. The reference dataset/value may be obtained from a large-scale study of subjects known to have a complement-related disorder, such as AMD, e.g. as described herein.

The reference value may be derived from one or more samples that have previously been obtained from one or more subjects that are in the same family as the subject of interest, or from one or more subjects that are not in the same family as the subject of interest.

The reference value may be derived from one or more samples that have previously been obtained and/or analysed from the individual/subject/patient to be tested, e.g. a sample was obtained from the individual when they were at an earlier stage of a complement-related disorder, or a sample was obtained from the individual before the onset of a complement-related disorder.

The reference value may be obtained by performing analysis of the sample taken from a control subject in parallel with a sample from the individual to be tested. Alternatively, the control value may be obtained from a database or other previously obtained value. The reference value may be determined concurrently with the methods disclosed herein, or may have been determined previously.

Control subjects from which samples are/have been obtained may have undergone treatment for a complement-related disorder and/or received a complement-related therapy/therapeutic agent.

Controls may be positive controls in which the target molecule is known to be present, or expressed at high level, or negative controls in which the target molecule is known to be absent or expressed at low level.

Samples from one or more control subjects may comprise any one, two, three, four, five, six of seven of FHR1, FHR2, FHR3, FHR4, FHR5, FH and/or FHL-1. In some cases each complement protein is in a separate control sample. In some cases a control sample contains multiple complement proteins. In some cases the methods described herein comprise comparing the level of one of more complement proteins determined as described herein to different, e.g. one or more, samples, each sample containing one or more complement proteins. In some cases the methods described herein comprise comparing the level of one or more complement proteins determined as described herein to a single sample, wherein the sample contains one or more complement proteins.

In some cases control samples are obtained from the same tissue(s) as the sample obtained from the individual to be tested. In some cases control samples are obtained from different tissue(s) as the sample obtained from the individual to be tested. Control samples may be obtained from control subjects at certain time(s) of day, or on certain days. Sample(s) obtained from the individual to be tested are preferably obtained at the same time(s) of day and/or day(s) as the control samples.

In some cases, an increase/decrease of a complement protein, e.g. as described herein, as compared to a reference value indicates an increased risk of developing a complement-related disorder. In some cases, an increase/decrease of a complement protein, e.g. as described herein, indicates an increased risk of developing the disorder when compared to a reference value taken from the same subject at an earlier stage of the disorder, e.g. in a sample from the same subject.

In some embodiments, a method described herein may comprise determining the level of two or more complement proteins and comparing their values e.g. concentrations. The values may be compared to each other, as well as to reference values, e.g. increased levels of C3 and C3b compared to stationary or decreased levels of iC3b and further C3b breakdown products may be indicative of a higher risk of development of a complement-related disorder and/or the need to treat a subject for a complement-related disorder. Decreased levels of C3 and C3b compared to stationary or increased levels of iC3b and further C3b breakdown products may be indicative of a lower risk of development of a complement-related disorder and/or that treatment for a complement-related disorder is effective.

In some cases, a method described herein may comprise comparing the levels of any one or more of FHR1, FHR2, FHR3, FHR4 and/or FHR5, to the level of FH and/or FHL-1 in the subject tested. For example, elevated levels of FHR1, FHR2, FHR3, FHR4 and/or FHR5, and optionally FHL-1, compared to stationary levels of FH (i.e. a statistically non-significant change) in a subject may be indicative of a higher risk of the subject developing a complement-related disorder and/or the need to treat the subject for a complement-related disorder.

In some embodiments, a method provided herein comprises a step of correlating the presence of an atypical or altered amount/level of a complement protein with an increased risk of the subject developing or having a complement-related disorder.

Examples of reference values for complement proteins in human subjects known not to have a complement-related disorder include:
a) FH: ˜150 to 500 pg/ml in human blood (Clark et al., J Immunol 2014. 193(10):4962-70 and unpublished data);
   Mean: 833 nM in human plasma, SD: 149 (derived from the mass spectrometry methods described herein);
b) FHL-1: ˜0.5 to 50 pg/ml in human blood (Clark et al., J Immunol 2014. 193(10):4962-70 and unpublished data);
   Mean: 10.9 nM in human plasma, SD: 2.4 (derived from the mass spectrometry methods described herein);
c) FHR1: ˜70 to 100 pg/ml in human plasma (Heinen, S et al., Blood 114, 2439-2447);
   Mean: 33.9 nM in human plasma, SD: 16.5 (derived from the mass spectrometry methods described herein);
d) FHR2: ˜15-50 pg/ml in human plasma, or about 1/10 of FH concentration (Skerka et al., Mol Immunol 2013, 56:170-180);
   Mean: 51.2 nM in human plasma, SD: 17.0 (derived from the mass spectrometry methods described herein);
e) FHR3: ˜70 to 100 pg/ml in human plasma (Fritsche, L. G. et al., Hum. Mol. Genet. 2010.19, 4694-4704);
   Mean: 20.6 nM in human plasma, SD: 13.8 (derived from the mass spectrometry methods described herein);
f) FHR4: ≤5 μg/ml in human blood (WO 2019/215330);
   Mean: 46.1 nM in human plasma, SD: 24.4 (derived from the mass spectrometry methods described herein);
g) FHR5: ˜1.5 μg/ml in human plasma (van Beek, A E et al., Front Immunol. 2017 Oct. 18; 8:1328);
   Mean: 29.2 nM in human plasma, SD: 8.7 (derived from the mass spectrometry methods described herein);
h) FI: ˜35 μg/ml in human plasma;
i) C3: ˜0.5-16 mg/ml in human plasma (Engström, G. et al., J Hum Hypertens. 2007 April; 21(4):276-82; Lee S H et al., Am J Respir Crit Care Med. 2006 Feb. 15; 173(4):370-8);
j) C3a: 46-157 ng/ml (Lee S H et al., Am J Respir Crit Care Med. 2006 Feb. 15; 173(4):370-8);
k) iC3b: ˜0.7-5 μg/ml (Kim A H J et al., Arthritis Rheumatol. 2019 March; 71(3):420-430).

In some cases, mean reference values for circulating FH, FHL-1 and FHR1-5 in human subjects known not to have a complement-related disorder, e.g. AMD, include the following (95% Cl in parentheses):
a) FH, nM: 737.3 (718.2-756.5)
b) FHL-1, nM: 10.4 (10.1-10.8)
c) FHR-1, nM: 31.2 (29.4-32.9)
d) FHR-2, nM: 45.3 (43.1-47.6)
e) FHR-3, nM: 24.1 (21.7-26.5)
f) FHR-4, nM: 46.1 (42.7-49.6)
g) FHR-5, nM: 25.5 (24.5-26.5).

The relative concentrations of one complement protein to another can be determined using their reference values. For example, the ratio of the level of one complement protein to the level of another, or others, can be inferred from the concentrations provided above, e.g. FH:FHL-1, C3:iC3b, C3:C3b etc. The relative concentrations and/or ratios of the level of one complement protein to another, or others, may be altered in complement-related disorders. In some embodiments the methods provided herein involve detecting two or more complement proteins and determining how the levels of the complement proteins change with respect to one another as compared to a reference value(s). For example, the level of a first complement protein may increase as compared to the level of a second complement protein, or vice versa, e.g. FHL-1 vs FH, FHR1 to FHR5 vs FH and/or FHL-1, C3 vs iC3b, C3 vs C3b.

### Methods for Detecting/Determining the Level of Complement Proteins

Aspects and embodiments of the present disclosure involve detecting the presence of, and/or determining the level of, one or more complement proteins using suitable analytical techniques, e.g. as described herein.

In some embodiments a method described herein comprises contacting the complement protein with endoproteinase GluC to obtain one or more peptides, and detecting the one or more peptides by mass spectrometry.

In some embodiments a method described herein comprises contacting, e.g. digesting, the protein with GluC to obtain one or more peptides, and determining the level of the one or more peptides by mass spectrometry. In some cases, the methods involves both detecting a complement protein and determining the level of a complement protein. The protein may be the same protein, or the methods may involve detection of a first complement protein and determining the level of a second complement protein.

In any and all methods described herein, the step of detecting/determining the level of the one or more peptides consists of detecting/determining the level of/measuring the peptide(s) by mass spectrometry. That is, the step of detecting/determining the level of/measuring the peptide(s) is performed by mass spectrometry only. Measuring the peptide(s) may include detecting the presence or absence of the one or more peptides, and/or determining the level, amount and/or concentration of each peptide in the sample.

In some embodiments, the step of determining in any method described herein involves:
(i) digesting at least one complement protein in a sample e.g. blood sample obtained from the subject with endoproteinase GluC to obtain one or more peptides;
(ii) determining the presence and/or level of the one or more peptides by mass spectrometry; and
(iii) using the results of (ii) to determine whether or not the level of the complement protein(s) is elevated, e.g. as compared to the level of that complement protein(s) in a blood sample in a control subject that does not have a complement-related disorder.

The complement protein(s) may be selected from one or more of FHR1, FHR2, FHR3, FHR4 and/or FHR5, in any combination as described herein. The presence and/or level of FH and/or FHL-1 may also be determined.

The term “digesting” as used herein refers to placing the protein in contact with GluC under suitable conditions, e.g. temperature, pH etc, and for a suitable time such that the protein is digested, i.e. cleaved, into two or more fragments. In some cases, the digesting involves incubating the protein with GluC under suitable conditions, e.g. as described herein.

In some aspects the provided is a method for preparing a complement protein for analysis, the method comprising contacting/digesting the protein with endoproteinase GluC to obtain one or more peptides. In some cases, the method comprises preparing a complement protein for subsequent analysis. The one or more peptides may then be subjected to an analytical technique, e.g. mass spectrometry or any other suitable analytical technique. In some cases the method comprises preparing a complement protein for analysis by mass spectrometry. The analytical technique may be used to detect the presence and/or level of the one or more peptides.

It will be appreciated that where “complement protein” is referred to herein in the singular (i.e. “a/the complement protein”), pluralities/groups/populations of different complement proteins are also contemplated. For example, any disclosure herein comprising a complement protein also comprises more than one complement protein, i.e. at least one protein, or one or more proteins. In all aspects and embodiments described herein, “a/the complement protein” may refer to “at least one complement protein”.

“Detecting” a protein as used herein refers to identifying/observing the presence or existence of the protein, e.g. in a sample, cell, tissue or subject.

The “level” of a complement protein used herein refers to the level, amount or concentration of said protein, e.g. in a sample, cell, tissue, organ or subject. The term “determining the level”, e.g. of a protein, used herein refers to the measurement and/or quantification of the level, amount or concentration of a protein. In some cases, “determining the level” includes calculating the level, amount or concentration of a protein in a sample. The sample may be from a subject. In some cases, “determining the level” includes calculating the level, amount or concentration of a protein in a subject, e.g. using a sample taken from the subject. “Determining the level” of a protein may include digesting the protein with GluC to obtain one or more peptides, detecting the one or more peptides as described herein and then calculating the level, amount or concentration of the protein/peptide, e.g. in a sample.

In some cases, “determining the level” comprises quantifying, i.e. measuring the quantity of, the level, amount or concentration of a protein e.g. in a sample or in a subject. “Determining the level” may include determining the concentration of a protein. Quantification/measuring may include comparing the level, amount or concentration of a protein with a reference value, and/or comparing the level, amount or concentration of a protein with that in a control sample e.g. taken from the subject at a different time point, or taken from a healthy subject, e.g. one known not to have a complement-related disorder.

In some embodiments the methods comprise detecting/determining the level of a complement protein in a sample. The sample may be in vitro or ex vivo. A sample may have been taken from a subject, e.g. from a subject of interest or from a control subject. A sample may be taken from any tissue or bodily fluid. In preferred arrangements the sample is taken from a bodily fluid, more preferably one that circulates through the body. The sample may be referred to as a circulating sample. Accordingly, the sample may be a blood sample or lymph sample. In a particularly preferred arrangement the sample is a blood sample or blood-derived sample. The blood-derived sample may be a selected fraction of a subject's blood, e.g. a selected cell-containing fraction or a plasma or serum fraction. A selected serum fraction may comprise the fluid portion of the blood obtained after removal of the fibrin clot and blood cells. Alternatively the sample may comprise or may be derived from a tissue sample, biopsy or isolated cells from said individual. The sample may be taken from the eye, kidney, brain or liver, e.g. comprising cells from the eye, kidney, brain or liver. The sample may comprise retinal tissue. The sample may comprise RPE cells or tissue from Bruch's membrane or the choroid. The sample may comprise drusen or other deposits of complement-related components.

In some embodiments the methods described herein comprise taking or obtaining a sample from a subject, e.g. blood, tissue etc. In some embodiments the methods described herein are performed on a sample that has been obtained/was obtained from a subject. In some cases the sample is a blood sample. The blood sample may undergo/have undergone processing to obtain a plasma sample or a serum sample. In some cases, the methods comprise obtaining a blood-derived sample from a subject. In some cases, the methods comprise obtaining a plasma or serum sample from a subject. In some embodiments the methods comprise isolating protein, e.g. total protein, from the sample. Suitable techniques to isolate protein from biological samples are well known in the field. In some embodiments the methods do not comprise isolating protein from the sample, e.g. the methods are performed on the unprocessed sample.

In some embodiments, the methods are performed in vitro. For example, the presence, level, amount and/or concentration of the complement protein(s) may be detected/determined in vitro.

In some cases the methods involve determining the presence, level, amount and/or concentration of the complement protein(s) in a subject. This may involve performing the methods described herein in vitro, and using the results to calculate the presence, level, amount and/or concentration of the protein(s) in the subject.

Also provided is a method for detecting at least one complement protein in a sample, the method comprising digesting the protein(s) in the sample with endoproteinase GluC to obtain one or more peptides; and using mass spectrometry to detect the one or more peptides in the sample. Any method described herein may comprise a step of detecting at least one complement protein, e.g. detecting the presence of the complement protein.

Also provided is a method for determining the level of at least one complement protein in a sample, the method comprising digesting the protein(s) in the sample with endoproteinase GluC to obtain one or more peptides and using mass spectrometry to determine the level of the one or more peptides in the sample.

Using mass spectrometry to detect one or more peptides in a sample, or detecting and/or determining the level of one or more peptides by mass spectrometry, e.g. by the methods described herein, may include applying a mass spectrometry technique to the sample, e.g. by putting the sample in a mass spectrometer, and instructing the mass spectrometer to analyse the sample. Various suitable mass spectrometry techniques are disclosed herein and are within the routine tasks of a skilled person.

In any aspect provided herein, the methods described herein may comprise both detecting at least one complement protein and determining the level of at least one complement protein. The complement protein may be the same protein, and/or the methods may comprise detecting a least a first complement protein and determining the level of at least a second complement protein.

In some embodiments, the methods described herein comprise detecting/determining the level of one complement protein. In some embodiments, the methods described herein comprise detecting/determining the level of at least one complement protein, one or more complement proteins, and/or groups or complement proteins e.g. as provided herein.

In some embodiments the complement protein is encoded from the RCA (regulators of complement) gene cluster, or RCA locus, on human chromosome 1. The RCA cluster is located on chromosome 1q32 and includes the CFH and CFHR1-5 genes. The gene cluster also includes the membrane bound proteins CR1 (CD35), CR2 (CD21), decay-accelerating factor (DAF; CD55), and membrane cofactor protein (MCP; CD46), as well as soluble C4b-binding protein (C4 bp).

The methods described herein are suitable for detecting/determining the level of multiple complement proteins via a single assay: i.e. using a single enzyme, GluC, to obtain analysable peptides and then using a single analytical technique, mass spectrometry, to detect and/or determine the levels of said peptides. In this way, the complementome of a sample or a subject can be determined via a single assay.

In some embodiments the methods described herein comprise detecting/determining the level of any one or more, e.g. any or all combinations, of FH, FHL-1, FHR1, FHR2, FHR3, FHR4, and/or FHR5. In some embodiments the complement protein(s) is/are selected from the group consisting of FH, FHL-1, FHR1, FHR2, FHR3, FHR4, and/or FHR5. In some cases the methods comprise detecting/determining the level of any one, two, three, four, five, six and/or seven of FH, FHL-1, FHR1, FHR2, FHR3, FHR4, and FHR5, alone or in combination. In some cases the methods described herein are able to differentiate (i.e. distinguish, discriminate, separate) between the presence of (or levels of) each of FH, FHL-1, FHR1, FHR2, FHR3, FHR4 and/or FHR5.

In some cases the complement protein is any one or more, e.g. any or all combinations, of FHR1, FHR2, FHR3, FHR4 and/or FHR5. In some cases the complement protein is FHR1. In some cases the complement protein is FHR2. In some cases the complement protein is FHR3. In some cases the complement protein is FHR4. In some cases the complement protein is FHR5. In some cases the methods described herein are able to differentiate (i.e. distinguish, discriminate, separate) between the presence of (or levels of) each of FHR1, FHR2, FHR3, FHR4 and/or FHR5. Exemplary combinations of FHR proteins that may be detected in the present disclosure are described herein.

In some cases, the methods described herein permit or allow the detection of FHR1 alone, i.e. without detecting FHR2-FHR5. In some cases, the methods described herein permit or allow the detection of FHR2 alone, i.e. without detecting FHR1 or FHR3-FHR5. In some cases, the methods described herein permit or allow the detection of FHR3 alone, i.e. without detecting FHR1, FHR2, FHR4, or FHR5. In some cases, the methods described herein permit or allow the detection of FHR4 alone, i.e. without detecting FHR1-FHR3 or FHR5. In some cases, the methods described herein permit or allow the detection of FHR5 alone, i.e. without detecting FHR1-FHR4.

In some cases the complement protein is to be detected/determine the level of FH and/or FHL-1. In some cases the methods described herein comprise detecting/determining the level of both FH and FHL-1. In some cases the methods described herein differentiate (i.e. distinguish, discriminate, separate) between the presence of FH and the presence of FHL-1 and/or between the level/concentration of FH and the level/concentration of FHL-1. In some cases the methods described herein permit or allow the detection of FH alone, i.e. without detecting FHL-1. In some cases the methods described herein permit or allow the detection of FHL-1 alone, i.e. without detecting FH.

In some embodiments the complement protein to be detected/the level of which is determined is involved with breakdown, turnover and/or inactivation of C3/C3b. In some embodiments, the complement protein is produced by the breakdown and/or inactivation of C3/C3b, i.e. is a product of C3b inactivation/breakdown. In some embodiments the methods described herein include determining the presence, rate and/or progression of C3b turnover. In some embodiments the methods described herein involve detecting/determining the level of a protein involved in, or produced as a result of, the complement amplification loop. In some embodiments the methods described herein involve detecting/determining the level of a protein involved in the generation or breakdown of C3 convertase. In some cases the protein is a cofactor for FI, e.g. FH, CR1, or the FHR proteins. Any method disclosed herein, e.g. a method for detecting at least one complement protein in a sample comprising digesting proteins with GluC and detecting the resulting peptides by mass spectrometry, may be described in the alternative as a method for detecting C3 turnover, a method for detecting C3 breakdown, a method for measuring C3b turnover or C3b breakdown, or a method for measuring the progress of C3b turnover or C3b breakdown.

Thus, in some aspects the present disclosure provides a method for detecting turnover or breakdown of C3b, comprising the steps described herein, e.g. digesting at least one complement protein with endoproteinase GluC to obtain one or more peptides and detecting the peptide(s) by mass spectrometry. In some cases the method comprises digesting and then detecting at least two, three, four or more, up to 16, of the 16 complement proteins described herein.

In some embodiments the methods described herein comprise/further comprise detecting/determining the level of FI, either alone or in combination with other complement proteins such as those described herein.

In some embodiments the methods described herein comprise detecting/determining the level of any one or more, e.g. any or all combinations, of C3, C3b, C3a, iC3b, C3f, C3c, C3dg, and/or C3d. In some embodiments the complement protein(s) is/are selected from the group consisting of C3, C3b, C3a, iC3b, C3f, C3c, C3dg, and/or C3d. In some cases the methods comprise detecting/determining the level of any one, two, three, four, five, six, seven and/or eight of C3, C3b, C3a, iC3b, C3f, C3c, C3dg, and/or C3d, in any combination. In some embodiments the methods described herein comprise detecting/determining the level of one or more of C3, C3a, C3f, C3c, and/or C3d. In some cases the methods described herein comprise determining the presence and/or level of C3b, iC3b, and/or C3dg, e.g. via the methodology in Table 3. In some cases the methods described herein comprise detecting/determining the level of C3, C3b and/or iC3b. In some cases the methods described herein are able to differentiate (i.e. distinguish, discriminate, separate) between the presence of (or levels of) two or more, or all, of C3, C3b, C3a, iC3b, C3f, C3c, C3dg, and/or C3d.

The methods described herein can detect multiple complement proteins, and distinguish between said complement proteins, using one enzyme e.g. GluC and one analytical method e.g. mass spectrometry. The methods described herein may be used to detect/determine the level of any one of the individual proteins described herein, as well as any and all combinations of FH, FHL-1, FHR1, FHR2, FHR3, FHR4, FHR5, FI, C3, C3b, C3a, iC3b, C3f, C3c, C3dg, and/or C3d, i.e. any one, two, three, four, five, six, seven, eight, nine, ten, eleven, twelve, thirteen, fourteen, fifteen and/or sixteen of these proteins in any combination. In some embodiments the complement protein(s) is/are selected from the group consisting of FH, FHL-1, FHR1, FHR2, FHR3, FHR4, FHR5, FI, C3, C3b, C3a, iC3b, C3f, C3c, C3dg, and/or C3d. In some cases the methods described herein may be used to detect/determine the level of FHL-1 and to detect/determine the level of any one or more of FH, FHR1, FHR2, FHR3, FHR4, FHR5, FI, C3, C3b, C3a, iC3b, C3f, C3c, C3dg, and/or C3d. In some cases the method comprises distinguishing (i.e. differentiating, discriminating, separating) between the presence/level of FHL-1 and the presence/level of any one or more of FH, FHR1, FHR2, FHR3, FHR4, FHR5, FI, C3, C3b, C3a, iC3b, C3f, C3c, C3dg, and/or C3d. The terms “distinguishing”, “differentiating”, “discriminating”, and “separating” are used interchangeably herein.

In some cases the methods provided herein allow for simultaneous detection of one or more of FH, FHL-1, FHR1, FHR2, FHR3, FHR4, FHR5, FI, C3, C3b, C3a, iC3b, C3f, C3c, C3dg, and/or C3d, including any combination thereof. In some cases the methods provided herein allow for detection/determination of the level of one or more of FH, FHL-1, FHR1, FHR2, FHR3, FHR4, FHR5, FI, C3, C3b, C3a, iC3b, C3f, C3c, C3dg, and/or C3d, including any combination thereof, in a single assay. The methods provided herein allow for distinct, separable and detectable peptides to be produced from every protein listed above such that the presence and/or level of each protein can be distinguished from the others.

In some cases the methods provided herein allow for simultaneous detection of one or more of FHR1, FHR2, FHR3, FHR4, FHR5, FH, and/or FHL-1, including any combination thereof. In some cases the methods provided herein allow for detection/determination of the level of one or more of FHR1, FHR2, FHR3, FHR4, FHR5, FH, and/or FHL-1, including any combination thereof, in a single assay.

In some cases, the present disclosure provides a method for detecting and/or determining the level of at least two complement proteins in a sample simultaneously and/or in one assay, the method comprising:
digesting the proteins with endoproteinase GluC to obtain one or more peptides; and
detecting and/or determining the level of the one or more peptides by mass spectrometry.

In any method described herein, the complement protein may be any protein involved in one or more of the complement system pathways. For example, the complement protein may be one or more of C1, C2, C4b2a C4, C4a, C5, C5a, FB, FD, C3Bb, MASP1, MASP2, C1q, C1r, C1s, C6, C7, C8, C9, CD59, Clusterin, Properdin, and/or Compstatin. In any embodiment described herein, the complement protein to be detected (or the protein whose level is determined) is not one or more of C1, C2, C4b2a C4, C4a, C5, C5a, FB, FD, C3Bb, MASP1, MASP2, Clq, C1r, C1s, C6, C7, C8, C9, CD59, Clusterin, Properdin, and/or Compstatin.

In some cases, the present disclosure provides endoproteinase GluC for preparing at least one complement protein for detection by mass spectrometry. In some cases the present disclosure provides endoproteinase GluC for preparing at least two, i.e. multiple or a plurality of, complement proteins for detection by mass spectrometry. The at least two complement proteins may be any two, three, four or more, up to 16, of FH, FHL-1, FHR1, FHR2, FHR3, FHR4, FHR5, FI, C3, C3b, C3a, iC3b, C3f, C3c, C3dg, and/or C3d, in any combination, as described herein.

Endoproteinase GluC, also known as glutamyl endopeptidase, is a serine proteinase which preferentially cleaves peptide bonds C-terminal to glutamic acid residues. It also cleaves at aspartic acid residues at a rate 100-300 times slower than at glutamic acid residues. The specificity of GluC depends on the pH and the buffer composition. At pH 4, the enzyme preferentially cleaves at the C terminus of E, whereas at pH 8 it additionally cleaves at D residues. The sequence of GluC is provided in SEQ ID NO:153 and 154.

In preferred embodiments, the methods described herein use GluC alone (i.e. only GluC) to digest the one or more complement proteins. In preferred embodiments, a step of digesting the protein(s) in the described methods consists of digesting the protein(s) with GluC. In preferred embodiments, any method described herein does not employ/use any other protease alone or in combination with GluC. For example, in some embodiments the digestion step of any method described herein does not use, or is not performed by, any one or more of the following enzymes or agents: trypsin, chymotrypsin (high specificity or low specificity), Lys-C, Lys-N, Arg-C, Asp-N, elastase, LysargiNase, pepsin, Sap9, OmpT, BNPS-skatole, any caspase, clostripain (clostridiopeptidase B), CNBr, enterokinase, factor Xa, granzymeB, neutrophil elastase, proteinase K, thermolysin, non-GluC glutamyl endopeptidase e.g. GluBI or GIuSGB, proline endopeptidase, TEV protease, thrombin, formic acid, hydroxylamine, iodosobenzoic acid, and/or NTCB (or any combination thereof).

GluC is obtainable from standard reagent providers e.g. Sigma Aldrich, NEB etc, and may be used according to the accompanying instructions or according to protocols well known in the field. An example protocol is described herein. Obtaining proteins from biological samples and suitable buffers to prepare samples/proteins for GluC digestion will also be known to the skilled person. An example cell lysis buffer comprises: 8 M urea (4.8 g per 10 ml) in 50 mM NH₄HCO₃ and 20 mM methylamine, diluted to a urea concentration of <2 M, pH 8 (40 mg per 10 ml), containing 1 tablet of cOmplete™ Mini EDTA-free protease inhibitor cocktail per 10 ml of lysis buffer.

In some cases, a complement protein is contacted/incubated/digested with GluC enzyme for at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 hours. In some cases a complement protein is contacted/incubated/digested with GluC enzyme for at least 12 hours. In some cases a complement protein is contacted/incubated/digested with GluC enzyme for about 12 hours, e.g. 12 hours. In some cases a complement protein is contacted/incubated/digested with GluC enzyme for about 16 hours, e.g. 16 hours. The terms contacted, incubated and digested are used interchangeably herein.

In some cases a complement protein is contacted/incubated/digested with GluC enzyme at a temperature of at least 20° C., at least 21° C., at least 22° C., at least 23° C., at least 24° C., at least 25° C., at least 26° C., at least 27° C., at least 28° C., at least 29° C., or at least 30° C. In some cases a complement protein is contacted/incubated/digested with GluC enzyme at a temperature of at least 25° C. In some cases a complement protein is contacted/incubated/digested with GluC enzyme at a temperature of about 25° C., e.g. 25° C.

In some cases a complement protein is contacted/incubated/digested with GluC enzyme at a pH of at least 7.0, at least 7.1, at least 7.2, at least 7.3, at least 7.4, at least 7.5, at least 7.6, at least 7.7, at least 7.8, at least 7.9, at least 8.0, at least 8.1, at least 8.2, at least 8.3, at least 8.4, at least 8.5, at least 8.6, at least 8.7, at least 8.8, at least 8.9, or at least 9.0. In some cases a complement protein is contacted/incubated/digested with GluC enzyme at a pH of at least 8.0. In some cases a complement protein is contacted/incubated/digested with GluC enzyme at a pH of about 8.0, e.g. a pH of 8.0.

In some cases the GluC enzyme and complement protein are contacted/incubated at a wt/wt ratio of 1/75. The incubation step may comprise gentle shaking, e.g. at 400 rpm.

The methods described herein may comprise a contacting/incubation/digestion step comprising any combination of temperature, pH, and/or time as described above. In some cases, contacting/incubating/digesting is performed at 25° C. at pH8 for 12 hours.

In some embodiments the present disclosure provides a method for detecting and/or determining the level of at least one complement protein e.g. in a sample, the method comprising:
digesting the protein(s) with endoproteinase GluC to obtain one or more peptides, the digesting comprising incubating the protein(s) with GluC at 25° C. at pH8 for up to 12 hours; and
detecting the one or more peptides by mass spectrometry.

In some embodiments the present disclosure provides a method for detecting and/or determining the level of at least one complement protein e.g. in a sample, the method comprising:
digesting the protein(s) with endoproteinase GluC to obtain one or more peptides, the digesting comprising incubating the protein(s) with GluC at 25° C. at pH8 for up to 16 hours; and
detecting the one or more peptides by mass spectrometry.

The following peptides may be produced by GluC digestion of complement proteins, e.g. as described herein. In some embodiments, the methods described herein comprise detecting/determining the level of any one or more of these peptides, i.e. any one or more of SEQ ID NO:20 to 141, or 155, 156 or 157, in any combination. All combinations of peptides are envisaged. The mass of peptides represented by SEQ ID NOs 20-27 can be found in Table 1.

In some embodiments, the FH peptide is VTYKCFE (SEQ ID NO:20).

In some embodiments the FH peptide is any one or more of SNTGSTTGSIVCGYNGWSDLPICYE (SEQ ID NO:112; mass 2623.1206), NGWSPTPRCIRVKTCSKSSIDIE (SEQ ID NO:113; mass 2576.2839), LPKIDVHLVPDRKKDQYKVGE (SEQ ID NO:114; mass 2476.3801), YYCNPRFLMKGPNKIQCVDGE (SEQ ID NO:115; mass 2474.1545), NYNIALRWTAKQKLYSRTGE (SEQ ID NO:116; mass 2411.2709), KWSHPPSCIKTDCLSLPSFE (SEQ ID NO:117; mass 2274.0813), HGWAQLSSPPYYYGDSVE (SEQ ID NO:118; mass 2054.9010), ISHGVVAHMSDSYQYGEE (SEQ ID NO:119; mass 2007.8632), FDHNSNIRYRCRGKE (SEQ ID NO:120; mass 1893.9016), ITCKDGRWQSIPLCVE (SEQ ID NO:121; mass 1846.9069), GWIHTVCINGRWDPE (SEQ ID NO:122; mass 1781.8307), KAKYQCKLGYVTADGE (SEQ ID NO:123; mass 1772.8767), TTCYMGKWSSPPQCE (SEQ ID NO:124; mass 1716.6946), SYAHGTKLSYTCE (SEQ ID NO:125, mass 1458.6449), RVRYQCRSPYE (SEQ ID NO:126; mass 1455.7041), GFGIDGPAIAKCLGE (SEQ ID NO:127; mass 1446.7176), HGTINSSRSSQE (SEQ ID NO:128; mass 1301.5960), YQCQNLYQLE (SEQ ID NO:129; mass 1300.5758), WTTLPVCIVEE (SEQ ID NO:130; mass 1288.6373), KIPCSQPPQIE (SEQ ID NO:131; mass 1238.6329), SQYTYALKE (SEQ ID NO:132; mass 1101.5342), QVQSCGPPPE (SEQ ID NO:133; mass 1040.4597), KKDVYKAGE (SEQ ID NO:134; mass 1036.5553), GLPCKSPPE (SEQ ID NO:135; mass 926.4531), KVSVLCQE (SEQ ID NO:136; mass 904.4688), HLKNKKE (SEQ ID NO:137; mass 895.5239), GGFRISEE (SEQ ID NO:138; mass 893.4243), LLNGNVKE (SEQ ID NO: 139; mass 885.4920), YPTCAKR (SEQ ID NO:140; mass 837.4167), or STCGDIPE (SEQ ID NO:141; mass 820.3273).

In some embodiments, the FHL-1 peptide is NGWSPTPRCIRVSFTL (SEQ ID NO:21).

In some embodiments, the FHR1 peptide is ATFCDFPKINHGILYGEE (SEQ ID NO:22).

In some embodiments the FHR1 peptide is NYNIALRWTAKQKLYLRTGE (SEQ ID NO:91; mass 2437.3230).

In some embodiments the FHR2 peptide is RGWSTPPKCRSTISAE (SEQ ID NO:23).

In some embodiments the FHR2 peptide is AMFCDFPKINHGILYDEE (SEQ ID NO:24). In some embodiments the FHR2 peptide is YNFVSPSKSFWTRITCAEE (SEQ ID NO:92; mass 2264.0572).

In some embodiments the FHR3 peptide is VACHPGYGLPKAQTTVTCTE (SEQ ID NO:25).

In some embodiments the FHR3 peptide is any one or more of KGWSPTPRCIRVRTCSKSDIE (SEQ ID NO:93; mass 2418.2260), NGYNQNYGRKFVQGNSTE (SEQ ID NO:94; mass 2074.9457), QVKPCDFPDIKHGGLFHE (SEQ ID NO:95; mass 2066.0043), FMCKLGYNANTSILSFQAVCRE (SEQ ID NO:96; mass 2494.1807), or YQCQPYYE (SEQ ID NO:97; mass 1092.4222).

In some embodiments the FHR4 peptide is YQCQSYYE (SEQ ID NO:26).

In some embodiments the FHR4 peptide is any one or more of NSRAKSNGMRFKLHDTLDYE (SEQ ID NO: 98; mass 2381.1546), DGWSHFPTCYNSSE (SEQ ID NO:99; mass 1628.6202), ISYGNTTGSIVCGE (SEQ ID NO:100; mass 1399.6289), or FMCKLGYNANTSVLSFQAVCRE (SEQ ID NO:101; mass 2480.1650).

In some embodiments the FHR5 peptide is RGWSTPPICSFTKGE (SEQ ID NO:27).

In some embodiments the FHR5 peptide is any one or more of GTLCDFPKIHHGFLYDEE (SEQ ID NO:102; mass 2119.9673), YAMIGNNMITCINGIWTE (SEQ ID NO:103; mass 2042.9264), YGYVQPSVPPYQHGVSVE (SEQ ID NO:104; mass 2004.9581), GDTVQIICNTGYSLQNNE (SEQ ID NO:105; mass 1967.8895), IVCKDGRWQSLPRCVE (SEQ ID NO:106; mass 1887.9447), DYNPFSQVPTGE (SEQ ID NO:107; mass 1352.5884), QVKTCGYIPE (SEQ ID NO:108; mass 1136.5536), ANVDAQPKKE (SEQ ID NO:109; mass 1098.5669), WTTLPTCVE (SEQ ID NO:110; mass 1048.4899), or KVAVLCKE (SEQ ID NO:111; mass 888.5102).

In some cases, the methods described herein comprise detecting/determining the presence and/or level of one or more of SEQ ID NOs 21-27, in any combination.

In some cases, any method described herein may comprise detecting/determining the level of one or more of SEQ ID NOs 28-37, 156 or 157, in any combination. In some cases, the methods provided herein are used to detect C3, C3b and breakdown products using one or more or all of the peptides in Table 2 in any combination, plus optionally SEQ ID NO:156 and/or 157, for example according to the methodology in Table 3.

In some embodiments the FI peptide is any one or more of VKLVDQDKTMFICKSSWSMRE (SEQ ID NO:45; mass 2531.2455), VKLISNCSKFYGNRFYE (SEQ ID NO:46; mass 2068.0320), CLHPGTKFLNNGTCTAE (SEQ ID NO:47; mass 1805.8309), NYNAGTYQNDIALIE (SEQ ID NO:48; mass 1698.7969), GKFSVSLKHGNTDSE (SEQ ID NO:49; mass 1605.7867), VGCAGFASVTQEE (SEQ ID NO:50; mass 1297.5729), VGCAGFASVTQE (SEQ ID NO:155; mass 1168.272), MKKDGNKKDCE (SEQ ID NO:51; mass 1295.6082), YVDRIIFHE (SEQ ID NO:52; mass 1191.6156), CLHVHCRGLE (SEQ ID NO:53; mass 1166.5557), RVFSLQWGE (SEQ ID NO:54; mass 1121.5738), ILTADMDAE (SEQ ID NO:55; mass 978.4448), or KVTYTSQE (SEQ ID NO:56; mass 955.4731).

In some embodiments the FI peptide is any one or more of CAGTYDGSIDACKGDSGGPLVCMDANNVTYVWGVVSWGE (SEQ ID NO:38; mass 3996.7183), GTCVCKLPYQCPKNGTAVCATNRRSFPTYCQQKSLE (SEQ ID NO:39; mass 3994.8853), FPGVYTKVANYFDWISYHVGRPFISQYNV (SEQ ID NO:40; mass 3467.7211), ANVACLDLGFQQGADTQRRFKLSDLSINSTE (SEQ ID NO:41, mass 3397.6804), LPRSIPACVPWSPYLFQPNDTCIVSGWGRE (SEQ ID NO:42, mass 3388.6605), KKCLAKKYTHLSCDKVFCQPWQRCIE (SEQ ID NO:43; mass 3155.5773), LCCKACQGKGFHCKSGVCIPSQYQCNGE (SEQ ID NO:44; mass 2991.2861), VKLVDQDKTMFICKSSWSMRE (SEQ ID NO:45; mass 2531.2455), VKLISNCSKFYGNRFYE (SEQ ID NO:46; mass 2068.0320), CLHPGTKFLNNGTCTAE (SEQ ID NO:47; mass 1805.8309), NYNAGTYQNDIALIE (SEQ ID NO:48; mass 1698.7969), GKFSVSLKHGNTDSE (SEQ ID NO:49; mass 1605.7867), VGCAGFASVTQEE (SEQ ID NO:50; mass 1297.5729), MKKDGNKKDCE (SEQ ID NO:51; mass 1295.6082), YVDRIIFHE (SEQ ID NO:52; mass 1191.6156), CLHVHCRGLE (SEQ ID NO:53; mass 1166.5557), RVFSLQWGE (SEQ ID NO:54; mass 1121.5738), ILTADMDAE (SEQ ID NO:55; mass 978.4448), KVTYTSQE (SEQ ID NO:56; mass 955.4731), VDCITGE (SEQ ID NO:57; mass 736.3182), NCGKPE (SEQ ID NO:58; mass 647.2817), TSLAE (SEQ ID NO:59; mass 520.2613), or KDNE (SEQ ID NO:60; mass 505.2252).

Peptides detected by the methods described herein may optionally have at least 70%, preferably one of 80%, 85%, 90%, 91%, 92%, 93%, 94%, 95%, 96%, 97%, 98%, 99% or 100% amino acid sequence identity to the amino acid sequences of the peptides described herein, e.g. any one of SEQ ID NOs 21-141. Other suitable peptides may be readily determined by a skilled person and may be employed in the methods described herein. In preferred embodiments, the peptides used according to the methods herein permit mass spectrometry techniques to distinguish or differentiate between two or more complement proteins in a sample.

### Mass Spectrometry

Methods provided herein comprising detecting and/or determining the levels of proteins, e.g. complement proteins, may involve using mass spectrometry to detect and/or determine the levels of proteins in a sample.

In preferred embodiments, any method described herein involves using only mass spectrometry (i.e. mass spectrometry alone) to detect/determine the level of the one or more peptides. That is, in some embodiments, the methods provided herein do not employ multiple analytical techniques and the peptide(s) are detected/determined/measured using a single assay. In preferred embodiments, the methods described herein do not detect/determine the level of/measure the peptide(s) using mass spectrometry in combination with another analytical technique suitable for detecting proteins/peptides. In preferred embodiments, detection/determination of the level of the one or more peptides is not performed at any stage using a non-mass spectrometry technique, e.g. detection/determination of the level of the peptide(s) is not performed using high performance liquid chromatography (HPLC), immunological-based methods such as quantitative enzyme-linked immunosorbent assays (ELISA), Western blotting, protein immunoprecipitation, dot blotting or immunoelectrophoresis, electrophoresis or autoradiography. In some cases, liquid chromatography-mass spectrometry (LC/MS) is not used.

As used herein, “detecting and/or determining the level of” e.g. a complement protein or peptide “by mass spectrometry” is the same as “using mass spectrometry to detect and/or determine the level of” e.g. a complement protein or peptide.

Mass spectrometry is a well-known analytical technique for analysing a sample that typically comprises generating ions from the sample, optionally fragmenting the ions, separating the ions according to their mass/charge ratio (in time and/or space), and detecting the ions to provide information regarding the content of the sample.

For the purpose of detecting and/or determining the levels of proteins, at least one fragmentation step may be included.

Mass spectrometry techniques are well-known in the field and any suitable mass spectrometry technique may be employed for detecting and/or determining the levels of proteins in a sample, e.g. LC/MS, GC/MS, tandem mass spectrometry (MS/MS), quadrupole MS e.g. triple quadrupole MS (TQMS), time-of-flight MS e.g. MALDI-TOF, targeted MS e.g. selected reaction monitoring MS (SRM-MS)/multiple reaction monitoring (MRM-MS), parallel-reaction monitoring (PRM-MS), trapped-ion based methods e.g. three-dimensional quadrupole ion traps (“dynamic” traps) and ion cyclotron resonance mass spectrometers (“static” traps), quadrupole trap MS, hybrid linear trap orbitrap MS, quadrupole-Orbitrap MS, electrospray Ionization mass spectrometry (ESI-MS), or electron transfer dissociation MS (ETD).

In some embodiments, the mass spectrometry technique may be a liquid chromatography-selected reaction monitoring mass spectrometry (LC-SRM-MS)-based assay.

Fragmenting the ions may be achieved using any suitable fragmentation technique, e.g. collision-induced dissociation (CID)/collisionally activated dissociation (CAD), electron-capture dissociation (ECD), electron transfer dissociation (ETD), in-source decay (ISD), infrared multiple photon dissociation (IRMPD) etc. Again, such techniques are well known.

The mass spectrometry techniques useful in connection with the present disclosure may comprise quantitative analysis. Mass spectrometry methods comprising quantitative analysis may comprise a targeted approach to detect and measure peptides of interest and their corresponding fragments. This may allow for greater specificity and sensitivity for quantification. Quantitative mass spectrometry in proteomics is reviewed in e.g. Bantscheff, M., et al. Anal Bioanal Chem 2007, 389, 1017-1031, which is hereby incorporated by reference in its entirety.

For example, input peptides may undergo fragmentation in a collision cell, thus generating product ions exclusive to the peptides. Both the intact peptide mass and one or more specific fragment ions of that peptides can be monitored over the course of an MS experiment e.g. using SRM/MRM, PRM etc.

The observed m/z ratio of a peptide and its corresponding product ion m/z ratio are referred to as a “transition”, i.e. a mass pair representing the m/z of an analyte (the parent ion) and the m/z of one of its product ions which is formed upon fragmentation of the parent ion.

It is well within the routine work of a skilled person to develop suitable transitions for quantitative mass spectrometry techniques, e.g. SRM/MRM-MS and PRM-MS. Mead et al., Mol Cell Proteomics. 2009 April; 8(4): 696-705, hereby incorporated by reference in its entirety, describes one such technique for designing transitions.

Tables 7 and 8 provide examples of transitions for the complement proteins described herein, based on fragmentation of synthetic versions of each peptide of interest. Suitable alternative transitions may also be used, the identification of which is well within the routine remit of a skilled person.

Quantitation can be achieved by ‘spiking’ the sample with known quantities of labelled synthetic peptides. The combination of retention time, peptide mass, and fragment mass practically eliminates ambiguities in peptide assignments and extends the quantification range to 4-5 orders of magnitude. In some cases, the methods provided herein comprise a step of determining optimised MS settings and/or quantitation reference values using stable isotopic standards.

Mass spectrometry techniques that may be used in the present disclosure may comprise targeted or semi-targeted MS workflows and/or data-dependent acquisition (DDA) or data-independent acquisition (DIA) techniques.

DDA uses knowledge obtained during the acquisition to decide which MS1 peptide precursors to subject for fragmentation (MS/MS) in the collision cell. DIA, in contrast, performs predefined MS/MS fragmentation and data collection regardless of sample content, which allows for more sensitive and accurate protein quantification compared to DDA. DIA strategies can be further segregated into targeted or untargeted acquisitions. Targeted DIA methods fragment predefined precursor ions that correspond to the peptide analytes, usually at known (measured or predicted) retention times. Targeted DIA has become widely used in academic, pharmaceutical, and biotechnology research for quantification of small molecules (metabolites), peptides, and post-translational modifications (PTMs). For example, selected-reaction monitoring (SRM), a type of targeted DIA, is currently considered the gold standard method for mass spectrometric quantification due to its high accuracy and precision. For a review of DIA techniques, see e.g. Meyer and Schilling, Expert Rev Proteomics. 2017 May; 14(5): 419-429, hereby incorporated in its entirety.

Other suitable DIA methods include e.g. Sequential Window Acquisition of All Theoretical mass spectrometry (SWATH MS; see e.g. Ludwig et al., Mol Syst Biol (2018)14:e8126), SONAR (Waters.com), or Online Parallel Accumulation-Serial Fragmentation (PASEF; see e.g. Meier et al., J Proteome Res. 2015 Dec. 4; 14(12):5378-87 and Meier et al., Mol Cell Proteomics. 2018 December; 17(12): 2534-2545).

### Subjects

The subject in accordance with the various aspects of the present disclosure may be any animal or human. Therapeutic and prophylactic applications may be in human or animals (veterinary use). The subject to be treated with a therapeutic substance described herein may be a subject in need thereof. The subject is preferably mammalian, more preferably human. The subject may be a non-human mammal, but is more preferably human. The subject may be male or female. The subject may be a patient.

A subject may have been diagnosed with a disease or condition requiring treatment, may be suspected of having such a disease/condition, or may be at risk of developing/contracting such a disease/condition.

In embodiments according to the present disclosure the subject is preferably a human subject. In some embodiments, the subject to be treated in accordance with a therapeutic or prophylactic method of the present disclosure is a subject having, or at risk of developing, a disease described herein. In embodiments according to the present disclosure, a subject may be selected for treatment according to the methods based on characterisation for certain markers of such disease/condition.

The subject may be identified, or may have been identified, as having a complement-related disorder or being at risk of developing a complement-related disorder, e.g. by a method described herein. The subject may be suspected of having or being at risk of developing a complement-related disorder, e.g. using the methods described herein.

A subject described herein may belong to a patient subpopulation i.e. the subject may be part of an identifiable, specific portion or subdivision of a population. The population and/or subpopulation may have or be suspected to have a complement-related disorder. The subpopulation may display atypical presence or levels of one or more complement proteins, e.g. detected/determined as described herein, as compared to the population as a whole. The population and/or subpopulation may have or be suspected to have AMD, EOMD or a macular dystrophy.

The subject may be characterised as having an atypical presence or level of one or more complement proteins, e.g. detected/determined/measured as described herein. A subject may have, have been determined to have, or be characterised as having elevated gene and/or protein expression of a Factor H family protein (e.g. a Factor H-related protein; e.g. selected from FHR1, FHR2, FHR3, FHR4, FHR5, FH and/or FHL-1).

### Kits

In some aspects of the present disclosure a kit of parts is provided. In some embodiments the kit may have at least one container having a predetermined quantity of an agent, inhibitory nucleic acid, nucleic acid, expression vector, cell or composition described herein.

In some embodiments, the kit may comprise materials for producing an agent, inhibitory nucleic acid, nucleic acid, expression vector, cell or composition described herein.

The kit may provide the agent, inhibitory nucleic acid, nucleic acid, expression vector, cell or composition together with instructions for administration to a patient in order to treat a specified disease/condition.

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease or condition.

Aspects of the disclosure include kits for use in performing in vitro diagnostic methods. In some embodiments, the present disclosure provides a kit comprising endoproteinase GluC for use in a method of detecting and/or determining the level of one or more complement protein(s) e.g. in a sample. The kit may be used for any of the methods described herein and/or for detecting/determining the level of any one or combination of proteins described herein. The kit may be suitable for, used for, or intended/sold/distributed for detecting at least one complement protein in a sample, determining the level of at least one complement protein in a sample, preparing at least one complement protein for analysis and/or detection, determining the presence and/or level of a complement protein in a subject, determining whether a subject is at risk of developing a complement-related disorder, identifying a subject having a complement-related disorder, selecting a subject for treatment of a complement-related disorder, and/or treating a subject who is suspected to have a complement-related disorder. The kit and components thereof may be suitable for use with MS techniques.

A kit provided herein comprises one, two, or more components suitable for performing the methods described herein, in whole or in part. The kit may comprise standards or controls, e.g. labelled peptide standard(s) for each protein to be detected using the kit. The kit may comprise a predetermined quantity of labelled peptide standards. The kit may comprise a predetermined quantity of GluC enzyme, optionally with the necessary buffers and reagents for enzyme digestion. The components of the kit may be provided in a single composition, or may be provided as plural compositions.

The kit may be suitable for a point-of-care in vitro diagnostic test. It may be a kit for laboratory-based testing.

Kits according to the present disclosure may include instructions for use, e.g. in the form of an instruction booklet or leaflet. The instructions may include a protocol for performing any one or more of the methods described herein (e.g. for enzyme digestion, recommended MS settings, and/or data analysis templates).

The kit may comprise components for separating proteins in a sample and/or performing MS techniques e.g. liquid chromatography columns.

Kits according to the present disclosure may be adapted for use with dry samples, wet samples, frozen samples, fixed samples, urine samples, saliva samples, tissue samples, blood samples, or any other type of sample, including any of the sample types disclosed herein. Kits may comprise a device for obtaining or processing a blood, serum, plasma, cell or tissue sample.

### Sequence Identity

As used herein, a nucleic acid, nucleotide, or amino acid sequence which corresponds to a reference nucleic acid, nucleotide or amino acid sequence may comprise at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the reference sequence.

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof. The invention includes the combination of the aspects and preferred features described except where such a combination is clearly impermissible or expressly avoided.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. By way of illustration, reference herein to “an inhibitory nucleic acid” is to be interpreted throughout to encompass reference to “a plurality of inhibitory nucleic acids”, unless context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The phase “and/or” as used herein encompasses each member of the list individually, as well as any combination of one or members of the list up to and including every member of the list.

Where a nucleic acid sequence is disclosed herein, the reverse complement thereof is also expressly contemplated. Where a nucleic acid sequence is disclosed herein, equivalent sequences encoding the same sequence of amino acids (as a consequence of redundancy of the genetic code) are also expressly contemplated.

Methods according to the present disclosure may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism. In some embodiments, the determining, detecting, measuring, quantifying, predicting and/or diagnosing steps of the methods provided herein are performed in vitro. In some aspects and embodiments, methods described herein may performed outside the human or animal body.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

Aspects and embodiments of the present disclosure will now be illustrated, by way of example, with reference to the accompanying figures. Further aspects and embodiments will be apparent to those skilled in the art. All documents mentioned in this text are incorporated herein by reference.

### Numbered Paragraphs

1. An inhibitory nucleic acid for reducing gene and/or protein expression of one or more Factor H family proteins.
2. The inhibitory nucleic acid according to paragraph 1, wherein the one or more Factor H family proteins are Factor H-related proteins, optionally wherein the Factor H-related proteins are selected from FHR1, FHR2, FHR3, FHR4 and FHR5.
3. The inhibitory nucleic acid according to paragraph 1 or paragraph 2, wherein the inhibitory nucleic acid comprises or encodes antisense nucleic acid targeting a nucleotide sequence of RNA encoded by one or more genes encoding the one or more Factor H family proteins.
4. The inhibitory nucleic acid according to any one of paragraphs 1 to 3, wherein the inhibitory nucleic acid comprises or encodes antisense nucleic acid targeting a nucleotide sequence comprising, or consisting of, SEQ ID NO:158, 159, 160, 161, 162, 163, 164, 165, 166, 167, 168, 169, 170, 171, 172, 173, 174, 175, 176 or 177.
5. The inhibitory nucleic acid according to any one of paragraphs 1 to 4, wherein the inhibitory nucleic acid comprises or encodes antisense nucleic acid comprising or consisting of a sequence having at least 75% sequence identity to SEQ ID NO:178, 179, 180, 181, 182, 183, 184, 185, 186, 187, 188, 189, 190, 191, 192, 193, 194, 195, 196 or 197.
6. The inhibitory nucleic acid according to any one of paragraphs 1 to 5, wherein the inhibitory nucleic acid is an siRNA.
7. A nucleic acid, optionally isolated, encoding an inhibitory nucleic acid according to any one of paragraphs 1 to 6.
8. An expression vector, comprising a nucleic acid according to paragraph 7.
9. A composition comprising an inhibitory nucleic acid according to any one of paragraphs 1 to 6, a nucleic acid according to paragraph 7, or an expression vector according to paragraph 8, and a pharmaceutically acceptable carrier, diluent, excipient or adjuvant.
10. A cell comprising an inhibitory nucleic acid according to any one of paragraphs 1 to 6, a nucleic acid according to paragraph 7, or an expression vector according to paragraph 8.
11. An in vitro or in vivo method for reducing gene and/or protein expression of one or more Factor H family proteins, comprising contacting a cell with an inhibitory nucleic acid according to any one of paragraphs 1 to 6, a nucleic acid according to paragraph 7, an expression vector according to paragraph 8, or a composition according to paragraph 9.
12. Use of an inhibitory nucleic acid according to any one of paragraphs 1 to 6, a nucleic acid according to paragraph 7, an expression vector according to paragraph 8, or a composition according to paragraph 9, to reduce gene and/or protein expression of one or more Factor H family proteins.
13. An inhibitory nucleic acid according to any one of paragraphs 1 to 6, a nucleic acid according to paragraph 7, an expression vector according to paragraph 8, or a composition according to paragraph 9, for use in a method of medical treatment or prophylaxis.
14. An inhibitory nucleic acid according to any one of paragraphs 1 to 6, a nucleic acid according to paragraph 7, an expression vector according to paragraph 8, or a composition according to paragraph 9, for use in a method of treating or preventing a complement-related disorder.
15. Use of an inhibitory nucleic acid according to any one of paragraphs 1 to 6, a nucleic acid according to paragraph 7, an expression vector according to paragraph 8, or a composition according to paragraph 9, in the manufacture of a medicament for treating or preventing a complement-related disorder.
16. A method of treating or preventing a complement-related disorder in a subject, comprising administering to a subject a therapeutically- or prophylactically-effective amount of an inhibitory nucleic acid according to any one of paragraphs 1 to 6, a nucleic acid according to paragraph 7, an expression vector according to paragraph 8, or a composition according to paragraph 9.
17. A method for selecting a subject to be administered an inhibitory nucleic acid according to any one of paragraphs 1 to 6, a nucleic acid according to paragraph 7, an expression vector according to paragraph 8, or a composition according to paragraph 9, the method comprising:
   (a) determining the level of a complement protein selected from one or more of FHR1, FHR2, FHR3, FHR4 and/or FHR5, and optionally FHL-1, in a blood sample obtained from the subject;
   (b) selecting the subject to be administered an inhibitory nucleic acid according to any one of paragraphs 1 to 6, a nucleic acid according to paragraph 7, an expression vector according to paragraph 8, or a composition according to paragraph 9, if the level of the complement protein determined in (a) is elevated as compared to the level of that complement protein in blood in a control subject that does not have a complement-related disorder.
18. An inhibitory nucleic acid according to any one of paragraphs 1 to 6, a nucleic acid according to paragraph 7, an expression vector according to paragraph 8, or a composition according to paragraph 9 for use in a method of treating or preventing a complement-related disorder in a subject, wherein the method comprises:
   (a) determining the level of a complement protein selected one or more of FHR1, FHR2, FHR3, FHR4 and/or FHR5, and optionally FHL-1, in a blood sample obtained from the subject;
   (b) determining whether the level of the complement protein(s) in (a) is elevated as compared to the level of that complement protein(s) in blood in a control subject that does not have a complement-related disorder; and
   (c) administering an inhibitory nucleic acid according to any one of paragraphs 1 to 6, a nucleic acid according to paragraph 7, an expression vector according to paragraph 8, or a composition according to paragraph 9 to the subject.
19. Use of an inhibitory nucleic acid according to any one of paragraphs 1 to 6, a nucleic acid according to paragraph 7, an expression vector according to paragraph 8, or a composition according to paragraph 9 in the manufacture of a medicament for treating or preventing a complement-related disorder in a subject, wherein the method comprises:
   (a) determining the level of a complement protein selected from one or more of FHR1, FHR2, FHR3, FHR4 and/or FHR5, and optionally FHL-1, in a blood sample obtained from the subject;
   (b) determining whether the level of the complement protein(s) in (a) is elevated as compared to the level of that complement protein(s) in blood in a control subject that does not have a complement-related disorder; and
   (c) administering an inhibitory nucleic acid according to any one of paragraphs 1 to 6, a nucleic acid according to paragraph 7, an expression vector according to paragraph 8, or a composition according to paragraph 9 to the subject.
20. A method of treating or preventing a complement-related disorder in a subject, comprising:
   (a) determining the level of a complement protein selected from one or more of FHR1, FHR2, FHR3, FHR4 and/or FHR5, and optionally FHL-1, in a blood sample obtained from the subject;
   (b) determining whether the level of the complement protein is elevated as compared to the level of that complement protein in blood in a control subject that does not have a complement-related disorder; and
   (c) administering an inhibitory nucleic acid according to any one of paragraphs 1 to 6, a nucleic acid according to paragraph 7, an expression vector according to paragraph 8, or a composition according to paragraph 9 to the subject.
21. The inhibitory nucleic acid, nucleic acid, expression vector or composition for use according to paragraph 14 or paragraph 18, the use according to paragraph 15 or paragraph 19, or the method according to paragraph 16 or paragraph 20, wherein the complement-related disorder is selected from: macular degeneration, age-related macular degeneration (AMD), geographic atrophy (‘dry’ or non-exudative AMD), early AMD, early onset macular degeneration (EOMD), intermediate AMD, late/advanced AMD, ‘wet’ (neovascular or exudative) AMD, choroidal neovascularisation (CNV), retinal dystrophy, autoimmune uveitis, haemolytic uremic syndrome (HUS), atypical HUS, deficiency of FHR plasma proteins and autoantibody positive form of Hemolytic Uremic Syndrome (DEAP HUS), a glomerular disease, membranoproliferative glomerulonephritis type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, Alzheimer's disease, dementia, Lewy body disease, amyotrophic lateral sclerosis (ALS), Huntington's disease, a prion disease, cancer, lung cancer, and glioblastoma multiforme (GBM).

### Sequences

| SEQ ID | | |
|---|---|---|
| No: | Description | Sequence |
| 1 | Human Complement Factor H | MRLLAKIICLMLWAICVAEDCNELPPRRNTEILTGSWSDQTYPEGTQAIYKCRPGYRS |
| | (FH) | LGNVIMVCRKGEWVALNPLRKCQKRPCGHPGDTPFGTFTLTGGNVFEYGVKAVYTC |
| | Uniprot: P08603-1 | NEGYQLLGEINYRECDTDGWTNDIPICEVVKCLPVTAPENGKIVSSAMEPDREYHFG |
| | Entry version 229 (11 Dec. 2019) | QAVRFVCNSGYKIEGDEEMHCSDDGFWSKEKPKCVEISCKSPDVINGSPISQKIIYKE |
| | Sequence version 4 (11 Sep. | NERFQYKCNMGYEYSERGDAVCTESGWRPLPSCEEKSCDNPYIPNGDYSPLRIKHR |
| | 2007) | TGDEITYQCRNGFYPATRGNTAKCTSTGWIPAPRCTLKPCDYPDIKHGGLYHENMR |
| | | RPYFPVAVGKYYSYYCDEHFETPSGSYWDHIHCTQDGWSPAVPCLRKCYFPYLEN |
| | | GYNQNYGRKFVQGKSIDVACHPGYALPKAQTTVTCMENGWSPTPRCIRVKTCSKSS |
| | | IDIENGFISESQYTYALKEKAKYQCKLGYVTADGETSGSITCGKDGWSAQPTCIKSCD |
| | | IPVFMNARTKNDFTWFKLNDTLDYECHDGYESNTGSTTGSIVCGYNGWSDLPICYER |
| | | ECELPKIDVHLVPDRKKDQYKVGEVLKFSCKPGFTIVGPNSVQCYHFGLSPDLPICKE |
| | | QVQSCGPPPELLNGNVKEKTKEEYGHSEVVEYYCNPRFLMKGPNKIQCVDGEWTTL |
| | | PVCIVEESTCGDIPELEHGWAQLSSPPYYYGDSVEFNCSESFTMIGHRSITCIHGVWT |
| | | QLPQCVAIDKLKKCKSSNLIILEEHLKNKKEFDHNSNIRYRCRGKEGWIHTVCINGRW |
| | | DPEVNCSMAQIQLCPPPPQIPNSHNMTTTLNYRDGEKVSVLCQENYLIQEGEEITCK |
| | | DGRWQSIPLCVEKIPCSQPPQIEHGTINSSRSSQESYAHGTKLSYTCEGGFRISEENE |
| | | TTCYMGKWSSPPQCEGLPCKSPPEISHGWVAHMSDSYQYGEEVTYKCFEGFGIDG |
| | | PAIAKCLGEKWSHPPSCIKTDCLSLPSFENAIPMGEKKDVYKAGEQVTYTCATYYKM |
| | | DGASNVTCINSRWTGRPTCRDTSCVNPPTVQNAYIVSRQMSKYPSGERVRYQCRS |
| | | PYEMFGDEEVMCLNGNWTEPPQCKDSTGKCGPPPPIDNGDITSFPLSVYAPASSVE |
| | | YQCQNLYQLEGNKRITCRNGQWSEPPKCLHPCVISREIMENYNIALRWTAKQKLYSR |
| | | TGESVEFVCKRGYRLSSRSHTLRTTCWDGKLEYPTCAKR |
| 2 | Human FH-like protein 1 (FHL- | MRLLAKIICLMLWAICVAEDCNELPPRRNTEILTGSWSDQTYPEGTQAIYKCRPGYRS |
| | 1) | LGNVIMVCRKGEWVALNPLRKCQKRPCGHPGDTPFGTFTLTGGNVFEYGVKAVYTC |
| | Uniprot: P08603-2 | NEGYQLLGEINYRECDTDGWTNDIPICEVVKCLPVTAPENGKIVSSAMEPDREYHFG |
| | Entry version 229 (11 Dec. 2019) | QAVRFVCNSGYKIEGDEEMHCSDDGFWSKEKPKCVEISCKSPDVINGSPISQKIIYKE |
| | Sequence version 4 (11 Sep. | NERFQYKCNMGYEYSERGDAVCTESGWRPLPSCEEKSCDNPYIPNGDYSPLRIKHR |
| | 2007) | TGDEITYQCRNGFYPATRGNTAKCTSTGWIPAPRCTLKPCDYPDIKHGGLYHENMR |
| | | RPYFPVAVGKYYSYYCDEHFETPSGSYWDHIHCTQDGWSPAVPCLRKCYFPYLEN |
| | | GYNQNYGRKFVQGKSIDVACHPGYALPKAQTTVTCMENGWSPTPRCIRVSFTL |
| 3 | Complement Factor H-related 1 | MWLLVSVILISRISSVGGEATFCDFPKINHGILYDEEKYKPFSQVPTGEVFYYSCEYNF |
| | (FHR1) | VSPSKSFWTRITCTEEGWSPTPKCLRLCFFPFVENGHSESSGQTHLEGDTVQIICNT |
| | Uniprot: Q03591 | GYRLQNNENNISCVERGWSTPPKCRSTDTSCVNPPTVQNAHILSRQMSKYPSGERV |
| | Entry version 166 (11 Dec. 2019) | RYECRSPYEMFGDEEVMCLNGNWTEPPQCKDSTGKCGPPPPIDNGDITSFPLSVYA |
| | Sequence version 2 (16 Dec. | PASSVEYQCQNLYQLEGNKRITCRNGQWSEPPKCLHPCVISREIMENYNIALRWTAK |
| | 2008) | QKLYLRTGESAEFVCKRGYRLSSRSHTLRTTCWDGKLEYPTCAKR |
| 4 | Complement Factor H-related 2 | MWLLVSVILISRISSVGGEAMFCDFPKINHGILYDEEKYKPFSQVPTGEVFYYSCEYN |
| | (FHR2), isoform 1 | FVSPSKSFWTRITCAEEGWSPTPKCLRLCFFPFVENGHSESSGQTHLEGDTVQIICN |
| | Uniprot: P36980-1 | TGYRLQNNENNISCVERGWSTPPKCRSTISAEKCGPPPPIDNGDITSFLLSVYAPGSS |
| | Entry version 168 (11 Dec. 2019) | VEYQCQNLYQLEGNNQITCRNGQWSEPPKCLDPCVISQEIMEKYNIKLKWTNQQKL |
| | Sequence version 1 (01 Jun. | YSRTGDIVEFVCKSGYHPTKSHSFRAMCQNGKLVYPSCEEK |
| | 1994) | YSRTGDIVEFVCKSGYHPTKSHSFRAMCQNGKLVYPSCEEK |
| 5 | Complement Factor H-related 2 | MWLLVSVILISRISSVGGEAMFCDFPKINHGILYDEEKYKPFSQVPTGEVFYYSCEYN |
| | (FHR2), isoform 2 | FVSPSKSFWTRITCAEEGWSPTPKCLRLCFFPFVENGHSESSGQTHLEGDTVQIICN |
| | Uniprot: P36980-2 Entry version | TGYRLQNNENNISCVERGWSTPPKCRSTSSSVEYQCQNLYQLEGNNQITCRNGQW |
| | 168 (11 Dec. 2019) | SEPPKCLDPCVISQEIMEKYNIKLKWTNQQKLYSRTGDIVEFVCKSGYHPTKSHSFRA |
| | Sequence version 1 (01 Jun. | MCQNGKLVYPSCEEK |
| | 1994) | |
| 6 | Complement Factor H-related 3 | MLLLINVILTLWVSCANGQVKPCDFPDIKHGGLFHENMRRPYFPVAVGKYYSYYCDE |
| | (FHR3), isoform 1 | HFETPSGSYWDYIHCTQNGWSPAVPCLRKCYFPYLENGYNQNYGRKFVQGNSTEV |
| | Uniprot: Q02985-1 | ACHPGYGLPKAQTTVTCTEKGWSPTPRCIRVRTCSKSDIEIENGFISESSSIYILNKEI |
| | Entry version 161 (11 Dec. 2019) | QYKCKPGYATADGNSSGSITCLQNGWSAQPICINSSEKCGPPPPISNGDTTSFLLKV |
| | Sequence version 2 (21 Feb. | YVPQSRVEYQCQPYYELQGSNYVTCSNGEWSEPPRCIHPCIITEENMNKNNIKLKGR |
| | 2001) | SDRKYYAKTGDTIEFMCKLGYNANTSILSFQAVCREGIVEYPRCE |
| 7 | Complement Factor H-related 3 | MLLLINVILTLWVSCANGQVKPCDFPDIKHGGLFHENMRRPYFPVAVGKYYSYYCDE |
| | (FHR3), isoform 2 | HFETPSGSYWDYIHCTQNGWSPAVPCLRKCYFPYLENGYNQNYGRKFVQGNSTEV |
| | Uniprot: Q02985-2 | ACHPGYGLPKAQTTVTCTEKGWSPTPRCIRVNSSEKCGPPPPISNGDTTSFLLKVYV |
| | Entry version 161 (11 Dec. 2019) | PQSRVEYQCQPYYELQGSNYVTCSNGEWSEPPRCIHPCIITEENMNKNNIKLKGRSD |
| | Sequence version 2 (21 Feb. | RKYYAKTGDTIEFMCKLGYNANTSILSFQAVCREGIVEYPRCE |
| | 2001) | |
| 8 | Complement Factor H-related 4 | MLLLINVILTLWVSCANGQEVKPCDFPEIQHGGLYYKSLRRLYFPAAAGQSYSYYCD |
| | (FHR4A) | QNFVTPSGSYWDYIHCTQDGWSPTVPCLRTCSKSDVEIENGFISESSSIYILNEETQY |
| | Uniprot: Q92496-1 | NCKPGYATAEGNSSGSITCLQNGWSTQPICIKFCDMPVFENSRAKSNGMWFKLHDT |
| | Entry version 157 (11 Dec. 2019) | LDYECYDGYESSYGNTTDSIVCGEDGWSHLPTCYNSSENCGPPPPISNGDTTSFPQ |
| | Sequence version 3 (22 Jan. | KVYLPWSRVEYQCQSYYELQGSKYVTCSNGDWSEPPRCISMKPCEFPEIQHGHLYY |
| | 2014) | ENTRRPYFPVATGQSYSYYCDQNFVTPSGSYWDYIHCTQDGWLPTVPCLRTCSKS |
| | | DIEIENGFISESSSIYILNKEIQYKCKPGYATADGNSSGSITCLQNGWSAQPICIKFCDM |
| | | PVFENSRAKSNGMRFKLHDTLDYECYDGYEISYGNTTGSIVCGEDGWSHFPTCYNS |
| | | SEKCGPPPPISNGDTTSFLLKVYVPQSRVEYQCQSYYELQGSNYVTCSNGEWSEPP |
| | | RCIHPCIITEENMNKNNIQLKGKSDIKYYA |
| | | KTGDTIEFMCKLGYNANTSVLSFQAVCREGIVEYPRCE |
| 9 | Complement Factor H-related 4 | MLLLINVILTLWVSCANGQEVKPCDFPEIQHGGLYYKSLRRLYFPAAAGQSYSYYCD |
| | (FHR4B) | QNFVTPSGSYWDYIHCTQDGWSPTVPCLRTCSKSDIEIENGFISESSSIYILNKEIQYK |
| | Uniprot: Q92496-3 | CKPGYATADGNSSGSITCLQNGWSAQPICIKFCDMPVFENSRAKSNGMRFKLHDTL |
| | Entry version 157 (11 Dec. 2019) | DYECYDGYEISYGNTTGSIVCGEDGWSHFPTCYNSSEKCGPPPPISNGDTTSFLLKV |
| | Sequence version 3 (22 Jan. | YVPQSRVEYQCQSYYELQGSNYVTCSNGEWSEPPRCIHPCIITEENMNKNNIQLKGK |
| | 2014) | SDIKYYAKTGDTIEFMCKLGYNANTSVLSFQAVCREGIVEYPRCE |
| 10 | Complement Factor H-related 5 | MLLLFSVILISWVSTVGGEGTLCDFPKIHHGFLYDEEDYNPFSQVPTGEVFYYSCEYN |
| | (FHR5) | FVSPSKSFWTRITCTEEGWSPTPKCLRMCSFPFVKNGHSESSGLIHLEGDTVQIICNT |
| | Uniprot: Q9BXR6 | GYSLQNNEKNISCVERGWSTPPICSFTKGECHVPILEANVDAQPKKESYKVGDVLKF |
| | | SCRKNLIRVGSDSVQCYQFGWSPNFPTCKGQVRSCGPPPQLSNGEVKEIRKEEYG |
| | | HNEVVEYDCNPNFIINGPKKIQCVDGEWTTLPTCVEQVKTCGYIPELEYGYVQPSVP |
| | | PYQHGVSVEVNCRNEYAMIGNNMITCINGIWTELPMCVATHQLKRCKIAGVNIKTLLK |
| | | LSGKEFNHNSRIRYRCSDIFRYRHSVCINGKWNPEVDCTEKREQFCPPPPQIPNAQN |
| | | MTTTVNYQDGEKVAVLCKENYLLPEAKEIVCKDGRWQSLPRCVESTAYCGPPPSIN |
| | | NGDTTSFPLSVYPPGSTVTYRCQSFYKLQGSVTVTCRNKQWSEPPRCLDPCVVSEE |
| | | NMNKNNIQLKWRNDGKLYAKTGDAVEFQCKFPHKAMISSPPFRAICQEGKFEYPICE |
| 11 | Complement Factor I (FI) | MKLLHVFLLFLCFHLRFCKVTYTSQEDLVEKKCLAKKYTHLSCDKVFCQPWQRCIEG |
| | Uniprot: P05156 | TCVCKLPYQCPKNGTAVCATNRRSFPTYCQQKSLECLHPGTKFLNNGTCTAEGKFS |
| | Entry version 147 (11 Dec. 2019) | VSLKHGNTDSEGIVEVKLVDQDKTMFICKSSWSMREANVACLDLGFQQGADTQRRF |
| | Sequence version 1 (01 Jun. | KLSDLSINSTECLHVHCRGLETSLAECTFTKRRTMGYQDFADVVCYTQKADSPMDD |
| | 2001) | FFQCVNGKYISQMKACDGINDCGDQSDELCCKACQGKGFHCKSGVCIPSQYQCNG |
| | | EVDCITGEDEVGCAGFASVTQEETEILTADMDAERRRIKSLLPKLSCGVKNRMHIRRK |
| | | RIVGGKRAQLGDLPWQVAIKDASGITCGGIYIGGCWILTAAHCLRASKTHRYQIWTTV |
| | | VDWIHPDLKRIVIEYVDRIIFHENYNAGTYQNDIALIEMKKDGNKKDCELPRSIPACVP |
| | | WSPYLFQPNDTCIVSGWGREKDNERVFSLQWGEVKLISNCSKFYGNRFYEKEMEC |
| | | AGTYDGSIDACKGDSGGPLVCMDANNVTYVWGVVSWGENCGKPEFPGVYTKVAN |
| | | YFDWISYHVGRPFISQYNV |
| 12 | Complement factor C3 | MGPTSGPSLLLLLLTHLPLALGSPMYSIITPNILRLESEETMVLEAHDAQGDVPVTVTV |
| | (Uniprot: P01024) | HDFPGKKLVLSSEKTVLTPATNHMGNVTFTIPANREFKSEKGRNKFVTVQATFGTQV |
| | Entry version 242 (11 Dec. 2019) | VEKVVLVSLQSGYLFIQTDKTIYTPGSTVLYRIFTVNHKLLPVGRTVMVNIENPEGIPVK |
| | Sequence version 2 (12 Dec. | QDSLSSQNQLGVLPLSWDIPELVNMGQWKIRAYYENSPQQVFSTEFEVKEYVLPSF |
| | 2006) | EVIVEPTEKFYYIYNEKGLEVTITARFLYGKKVEGTAFVIFGIQDGEQRISLPESLKRIPI |
| | | EDGSGEVVLSRKVLLDGVQNPRAEDLVGKSLYVSATVILHSGSDMVQAERSGIPIVT |
| | | SPYQIHFTKTPKYFKPGMPFDLMVFVTNPDGSPAYRVPVAVQGEDTVQSLTQGDGV |
| | | AKLSINTHPSQKPLSITVRTKKQELSEAEQATRTMQALPYSTVGNSNNYLHLSVLRTE |
| | | LRPGETLNVNFLLRMDRAHEAKIRYYTYLIMNKGRLLKAGRQVREPGQDLVVLPLSIT |
| | | TDFIPSFRLVAYYTLIGASGQREVVADSVWVDVKDSCVGSLVVKSGQSEDRQPVPG |
| | | QQMTLKIEGDHGARVVLVAVDKGVFVLNKKNKLTQSKIWDVVEKADIGCTPGSGKDY |
| | | AGVFSDAGLTFTSSSGQQTAQRAELQCPQPAARRRRSVQLTEKRMDKVGKYPKEL |
| | | RKCCEDGMRENPMRFSCQRRTRFISLGEACKKVELDCCNYITELRRQHARASHLGL |
| | | ARSNLDEDIIAEENIVSRSEFPESWLWNVEDLKEPPKNGISTKLMNIFLKDSITTWEILA |
| | | VSMSDKKGICVADPFEVTVMQDFFIDLRLPYSVVRNEQVEIRAVLYNYRQNQELKVR |
| | | VELLHNPAFCSLATTKRRHQQTVTIPPKSSLSVPYVIVPLKTGLQEVEVKAAVYHHFIS |
| | | DGVRKSLKVVPEGIRMNKTVAVRTLDPERLGREGVQKEDIPPADLSDQVPDTESETR |
| | | ILLQGTPVAQMTEDAVDAERLKHLIVTPSGCGEQNMIGMTPTVIAVHYLDETEQWEK |
| | | FGLEKRQGALELIKKGYTQQLAFRQPSSAFAAFVKRAPSTWLTAYVVKVFSLAVNLIA |
| | | IDSQVLCGAVKWLILEKQKPDGVFQEDAPVIHQEMIGGLRNNNEKDMALTAFVLISLQ |
| | | EAKDICEEQVNSLPGSITKAGDFLEANYMNLQRSYTVAIAGYALAQMGRLKGPLLNK |
| | | FLTTAKDKNRWEDPGKQLYNVEATSYALLALLQLKDFDFVPPVVRWLNEQRYYGGG |
| | | YGSTQATFMVFQALAQYQKDAPDHQELNLDVSLQLPSRSSKITHRIHWESASLLRSE |
| | | ETKENEGFTVTAEGKGQGTLSVVTMYHAKAKDQLTCNKFDLKVTIKPAPETEKRPQD |
| | | AKNTMILEICTRYRGDQDATMSILDISMMTGFAPDTDDLKQLANGVDRYISKYELDKA |
| | | FSDRNTLIIYLDKVSHSEDDCLAFKVHQYFNVELIQPGAVKVYAYYNLEESCTRFYHP |
| | | EKEDGKLNKLCRDELCRCAEENCFIQKSDDKVTLEERLDKACEPGVDYVYKTRLVKV |
| | | QLSNDFDEYIMAIEQTIKSGSDEVQVGQQRTFISPIKCREALKLEEKKHYLMWGLSSD |
| | | FWGEKPNLSYIIGKDTWVEHWPEEDECQDEENQKQCQDLGAFTESMVVFGCPN |
| 13 | Human C3 β chain UniProt: | SPMYSIITPNILRLESEETMVLEAHDAQGDVPVTVTVHDFPGKKLVLSSEKTVLTPATN |
| | P01024; Entry version 242 (11 | HMGNVTFTIPANREFKSEKGRNKFVTVQATFGTQVVEKVVLVSLQSGYLFIQTDKTIY |
| | Dec. 2019) | TPGSTVLYRIFTVNHKLLPVGRTVMVNIENPEGIPVKQDSLSSQNQLGVLPLSWDIPE |
| | Sequence version 2 (12 Dec. | LVNMGQWKIRAYYENSPQQVFSTEFEVKEYVLPSFEVIVEPTEKFYYIYNEKGLEVTI |
| | 2006); residues 23-667 | TARFLYGKKVEGTAFVIFGIQDGEQRISLPESLKRIPIEDGSGEVVLSRKVLLDGVQNP |
| | | RAEDLVGKSLYVSATVILHSGSDMVQAERSGIPIVTSPYQIHFTKTPKYFKPGMPFDL |
| | | MVFVTNPDGSPAYRVPVAVQGEDTVQSLTQGDGVAKLSINTHPSQKPLSITVRTKKQ |
| | | ELSEAEQATRTMQALPYSTVGNSNNYLHLSVLRTELRPGETLNVNFLLRMDRAHEAK |
| | | IRYYTYLIMNKGRLLKAGRQVREPGQDLVVLPLSITTDFIPSFRLVAYYTLIGASGQRE |
| | | WADSVWVDVKDSCVGSLVVKSGQSEDRQPVPGQQMTLKIEGDHGARVVLVAVDK |
| | | GVFVLNKKNKLTQSKIWDVVEKADIGCTPGSGKDYAGVFSDAGLTFTSSSGQQTAQ |
| | | RAELQCPQPAA |
| 14 | Human C3 α′ chain UniProt: | SNLDEDIIAEENIVSRSEFPESWLWNVEDLKEPPKNGISTKLMNIFLKDSITTWEILAVS |
| | P01024; Entry version 242 (11 | MSDKKGICVADPFEVTVMQDFFIDLRLPYSVVRNEQVEIRAVLYNYRQNQELKVRVE |
| | Dec. 2019) | LLHNPAFCSLATTKRRHQQTVTIPPKSSLSVPYVIVPLKTGLQEVEVKAAVYHHFISDG |
| | Sequence version 2 (12 Dec. | VRKSLKVVPEGIRMNKTVAVRTLDPERLGREGVQKEDIPPADLSDQVPDTESETRILL |
| | 2006); residues 749-1663 | QGTPVAQMTEDAVDAERLKHLIVTPSGCGEQNMIGMTPTVIAVHYLDETEQWEKFG |
| | | LEKRQGALELIKKGYTQQLAFRQPSSAFAAFVKRAPSTWLTAYVVKVFSLAVNLIAID |
| | | SQVLCGAVKWLILEKQKPDGVFQEDAPVIHQEMIGGLRNNNEKDMALTAFVLISLQE |
| | | AKDICEEQVNSLPGSITKAGDFLEANYMNLQRSYTVAIAGYALAQMGRLKGPLLNKFL |
| | | TTAKDKNRWEDPGKQLYNVEATSYALLALLQLKDFDFVPPVVRWLNEQRYYGGGY |
| | | GSTQATFMVFQALAQYQKDAPDHQELNLDVSLQLPSRSSKITHRIHWESASLLRSEE |
| | | TKENEGFTVTAEGKGQGTLSVVTMYHAKAKDQLTCNKFDLKVTIKPAPETEKRPQDA |
| | | KNTMILEICTRYRGDQDATMSILDISMMTGFAPDTDDLKQLANGVDRYISKYELDKAF |
| | | SDRNTLIIYLDKVSHSEDDCLAFKVHQYFNVELIQPGAVKVYAYYNLEESCTRFYHPE |
| | | KEDGKLNKLCRDELCRCAEENCFIQKSDDKVTLEERLDKACEPGVDYVYKTRLVKVQ |
| | | LSNDFDEYIMAIEQTIKSGSDEVQVGQQRTFISPIKCREALKLEEKKHYLMWGLSSDF |
| | | WGEKPNLSYIIGKDTWVEHWPEEDECQDEENQKQCQDLGAFTESMVVFGCPN |
| 15 | Human C3a | SVQLTEKRMDKVGKYPKELRKCCEDGMRENPMRFSCQRRTRFISLGEACKKVFLDC |
| | UniProt: P01024; Entry version | CNYITELRRQHARASHLGLAR |
| | 242 (11 Dec. 2019) | |
| | Sequence version 2 (12 Dec. | |
| | 2006); residues 672-748 | |
| 16 | Human C3b α′ chain fragment 1 | SNLDEDIIAEENIVSRSEFPESWLWNVEDLKEPPKNGISTKLMNIFLKDSITTWEILAVS |
| | UniProt: P01024; | MSDKKGICVADPFEVTVMQDFFIDLRLPYSVVRNEQVEIRAVLYNYRQNQELKVRVE |
| | Entry version 242 (11 Dec. 2019) | LLHNPAFCSLATTKRRHQQTVTIPPKSSLSVPYVIVPLKTGLQEVEVKAAVYHHFISDG |
| | Sequence version 2 (12 Dec. | VRKSLKVVPEGIRMNKTVAVRTLDPERLGREGVQKEDIPPADLSDQVPDTESETRILL |
| | 2006); | QGTPVAQMTEDAVDAERLKHLIVTPSGCGEQNMIGMTPTVIAVHYLDETEQWEKFG |
| | residues 749-1663 | LEKRQGALELIKKGYTQQLAFRQPSSAFAAFVKRAPSTWLTAYVVKVFSLAVNLIAID |
| | | SQVLCGAVKWLILEKQKPDGVFQEDAPVIHQEMIGGLRNNNEKDMALTAFVLISLQE |
| | | AKDICEEQVNSLPGSITKAGDFLEANYMNLQRSYTVAIAGYALAQMGRLKGPLLNKFL |
| | | TTAKDKNRWEDPGKQLYNVEATSYALLALLQLKDFDFVPPVVRWLNEQRYYGGGY |
| | | GSTQATFMVFQALAQYQKDAPDHQELNLDVSLQLPSRSSKITHRIHWESASLLRSEE |
| | | TKENEGFTVTAEGKGQGTLSVVTMYHAKAKDQLTCNKFDLKVTIKPAPETEKRPQDA |
| | | KNTMILEICTRYRGDQDATMSILDISMMTGFAPDTDDLKQLANGVDRYISKYELDKAF |
| | | SDRNTLIIYLDKVSHSEDDCLAFKVHQYFNVELIQPGAVKVYAYYNLEESCTRFYHPE |
| | | KEDGKLNKLCRDELCRCAEENCFIQKSDDKVTLEERLDKACEPGVDYVYKTRLVKVQ |
| | | LSNDFDEYIMAIEQTIKSGSDEVQVGQQRTFISPIKCREALKLEEKKHYLMWGLSSDF |
| | | WGEKPNLSYIIGKDTWVEHWPEEDECQDEENQKQCQDLGAFTESMVVFGCPN |
| 17 | Human C3 α′ chain fragment 2 | SEETKENEGFTVTAEGKGQGTLSVVTMYHAKAKDQLTCNKFDLKVTIKPAPETEKRP |
| | UniProt: P01024; Entry version | QDAKNTMILEICTRYRGDQDATMSILDISMMTGFAPDTDDLKQLANGVDRYISKYELD |
| | 242 (11 Dec. 2019) | KAFSDRNTLIIYLDKVSHSEDDCLAFKVHQYFNVELIQPGAVKVYAYYNLEESCTRFY |
| | Sequence version 2 (12 Dec. | HPEKEDGKLNKLCRDELCRCAEENCFIQKSDDKVTLEERLDKACEPGVDYVYKTRLV |
| | 2006); | KVQLSNDFDEYIMAIEQTIKSGSDEVQVGQQRTFISPIKCREALKLEEKKHYLMWGLS |
| | residues 1321-1663 | SDFWGEKPNLSYIIGKDTWVEHWPEEDECQDEENQKQCQDLGAFTESMVVFGCPN |
| 18 | C3f | SSKITHRIHWESASLLR |
| | UniProt: P01024; | |
| | Entry version 242 (11 Dec. 2019) | |
| | Sequence version 2 (12 Dec. | |
| | 2006) | |
| | residues 1304-1320 | |
| 19 | Human C3c α′ chain fragment 1 | SNLDEDIIAEENIVSRSEFPESWLWNVEDLKEPPKNGISTKLMNIFLKDSITTWEILAVS |
| | UniProt: P01024; | MSDKKGICVADPFEVTVMQDFFIDLRLPYSVVRNEQVEIRAVLYNYRQNQELKVRVE |
| | Entry version 242 (11 Dec. 2019) | LLHNPAFCSLATTKRRHQQTVTIPPKSSLSVPYVIVPLKTGLQEVEVKAAVYHHFISDG |
| | Sequence version 2 (12 Dec. | VRKSLKVVPEGIRMNKTVAVRTLDPERLGR |
| | 2006); | |
| | residues 749-954 | |
| 20 | FH MS peptide | VTYKCFE |
| 21 | FHL-1 MS peptide | NGWSPTPRCIRVSFTL |
| 22 | FHR1 MS peptide | ATFCDFPKINHGILYDEE |
| 23 | FHR2 MS peptide 1 | RGWSTPPKCRSTISAE |
| 24 | FHR2 MS peptide 2 | AMFCDFPKINHGILYDEE |
| 25 | FHR3 MS peptide | VACHPGYGLPKAQTTVTCTE |
| 26 | FHR4 MS peptide | YQCQSYYE |
| 27 | FHR5 MS peptide | RGWSTPPICSFTKGE |
| 28 | C3.1 peptide | GTAFVIFGIQDGE |
| 29 | C3.2 peptide | LRRQHARASHLGLARSNLDE |
| 30 | C3.3 peptide | LRRQHARASHLGLAR |
| 31 | C3.4 peptide | LNLDVSLQLPSRSSKITHRIHWE |
| 32 | C3.5 peptide | LNLDVSLQLPSR |
| 33 | C3.6 peptide | RLGRE |
| 34 | C3.7 peptide | SSKITHRIHWE |
| 35 | C3.8 peptide | SASLLR |
| 36 | C3.9 peptide | RLGR |
| 37 | C3.10 peptide | HLIVTPSGCGE |
| 38 | FI peptide 1 | CAGTYDGSIDACKGDSGGPLVCMDANNVTYVWGVVSWGE |
| 39 | FI peptide 2 | GTCVCKLPYQCPKNGTAVCATNRRSFPTYCQQKSLE |
| 40 | FI peptide 3 | FPGVYTKVANYFDWISYHVGRPFISQYNV |
| 41 | FI peptide 4 | ANVACLDLGFQQGADTQRRFKLSDLSINSTE |
| 42 | FI peptide 5 | LPRSIPACVPWSPYLFQPNDTCIVSGWGRE |
| 43 | FI peptide 6 | KKCLAKKYTHLSCDKVFCQPWQRCIE |
| 44 | FI peptide 7 | LCCKACQGKGFHCKSGVCIPSQYQCNGE |
| 45 | FI peptide 8 | VKLVDQDKTMFICKSSWSMRE |
| 46 | FI peptide 9 | VKLISNCSKFYGNRFYE |
| 47 | FI peptide 10 | CLHPGTKFLNNGTCTAE |
| 48 | FI peptide 11 | NYNAGTYQNDIALIE |
| 49 | FI peptide 12 | GKFSVSLKHGNTDSE |
| 50 | FI peptide 13 | VGCAGFASVTQEE |
| 51 | FI peptide 14 | MKKDGNKKDCE |
| 52 | FI peptide 15 | YVDRIIFHE |
| 53 | FI peptide 16 | CLHVHCRGLE |
| 54 | FI peptide 17 | RVFSLQWGE |
| 55 | FI peptide 18 | ILTADMDAE |
| 56 | FI peptide 19 | KVTYTSQE |
| 57 | FI peptide 20 | VDCITGE |
| 58 | FI peptide 21 | NCGKPE |
| 59 | FI peptide 22 | TSLAE |
| 60 | FI peptide 23 | KDNE |
| 61 | C3.1.1 | AKIRYYTYLIMNKGRLLKAGRQVREPGQDLVVLPLSITTDFIPSFRLVAYYTLIGASGQ |
| | | RE |
| 62 | C3.1.2 | RSGIPIVTSPYQIHFTKTPKYFKPGMPFDLMVFVTNPDGS PAYRVPVAVQGE |
| 63 | C3.1.3 | KVVLVSLQSGYLFIQTDKTIYTPGSTVLYRIFTVNHKLLPVGRTVMVNIE |
| 64 | C3.1.4 | DTVQSLTQGDGVAKLSINTHPSQKPLSITVRTKKQE |
| 65 | C3.1.5 | GDHGARVVLVAVDKGVFVLNKKNKLTQSKIWDVVE |
| 66 | C3.1.6 | KADIGCTPGSGKDYAGVFSDAGLTFTSSSGQQTAQRAE |
| 67 | C3.1.7 | QATRTMQALPYSTVGNSNNYLHLSVLRTE |
| 68 | C3.1.8 | AHDAQGDVPVTVTVHDFPGKKLVLSSE |
| 69 | C3.1.9 | GIPVKQDSLSSQNQLGVLPLSWDIPE |
| 70 | C3.1.10 | WVADSVWVDVKDSCVGSLVVKSGQSE |
| 71 | C3.1.11 | DLVGKSLYVSATVILHSGSDMVQAE |
| 72 | C3.1.12 | KTVLTPATNHMGNVTFTIPANRE |
| 73 | C3.1.13 | KGRNKFVTVQATFGTQVVE |
| 74 | C3.1.14 | WVLSRKVLLDGVQNPRAE |
| 75 | C3.1.15 | TLNVNFLLRMDRAHE |
| 76 | C3.1.16 | LVNMGQWKIRAYYE |
| 77 | C3.1.17 | SPMYSIITPNILRLE |
| 78 | C3.1.18 | DRQPVPGQQMTLKIE |
| 79 | C3.1.19 | VTITARFLYGKKVE |
| 80 | C3.1.20 | GTAFVIFGIQDGE |
| 81 | C3.1.21 | KFYYIYNE |
| 82 | C3.1.22 | NSPQQVFSTE |
| 83 | C3.1.23 | SLKRIPIE |
| 84 | C3.1.24 | YVLPSFE |
| 85 | C3.1.25 | QRISLPE |
| 86 | C3.1.26 | LQCPQPAA |
| 87 | C3.1.27 | VIVEPTE |
| 88 | C3.1.28 | TMVLE |
| 89 | C3.1.29 | LRPGE |
| 90 | C3.1.30 | FKSE |
| 91 | FHR1 peptide 2 | NYNIALRWTAKQKLYLRTGE |
| 92 | FHR2 peptide 3 | YNFVSPSKSFWTRITCAEE |
| 93 | FHR3 peptide 2 | KGWSPTPRCIRVRTCSKSDIE |
| 94 | FHR3 peptide 3 | NGYNQNYGRKFVQGNSTE |
| 95 | FHR3 peptide 4 | QVKPCDFPDIKHGGLFHE |
| 96 | FHR3 peptide 5 | FMCKLGYNANTSILSFQAVCRE |
| 97 | FHR3 peptide 6 | YQCQPYYE |
| 98 | FHR4 peptide 2 | NSRAKSNGMRFKLHDTLDYE |
| 99 | FHR4 peptide 3 | DGWSHFPTCYNSSE |
| 100 | FHR4 peptide 4 | ISYGNTTGSIVCGE |
| 101 | FHR4 peptide 5 | FMCKLGYNANTSVLSFQAVCRE |
| 102 | FHR5 peptide 2 | GTLCDFPKIHHGFLYDEE |
| 103 | FHR5 peptide 3 | YAMIGNNMITCINGIWTE |
| 104 | FHR5 peptide 4 | YGYVQPSVPPYQHGVSVE |
| 105 | FHR5 peptide 5 | GDTVQIICNTGYSLQNNE |
| 106 | FHR5 peptide 6 | IVCKDGRWQSLPRCVE |
| 107 | FHR5 peptide 7 | DYNPFSQVPTGE |
| 108 | FHR5 peptide 8 | QVKTCGYIPE |
| 109 | FHR5 peptide 9 | ANVDAQPKKE |
| 110 | FHR5 peptide 10 | WTTLPTCVE |
| 111 | FHR5 peptide 11 | KVAVLCKE |
| 112 | FH peptide 2 | SNTGSTTGSIVCGYNGWSDLPICYE |
| 113 | FH peptide 3 | NGWSPTPRCIRVKTCSKSSIDIE |
| 114 | FH peptide 4 | LPKIDVHLVPDRKKDQYKVGE |
| 115 | FH peptide 5 | YYCNPRFLMKGPNKIQCVDGE |
| 116 | FH peptide 6 | NYNIALRWTAKQKLYSRTGE |
| 117 | FH peptide 7 | KWSHPPSCIKTDCLSLPSFE |
| 118 | FH peptide 8 | HGWAQLSSPPYYYGDSVE |
| 119 | FH peptide 9 | ISHGVVAHMSDSYQYGEE |
| 120 | FH peptide 10 | FDHNSNIRYRCRGKE |
| 121 | FH peptide 11 | ITCKDGRWQSIPLCVE |
| 122 | FH peptide 12 | GWIHTVCINGRWDPE |
| 123 | FH peptide 13 | KAKYQCKLGYVTADGE |
| 124 | FH peptide 14 | TTCYMGKWSSPPQCE |
| 125 | FH peptide 15 | SYAHGTKLSYTCE |
| 126 | FH peptide 16 | RVRYQCRSPYE |
| 127 | FH peptide 17 | GFGIDGPAIAKCLGE |
| 128 | FH peptide 18 | HGTINSSRSSQE |
| 129 | FH peptide 19 | YQCQNLYQLE |
| 130 | FH peptide 20 | WTTLPVCIVEE |
| 131 | FH peptide 21 | KIPCSQPPQIE |
| 132 | FH peptide 22 | SQYTYALKE |
| 133 | FH peptide 23 | QVQSCGPPPE |
| 134 | FH peptide 24 | KKDVYKAGE |
| 135 | FH peptide 25 | GLPCKSPPE |
| 136 | FH peptide 26 | KVSVLCQE |
| 137 | FH peptide 27 | HLKNKKE |
| 138 | FH peptide 28 | GGFRISEE |
| 139 | FH peptide 29 | LLNGNVKE |
| 140 | FH peptide 30 | YPTCAKR |
| 141 | FH peptide 31 | STCGDIPE |
| 142 | C3dg | EGVQKEDIPPADLSDQVPDTESETRILLQGTPVAQMTEDAVDAERLKHLIVTPSGCG |
| | UniProt: P01024; | EQNMIGMTPTVIAVHYLDETEQWEKFGLEKRQGALELIKKGYTQQLAFRQPSSAFAA |
| | Entry version 242 (11 Dec. 2019) | FVKRAPSTWLTAYVVKVFSLAVNLIAIDSQVLCGAVKWLILEKQKPDGVFQEDAPVIH |
| | Sequence version 2 (12 Dec. | QEMIGGLRNNNEKDMALTAFVLISLQEAKDICEEQVNSLPGSITKAGDFLEANYMNLQ |
| | 2006) | RSYTVAIAGYALAQMGRLKGPLLNKFLTTAKDKNRWEDPGKQLYNVEATSYALLALL |
| | residues 955-1303 | QLKDFDFVPPVVRWLNEQRYYGGGYGSTQATFMVFQALAQYQKDAPDHQELNLDV |
| | | SLQLPSR |
| 143 | C3g | EGVQKEDIPPADLSDQVPDTESETRILLQGTPVAQMTEDAVDAERLK |
| | UniProt: P01024; | |
| | Entry version 242 (11 Dec. 2019) | |
| | Sequence version 2 (12 Dec. | |
| | 2006) | |
| | residues 955-1001 | |
| 144 | C3d | HLIVTPSGCGEQNMIGMTPTVIAVHYLDETEQWEKFGLEKRQGALELIKKGYTQQLA |
| | UniProt: P01024; | FRQPSSAFAAFVKRAPSTWLTAYVVKVFSLAVNLIAIDSQVLCGAVKWLILEKQKPDG |
| | Entry version 242 (11 Dec. 2019) | VFQEDAPVIHQEMIGGLRNNNEKDMALTAFVLISLQEAKDICEEQVNSLPGSITKAGD |
| | Sequence version 2 (12 Dec. | FLEANYMNLQRSYTVAIAGYALAQMGRLKGPLLNKFLTTAKDKNRWEDPGKQLYNV |
| | 2006) | EATSYALLALLQLKDFDFVPPVVRWLNEQRYYGGGYGSTQATFMVFQALAQYQKDA |
| | residues 1002-1303 | PDHQELNLDVSLQLPSR |
| 145 | Human Complement Receptor | GHCQAPDHFLFAKLKTQTNASDFPIGTSLKYECRPEYYGRPFSITCLDNLVWSSPKD |
| | 1; consensus sequence for | VCKRKSCKTPPDPVNGMVHVITDIQVGSRINYSCTTGHRLIGHSSAECILSGNX₁AHW |
| | CCPs 8-10, 15-17 | STKPPICQRIPCGLPPTIANGDFISTNRENFHYGSVVTYRCNX₂GSX₃GRKVFELVGEP |
| | (UniProt: P17927 residues 491 | SIYCTSNDDQVGIWSGPAPQCII |
| | to 684; residues 941 to 1134) | |
| | Without leader sequence | |
| 146 | Human Complement Receptor 1 | GHCQAPDHFLFAKLKTQTNASDFPIGTSLKYECRPEYYGRPFSITCLDNLVWSSPKD |
| | CCPs 8-10 (UniProt: P17927 | VCKRKSCKTPPDPVNGMVHVITDIQVGSRINYSCTTGHRLIGHSSAECILSGNAAHW |
| | residues 491 to 684) | STKPPICQRIPCGLPPTIANGDFISTNRENFHYGSVVTYRCNPGSGGRKVFELVGEPS |
| | Without leader sequence | IYCTSNDDQVGIWSGPAPQCII |
| 147 | Human Complement Receptor 1 | GHCQAPDHFLFAKLKTQTNASDFPIGTSLKYECRPEYYGRPFSITCLDNLVWSSPKD |
| | CCPs 15-17 (UniProt: P17927 | VCKRKSCKTPPDPVNGMVHVITDIQVGSRINYSCTTGHRLIGHSSAECILSGNTAHW |
| | residues 941 to 1134) | STKPPICQRIPCGLPPTIANGDFISTNRENFHYGSVVTYRCNLGSRGRKVFELVGEPS |
| | Without leader sequence | IYCTSNDDQVGIWSGPAPQCII |
| 148 | Human Complement Receptor 1 | GHCQAPDHFLFAKLKTQTNASDFPIGTSLKYECRPEYYGRPFSITCLDNLVWSSPKD |
| | CCPs 8-10 and 15-17 (contiguous; without leader | 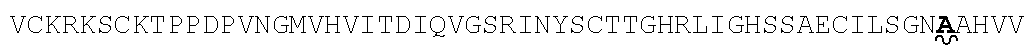 |
| | sequence) [amino acid differences between | 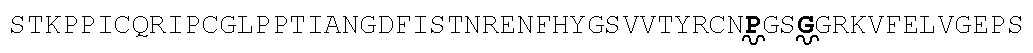 |
| | CCPs 8-10 and 15-17 indicated | IYCTSNDDQVGIWSGPAPQCIIGHCQAPDHFLFAKLKTQTNASDFPIGTSLKYECRPE |
| | with wavy underline] | YYGRPFSITCLDNLVWSSPKDVCKRKSCKTPPDPVNGMVHVITDIQVGSRINYSCTT |
| | | 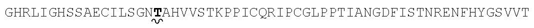 |
| | | 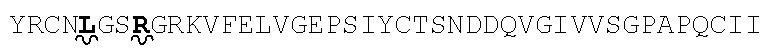 |
| 149 | Human Complement Factor I | IVGGKRAQLGDLPWQVAIKDASGITCGGIYIGGCWILTAAHCLRASKTHRYQIWTTVV |
| | proteolytic domain (UniProt: | DWIHPDLKRIVIEYVDRIIFHENYNAGTYQNDIALIEMKKDGNKKDCELPRSIPACVPW |
| | P05156 residues 340-574) | SPYLFQPNDTCIVSGWGREKDNERVFSLQWGEVKLISNCSKFYGNRFYEKEMECAG |
| | | TYDGSIDACKGDSGGPLVCMDANNVTYVWGVVSWGENCGKPEFPGVYTKVANYFD |
| | | WISYHVG |
| 150 | Human Complement Factor H | EDCNELPPRRNTEILTGSWSDQTYPEGTQAIYKCRPGYRSLGNVIMVCRKGEWVAL |
| | co-factor region (UniProt: | NPLRKCQKRPCGHPGDTPFGTFTLTGGNVFEYGVKAVYTCNEGYQLLGEINYRECD |
| | P08603 residues 19-264) | TDGWTNDIPICEVVKCLPVTAPENGKIVSSAMEPDREYHFGQAVRFVCNSGYKIEGD |
| | | EEMHCSDDGFWSKEKPKCVEISCKSPDVINGSPISQKIIYKENERFQYKCNMGYEYS |
| | | ERGDAVCTESGWRPLPSCEE |
| 151 | Human Complement Receptor 1 | GHCQAPDHFLFAKLKTQTNASDFPIGTSLKYECRPEYYGRPFSITCLDNLVWSSPKD |
| | CCPs 8-10 (UniProt: P17927 | VCKRKSCKTPPDPVNGMVHVITDIQVGSRINYSCTTGHRLIGHSSAECILSGNAAHW |
| | residues 491 to 684) | STKPPICQRIPCGLPPTIANGDFISTNRENFHYGSVVTYRCNPGSGGRKVFELVGEPS |
| | | IYCTSNDDQVGIWSGPAPQCII |
| 152 | Human Complement Receptor 1 | GHCQAPDHFLFAKLKTQTNASDFPIGTSLKYECRPEYYGRPFSITCLDNLVWSSPKD |
| | CCPs 15-17 (UniProt: P17927 | VCKRKSCKTPPDPVNGMVHVITDIQVGSRINYSCTTGHRLIGHSSAECILSGNTAHW |
| | residues 941 to 1134) | STKPPICQRIPCGLPPTIANGDFISTNRENFHYGSVVTYRCNLGSRGRKVFELVGEPS |
| | | IYCTSNDDQVGIWSGPAPQCII |
| 153 | GluC | MKGKFLKVSSLFVATLTTATLVSSPAANALSSKAMDNHPQQTQSSKQQTPKIKKGGN |
| | (Uniprot Q7A6A6 including N- | LKPLEQREHANVILPNNDRHQITDTTNGHYAPVTYIQVEAPTGTFIASGVVVGKDTLLT |
| | terminal signal peptide residues | NKHVVDATHGDPHALKAFPSAINQDNYPNGGFTAEQITKYSGEGDLAIVKFSPNEQN |
| | 1-29 and propeptide residues | KHIGEVVKPATMSNNAETQVNQNITVTGYPGDKPVATMWESKGKITYLKGEAMQYD |
| | 30-68) | LSTTGGNSGSPVFNEKNEVIGIHWGGVPNEFNGAVFINENVRNFLKQNIEDIHFANDD |
| | | QPNNPDNPDNPNNPDNPNNPDNPNNPDEPNNPDNPNNPDNPDNGDNNNSDNPDA |
| | | A |
| 154 | GluC (Uniprot Q7A6A6; without | VILPNNDRHQITDTTNGHYAPVTYIQVEAPTGTFIASGVVVGKDTLLTNKHVVDATHG |
| | signal peptide or propeptide) | DPHALKAFPSAINQDNYPNGGFTAEQITKYSGEGDLAIVKFSPNEQNKHIGEVVKPAT |
| | | MSNNAETQVNQNITVTGYPGDKPVATMWESKGKITYLKGEAMQYDLSTTGGNSGS |
| | | PVFNEKNEVIGIHWGGVPNEFNGAVFINENVRNFLKQNIEDIHFANDDQPNNPDNPD |
| | | NPNNPDNPNNPDNPNNPDEPNNPDNPNNPDNPDNGDNNNSDNPDAA |
| 155 | FI peptide 24 | VGCAGFASVTQE |
| 156 | C3a-desArg | LRRQHARASHLGLA |
| 157 | C3f-desArg | SASLL |
| 158 | siRNA target 1 (CFHR1, | GCUCCUGGUCAGUGUAAUUCU |
| | CFHR2) | |
| 159 | siRNA target 2 (CFHR1) | GGUGAGAGAGUACGUUAUGAA |
| 160 | siRNA target 3 (CFHR1) | GAAUGUAGGAGCCCUUAUGAA |
| 161 | siRNA target 4 (CFHR3) | GUACCAAUGCCAGCCCUACUA |
| 162 | siRNA target 5 (CFHR3, | GGAACCACCAAGAUGCAUACA |
| | CFHR4) | |
| 163 | siRNA target 6 (CFHR3) | GCAUAUGGUUAGUGAGAAGUU |
| 164 | siRNA target 7 (CFHR4) | GAAUCACACUUGGUAACUAAU |
| 165 | siRNA target 8 (CFHR4) | GGUCAAGAGUCGAGUACCAGU |
| 166 | siRNA target 9 (CFHR3, | GAAUGCUACGAUGGAUAUGAA |
| | CFHR4) | |
| 167 | siRNA target 10 (CFHR5) | GAAUCUUCAGGACUAAUACAU |
| 168 | siRNA target 11 (CFHR5) | GUAAAUGUUCUCGCUUCAGUA |
| 169 | siRNA target 12 (CFH, CFHR1, | GUACCAAUGCCAGAACUUGUA |
| | CFHR2) | |
| 170 | siRNA target 13 (CFH, CFHR1) | GAGGGUAACAAGCGAAUAACA |
| 171 | siRNA target 14 (CFH, CFHR1) | GGUAACAAGCGAAUAACAUGU |
| 172 | siRNA target 15 (CFH, CFHR2) | GCUUUAUUCAAGAACAGGUGA |
| 173 | siRNA target 16 (CFHR2) | GAAAUGUCAAUUCCUAACAGU |
| 174 | siRNA target 17 (CFHR3) | GUGAGAAGUUGCAGGGUAAGA |
| 175 | siRNA target 18 (CFHR3) | GUUUCCCCAUACCCCACUCAU |
| 176 | siRNA target 19 (CFHR3) | GCCACCAUGUAAGAUGUACCU |
| 177 | siRNA target 20 (CFHR4) | GGAUAUAAUGCGAAUACAUCA |
| 178 | Antisense target 1 | AGAAUUACACUGACCAGGAGC |
| 179 | Antisense target 2 | UUCAUAACGUACUCUCUCACC |
| 180 | Antisense target 3 | UUCAUAAGGGCUCCUACAUUC |
| 181 | Antisense target 4 | UAGUAGGGCUGGCAUUGGUAC |
| 182 | Antisense target 5 | UGUAUGCAUCUUGGUGGUUCC |
| 183 | Antisense target 6 | AACUUCUCACUAACCAUAUGC |
| 184 | Antisense target 7 | AUUAGUUACCAAGUGUGAUUC |
| 185 | Antisense target 8 | ACUGGUACUCGACUCUUGACC |
| 186 | Antisense target 9 | UUCAUAUCCAUCGUAGCAUUC |
| 187 | Antisense target 10 | AUGUAUUAGUCCUGAAGAUUC |
| 188 | Antisense target 11 | UACUGAAGCGAGAACAUUUAC |
| 189 | Antisense target 12 | UACAAGUUCUGGCAUUGGUAC |
| 190 | Antisense target 13 | UGUUAUUCGCUUGUUACCCUC |
| 191 | Antisense target 14 | ACAUGUUAUUCGCUUGUUACC |
| 192 | Antisense target 15 | UCACCUGUUCUUGAAUAAAGC |
| 193 | Antisense target 16 | ACUGUUAGGAAUUGACAUUUC |
| 194 | Antisense target 17 | UCUUACCCUGCAACUUCUCAC |
| 195 | Antisense target 18 | AUGAGUGGGGUAUGGGGAAAC |
| 196 | Antisense target 19 | AGGUACAUCUUACAUGGUGGC |
| 197 | Antisense target 20 | UGAUGUAUUCGCAUUAUAUCC |
| 198 | Scramble antisense target 1 | GGUUGCAUUAGGCUAUCCUCU |
| 199 | Scramble antisense target 2 | GAAUAGGGUCGGAAGUUAUGA |
| 200 | Scramble antisense target 3 | GUGCAUAACGGUAGAUUCAGA |
| 201 | Scramble antisense target 4 | GAGCCUCACGCUACUAACCUA |
| 202 | Scramble antisense target 5 | GAACAACACGGACGCUCAAUA |
| 203 | Scramble antisense target 6 | GAUUGUGAGAGGUUCGUAAUA |
| 204 | Scramble antisense target 7 | GGAAUUCUAAUCGAAACUACU |
| 205 | Scramble antisense target 8 | GGUCAACCGUAAUGGAGGUCA |
| 206 | Scramble antisense target 9 | GAGUUAGAGCGCAUAGAAUAU |
| 207 | Scramble antisense target 10 | GAUCCAAAUGUACCGAAUUAU |
| 208 | Scramble antisense target 11 | GAAAUUCUUCCGAUUGAGUCU |
| 209 | Scramble antisense target 12 | GAAAGUCACGAUAUCCUGUCA |
| 210 | Scramble antisense target 13 | GAGAAACAAGAAGGCGUAACU |
| 211 | Scramble antisense target 14 | GGAGUACUUAGCGAAUAAACA |
| 212 | Scramble antisense target 15 | GCAAUUAGUUAGGACGAUACU |
| 213 | Scramble antisense target 16 | GUCAACAUCGCAUAGAUAUUA |
| 214 | Scramble antisense target 17 | GAAGCGAAUGGGUAGGUUAGA |
| 215 | Scramble antisense target 18 | GCUCCCUUUCCCCUAAUCACA |
| 216 | Scramble antisense target 19 | GGAUCACACUGCCUACGUAAU |
| 217 | Scramble antisense target 20 | GAAAGAGACGAUUAUUCAACU |
| 218 | Homo sapiens complement | CAGTTAGTACACTGAAATTCAAAGTCATGCTCATAACTGTTAATGAAAGCAGATTC |
| | factor H related 1 (CFHR1), | AAAGCAACACCACCACCACTGAAGTATTTTTAGTTATATAAGATTGGAACTACCAA |
| | transcript variant 1, mRNA | GCATGTGGCTCCTGGTCAGTGTAATTCTAATCTCACGGATATCCTCTGTTGGGGG |
| | NCBI Reference Sequence: | AGAAGCAACATTTTGTGATTTTCCAAAAATAAACCATGGAATTCTATATGATGAAG |
| | NM_002113.3 | AAAAATATAAGCCATTTTCCCAGGTTCCTACAGGGGAAGTTTTCTATTACTCCTGT |
| | GI: 1826689168 | GAATATAATTTTGTGTCTCCTTCAAAATCATTTTGGACTCGCATAACATGCACAGA |
| | | AGAAGGATGGTCACCAACACCAAAGTGTCTCAGACTGTGTTTCTTTCCTTTTGTG |
| | | GAAAATGGTCATTCTGAATCTTCAGGACAAACACATCTGGAAGGTGATACTGTGC |
| | | AAATTATTTGCAACACAGGATACAGACTTCAAAACAATGAGAACAACATTTCATGT |
| | | GTAGAACGGGGCTGGTCCACCCCTCCCAAATGCAGGTCCACTGACACTTCCTGT |
| | | GTGAATCCGCCCACAGTACAAAATGCTCATATACTGTCGAGACAGATGAGTAAAT |
| | | ATCCATCTGGTGAGAGAGTACGTTATGAATGTAGGAGCCCTTATGAAATGTTTGG |
| | | GGATGAAGAAGTGATGTGTTTAAATGGAAACTGGACAGAACCACCTCAATGCAAA |
| | | GATTCTACGGGAAAATGTGGGCCCCCTCCACCTATTGACAATGGGGACATTACTT |
| | | CATTCCCGTTGTCAGTATATGCTCCAGCTTCATCAGTTGAGTACCAATGCCAGAA |
| | | CTTGTATCAACTTGAGGGTAACAAGCGAATAACATGTAGAAATGGACAATGGTCA |
| | | GAACCACCAAAATGCTTACATCCGTGTGTAATATCCCGAGAAATTATGGAAAATTA |
| | | TAACATAGCATTAAGGTGGACAGCCAAACAGAAGCTTTATTTGAGAACAGGTGAA |
| | | TCAGCTGAATTTGTGTGTAAACGGGGATATCGTCTTTCATCACGTTCTCACACATT |
| | | GCGAACAACATGTTGGGATGGGAAACTGGAGTATCCAACTTGTGCAAAAAGATA |
| | | GAATCAATCATAAAATGCACACCTTTATTCAGAACTTTAGTATTAAATCAGTTCTTA |
| | | ATTTCATTTTTAAGTATTGTTTTACTCCTTTTTATTCATACGTAAAATTTTGGATTAA |
| | | TTTGTGAAAATGTAATTATAAGCTGAGACCGGTGGCTCTCTTCTTAAAAGCACCAT |
| | | ATTAAAACTTGGAAAACTAA |
| 219 | Homo sapiens complement | ACCACAAAGGACTTTACTAAACTAGCTTCCAGTTAGTACACTGAAATTCAAAGTCA |
| | factor H related 2 (CFHR2), | TGCTCATAACTGTTAATGAAAGCAGATTCAAAGCAACACCACCACCACTGAAGTA |
| | transcript variant 1, mRNA | TTTTTAGTTATATAAGATTGGAACTACCAAGCATGTGGCTCCTGGTCAGTGTAATT |
| | NCBI Reference Sequence: | CTAATCTCACGGATATCCTCTGTTGGGGGAGAAGCAATGTTCTGTGATTTTCCAA |
| | NM_005666.4 | AAATAAACCATGGAATTCTATATGATGAAGAAAAATATAAGCCATTTTCCCAAGTT |
| | GI: 1389570449 | CCTACAGGGGAAGTTTTCTATTACTCCTGTGAATATAATTTTGTGTCTCCTTCAAA |
| | | ATCCTTTTGGACTCGCATAACGTGCGCAGAAGAAGGATGGTCACCAACACCAAA |
| | | GTGTCTCAGACTGTGTTTCTTTCCTTTTGTGGAAAATGGTCATTCTGAATCTTCAG |
| | | GACAAACACATCTGGAAGGTGATACTGTACAAATTATTTGCAACACAGGATACAG |
| | | ACTTCAAAACAATGAGAACAACATTTCATGTGTAGAACGGGGCTGGTCCACTCCT |
| | | CCCAAATGCAGGTCCACTATTTCTGCAGAAAAATGTGGGCCCCCTCCACCTATTG |
| | | ACAATGGAGACATTACTTCATTCCTGTTGTCAGTATATGCTCCAGGTTCATCAGTT |
| | | GAGTACCAGTGCCAGAACTTGTATCAACTTGAGGGTAACAATCAAATAACATGTA |
| | | GAAACGGACAATGGTCAGAACCACCAAAATGCTTAGATCCATGTGTAATATCACA |
| | | AGAAATTATGGAAAAATATAACATAAAATTAAAGTGGACAAACCAACAAAAGCTTT |
| | | ATTCAAGAACAGGTGACATAGTTGAATTTGTTTGTAAATCTGGATATCATCCAACA |
| | | AAATCTCATTCATTTCGAGCAATGTGTCAGAATGGGAAACTGGTATATCCCAGTT |
| | | GTGAAGAAAAATAGAATCAATGGCATTACTATTAGTAAAATGCACACCTTTTTCTG |
| | | AATTTACTATTATATTTGTTTTCAATTTCATTTTTCAAGTACTGTTTTACTCATTTTTA |
| | | TTCATAAATAAAGTTTTGTGTTGATTTGTGAAAATGCAATTACAATCTGAGATGTGT |
| | | CACAATGGTGAGGACTATCTTCACCAAATCTAAGTAACAACCTAGGAATTGTCTTT |
| | | TTTTTTCTTTTTAAAAAAATTGACAATAACTGTATATATTCATGGAGTACATAGTAA |
| | | TGTTTCCATATATATAATGTATAATGGTCAGTTAGGGTAATTAGTATATCCATTATC |
| | | TCAAACATTTTTCATTTCTTTGGGTTAGGAGCATTAAATATTCTCCTTCCAGCTATT |
| | | TGGTACTTCATAGTATATTACTGGTAACTGAAGGAATTATATCTAGACGTTACCCC |
| | | AGGTATCTTGAAATGTCAATTCCTAACAGTCACAGCCTGGGAGCTCATGTTTGCC |
| | | TTCTTTCAGAGCTTGTAACTATGTATATCCACATAAATAATCAAAA |
| 220 | Homo sapiens complement | AGTGCAACTGAAACTTTTGTATTAGCATACTACTGAGAATATCTAACATGTTGTTA |
| | factor H related 3 (CFHR3), | CTAATCAATGTCATTCTGACCTTGTGGGTTTCCTGTGCTAATGGACAAGTGAAAC |
| | transcript variant 1, mRNA | CTTGTGATTTTCCAGACATTAAACATGGAGGTCTATTTCATGAGAATATGCGTAGA |
| | NCBI Reference Sequence: | CCATACTTTCCAGTAGCTGTAGGAAAATATTACTCCTATTACTGTGATGAACATTT |
| | NM_021023.6 | TGAGACTCCTTCAGGAAGTTACTGGGATTACATTCATTGCACACAAAATGGGTGG |
| | GI: 1780222577 | TCACCAGCAGTACCATGTCTCAGAAAATGTTATTTTCCTTATTTGGAAAATGGATA |
| | | TAATCAAAATTATGGAAGAAAGTTTGTACAGGGTAACTCTACAGAAGTTGCCTGC |
| | | CATCCTGGCTACGGTCTTCCAAAAGCGCAGACCACAGTTACATGTACGGAGAAA |
| | | GGCTGGTCTCCTACTCCCAGATGCATCCGTGTCAGAACATGCTCAAAATCAGATA |
| | | TAGAAATTGAAAATGGATTCATTTCCGAATCTTCCTCTATTTATATTTTAAATAAAG |
| | | AAATACAATATAAATGTAAACCAGGATATGCAACAGCAGATGGAAATTCTTCAGG |
| | | ATCAATTACATGTTTGCAAAATGGATGGTCAGCACAACCAATTTGCATTAATTCTT |
| | | CAGAAAAGTGTGGGCCTCCTCCACCTATTAGCAATGGTGATACCACCTCCTTTCT |
| | | ACTAAAAGTGTATGTGCCACAGTCAAGAGTCGAGTACCAATGCCAGCCCTACTAT |
| | | GAACTTCAGGGTTCTAATTATGTAACATGTAGTAATGGAGAGTGGTCGGAACCAC |
| | | CAAGATGCATACATCCATGTATAATAACTGAAGAAAACATGAATAAAAATAACATA |
| | | AAGTTAAAAGGAAGAAGTGACAGAAAATATTATGCAAAAACAGGGGATACCATTG |
| | | AATTTATGTGTAAATTGGGATATAATGCAAATACATCAATTCTATCATTTCAAGCAG |
| | | TGTGTCGGGAAGGGATAGTGGAATACCCCAGATGCGAATAAGGCAGCATTGTTA |
| | | CCCTAAATGTATGTCCAACTTCCACTTTTCCACTTCTCACTCTTATGGTCTCAAAG |
| | | CTTGCAAAGATAGCTTCTGATATTGTTGTAATTTCTACTTTATTTCAAAGAAAATTA |
| | | ATATAATAGTTTCAATTTGCAACTTAATATATTCTCAAAAATATATTAAAACAAACTA |
| | | AATTATTGCTTATGCTTGTACTAAAATAATAAAAACTACTCTTATATTGGACTTCTT |
| | | ATCAATGAATTAGTAAGTATAGAGACAGACAGCTGAATGGCTTTCTGCATATTGTA |
| | | TAGTATACCTAGACATAGAAACAAAATGACTTTAGATTTTATTTGGGGAAGTAATA |
| | | ATACCATAAAATTAGATATTAAAATTGTAAGTGAAGATAAACACACTATAGTATTCC |
| | | CTTATTGTAGCCATGGTCCTCTAGATGCAGTTAACCAAATAGGGTCATTTTTATTA |
| | | AAAGTAGTGTTTCCTGGCAAACACTGACATTACATCATTATCATGATTTAAAGGAA |
| | | ATAGTACTAGAGAAGGTGAATTATTATCATTTTCCTGTGAAAAAAGAAAAGAGGTT |
| | | TTGCTAACCCTTTCAGAGCATTGGGAACACAGCCAGAAGTGCATTAAATGTATAT |
| | | ATTAACTTGGGCAATGTTGACACTTTAGGATGCTGAAGCCAGGTGCAGTGGCAC |
| | | ACGCCTGTAATCCTAGCACTTTGAGAGGCCAAGCTGGCAATCATCAGAGGTCAA |
| | | AAGTTCAAGACCAGCCTGTTCAACACGGTGAAACCCTGTCTCTACTAAAAATAGA |
| | | AAAACTAGCTCGGCATGATGGCGTGCACCTGTAGTCCCAGCTACTCAGGAGGCT |
| | | GAGGTGGGAGAATCACTTTAACCAGTGGGGCAGAGGTTGAACTGAGCCAAGATA |
| | | GTGCCACTGCACTCCAGCCTGGGCAATAGAGTGAGACTCTGTCTTAAAAATAAAT |
| | | AAATAGGATGCTGAAAATTCCTATTTAAGGAAATAATTCTTTTTCTGATTTATTTAA |
| | | GGCTACATTTGTATTTTCTTAATACAAATATTTTGAAAGTTTCTTCATATATGGTTTT |
| | | TTGCATTTCTTATTAAGTTTTGCTTCAGATTTTTTTTTGTCTTTTCTCCTGTTAATTG |
| | | CCTTCACTCTCTTTCTGTTCTGTACTTTTCTTTGTATATAAGGATTTTTTGAAAAGA |
| | | TTGTATATCCCTGTTAACAAATTGAAATCTCTATTCACTTTAATAGATTTTTACCCC |
| | | CAGTTAGCATATGGTTAGTGAGAAGTTGCAGGGTAAGAAGAAAACAATGTTCCCT |
| | | TTCCCAACACTTTTCCTGATTATAGAGAAAAAAGCATGAACTTGTATAATGATCTA |
| | | GTCCTGTACAGAATGAGAAATATTAATTGCTAGACTGAGAATGTTTTGGTGGCTA |
| | | AAGCTAGGAATAACTTTTTAAGACTGAAGAATGATATAAGCTATCAAATCTAACTC |
| | | AGTTTTCAAATAACAGGTTTGCAGAGACTGGAGAATAAAAAGTAAAAAATTGTTTT |
| | | ATTAATTCCAGTGACTAATTCTACTTCATCAACATACATCATAAATAAAATTTCAAA |
| | | AATAATTGCTGATATGGTTTGGCTGTGTCCCACCCAATTTTCACCTTGAATTATAT |
| | | AATCACCATGCATCAAAGGTAGGGCCAGGTGGAGATAATGGAATCATGGGAGCA |
| | | GTTTCCCCATACCCCACTCATGGTAGTAAATACATCTCATGAGATCTAATGGTTTT |
| | | ATAAATGAAAGTTCCCCTGGACAAGTTCTCTTGCCTGCCACCATGTAAGATGTAC |
| | | CTTTGCTACTCATTCACCTTCTGTCATGATGGTGAGGCCTTCCCAGCAATGTGGA |
| | | ACTGTGAGTCCATTAAACCTCTTTCCTTTATAAATTA |
| 221 | Homo sapiens complement | AAATTCAGAATCACACTTGGTAACTAATAATGAAAGATTTCAAACCCCAAACAGTG |
| | factor H related 4 (CFHR4), | CAACTGAAACTTTTGCATTACTATACTACTGAGAATATCTAACATGTTGTTACTAAT |
| | transcript variant 1, mRNA | CAATGTCATTCTGACCTTGTGGGTTTCCTGTGCTAATGGACAAGAAGTGAAACCT |
| | NCBI Reference Sequence: | TGTGATTTTCCAGAAATTCAACATGGAGGTCTATATTATAAGAGTTTGCGTAGACT |
| | NM_001201550.3 | ATACTTTCCAGCAGCTGCAGGACAATCTTATTCCTATTACTGTGATCAAAATTTTG |
| | GI: 1393568848 | TGACTCCTTCAGGAAGTTACTGGGATTACATTCATTGCACACAAGATGGTTGGTC |
| | | ACCAACGGTCCCATGCCTCAGAACATGCTCAAAATCAGATGTAGAAATTGAAAAT |
| | | GGATTCATTTCTGAATCTTCCTCTATTTATATTTTAAATGAAGAAACACAATATAAT |
| | | TGTAAACCAGGATATGCAACAGCAGAGGGAAATTCTTCAGGATCAATTACATGTT |
| | | TGCAAAATGGATGGTCAACACAACCAATTTGCATTAAATTTTGTGATATGCCTGTT |
| | | TTTGAGAATTCCAGAGCCAAGAGTAATGGCATGTGGTTTAAGCTCCATGACACAT |
| | | TGGACTATGAATGCTATGATGGATATGAAAGCAGTTATGGAAACACCACAGATTC |
| | | CATAGTGTGTGGTGAAGATGGCTGGTCCCATTTGCCAACATGCTATAATTCTTCA |
| | | GAAAACTGTGGGCCTCCTCCACCTATTAGCAATGGAGATACCACGTCCTTCCCG |
| | | CAAAAAGTGTATCTGCCATGGTCAAGAGTCGAGTACCAGTGCCAGTCCTACTATG |
| | | AACTTCAGGGTTCTAAATATGTAACATGTAGTAATGGAGACTGGTCAGAACCACC |
| | | AAGATGCATATCAATGAAACCTTGTGAGTTTCCAGAAATTCAACATGGACATCTAT |
| | | ATTATGAGAATACGCGTAGACCATACTTTCCAGTAGCTACAGGACAATCTTACTC |
| | | CTATTACTGTGACCAAAATTTTGTGACTCCTTCAGGAAGTTACTGGGATTACATTC |
| | | ACTGCACACAAGATGGGTGGTTGCCAACAGTCCCATGCCTCAGAACATGCTCAA |
| | | AATCAGATATAGAAATTGAAAATGGATTCATTTCTGAATCTTCCTCTATTTATATTT |
| | | TAAATAAAGAAATACAATATAAATGTAAACCAGGATATGCAACAGCAGATGGAAAT |
| | | TCTTCAGGTTCAATTACATGTTTGCAAAATGGATGGTCAGCACAACCAATTTGCAT |
| | | TAAATTTTGTGATATGCCTGTTTTTGAGAATTCCAGAGCCAAGAGTAATGGCATGC |
| | | GGTTTAAGCTCCATGACACATTGGACTACGAATGCTACGATGGATATGAAATCAG |
| | | TTATGGAAACACCACAGGTTCCATAGTGTGTGGTGAAGATGGGTGGTCCCATTTC |
| | | CCAACATGTTATAATTCTTCAGAAAAGTGTGGGCCTCCTCCACCTATTAGCAATG |
| | | GTGATACCACCTCCTTTCTACTAAAAGTGTATGTGCCACAGTCAAGAGTCGAGTA |
| | | CCAATGCCAGTCCTACTATGAACTTCAGGGTTCTAATTATGTAACATGTAGTAATG |
| | | GAGAGTGGTCGGAACCACCAAGATGCATACATCCATGTATAATAACTGAAGAAAA |
| | | CATGAATAAAAATAACATACAGTTAAAAGGAAAAAGTGACATAAAATATTATGCAA |
| | | AAACAGGGGATACCATTGAATTTATGTGTAAATTGGGATATAATGCGAATACATCA |
| | | GTTCTATCATTTCAAGCAGTGTGTAGGGAAGGCATAGTGGAATACCCCAGATGC |
| | | GAATAAGGCAGCATTGTTACCCTAAATGTATGTCCAACTTCCACTTCTCACTCTTA |
| | | TGGTCTCAAAGCTTGCAAAGATAGCTTCTGATATTGTTGTAATTTCTACTTTATTTC |
| | | AAAGAAAATTAATATAATAGTTTCAATTTGCAACTTAATATGTTCTCAAAAATATGT |
| | | TAAAACAAACTAAATTATTGCTTATGCTTGTACTAAAATAATAAAAACTACCCTTAT |
| | | ATTGGA |
| 222 | Homo sapiens complement | AGTACATTGAAATTCAAAGTCATGCTTGTAACTGTTAATGAAAGCAGATTTAAAGC |
| | factor H related 5 (CFHR5), | AACACCACCATCACTGGAGTATTTTTAGTTATATACGATTGAGACTACCAAGCATG |
| | mRNA | TTGCTCTTATTCAGTGTAATCCTAATCTCATGGGTATCCACTGTTGGGGGAGAAG |
| | NCBI Reference Sequence: | GAACACTTTGTGATTTTCCAAAAATACACCATGGATTTCTGTATGATGAAGAAGAT |
| | NM_030787.4 | TATAACCCTTTTTCCCAAGTTCCTACAGGGGAAGTTTTCTATTACTCCTGTGAATA |
| | GI: 1732746279 | TAATTTTGTGTCTCCTTCAAAATCCTTTTGGACTCGCATAACATGCACAGAAGAAG |
| | | GATGGTCACCAACACCGAAGTGTCTCAGAATGTGTTCCTTTCCTTTTGTGAAAAA |
| | | TGGTCATTCTGAATCTTCAGGACTAATACATCTGGAAGGTGATACTGTACAAATTA |
| | | TTTGCAACACAGGATACAGCCTTCAAAACAATGAGAAAAACATTTCGTGTGTAGA |
| | | ACGGGGCTGGTCCACTCCTCCCATATGCAGCTTCACTAAAGGAGAATGTCATGTT |
| | | CCAATTTTAGAAGCCAATGTAGATGCTCAGCCAAAAAAAGAAAGCTACAAAGTTG |
| | | GAGACGTGTTGAAATTCTCCTGCAGAAAAAATCTTATAAGAGTTGGATCAGACTC |
| | | AGTTCAATGTTACCAATTTGGGTGGTCACCTAACTTTCCAACATGCAAAGGACAA |
| | | GTACGATCATGTGGTCCACCTCCTCAACTCTCCAATGGTGAAGTTAAGGAGATAA |
| | | GAAAAGAGGAATATGGACACAATGAAGTAGTGGAATATGATTGCAATCCTAATTT |
| | | TATAATAAACGGGCCTAAGAAAATACAATGTGTGGATGGAGAATGGACAACTTTA |
| | | CCCACTTGTGTTGAACAAGTGAAAACATGTGGATACATACCTGAACTCGAGTACG |
| | | GTTATGTTCAGCCGTCTGTCCCTCCCTATCAACATGGAGTTTCAGTCGAGGTGAA |
| | | TTGCAGAAATGAATATGCAATGATTGGAAATAACATGATTACCTGTATTAATGGAA |
| | | TATGGACAGAGCTTCCTATGTGTGTTGCAACACACCAACTTAAGAGGTGCAAAAT |
| | | AGCAGGAGTTAATATAAAAACATTACTCAAGCTATCTGGGAAAGAATTTAATCATA |
| | | ATTCTAGAATACGTTACAGATGTTCAGACATCTTCAGATACAGGCACTCAGTCTGT |
| | | ATAAACGGGAAATGGAATCCTGAAGTAGACTGCACAGAAAAAAGGGAACAATTCT |
| | | GCCCACCGCCACCTCAGATACCTAATGCTCAGAATATGACAACCACAGTGAATTA |
| | | TCAGGATGGAGAAAAAGTAGCTGTTCTCTGTAAAGAAAACTATCTACTTCCAGAA |
| | | GCAAAAGAAATTGTATGTAAAGATGGACGATGGCAATCATTACCACGCTGTGTTG |
| | | AGTCTACTGCATATTGTGGGCCCCCTCCATCTATTAACAATGGAGATACCACCTC |
| | | ATTCCCATTATCAGTATATCCTCCAGGGTCAACAGTGACGTACCGTTGCCAGTCC |
| | | TTCTATAAACTCCAGGGCTCTGTAACTGTAACATGCAGAAATAAACAGTGGTCAG |
| | | AACCACCAAGATGCCTAGATCCATGTGTGGTATCTGAAGAAAACATGAACAAAAA |
| | | TAACATACAGTTAAAATGGAGAAACGATGGAAAACTCTATGCAAAAACAGGGGAT |
| | | GCTGTTGAATTCCAGTGTAAATTCCCACATAAAGCGATGATATCATCACCACCATT |
| | | TCGAGCAATCTGTCAGGAAGGGAAATTTGAATATCCTATATGTGAATGAAGCAAG |
| | | CATAATTTTCCTGAATATATTCTTCAAACATCCATCTATGCTAAAAGTAGCCATTAT |
| | | GTAGCCAATTCTGTAGTTACTTCTTTTATTCTTTCAGGTGTTGTTTAACTCAGTTTT |
| | | ATTTAGAACTCTGGATTTTTAGAGCTTTAGAAATTTGTAAGCTGAGAGAACAATGT |
| | | TTCACTTAATAGGAGGGTGTCTTAGTCCATATTACATTGTTATAACAGAGTATCAC |
| | | AGACTGGATAACTTCTAACCAATAGTTTATTTGTTTCATAAATCTAAAAGCTGAGA |
| | | AGTCCAAGATGGTGGGGCTGCCTCTGGTGAGGGTCTTCTCGAAGCATCATAATA |
| | | TGCTGGAAGGCATCACAACATGGTGGAAGGGATCACGTGGCAAAAGAGCATGTA |
| | | CATGGGAGTGAGAGAAAAAGAGAGAGAGAGACAGAGTGGGGGGGGGGGGAG |
| | | GAGCGCAAACTCATCCTTTATAAAGACACCACTCCTGAGATAACAATCCAATCCC |
| | | ATGATAATGACATTAATCCATTCAAGAAGATAGAGCTCTCGTGACTTAATCACCTT |
| | | CTAAAGATCTCACCTGACAACACTGTTGCATTGGCAGTTAAGTTTCCACGTAAAC |
| | | TTTCGGGGACACATTCAAACCACAGGAGAAACTCAAATTGTTCCTGGGCAAATCA |
| | | CAACATGGGGAATTTTATTCATAAATGTCCACAGAAACAGTAAATGTTCTCGCTTC |
| | | AGTACTTAATTCATCTAATCCCTCCTGTTTGTCTCAAATTATAGGATAACTTTGAAA |
| | | CTTTCTGAATTAACGTTATTTAAAAGGAAATGTAGATGTTATTTTAGTCTCTATCTT |
| | | CATGTTATTATCACTTAAAAACCTGCGAAAGCTGTCAACTTTTGTGGTTGTAGCAA |
| | | GTATTAATAAATATTTATAAATCCTCTAATGTAAGTCTAGCTACCTATCCAATACTA |
| | | AATACCCCTTAAAGTATTAAATGCACTATCTGCTGTAAAAGAA |
| 223 | Homo sapiens complement | ACAATTCTTGGAAGAGGAGAACTGGACGTTGTGAACAGAGTTAGCTGGTAAATGT |
| | factor H (CFH), transcript variant | CCTCTTAAAAGATCCAAAAAATGAGACTTCTAGCAAAGATTATTTGCCTTATGTTA |
| | 1, mRNA | TGGGCTATTTGTGTAGCAGAAGATTGCAATGAACTTCCTCCAAGAAGAAATACAG |
| | NCBI Reference Sequence: | AAATTCTGACAGGTTCCTGGTCTGACCAAACATATCCAGAAGGCACCCAGGCTAT |
| | NM_000186.4 | CTATAAATGCCGCCCTGGATATAGATCTCTTGGAAATGTAATAATGGTATGCAGG |
| | GI: 1732746315 | AAGGGAGAATGGGTTGCTCTTAATCCATTAAGGAAATGTCAGAAAAGGCCCTGTG |
| | | GACATCCTGGAGATACTCCTTTTGGTACTTTTACCCTTACAGGAGGAAATGTGTTT |
| | | GAATATGGTGTAAAAGCTGTGTATACATGTAATGAGGGGTATCAATTGCTAGGTG |
| | | AGATTAATTACCGTGAATGTGACACAGATGGATGGACCAATGATATTCCTATATGT |
| | | GAAGTTGTGAAGTGTTTACCAGTGACAGCACCAGAGAATGGAAAAATTGTCAGTA |
| | | GTGCAATGGAACCAGATCGGGAATACCATTTTGGACAAGCAGTACGGTTTGTATG |
| | | TAACTCAGGCTACAAGATTGAAGGAGATGAAGAAATGCATTGTTCAGACGATGGT |
| | | TTTTGGAGTAAAGAGAAACCAAAGTGTGTGGAAATTTCATGCAAATCCCCAGATG |
| | | TTATAAATGGATCTCCTATATCTCAGAAGATTATTTATAAGGAGAATGAACGATTT |
| | | CAATATAAATGTAACATGGGTTATGAATACAGTGAAAGAGGAGATGCTGTATGCA |
| | | CTGAATCTGGATGGCGTCCGTTGCCTTCATGTGAAGAAAAATCATGTGATAATCC |
| | | TTATATTCCAAATGGTGACTACTCACCTTTAAGGATTAAACACAGAACTGGAGATG |
| | | AAATCACGTACCAGTGTAGAAATGGTTTTTATCCTGCAACCCGGGGAAATACAGC |
| | | AAAATGCACAAGTACTGGCTGGATACCTGCTCCGAGATGTACCTTGAAACCTTGT |
| | | GATTATCCAGACATTAAACATGGAGGTCTATATCATGAGAATATGCGTAGACCAT |
| | | ACTTTCCAGTAGCTGTAGGAAAATATTACTCCTATTACTGTGATGAACATTTTGAG |
| | | ACTCCGTCAGGAAGTTACTGGGATCACATTCATTGCACACAAGATGGATGGTCG |
| | | CCAGCAGTACCATGCCTCAGAAAATGTTATTTTCCTTATTTGGAAAATGGATATAA |
| | | TCAAAATCATGGAAGAAAGTTTGTACAGGGTAAATCTATAGACGTTGCCTGCCAT |
| | | CCTGGCTACGCTCTTCCAAAAGCGCAGACCACAGTTACATGTATGGAGAATGGC |
| | | TGGTCTCCTACTCCCAGATGCATCCGTGTCAAAACATGTTCCAAATCAAGTATAG |
| | | ATATTGAGAATGGGTTTATTTCTGAATCTCAGTATACATATGCCTTAAAAGAAAAA |
| | | GCGAAATATCAATGCAAACTAGGATATGTAACAGCAGATGGTGAAACATCAGGAT |
| | | CAATTACATGTGGGAAAGATGGATGGTCAGCTCAACCCACGTGCATTAAATCTTG |
| | | TGATATCCCAGTATTTATGAATGCCAGAACTAAAAATGACTTCACATGGTTTAAGC |
| | | TGAATGACACATTGGACTATGAATGCCATGATGGTTATGAAAGCAATACTGGAAG |
| | | CACCACTGGTTCCATAGTGTGTGGTTACAATGGTTGGTCTGATTTACCCATATGT |
| | | TATGAAAGAGAATGCGAACTTCCTAAAATAGATGTACACTTAGTTCCTGATCGCAA |
| | | GAAAGACCAGTATAAAGTTGGAGAGGTGTTGAAATTCTCCTGCAAACCAGGATTT |
| | | ACAATAGTTGGACCTAATTCCGTTCAGTGCTACCACTTTGGATTGTCTCCTGACC |
| | | TCCCAATATGTAAAGAGCAAGTACAATCATGTGGTCCACCTCCTGAACTCCTCAA |
| | | TGGGAATGTTAAGGAAAAAACGAAAGAAGAATATGGACACAGTGAAGTGGTGGA |
| | | ATATTATTGCAATCCTAGATTTCTAATGAAGGGACCTAATAAAATTCAATGTGTTG |
| | | ATGGAGAGTGGACAACTTTACCAGTGTGTATTGTGGAGGAGAGTACCTGTGGAG |
| | | ATATACCTGAACTTGAACATGGCTGGGCCCAGCTTTCTTCCCCTCCTTATTACTAT |
| | | GGAGATTCAGTGGAATTCAATTGCTCAGAATCATTTACAATGATTGGACACAGAT |
| | | CAATTACGTGTATTCATGGAGTATGGACCCAACTTCCCCAGTGTGTGGCAATAGA |
| | | TAAACTTAAGAAGTGCAAATCATCAAATTTAATTATACTTGAGGAACATTTAAAAAA |
| | | CAAGAAGGAATTCGATCATAATTCTAACATAAGGTACAGATGTAGAGGAAAAGAA |
| | | GGATGGATACACACAGTCTGCATAAATGGAAGATGGGATCCAGAAGTGAACTGC |
| | | TCAATGGCACAAATACAATTATGCCCACCTCCACCTCAGATTCCCAATTCTCACAA |
| | | TATGACAACCACACTGAATTATCGGGATGGAGAAAAAGTATCTGTTCTTTGCCAA |
| | | GAAAATTATCTAATTCAGGAAGGAGAAGAAATTACATGCAAAGATGGAAGATGGC |
| | | AGTCAATACCACTCTGTGTTGAAAAAATTCCATGTTCACAACCACCTCAGATAGAA |
| | | CACGGAACCATTAATTCATCCAGGTCTTCACAAGAAAGTTATGCACATGGGACTA |
| | | AATTGAGTTATACTTGTGAGGGTGGTTTCAGGATATCTGAAGAAAATGAAACAAC |
| | | ATGCTACATGGGAAAATGGAGTTCTCCACCTCAGTGTGAAGGCCTTCCTTGTAA |
| | | ATCTCCACCTGAGATTTCTCATGGTGTTGTAGCTCACATGTCAGACAGTTATCAG |
| | | TATGGAGAAGAAGTTACGTACAAATGTTTTGAAGGTTTTGGAATTGATGGGCCTG |
| | | CAATTGCAAAATGCTTAGGAGAAAAATGGTCTCACCCTCCATCATGCATAAAAAC |
| | | AGATTGTCTCAGTTTACCTAGCTTTGAAAATGCCATACCCATGGGAGAGAAGAAG |
| | | GATGTGTATAAGGCGGGTGAGCAAGTGACTTACACTTGTGCAACATATTACAAAA |
| | | TGGATGGAGCCAGTAATGTAACATGCATTAATAGCAGATGGACAGGAAGGCCAA |
| | | CATGCAGAGACACCTCCTGTGTGAATCCGCCCACAGTACAAAATGCTTATATAGT |
| | | GTCGAGACAGATGAGTAAATATCCATCTGGTGAGAGAGTACGTTATCAATGTAGG |
| | | AGCCCTTATGAAATGTTTGGGGATGAAGAAGTGATGTGTTTAAATGGAAACTGGA |
| | | CGGAACCACCTCAATGCAAAGATTCTACAGGAAAATGTGGGCCCCCTCCACCTA |
| | | TTGACAATGGGGACATTACTTCATTCCCGTTGTCAGTATATGCTCCAGCTTCATCA |
| | | GTTGAGTACCAATGCCAGAACTTGTATCAACTTGAGGGTAACAAGCGAATAACAT |
| | | GTAGAAATGGACAATGGTCAGAACCACCAAAATGCTTACATCCGTGTGTAATATC |
| | | CCGAGAAATTATGGAAAATTATAACATAGCATTAAGGTGGACAGCCAAACAGAAG |
| | | CTTTATTCGAGAACAGGTGAATCAGTTGAATTTGTGTGTAAACGGGGATATCGTC |
| | | TTTCATCACGTTCTCACACATTGCGAACAACATGTTGGGATGGGAAACTGGAGTA |
| | | TCCAACTTGTGCAAAAAGATAGAATCAATCATAAAGTGCACACCTTTATTCAGAAC |
| | | TTTAGTATTAAATCAGTTCTCAATTTCATTTTTTATGTATTGTTTTACTCCTTTTTAT |
| | | TCATACGTAAAATTTTGGATTAATTTGTGAAAATGTAATTATAAGCTGAGACCGGT |
| | | GGCTCTCTTCTTAAAAGCACCATATTAAATCCTGGAAAACTAA |
| 224 | guideRNA 1 | AGATTAGAATTACACTGACCAGG |
| 225 | guideRNA 2 | TAATAGAAAACTTCCCCTGTAGG |
| 226 | guideRNA 3 | AGACACAAAATTATATTCACAGG |
| 227 | guideRNA 4 | AATGGTCATTCTGAATCTTCAGG |

### FIGURES

Embodiments and experiments illustrating the principles of the invention will now be discussed with reference to the accompanying figures in which:

FIG. 1. Schematic showing the C3 proteolytic cascade and the proteolytic events leading to the generation, breakdown and inactivation of C3b (modified from Maillard et al, J Am Soc Nephrol. 2015 July; 26(7):1503-12). Proteoform-specific peptides for mass spectrometry are underlined.

FIG. 2. LC-SRM Trace showing detection of the heavy-labelled synthetic standard peptides of each individual RCA locus protein from a plasma sample.

FIG. 3. Linearity data for peptides derived from FH, FHL-1, and FHR1-5.

FIGS. 4A to 4D. Data confirming that C3 and C3 breakdown products in human plasma can be detected by MS with sufficient specificity and sensitivity. 4A: Total ion chromatograph from SRM-MS analysis showing specific and simultaneous detection of C3b fragment-specific peptides. 4B: Linearity data for seven of the ten peptides spiked into a plasma background. 4C: Coomassie-stained electrophoresis gel of C3 breakdown products obtained in vitro. 4D: MS quantification of key C3 fragments from the in vitro assay products shown in 4C.

FIG. 5. Correlation matrix showing the Pearson correlation coefficients between the different variables (absolute concentration levels of various studied proteins).

FIG. 6. Scatterplot showing differences in protein levels between subjects with AMD and control individuals (mean and p values shown (p<0.05 considered statistically significant)).

FIG. 7. Area under the Receiver Operating Characteristic curve for various models.

FIG. 8. Receiver Operating Characteristic curve for a model that uses FHR1, FHR2, FHR3, FHR4, FHR5, FHL-1 & CFH levels (with 2- & 3-way interactions) to predict whether an individual is an AMD case or a control subject.

FIG. 9. GWASs of circulating FHR-1 to FHR-5 protein levels reveal a strong genome-wide significant signal spanning the CFH locus. Regional plots show the genome-wide significant (P-value≤5×10⁻⁸) association signals from the GWASs of FHR-1 to FHR-5 protein levels (panels A-E) at the CFH locus on chromosome 1q31.3. Panel F shows the equivalent CFH region for the GWAS of FHL-1 protein levels (no genome-wide significant association regions observed). The most associated variant is denoted by a purple diamond and is labelled by its rs-number. The other surrounding variants are shown by circles coloured to reflect the extent of linkage disequilibrium with the most associated variant (based on the European (EUR) population genotype data originated from the 1000 Genomes Project, November 2014). A diagram of the genes within the relevant regions is depicted below each plot. Physical positions are based on NCBI RefSeq hg19 human genome reference assembly.

FIG. 10. Established AMD risk variants at the CFH locus are associated at genome-wide significance level with circulating FHR-1, FHR-2, FHR-3 and FHR-4 protein levels in 252 Cambridge controls. Box plots of FHR protein levels by variant genotype for those established AMD risk variants at the CFH locus from the IAMDGC study that showed genome-wide significant (P-value 5×10⁻⁸) associations in 252 controls from the Cambridge AMD cohort (Table 2). P-values and Beta values from Wald tests using linear regression models adjusted for sex, age and the first two genetic principal components (as estimated within the IAMDGC study) are indicated in the note at the bottom of each plot.

FIG. 11. Mendelian randomization analysis shows highly significant elevation of circulating FHR-1, FHR-2, FHR-4 and FHR-5 protein levels in advanced AMD. Mendelian randomization estimates of the association of FHR-1 (panel A), FHR-2 (panel B), FHR-3 (panel C), FHR-4 (panel D) and FHR-5 (panel E) are presented together with the corresponding traditional epidemiologic odds ratio (OR) estimates obtained from logistic regression models (352 advanced AMD cases and 252 controls from the Cambridge AMD study). The Mendelian randomization estimates were obtained using the Wald ratio (if a single instrument was available; FHR1, FHR2, FHR4, FHR5) or the inverse-variance weighted (IVW) method under a fixed-effect model (if multiple instruments were available; FHR3). Raw data used to calculate the Mendelian randomization estimates are provided in Table S7. The variance of each protein explained by its genetic instrument(s) is indicated in the note at the bottom of each plot.

FIGS. 12A to 12D. Graphs showing suppression of CFH family gene expression in huH1 cells by CFHR siRNA 1, 2 and 3. (A) and (B) Relative quantification of CFHR transcript was assessed in huH1 cells after first transfection with CFHR siRNA1, CFHR siRNA2, CFHR siRNA3 (10 nm each) treatment when compared to their respective Scrambled/control siRNA (10 nm) treatment for 24 h; (C) Second transfection result after CFHR siRNA1, CFHR siRNA2, CFHR siRNA3 (10 nm each) and their respective Scrambled/control siRNA (10 nm) treatment after 24 h; (D) Combined results for relative quantification of CFHR transcript from three separate transfections by CFHR siRNA1 (10 nm) treatment when compared to Scrambled 1/control siRNA (10 nm) treatment for 24 h, each in triplicate (n=9). *** denotes p<0.005; * denotes p<0.05, Bar graphs illustrate fold change (2^{-ΔΔCT})±SEM).

FIG. 13. Graphs showing suppression of CFHR gene expression in huH1 cells by CFHR siRNA 4, 5 and 6. Relative quantification of CFHR transcript was assessed in huH1 cells after transfection with CFHR siRNA4, CFHR siRNA5, CFHR siRNA6 (10 nm each) treatment when compared to their respective Scrambled/control siRNA (10 nm) treatment for 24 h. *** denotes p<0.005; * denotes p<0.05, Bar graphs illustrate fold change (2^{-ΔΔCT})±SEM).

FIG. 14. Graphs showing suppression of CFHR gene expression in huH1 cells by CFHR siRNA 7, 8 and 9. Relative quantification of CFHR transcript was assessed in huH1 cells after transfection with CFHR siRNA7, CFHR siRNA8, CFHR siRNA9 (10 nm each) treatment when compared to their respective Scrambled/control siRNA (10 nm) treatment for 24 h. *** denotes p<0.005; * denotes p<0.05, Bar graphs illustrate fold change (2^{-ΔΔCT})±SEM).

### EXAMPLES

### Example 1: Generation of Peptides from Complement Proteins for Mass Spectrometry

GluC digestion was performed on FH, FHL-1, FHR1-5, FI, C3, C3b and C3b breakdown products to achieve distinct peptides for mass spectrometry. GluC digestion is described in Example 2.2.

Peptides that can be used to detect each protein or protein fragment are set out in Tables 1-4 below.

TABLE 1
Distinct FH family peptides after
GluC digestion.

| | | | SEQ |
|---|---|---|---|
| | | | ID |
| Protein | Peptide Sequence | Mass | No: |
| Factor H | VTYKCFE | 888.4051 | 20 |
| FHL1 | NGWSPTPRCIRVSFTL | 1832.9355 | 21 |
| FHR1 | ATFCDFPKINHGILYDEE | 2110.9669 | 22 |
| FHR2 | RGWSTPPKCRSTISAE | 1774.8784 | 23 |
| | and | | |
| | AMFCDFPKINHGILYDEE | 2140.9598 | 24 |
| FHR3 | VACHPGYGLPKAQTTVTCTE | 2074.9816 | 25 |
| FHR4 | YQCQSYYE | 1082.4015 | 26 |
| FHR5 | RGWSTPPICSFTKGE | 1664.7980 | 27 |

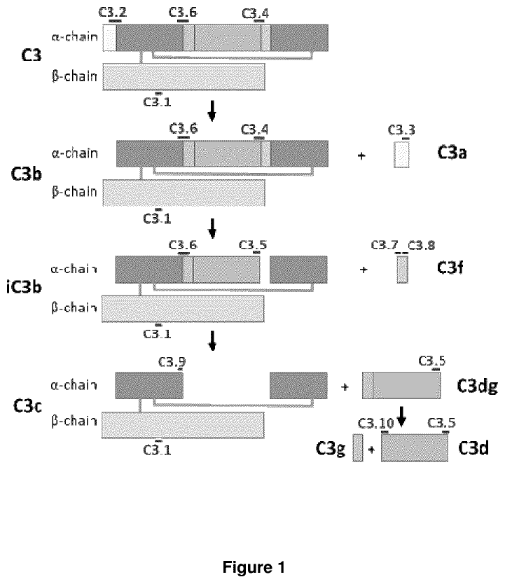

The series of proteolytic events leading to the generation, breakdown and inactivation of C3 are shown in FIG. 1. Proteoform-specific peptides produced by GluC digestion are underlined in FIG. 1 and are shown in Table 2. Table 3 shows how each protein can be detected individually using the peptides in Table 2.

TABLE 2
Peptide sequences for MS resulting from
GluC digestion of C3, C3b and breakdown
products.

| | | | SEQ |
|---|---|---|---|
| | Peptide | Contained in | ID |
| Peptide | sequence | C3b products | No: |
| C3.1 | GTAFVIFGIQDGE | C3 + C3b + | 28 |
| | | iC3b + C3c | |
| C3.2 | LRRQHARASHLG | C3 only | 29 |
| | LARSNLDE | | |
| C3.3 | LRRQHARASHL | C3a only | 30 |
| | GLAR | | |
| C3.4 | LNLDVSLQLPSR | C3 + C3b | 31 |
| | SSKITHRIHWE | | |
| C3.5 | LNLDVSLQLPSR | iC3b + C3dg + | 32 |
| | | C3d | |
| C3.6 | RLGRE | C3 + C3b + | 33 |
| | | iC3b | |
| C3.7 | SSKITHRIHWE | C3f | 34 |
| C3.8 | SASLLR | C3f | 35 |
| C3.9 | RLGR | C3c | 36 |
| C3.10 | HLIVTPSGCGE | C3d | 37 |

TABLE 3
Methodology for determining concentration of all C3/C3b breakdown
products using GluC digestion peptides of Table 2.

| Protein or fragment | Peptide(s) |
|---|---|
| C3 (total) | C3.1 |
| C3 only | C3.2 |
| C3a | C3.3 |
| C3b | C3.4-C3.2 |
| iC3b | C3.6-C3.4 |
| C3f | C3.7 or C3.8 |
| C3c | C3.9 |
| C3dg | C3.5-C3.10-iC3b [C3.6-C3.4] |
| C3d | C3.10 |

TABLE 4
Alternative peptides for C3.1 resulting from
GluC digestion, to measure total C3 content.

| | | | | SED |
|---|---|---|---|---|
| Peptide | Mass | Position | Peptide sequence | ID No. |
| C3.1.1 | 6957.8210 | 463-523 | AKIRYYTYLIMNKGRLLKAGRQVREPGQDLV | 61 |
| | | | VLPLSITTDFIPSFRLVAYYTLIGASGQRE | |
| C3.1.2 | 5778.9734 | 321-372 | RSGIPIVTSPYQIHFTKTPKYFKPGMPFDL | 62 |
| | | | MVFVTNPDGSPAYRVPVAVQGE | |
| C3.1.3 | 5650.1364 | 97-146 | KVVLVSLQSGYLFIQTDKTIYTPGSTVLYRI | 63 |
| | | | FTVNHKLLPVGRTVMVNIE | |
| C3.1.4 | 3904.1017 | 373-408 | DTVQSLTQGDGVAKLSINTHPSQKPLSITVR | 64 |
| | | | TKKQE | |
| C3.1.5 | 3861.1628 | 565-599 | GDHGARVVLVAVDKGVFVLNKKNKLTQSKI | 65 |
| | | | WDVVE | |
| C3.1.6 | 3807.7645 | 600-637 | KADIGCTPGSGKDYAGVFSDAGLTFTSSSG | 66 |
| | | | QQTAQRAE | |
| C3.1.7 | 3263.6357 | 414-442 | QATRTMQALPYSTVGNSNNYLHLSVLRTE | 67 |
| C3.1.8 | 2845.4609 | 24-50 | AHDAQGDVPVTVTVHDFPGKKLVLSSE | 68 |
| C3.1.9 | 2819.4705 | 150-175 | GIPVKQDSLSSQNQLGVLPLSWDIPE | 69 |
| C3.1.10 | 2691.3425 | 524-549 | VVADSVWVDVKDSCVGSLVVKSGQSE | 70 |
| C3.1.11 | 2618.3261 | 296-320 | DLVGKSLYVSATVILHSGSDMVQAE | 71 |
| C3.1.12 | 2511.2904 | 51-73 | KTVLTPATNHMGNVTFTIPANRE | 72 |
| C3.1.13 | 2108.1378 | 78-96 | KGRNKFVTVQATFGTQVVE | 73 |
| C3.1.14 | 1992.1480 | 278-295 | VVLSRKVLLDGVQNPRAE | 74 |
| C3.1.15 | 1827.9414 | 448-462 | TLNVNFLLRMDRAHE | 75 |
| C3.1.16 | 1769.8923 | 176-189 | LVNMGQWKIRAYYE | 76 |
| C3.1.17 | 1745.9386 | 1-15 | SPMYSIITPNILRLE | 77 |
| C3.1.18 | 1738.9036 | 550-564 | DRQPVPGQQMTLKIE | 78 |
| C3.1.19 | 1623.9348 | 231-244 | VTITARFLYGKKVE | 79 |
| C3.1.20 | 1352.6612 | 245-257 | GTAFVIFGIQDGE | 80 |
| C3.1.21 | 1138.5335 | 219-226 | KFYYIYNE | 81 |
| C3.1.22 | 1135.5146 | 190-199 | NSPQQVFSTE | 82 |
| C3.1.23 | 954.5862 | 265-272 | SLKRIPIE | 83 |
| C3.1.24 | 853.4221 | 205-211 | YVLPSFE | 84 |
| C3.1.25 | 841.4657 | 258-264 | QRISLPE | 85 |
| C3.1.26 | 826.4007 | 638-645 | LQCPQPAA | 86 |
| C3.1.27 | 785.4171 | 212-218 | VIVEPTE | 87 |
| C3.1.28 | 591.2938 | 19-23 | TMVLE | 88 |
| C3.1.29 | 570.3125 | 443-447 | LRPGE | 89 |
| C3.1.30 | 509.2485 | 74-77 | FKSE | 90 |

GluC digestion of Factor I (FI) produced the candidate peptides in Table 5 for MS analysis. SEQ ID NO:45 to 56 and 155 contain 8-21 amino acids and are a good length for MS analysis.

TABLE 5
Peptide sequences resulting from GluC digestion of FI.

| | | | | SEQ |
|---|---|---|---|---|
| Peptide | Mass | Position | Peptide sequence | ID NO: |
| 1 | 3996.7183 | 510-548 | CAGTYDGSIDACKGDSGGPL | 38 |
| | | | VCMDANNVTYVWGVVSWGE | |
| 2 | 3994.8853 | 57-92 | GTCVCKLPYQCPKNGTAVCA | 39 |
| | | | TNRRSFPTYCQQKSLE | |
| 3 | 3467.7211 | 555-583 | FPGVYTKVANYFDWISYHVG | 40 |
| | | | RPFISQYNV | |
| 4 | 3397.6804 | 150-180 | ANVACLDLGFQQGADTQRRF | 41 |
| | | | KLSDLSINSTE | |
| 5 | 3388.6605 | 446-475 | LPRSIPACVPWSPYLFQPND | 42 |
| | | | TCIVSGWGRE | |
| 6 | 3155.5773 | 31-56 | KKCLAKKYTHLSCDKVFCQP | 43 |
| | | | WQRCIE | |
| 7 | 2991.2861 | 254-281 | LCCKACQGKGFHCKSGVCIP | 44 |
| | | | SQYQCNGE | |
| 8 | 2531.2455 | 129-149 | VKLVDQDKTMFICKSSWSMR | 45 |
| | | | E | |
| 9 | 2068.0320 | 489-505 | VKLISNCSKFYGNRFYE | 46 |
| 10 | 1805.8309 | 93-109 | CLHPGTKFLNNGTCTAE | 47 |
| 11 | 1698.7969 | 420-434 | NYNAGTYQNDIALIE | 48 |
| 12 | 1605.7867 | 110-124 | GKFSVSLKHGNTDSE | 49 |
| 13 | 1297.5729 | 291-303 | VGCAGFASVTQEE | 50 |
| 14 | 1168.272 | 291-302 | VGCAGFASVTQE | 155 |
| 15 | 1295.6082 | 435-445 | MKKDGNKKDCE | 51 |
| 16 | 1191.6156 | 411-419 | YVDRIIFHE | 52 |
| 17 | 1166.5557 | 181-190 | CLHVHCRGLE | 53 |
| 18 | 1121.5738 | 480-488 | RVFSLQWGE | 54 |
| 19 | 978.4448 | 306-314 | ILTADMDAE | 55 |
| 20 | 955.4731 | 19-26 | KVTYTSQE | 56 |
| 21 | 736.3182 | 282-288 | VDCITGE | 57 |
| 22 | 647.2817 | 549-554 | NCGKPE | 58 |
| 23 | 520.2613 | 191-195 | TSLAE | 59 |
| 24 | 505.2252 | 476-479 | KDNE | 60 |

### Example 2: Mass Spectrometry

### 2.1 Preparation of Stable Isotopic Standards (SIS) Spiking Solution

High purity heavy-labelled synthetic standards, with S-carboxymethylated (CAM) cysteine residues, were obtained (Cambridge Research Biochemicals, Cambridge, UK) and diluted to 1 μg/μL with 50:50 acetonitrile:water+0.1% formic acid (Table 6).

A mixed SIS solution was prepared by firstly diluting stock solution of FHL-1, FHR1, FHR2, FHR3, FHR4 and FHR5 by tenfold (no dilution of CFH stock was required), then adding the appropriate amounts of each individual diluted solution to a final volume of 200 μL in 0.1% TFA. This was then stored at −80° C. in 5 μL aliquots for further dilution immediately prior to spiking.

Spiking solution was prepared immediately prior to sample addition by adding 195 μL 50:50 acetonitrile:water to a 5 μL aliquot of the mixed SIS solution. 2 μL of this was carefully added to each digested sample prior to drying down.

TABLE 6
Stock solutions of stable isotopic standards
(SIS) at 1 μg/MI. The residues in bold were
chosen to carry a stable-heavy isotype to
enable quantitation. Lower case ‘c’ denotes
a S-carboxymethylated (CAM) cysteine residue.
Residue in bold type contained an
isotopically heavy amino acid,
where K(+8), R(+10), F(+10) and Y(+10).

| | | | | Net | Solvent | Conc, |
|---|---|---|---|---|---|---|
| Pro- | Peptide | MW, | Purity, | Content, | Volume, | pmol/ |
| tein | Sequence | g | % | % | μl | μL |
| CFH | VTYK | 953.4 | 98.6 | 72.6 | 716 | 1050 |
| | CFE | | | | | |
| FHL-1 | NGWS | 1900 | 97.4 | 78.9 | 768 | 526.3 |
| | PTPR | | | | | |
| | cIRV | | | | | |
| | SFTL | | | | | |
| FHR1 | ATFcD | 2178 | 100.0 | 80.7 | 807 | 459.1 |
| | FPKIN | | | | | |
| | HGILY | | | | | |
| | DEE | | | | | |
| FHR2 | RGWST | 1845.9 | 100.0 | 55.5 | 555 | 541.7 |
| | PPKcR | | | | | |
| | STISA | | | | | |
| | E | | | | | |
| FHR3 | VAcHPG | 2207.1 | 98.4 | 75.0 | 738 | 453.1 |
| | YGLPKA | | | | | |
| | QTTVTC | | | | | |
| | TE | | | | | |
| FHR4 | YQcQSY | 1149.7 | 97.6 | 84.1 | 821 | 869.8 |
| | YE | | | | | |
| FHR5 | RGWSTP | 1739.8 | 95.6 | 74.0 | 707 | 574.8 |
| | PICSFT | | | | | |
| | KGE | | | | | |

### 2.2 Preparation of Samples for Analysis by LC-MS/MS

Frozen plasma samples were allowed to thaw to room temperature before being vortexed hard for 5 minutes to dissolve any soluble material, then centrifuged at 13,300g for 30 min to settle any insoluble material.

To a 5 μL plasma aliquot (equivalent to approximately 350 μL protein), 90 μL of 50 mM ammonium bicarbonate (pH 7.8), 2 μL of ProteaseMAX™ (Promega, Southampton, UK) solution (1% w/v in 50 mM ammonium bicarbonate) and 1 μL of 500 mM dithiothreitol prepared in 50 mM ammonium bicarbonate was added. This was vortexed briefly to mix, then given a pulse spin before incubating at 56° C. for 25 min.

After cooling to room temperature, 3 μL 500 mM iodoacetamide (prepared in 50 mM ammonium bicarbonate) was added. This was vortexed briefly to mix, then given a pulse spin before incubating at room temperature and in the dark for 15 min.

A further 1 μL of ProteaseMAX solution (1% w/v in 50 mM ammonium bicarbonate) and 5 μL of 1 μg/uL endoproteinase GluC (Roche, Mannheim, Germany) were added. The mixture was vortexed briefly, then given a pulse spin before incubating for 16 hours at 25° C. with slight shaking (400 rpm).

To the digested peptide mix obtained, 6 μL 10% v/v trifluoroacetic acid (TFA) and 2 μL of SIS spiking solution were added, vortexed briefly to mix, then pulse spin. The solution was placed into an evaporator and dried. Finally the peptides were reconstituted in 50 μL 0.1% TFA and vortexed to dissolve any residue before centrifuging at 13,300g for 30 min to settle any insoluble/particulate material. Approximately 48 μL (taking care to leave behind any precipitated material) was transferred to a LC autosampler vial for subsequent analysis by LC-MS/MS.

### 2.3 LC-SRM/MS Analysis of Plasma Digests

SRM analyses of plasma digests were performed on a 6495 triple quadrupole mass spectrometer with Funnel-equipped electrospray ion source (Agilent, Santa Clara, CA, USA) coupled to an Infinity 1200 Series liquid chromatography system consisting of 1290 autosampler, 1260 Quat Pump VL pump and TCC column oven modules (Agilent, Santa Clara, CA, USA). Samples were injected directly (4 μL) onto a C18 column (250 mm×2.1 mm i.d., Thermo Scientific Acclaim 120, 3 μm particle size) that was maintained at a column temperature of 50° C. Compounds were developed using a gradient elution of increasing acetonitrile concentration with Buffer A consisting of Water+0.1% formic acid and Buffer B being Acetonitrile+0.1% formic acid. The flow rate was maintained at 250 μL/min with an initial composition of 5% Buffer B.

The following gradient elution profile was used to separate the peptides (time: % B): 0 min: 5% B; 2 min: 5% B; 3 min: 12% B; 12 min: 15% B; 15 min: 20% B; 30 min: 25% B; 31 min: 90% B; 39 min:90% B; 40 min: 5% B; 49 min: 5% B.

Optimized SRM settings were determined using SIS solutions and are provided in Table 7.

TABLE 7
SRM transitions and optimal collision energies for
FH family peptides (Quantitation ions in bold).

| | | Precursor | Product | Collision |
|---|---|---|---|---|
| Pro- | | ion | ions | energy, |
| tein | Peptide Sequence | m/z | m/z | eV |
| CFH | VTYKcFE (Light) | 473.7 | 583.3, 847.4, | 16, 16, 16 |
| | | | 746.3 | |
| CFH | VTYKcFE (Heavy) | 477.7 | 591.3, 855.4, | 16, 16, 16 |
| | | | 754.3 | |
| FHL-1 | NGWSPTPRcIRVSFTL | 631.2 | 723.9, 860.5, | 19, 19, 19 |
| | (Light) | | 767.4 | |
| FHL-1 | NGWSPTPRcIRVSFTL | 634.3 | 728.9, 865.5, | 19, 19, 19 |
| | (Heavy) | | 772.4 | |
| FHR1 | ATFcDFPKINHGILYDEE | 724.2 | 925.6, 1011.9, | 20, 16, 20 |
| | (Light) | | 947.1 | |
| FHR1 | ATFcDFPKINHGILYDEE | 727.2 | 930.6, 1016.9, | 20, 16, 20 |
| | (Heavy) | | 952.1 | |
| FHR2 | RGWSTPPKcRSTISAE | 459.1, 611.8 | 539.2, 798.9 | 8, 26 |
| | (Light) | | | |
| FHR2 | RGWSTPPKcRSTISAE | 462.6, 616.3 | 543.9, 806.0 | 8, 26 |
| | (Heavy) | | | |
| FHR3 | VAcHPGYGLPKAQTTVTCTE | 730.7 | 1022.4, 971.7 | 16, 18 |
| | (Light) | | | |
| FHR3 | VAcHPGYGLPKAQTTVTCTE | 736.7 | 1031.4, 980.7 | 16, 18 |
| | (Heavy) | | | |
| FHR4 | YQcQSYYE (Light) | 570.7 | 830.3, 993.1, | 11, 10, 14 |
| | | | 311.1 | |
| FHR4 | YQcQSYYE (Heavy) | 575.7 | 840.3, 1003.1, | 11, 10, 14 |
| | | | 311.1 | |
| FHR5 | RGWSTPPICSFTKGE | 575.2 | 828.4, 895.5, | 16, 15, 20 |
| | (Light) | | 588.3 | |
| FHR5 | RGWSTPPICSFTKGE | 581.2 | 836.4, 905.5, | 16, 15, 20 |
| | (Heavy) | | 598.3 | |

TABLE 8
Peptides and transitions for quantitation
of C3/C3b breakdown products.

| Pep- | Se- | Contained | Transitions |
|---|---|---|---|
| tide | quence | in . . . | |
| C3.1 | GTAFVIF | C3 + C3b + | 569.0/483.2 |
| | GIQDGE | iC3b + C3c | 569.0/654.3 |
| | | | 569.0/801.4 |
| C3.2 | LRRQHAR | C3 only | 384.2/431.6 |
| | ASHLGL | | 384.2/510.4 |
| | ARSNLDE | | 461.0/503.3 |
| C3.3 | LRRQHAR | C3a only | 291.2/304.3 |
| | ASHLG | | 291.2/395.8 |
| | LAR | | 436.3/598.7 |
| C3.4 | LNLDVSL | C3 + C3b | 546.8/578.7 |
| | QLPSRSS | | 683.2/797.1 |
| | KITHRIH | | 683.2/834.9 |
| | WE | | |
| C3.5 | LNLDVS | iC3b + C3dg + | 677.9/1014.4 |
| | LQLPSR | C3d | 677.9/359.1 |
| | | | 677.9/800.2 |
| C3.6 | RLGRE | C3 + C3b + | 251.3/175.1 |
| | | iC3b | 251.3/232.1 |
| | | | 251.3/345.2 |
| C3.7 | SSKITH | C3f | 349.3/354.2 |
| | RIHWE | | 349.3/436.4 |
| | | | 349.3/489.8 |
| C3.8 | SASLLR | C3f | 323.7/159.1 |
| | | | 323.7/288.2 |
| | | | 323.7/488.3 |
| C3.9 | RLGR | C3c | 251.3/175.1 |
| | | | 251.3/232.1 |
| | | | 251.3/345.2 |
| C3.10 | HLIVT | C3d | 585.3/251.1 |
| | PSGCG | | 585.3/394.5 |
| | E | | 585.3/404 |

In order to protect the source region from unwanted contaminants, a switching valve located between the column and source was diverted to the waste position at points in the chromatogram when the analyte peptides were not eluting. This allowed for six windows (two of the peptides, FHR-2 and FHL-1, eluted within the same window) of acquisition, of approximately one minute each, to be acquired with the column on-line to the mass spectrometer. SRM data was processed using a dedicated project in Skyline (v19.1.0.193; www.skyline.ms).

### 2.4 Results

### FH Family Proteins

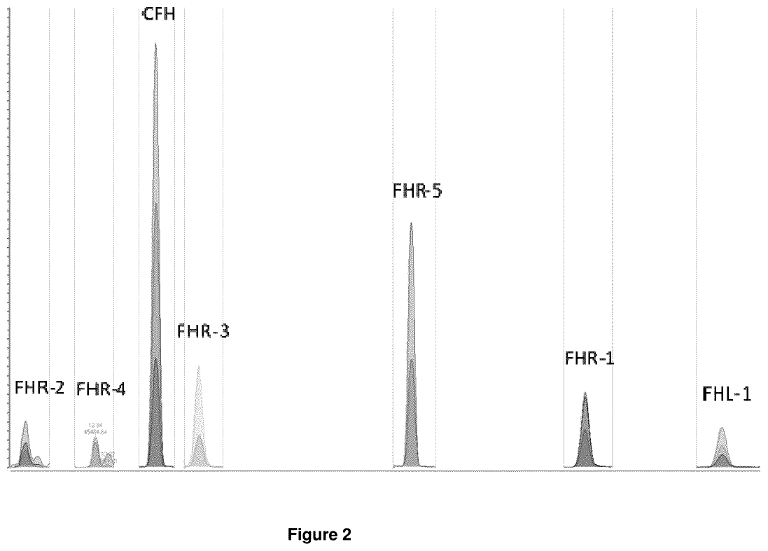

FIG. 2 shows a LC-SRM Trace showing detection of the heavy-labelled synthetic standards of each individual RCA locus protein from a plasma sample. This demonstrates that the method is feasible, specific and has the required sensitivity to distinguish between peptides from these seven proteins, in particular between splice variants FH and FHL-1.

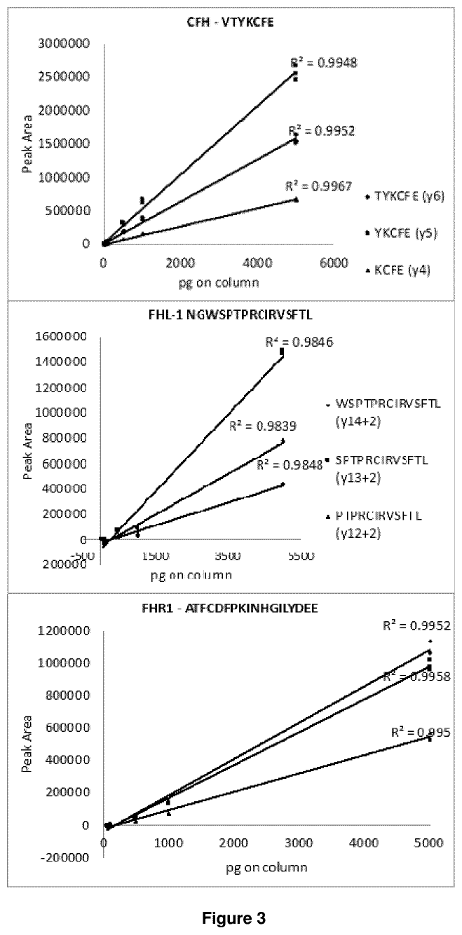

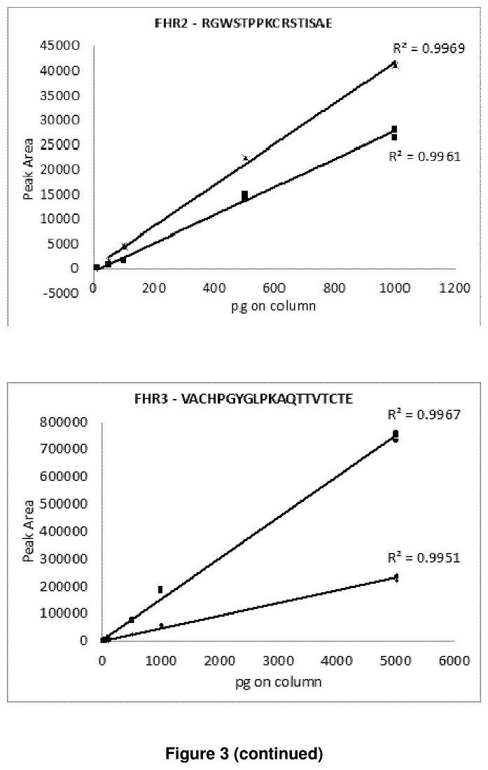

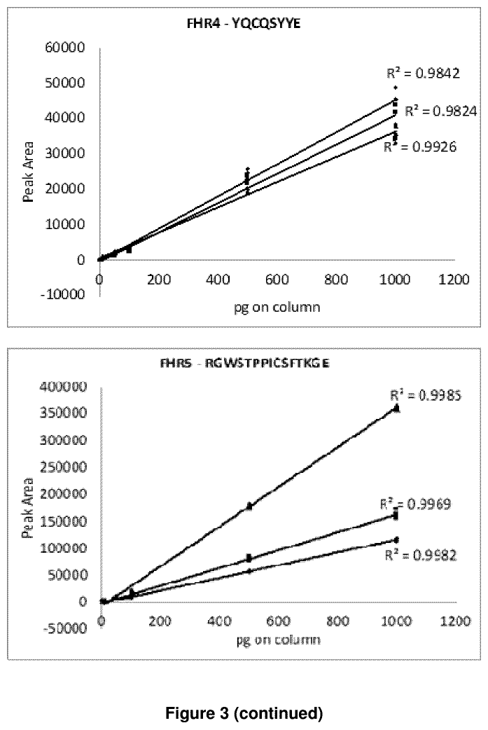

FIG. 3 shows linearity data for FH, FHL-1, and FHR1-5. This demonstrates that the GluC digestion produces peptides that can be detected individually and specifically in native serum at endogenous levels. It also shows that the assay is capable of quantifying the level of each protein in the sample. Increasing amounts of protein increase the signal in a predictable manner, allowing determination of the levels, as well as the presence, of each of the proteins. Also demonstrated is that the assay is free from interference.

Lower limits of quantitation were defined as plasma concentrations of FH=25 nM, FHL-1=0.25 nM, FHR-1=2 nM, FHR-2=1 nM, FHR-3=1 nM, FHR-4=4 nM and FHR-5=3 nM.

### C3 and C3 Breakdown Products

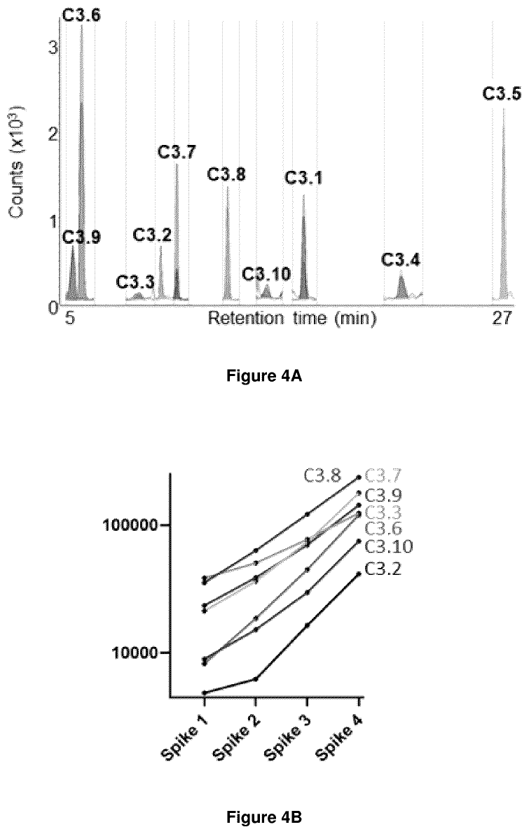

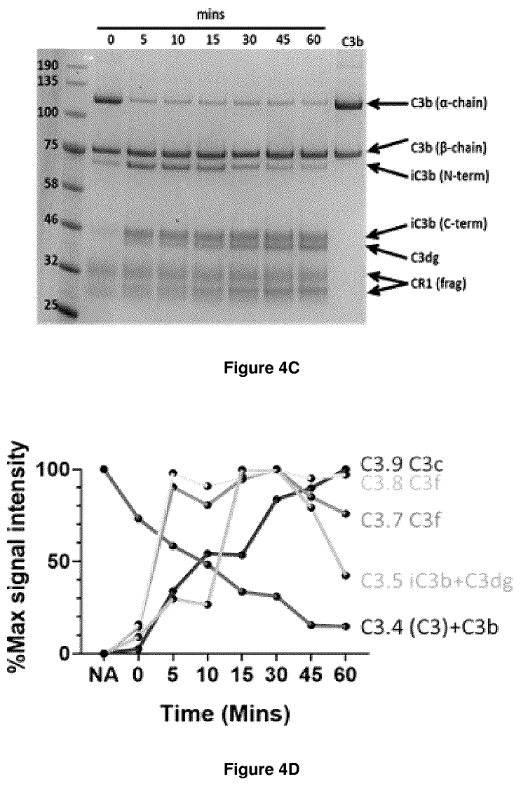

Synthetic versions of the peptides in Table 2 were synthesised to confirm and optimise their detection by MS to confirm that they could be quantified in a linear manner, and to demonstrate that they could be detected at endogenous levels in a serum or plasma sample. This is shown by FIGS. 4A to 4D.

FIG. 4A shows that all peptides in Table 2 can be detected individually in a plasma sample by SRM-MS using at least three transitions. The specificity of the assay for the peptides of interest is confirmed by the relative intensities of the transitions matching the relative intensity of the relevant product ions in an MS/MS scan. FIG. 4B confirms the specificity of the peptides, showing experiments in which the plasma sample was spiked with crude synthetic peptide which demonstrated the appropriate increase in signal.

C3b breakdown was further analysed in an in vitro assay. C3b was incubated along with FI and a fragment of cofactor CR1, selected over FH as CR1 drives the reaction to cleavage of iC3b to C3c+C3dg, whereas FH will only support cleavage of C3b to iC3b. Sequential samples were taken from the reaction and stopped by boiling.

FIG. 4C shows the time course of the C3b breakdown via gel electrophoresis. Analysis using MS and the peptides of Table 2 demonstrates that the formation of C3b fragments iC3b, C3f and C3c, and loss of intact C3b can be clearly detected over time (FIG. 4D). Not all peptides are shown since some (e.g. C3a) will not be present in the in vitro set-up, and others represent multiple products.

These data demonstrate that C3/C3b breakdown can be measured in a quantitative manner using GluC-derived peptides and MS. This enables the presence and levels of complement proteins to be detected in complement-related diseases such as AMD, as well as providing information as to successful treatment outcomes.

A single assay which can measure all FH family, C3 fragments and FI proteins allows for the simultaneous analysis of all key proteins in the complement amplification loop from just one sample and with efficient throughput.

### Example 3: Detection of the Complementome in AMD Patients

### 3.1 Sample Collection and Processing

Plasma samples were collected during the Cambridge AMD study; a case-control study with subjects recruited from the southeast and northwest of England between 2002 and 2006. The original study and its results are described in Yates et al., (2007). N. Engl. J. Med. 357, 553-561. All 246 affected subjects had advanced, end-stage AMD (choroidal neovascularization and/or geographic atrophy). 166 control subjects were spouses, partners or friends of index patients. Blood samples were obtained at the time of interview; EDTA and lithium-heparin plasma samples were used for the measurement of CFH, FHL-1 and the FHR proteins.

Analysis of plasma samples by Mass Spectrometry was performed as per Example 2 to determine levels of CFH, FHL-1 and FHR proteins. Samples were prepared for LC-MS/MS analysis by digestion and addition of SIS spiking solution as described in (2.1 and 2.2). Samples were then analysed by LC-SRM/MS as described in (2.3).

All statistical analyses were performed using GraphPad Prism (v8.4.3).

### 3.2 Results

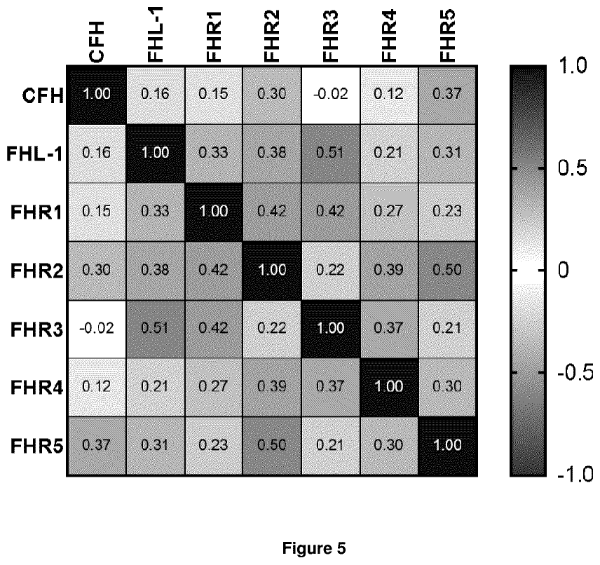

The data was analysed for evidence of correlation between the levels of the studied proteins. All Pearson correlation coefficients were found to be weaker than +/−0.55. It was therefore concluded that there is no strong linear relationship between different protein levels and little pair-wise correlation. Notably, inspection of the scatterplots did not reveal evidence of nonlinear relationship between variables. The relevant correlation matrix is shown in FIG. 5.

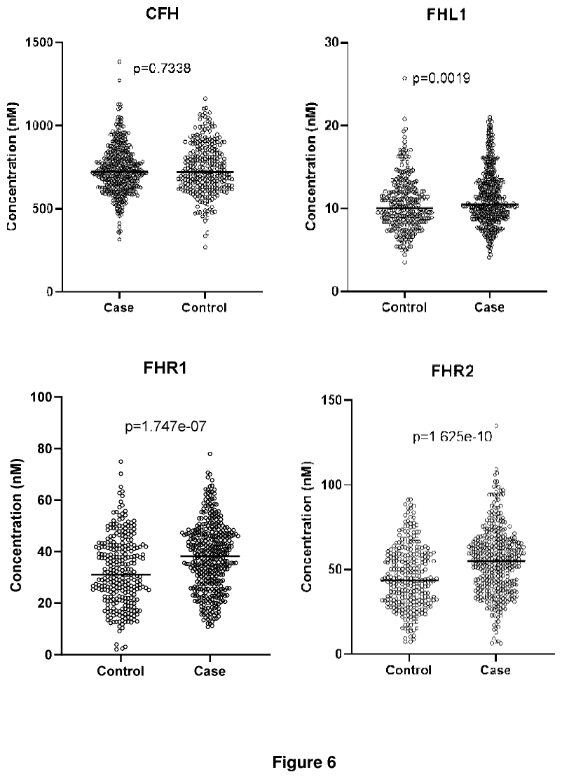

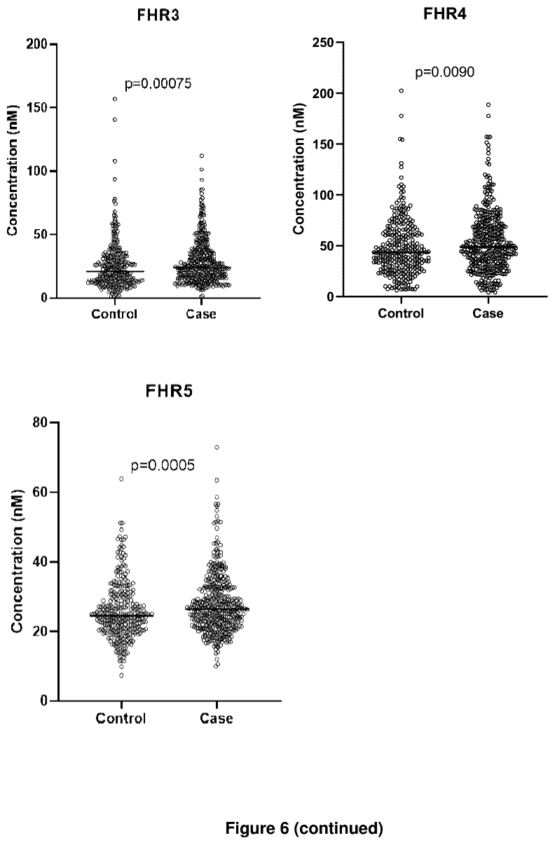

Protein levels were then compared between AMD cases (n=399; subjects having CNV, geographic atrophy or a mixed phenotype) and controls (n=298). Non-parametric tests (Mann-Whitney test) for the absolute protein levels were performed and statistically significant differences were detected between cases and controls for FHR1, FHR2, FHR3, FHR4, FHR5 and FHL-1 (FIG. 6); p<0.05. Thus, circulating FHL-1 and FHR-1 to FHR-5 levels are higher in people with advanced AMD.

The association of advanced AMD with each of the FH, FHL-1 and five FHR levels was assessed via Wald tests using linear regression models adjusted for sex, age and the first two genetic principal components (as estimated within the IAMDGC study). The association of levels with advanced AMD was also reported via odds ratio (OR) expressed as per one standard deviation (SD) change of log-levels using logistic regression models adjusted for sex, age and the first two genetic principal components. The results are displayed in Table 9.

TABLE 9
Demographics of study samples and association analyses between
AMD and circulating FH, FHL-1, FHR-1 to FHR-5 protein levels.

| | Controls | Cases | |
|---|---|---|---|
| N | 252 | 352 | |
| Age, ys (SD) | 75.2 (7.9) | 73.9 (8.3) | |
| Male (%) | 39.3 | 45.7 | |
| AMD phenotype | | | |
| CNV only | | 218 | |
| GA only | | 73 | |
| Mixed | | 61 | |
| | | | OR (95% CI)^{b} |
| Mean FH levels, nM (95% CI) | 737.3 (718.2-756.5) | 736.5 (721.3-751.6) | |
| Association with AMD, | 0.005, 0.23, 0.982 | | 1.01 (0.86-1.20) |
| Beta, SE, P^{a} | (0.02, 0.23, 0.936) | | |
| Mean FHL-1 levels, nM (95% CI) | 10.4 (10.1-10.8) | 11.3 (11.0-11.7) | |
| Association with AMD, | 0.08, 0.02, 1.4 × 10⁻³ | | 1.35 (1.14-1.60) |
| Beta, SE, P^{a} | (0.08, 0.02, 4.9 × 10⁻⁴) | | |
| Mean FHR-1 levels, nM (95% CI) | 31.2 (29.4-32.9) | 38.4 (37.0-39.8) | |
| Association with AMD, | 7.22, 1.12, 2.1 × 10⁻¹⁰ | | 1.81 (1.47-2.24) |
| Beta, SE, P^{a} | (7.21, 1.12, 2.4 × 10⁻¹⁰) | | |
| Mean FHR-2 levels, nM (95% CI) | 45.3 (43.1-47.6) | 55.3 (53.2-57.4) | |
| Association with AMD, | 0.71, 0.12. 1.9 × 10⁻⁹ | | 1.66 (1.38-1.98) |
| Beta, SE, P^{a} | (0.74, 0.12, 6.0 × 10⁻¹⁰) | | |
| Mean FHR-3 levels, nM (95% CI) | 24.1 (21.7-26.5) | 28.9 (27.1-30.8) | |
| Association with AMD, | 0.55, 0.13, 4.4 × 10⁻⁵ | | 1.54 (1.29-1.84) |
| Beta, SE, P^{a} | (0.59, 0.13, 1.4 × 10⁻⁵) | | |
| Mean FHR-4 levels, nM (95% CI) | 46.1 (42.7-49.6) | 53.8 (50.5-57.1) | |
| Association with AMD, | 0.53, 0.17, 2.1 × 10⁻³ | | 1.27 (1.08-1.50) |
| Beta, SE, P^{a} | (0.56, 0.17, 1.3 × 10⁻³) | | |
| Mean FHR-5 levels, nM (95% CI) | 25.5 (24.5-26.5) | 27.9 (27.0-28.9) | |

| | | |
|---|---|---|
| Association with AMD, | 0.09, 0.03, 1.9 × 10⁻⁴ | 1.38 (1.16-1.63) |
| Beta, SE, P^{a} | (0.10, 0.03, 1.9 × 10⁻⁴) | |

^{a}Wald tests using linear regression models; adjusted P-values for sex, age and first two genetic principal components as estimated in Fritsche et al.² are displayed in parentheses;
^{b}Odds ratio (OR) of advanced disease expressed as per standard deviation change of log-levels using logistic regression models adjusted for sex, age and the first two genetic principal components. AMD = age-related macular degeneration; CNV = choroidal neovascularization; GA = geographic atrophy; SE = standard error; CI = confidence interval.

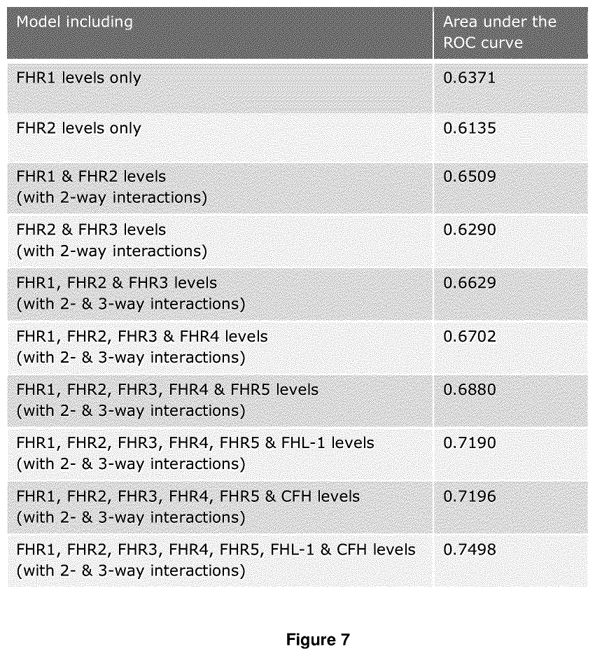

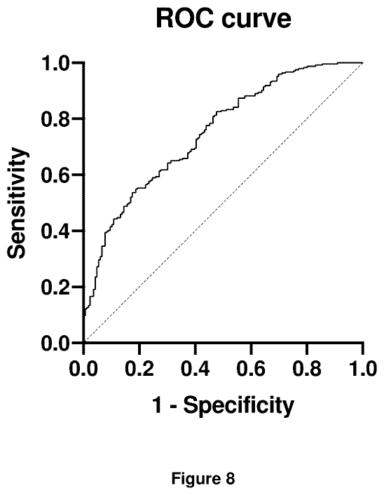

The data were examined to determine to what extent the clinical outcome (AMD or no AMD) could be predicted based on the different protein level values. Multiple logistic regression analysis was used for this purpose and the findings for different models are shown in FIG. 7. It was found that a model including all studied proteins (FHR1, FHR2, FHR3, FHR4, FHR5, FHL-1 & CFH protein levels) had the highest discrimination ability (AUROC (area under ROC curve) of 0.7498; FIG. 8), although all of the models tested were capable of discriminating between subjects with AMD and control subjects.

### Genotype Data and Genome-Wide Association Analysis

Genome-wide association analyses were performed of the protein levels that were found to be elevated in advanced AMD cases (i.e., FHL-1 and FHR-1 to FHR-5).

All individuals included in the study had been previously genotyped with a custom-modified Illumina HumanCoreExome array at the Centre for Inherited Disease Research (CIDR, Baltimore, Maryland, USA) and analysed within the International AMD Genomics Consortium (IAMDGC) GWAS (43,566 subjects; 16,144 advanced AMD cases and 17,832 controls of European ancestry in the primary analysis dataset). Quality control and genotype imputation using the 1000 Genomes Project (Abecasis et al., Nature 2012, 491, 56-65) reference panel were performed by the IAMDGC as described in Fritsche et al., Nature Genetics, 2016, 48, 134-143.

GWASs were performed for FH, FHL-1 and the five FHR levels (transformed to ensure normality) in controls only, using linear regression models adjusted for sex, age and the first two genetic principal components, and variants with Minor Allele Frequency, MAF≥1%. The GWASs were carried out using the EPACTS software (http://genome.sph.umich.edu/wiki/EPACTS, version 3.3.2) and Wald tests were performed on the variant genotypes coded as 0, 1 and 2 according to the number of minor alleles for the directly typed variants or allele dosages for the imputed variants. Manhattan and Q-Q plots were generated using the qqman R package (version 0.1.4). Regional plots of association were generated using LocusZoom.org. Finally, linkage disequilibrium (LD) measures (R² and D′) were calculated using LDlink (https://Ildlink.nci.nih.gov/), based on the European (EUR) population genotype data originated from the Phase 3 (Version 5) of the 1000 Genomes Project.

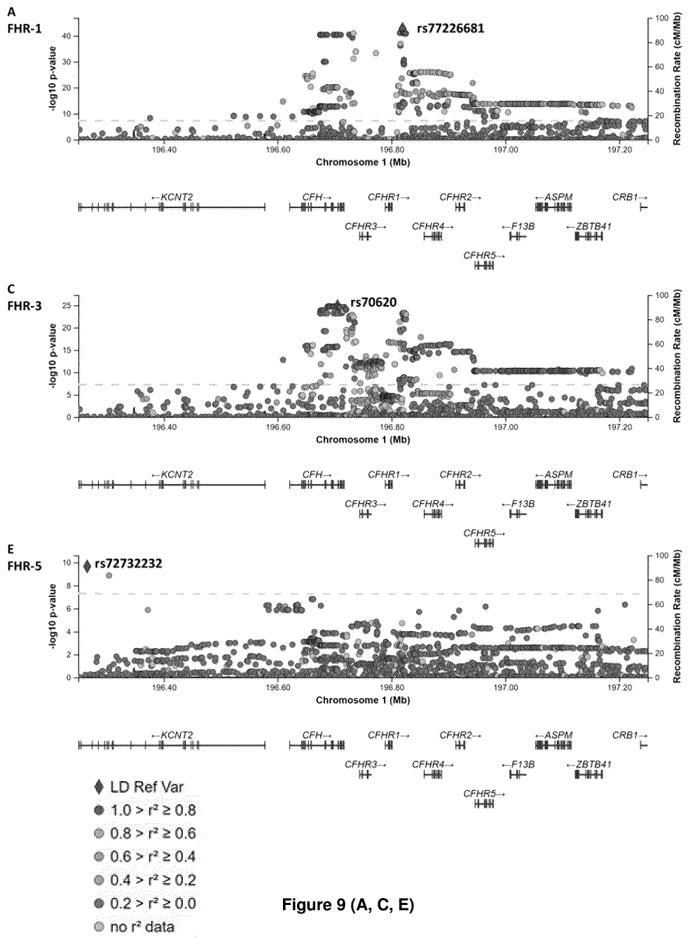

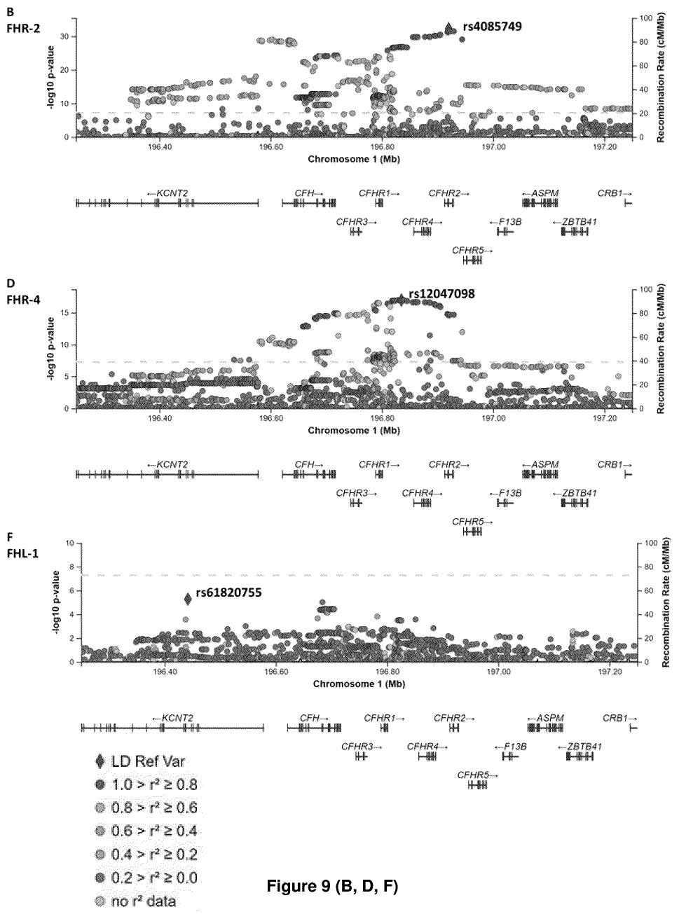

Remarkably, all GWASs of the five FHR protein levels in 252 controls showed a genome-wide significant (P≤5×10⁻⁸) peak at the CFH locus, see FIG. 9. For FHR-1, FHR-2, FHR4 and FHR-5, the CFH locus was the only genome-wide significant peak observed.

FHR-3 showed a more polygenic profile, with genome-wide significant signals at rs113721756 on chromosome 10 (P-value=1.7×10⁻⁸), rs111260777 on chromosome 11 (P-value=1.5×10⁻⁹), rs117468955 on chromosome 12 (P-value=3.0×10⁻⁸), rs4790395 on chromosome 17 (P-value=3.6×10⁻⁸) and rs117115124 on chromosome 19 (P-value=2.5×10⁻⁸) in addition to the CFH locus. The strongest signal from the GWAS of FHL-1 levels was observed at rs200404865 on chromosome 13 (P-value=9.6×10⁻⁷), with the strongest signal at the CFH locus observed at intronic KCNT2 rs61820755 (P-value=5.3×10⁻⁶).

The CFH locus genome-wide significant regions from the analyses of FHR-1 to FHR-5 levels overlapped among the different levels, but showed nominally different top signals (i.e., intergenic CFHR1/CFHR4 rs149369377 for FHR-1 with P-value=2.6×10⁻⁴³ and β=−18.2, synonymous CFHR2 rs4085749 for FHR-2 with P-value=6.3×10⁻³³ and β=−1.5, intronic CFH rs70620 for FHR-3 with P-value=1.5×10⁻²⁵ and β=2.0, intergenic CFHR1-CFHR4 rs12047098 for FHR-4 with P-value=1.1×10⁻¹⁷ and β=−1.7, and intronic KCNT2 rs72732232 for FHR-5 with P-value=2.2×10⁻¹⁰ and β=−0.5). These top signals are not in high LD with each other, except for rs4085749 of FHR-2 and rs12047098 of FHR-4 (R²=0.83, D′=0.95).

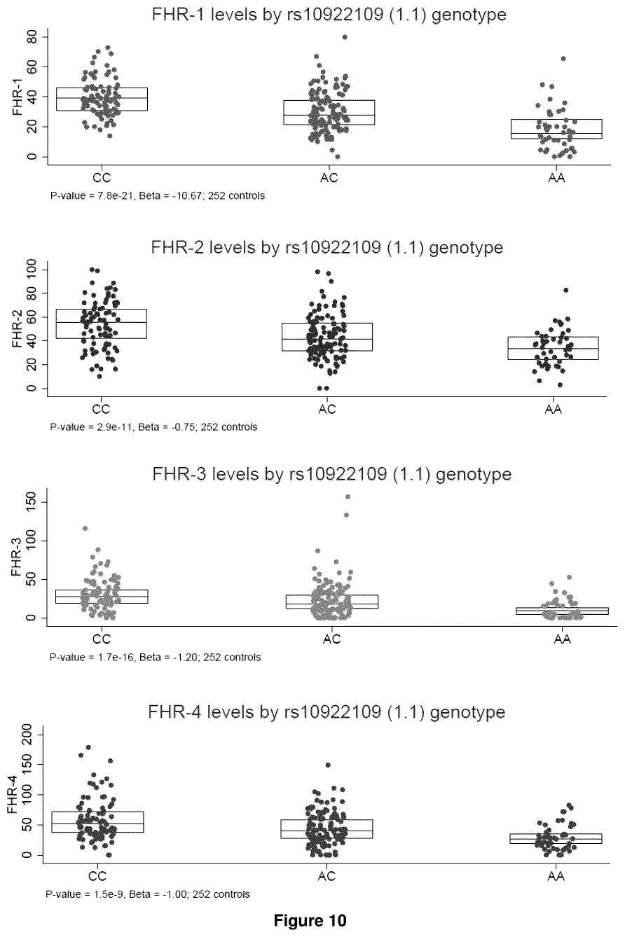

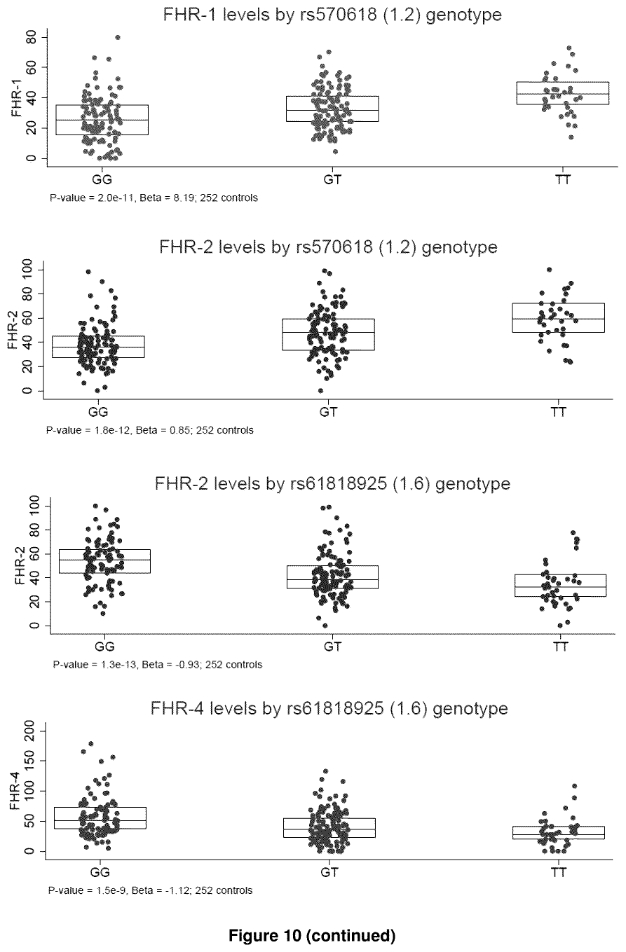

Next, it was assessed whether the GWAS top signals of FHR-1 to FHR-5 protein levels were in LD with any of the independently AMD-associated variants at the CFH locus reported by the IAMDGC GWAS, which also included the Cambridge samples analysed in this study (i.e., intronic CFH rs10922109 [1.1]; intronic CFH rs570618 [1.2], proxy for Y402H; CFH R1210C, rs121913059 [1.3]; intergenic rs148553336 [1.4], 8kb upstream CFH/35 kb downstream KCNT2; intronic KCNT2 rs187328863 [1.5]; intergenic rs61818925 [1.6], 14 kb downstream CFHR1/156 kb upstream CFHR4; intronic CFH rs35292876 [1.7]; intronic CFHR5 rs191281603 [1.8]; see Table 10 and FIG. 10). The rare CFH variant R1210C, rs121913059 [1.3] was present heterozygously in a single Cambridge case and excluded from this analysis.

The top signal for FHR-1 was in modest LD with the top AMD-associated variant 1.1 (R²=0.30) and low LD with the proxy for Y402H 1.2 (R²=0.12); the top signal for FHR-2 was in modest LD with 1.1 (R²=0.35) and 1.6 (R²=0.36), and low LD with 1.2 (R²=0.16); similarly for the top signal of FHR-4 (R² equal to 0.38, 0.42 and 0.16 with 1.1, 1.6 and 1.2, respectively); low LD was seen with 1.1, 1.2 and 1.6 (R² equal to 0.16, 0.12 and 0.11, respectively) for the top signal of FHR-3, while the top signal of FHR-5 was in low/modest LD with 1.4 (R²=0.26).

Furthermore, genome-wide significant associations were observed at the top IAMDGC variant rs10922109 (1.1) with P-values 8.6×10⁻²¹, 2.9×10⁻¹⁰, 2.2×10⁻¹⁶ and 1.7×10⁻⁹ for FHR-1, FHR-2, FHR-3 and FHR-4, respectively, at the proxy for Y402H 1.2 with P-values 2.0×10⁻¹¹ and 1.8×10⁻¹² for FHR-1 and FHR-2, respectively, and at the variant 1.6 with P-values 1.8×10⁻¹¹ and 2.4×10⁻⁹ for FHR-2 and FHR-4, respectively. All these genetic associations showed direction of allelic effect on levels concordant with that on disease as estimated in the IAMDGC GWAS study (Table 10, FIG. 10). Altogether, these GWAS findings support that the CFH locus AMD-risk variants increase disease risk through increase of FHR protein levels.

### Mendelian Randomization Estimates of the Effects of Circulating Levels of Complement Regulatory Proteins on Susceptibility to AMD

A Mendelian randomization approach was used to test if genetically proxied FHR protein levels are associated with risk of AMD.

Independent (LD, R2<0.001) genetic variants associated with the exposure were selected as instrumental variables (a protein at a time) at genome-wide significance level in controls only. If a single instrument was available, the ratio of coefficients method was used, also known as the Wald method, to estimate the effect of genetically proxied protein levels on the disease risk. The Wald ratio for a single genetic variant as instrumental variable is defined as its genetic association with the outcome (i.e. risk of AMD) over the genetic association with the exposure (i.e. protein level). Using a one-sample approach, the genetic association was derived with the exposure from the GWASs based on linear regression models for the FHR protein levels conducted on the Cambridge controls only (n=252). The genetic associations with the outcome were obtained from the GWAS based on a logistic regression model with AMD status as outcome conducted on the Cambridge samples (252 controls and 353 cases). If multiple instruments were available for a protein, the inverse-variance weighted (IVW) method was used under a fixed-effect model. Instrument strength was evaluated using R2 as the proportion of the variance of the protein explained by the genetic variant(s). The Mendelian randomization analysis was performed using the MendelianRandomization (version 0.4.2) and TwoSampleMR (version 0.5.5) R packages.

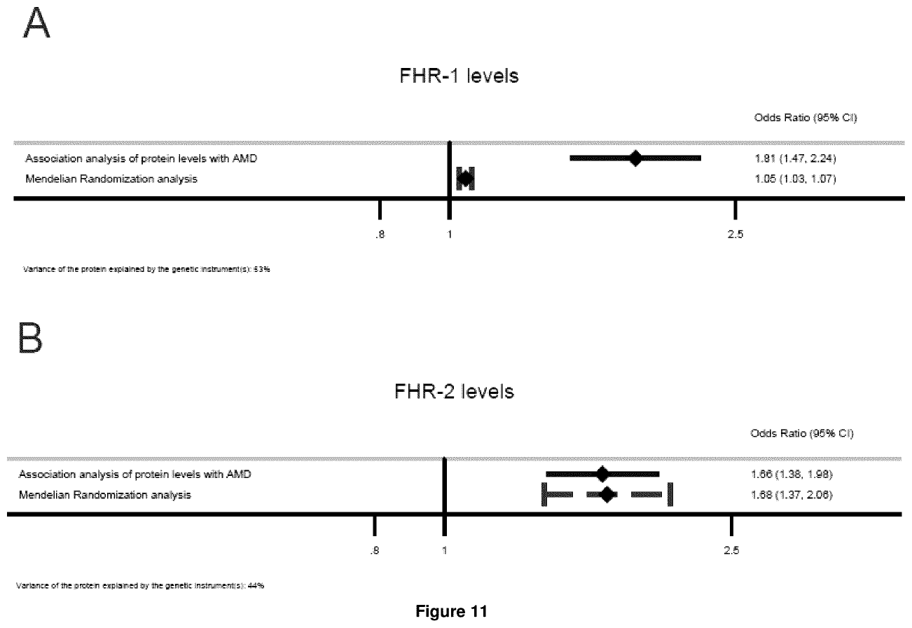

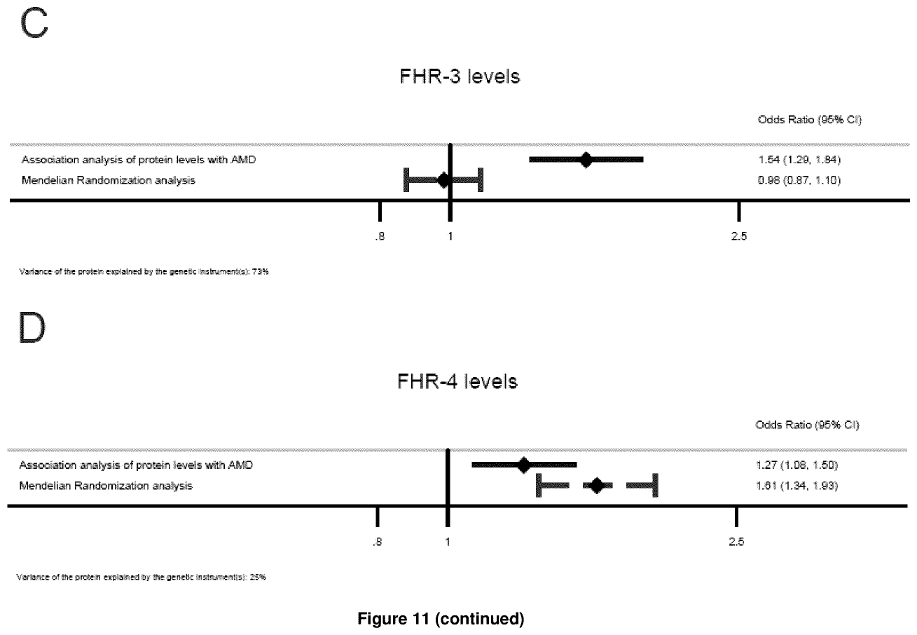

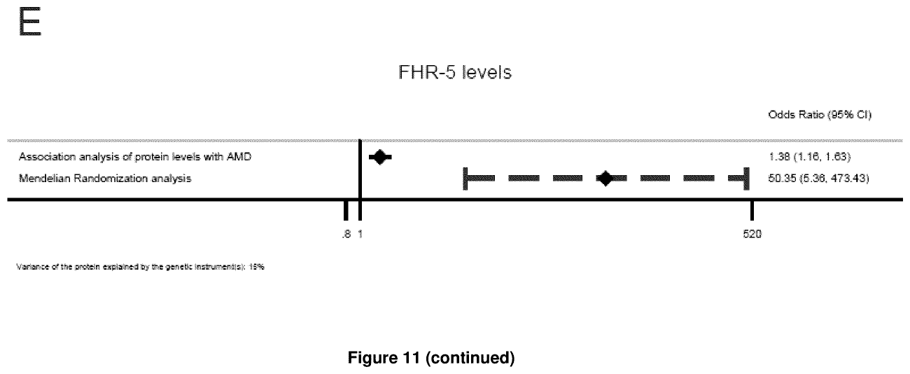

FIG. 11 shows the Mendelian randomization estimates of the FHR protein levels obtained using the (one-sample) Wald ratio (if a single instrument was available; FHR1, FHR2, FHR4, FHR5) or the IVW method (if multiple instruments were available; FHR3) together with the traditional epidemiologic estimates of the association of the levels with AMD obtained from logistic regression models and ORs (Table 9).

The variance of the FHR protein levels explained by the corresponding genetic instrument(s) varied from 15% for FHR5 to 73% for FHR3. The Mendelian randomization estimates were statistically significant and of concordant direction with the observational OR estimates for FHR-1, FHR-2, FHR-4 and FHR-5, providing evidence in support of a causal effect (FIG. 11). For FHR3, the Mendelian randomization estimate did not support an association of the protein levels with the disease (0.98, 95% Cl 0.87-1.10). The GWAS of FHL-1 levels did not show any genome-wide significant signals to use as genetic instruments in the Mendelian randomization analysis. It is worth noticing that the strongest FHL-1 GWAS signal at the CFH locus was observed at rs61820755 (P-value=5.3×10⁻⁶, β=0.22) and that this variant did not show association with AMD in the Cambridge samples (P-value=0.32; β=0.22).

As such, while these data strongly support a causal role of increased FHR-1, FHR-2, FHR4 and FHR-5, the consequences of the observed increase in FHL-1 and FHR-3 circulating levels in individuals with AMD remain less clear.

### Conclusions

Using 252 non-AMD controls to get insights into the genetic determinants of the circulating protein levels measured in this study, it was discovered that variants at the CFH locus regulate all five FHR protein levels (with genome-wide significant associations in our analyses of the FHR protein levels overlapping with the AMD-associated CFH region). Notably, established genetic associations with AMD risk at the non-coding variants 1.1, proxy for Y402H 1.2 and 1.6 translated into genetic associations at genome-wide significant level with FHR-1, FHR-2, FHR-3 and FHR-4 from the GWASs in our control group.

The identification of the CFH locus as a cis protein quantitative trait locus (cis-pQTL) for the five FHR levels prompted the use of the available genetic data in a Mendelian randomization fashion to triangulate this evidence. For FHR-1, FHR-2, FHR-4 and FHR-5, the support provided by Mendelian randomization analyses for a potential casual role in susceptibility to AMD is striking, with Mendelian randomization estimates corroborating the preliminary evidence shown by the observational OR estimates (see Table 9).

This reframes our understanding of the aetiology of AMD, and the role of the non-coding risk variants on chromosome 1q31.3, demonstrating a prominent role for the FHR proteins. It also demonstrates that targeting (and lowering) of FHR proteins in the circulation as a viable therapeutic avenue for AMD, including systemic therapeutic strategies.

Identifying patients with risk factors for AMD will allow patients to avoid surgical procedures, especially in the early stages of disease before the loss of visual acuity, where therapeutic intervention may yield the most benefit. Patient stratification will be important as only a proportion of AMD patients are likely to suffer from FHR-mediated disease. However, as demonstrated above a patient's genetic-risk profile, coupled with measurements of their circulating FHR protein levels, is able to identify and stratify those patients most likely to benefit from such treatments, and to monitor their response to FHR-lowering agents.

TABLE 10
Single-variant association analyses for the 8 AMD independently associated variants at the
CFH locus from the IAMDGC study with FH, FHL-1, FHR-1 to FHR-5 levels in 252 controls.

| IAMDGC | dbSNP ID | | | Association with levels in Cambridge controls^{a} | | | | | | |
|---|---|---|---|---|---|---|---|---|---|---|
| association | (Chr:Position)^{c} | | | FH | FHL-1 | FHR-1 | FHR-2 | FHR-3 | FHR-4 | FHR-5 |
| signal | Major/Minor | IAMDGC OR | MAF | Beta | Beta | Beta | Beta | Beta | Beta | Beta |
| number | allele | (MAF in | Cambridge | (SE) | (SE) | (SE) | (SE) | (SE) | (SE) | (SE) |
| (direction^{b}) | (Imputation R²)^{d} | controls) | controls | P | P | P | P | P | P | P |
| 1.1 | rs10922109 | 0.38 | 0.422 | 0.49 | −0.10 | −10.67 | −0.75 | −1.20 | −1.0 | −0.04 |
| (−) | (1:196704632) | (0.426) | | (0.25) | (0.02) | (1.04) | (0.11) | (0.14) | (0.17) | (0.03) |
| | C/A (1.00) | | | 0.056 | 3.7 × | 7.8 × | 2.9 × | 1.7 × | 1.5 × | 0.184 |
| | | | | | 10⁻⁵ | 10⁻²¹ | 10⁻¹¹ | 10⁻¹⁶ | 10⁻⁹ | |
| 1.2 | rs570618 | 2.38 | 0.357 | 0.27 | 0.05 | 8.19 | 0.85 | 0.16 | 0.62 | 0.10 |
| (+) | (1:196657064) | (0.364) | | (0.26) | (0.03) | (1.17) | (0.11) | (0.16) | (0.18) | (0.03) |
| | G/T (1.00) | | | 0.296 | 0.046 | 2.0 × | 1.8 × | 0.304 | 6.8 × | 7.8 × |
| | | | | | | 10⁻¹¹ | 10⁻¹² | | 10⁻⁴ | 10⁻⁴ |
| 1.3 | rs121913059 | 20.28 | 0 | No control carrier observed; not analyzed | | | | | | |
| (+) | (1:196716375) | (0.00014) | | | | | | | | |
| | C/T (Genotyped) | | | | | | | | | |
| 1.4 | rs148553336 | 0.29 | | −2.29 | −0.09 | 0.06 | −1.99 | 0.33 | 0.74 | −0.55 |
| (−) | (1:196613173) | (0.009) | 0.017 | (1.02) | (0.10) | (5.00) | (0.48) | (0.62) | (0.72) | (0.11) |
| | T/C (Genotyped) | | | 0.025 | 0.353 | 0.990 | 4.6 × | 0.603 | 0.302 | 6.3 × |
| | | | | | | | 10⁻⁵ | | | 10⁻⁷ |
| 1.5 | rs187328863 | 2.27 | 0.013 | 1.12 | 0.06 | 0.75 | 1.13 | −0.51 | 0.61 | 0.01 |
| (+) | (1:196380158) | (0.028) | | (1.34) | (0.13) | (6.52) | (0.64) | (0.81) | (0.94) | (0.15) |
| | C/T (0.83) | | | 0.404 | 0.660 | 0.908 | 0.080 | 0.536 | 0.515 | 0.956 |
| 1.6 | rs61818925 | 0.60 | 0.405 | −0.42 | 0.001 | 0.99 | −0.93 | 0.85 | −1.12 | −0.07 |
| (−) | (1:196815450) | (0.385) | | (0.27) | (0.03) | (1.33) | (0.12) | (0.16) | (0.18) | (0.03) |
| | G/T (0.87) | | | 0.124 | 0.962 | 0.459 | 1.3 × | 1.7 × | 1.5 × | 0.014 |
| | | | | | | | 10⁻¹³ | 10⁻⁷ | 10⁻⁹ | |
| 1.7 | rs35292876 | 2.42 | 0.004 | MAF <= 1%; not analyzed | | | | | | |
| (+) | (1:196706642) | (0.009) | | | | | | | | |
| | C/T (Genotyped) | | | | | | | | | |
| 1.8 | rs191281603 | 1.07 | 0.008 | MAF <= 1%; not analyzed | | | | | | |
| (+) | (1:196958651) | (0.006) | | | | | | | | |
| | C/G (0.42) | | | | | | | | | |

### Example 4: siRNA Knockdown of Expression of Genes Encoding Factor H Family Proteins

siRNA molecules targeting different regions of genes encoding Factor H family proteins were designed and are evaluated for their effect on expression of the relevant target proteins in human liver cells.

The mRNA sequences of Factor H family proteins targeted by the siRNAs are shown in SEQ ID NOs:158 to 177. The nucleotide sequences of the antisense nucleic acids of the siRNAs (i.e. the guide strands) targeting SEQ ID NOs:158 to 177 are shown in SEQ ID NOs:178 to 197. Control, scrambled sequences for the respective antisense sequences are shown in SEQ ID NOs:198 to 217.

Table 11 shows the percentage identity between SEQ ID NOs:158-177 and FH family proteins, demonstrating that siRNAs comprising SEQ ID NOs:158-177 and 178-197 each target one or more of FHR1, FHR2, FHR3, FHR4, FHR5 and/or FH.

HuH cells from a human liver carcinoma cell line are cultured in Dulbecco's Modified Eagle's Medium with low glucose (DMEM, Sigma, catalogue number D6046) supplemented with 10% fetal fovine serum (FBS, Sigma, catalogue number F9665) and 1% penicillin streptomycin (Pen/Strep, Sigma P0781) in 5% CO₂ incubator at 37° C.

The human liver carcinoma cells are seeded in 24 well plates (50,000 cells/well) and cultured. After 24 hrs, the cells are transfected with either (i) 10 nM of siRNA according to SEQ ID NOs:178 to 197, or (ii) their corresponding scrambled siRNA control, using 1 μl of Lipofectamine RNAimax (Invitrogen, catalogue number 13778-075) for 24 hours. All reactions are carried out in triplicate.

After 24 hours post-transfection, RNA is extracted using the Isolate RNA Mini Kit (Bioline, catalogue number BIO-52072) and cDNA is synthesised using the High-Capacity cDNA Reverse Transcription Kit (Applied Biosystems, catalogue number 4368814).

Quantitative PCR reactions are performed using pre-designed FAM-labeled TaqMan probes (Integrated DNA Technologies) following the manufacturer's instructions. In brief, 100 ng of cDNA is resuspended in a reaction mix including 0.5 μl of either TaqMan probe for the relevant gene(s) (i.e. CFHR1, CFHR2, CFHR3, CFHR4, CFHR5 or CFH) or GAPDH TaqMan probe (Hs02758991_g1), 10 μl of 2× reaction mastermix (Applied Biosystems™, Thermofischer Scientific, cat no. 4440040), in a final reaction volume of 20 μl in a 96-well plate. Samples are run in triplicate using an ABI Step One thermocycler (Applied Biosystems) using the following thermal cycling conditions: 42° C. for 5 minutes, 95° C. for 10 seconds and 40 cycles of 95° C. for 5 seconds and 60° C. for 34 seconds. CFHR4 gene expression is normalised to GAPDH expression, and relative expression is determined by the AACt method.

### Expected Results:

siRNA targeting target 1 is found to significantly reduce expression of CFHR1 and CFHR2. siRNA targeting target 2 is found to significantly reduce expression of CFHR1. siRNA targeting target 3 is found to significantly reduce expression of CFHR1. siRNA targeting target 4 is found to significantly reduce expression of CFHR3. siRNA targeting target 5 is found to significantly reduce expression of CFHR3 and CFHR4. siRNA targeting target 6 is found to significantly reduce expression of CFHR3. siRNA targeting target 7 is found to significantly reduce expression of CFHR4. siRNA targeting target 8 is found to significantly reduce expression of CFHR4. siRNA targeting target 9 is found to significantly reduce expression of CFHR3 and CFHR4. siRNA targeting target 10 is found to significantly reduce expression of CFHR5. siRNA targeting target 11 is found to significantly reduce expression of CFHR5. siRNA targeting target 10 is found to significantly reduce expression of CFHR5. siRNA targeting target 12 is found to significantly reduce expression of CFH, CFHR1 and CFHR2. siRNA targeting target 13 is found to significantly reduce expression of CFH and CFHR1. siRNA targeting target 14 is found to significantly reduce expression of CFH and CFHR4. siRNA targeting target 15 is found to significantly reduce expression of CFH and CFHR2. siRNA targeting target 16 is found to significantly reduce expression of CFHR2. siRNA targeting target 17 is found to significantly reduce expression of CFHR3. siRNA targeting target 18 is found to significantly reduce expression of CFHR3. siRNA targeting target 19 is found to significantly reduce expression of CFHR3. siRNA targeting target 20 is found to significantly reduce expression of CFHR4.

The scrambled siRNAs are found to have no effect on expression of CFHR1, CFHR2, CFHR3, CFHR4, CFHR5 and CFH.

### Results:

| | Column A | Column B | |
|---|---|---|---|
| CFHR | siRNA target | siRNA antisense | Expected gene(s) |
| siRNA # | sequence | sequence | to be targeted |
| 1 | SEQ ID NO: 158 | SEQ ID NO: 178 | CFHR1 and CFHR2 |
| 2 | SEQ ID NO: 159 | SEQ ID NO: 179 | CFHR1 |
| 3 | SEQ ID NO: 160 | SEQ ID NO: 180 | CFHR1 |
| 4 | SEQ ID NO: 161 | SEQ ID NO: 181 | CFHR3 |
| 5 | SEQ ID NO: 162 | SEQ ID NO: 182 | CFHR3 and CFHR4 |
| 6 | SEQ ID NO: 163 | SEQ ID NO: 183 | CFHR3 |
| 7 | SEQ ID NO: 164 | SEQ ID NO: 184 | CFHR4 |
| 8 | SEQ ID NO: 165 | SEQ ID NO: 185 | CFHR4 |
| 9 | SEQ ID NO: 166 | SEQ ID NO: 186 | CFHR3 and CFHR4 |
| 10 | SEQ ID NO: 167 | SEQ ID NO: 187 | CFHR5 |
| 11 | SEQ ID NO: 168 | SEQ ID NO: 188 | CFHR5 |
| 12 | SEQ ID NO: 169 | SEQ ID NO: 189 | CFHR1, CFHR2, CFH |
| 13 | SEQ ID NO: 170 | SEQ ID NO: 190 | CFHR1, CFH |
| 14 | SEQ ID NO: 171 | SEQ ID NO: 191 | CFHR1, CFH |
| 15 | SEQ ID NO: 172 | SEQ ID NO: 192 | CFHR2 |
| 16 | SEQ ID NO: 173 | SEQ ID NO: 193 | CFHR2 |
| 17 | SEQ ID NO: 174 | SEQ ID NO: 194 | CFHR3 |
| 18 | SEQ ID NO: 175 | SEQ ID NO: 195 | CFHR3 |
| 19 | SEQ ID NO: 176 | SEQ ID NO: 196 | CFHR3 |
| 20 | SEQ ID NO: 177 | SEQ ID NO: 197 | CFHR4 |

The experiments were performed as described above. Comparison of CFHR1, CFHR2, CFHR3, CFHR4, CFHR5 and CFH expression between siRNA transfected cells and controls (scrambled) transfected cells were calculated using the unpaired Student's t-test. A p-value<0.05 was considered to be statistically significant.

Surprisingly the results showed that CFHR siRNAs 1-3 were able to suppress the expression of more CHFR genes than expected. siRNA1 was designed to knock down CFHR1 and CFHR2 expression, while siRNA2 and siRNA3 were designed to knock down CFHR1 expression only.

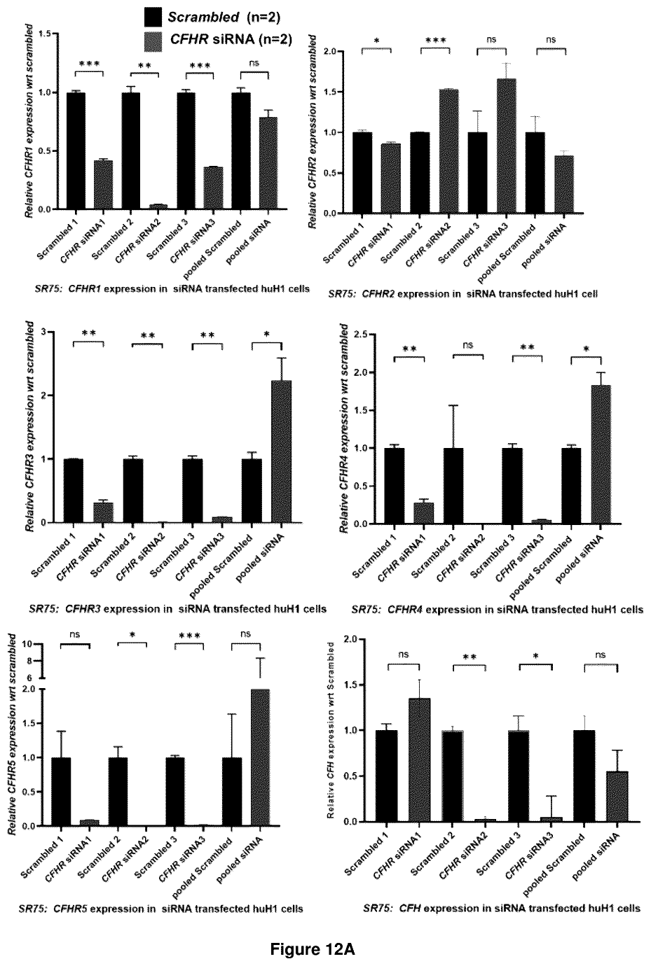

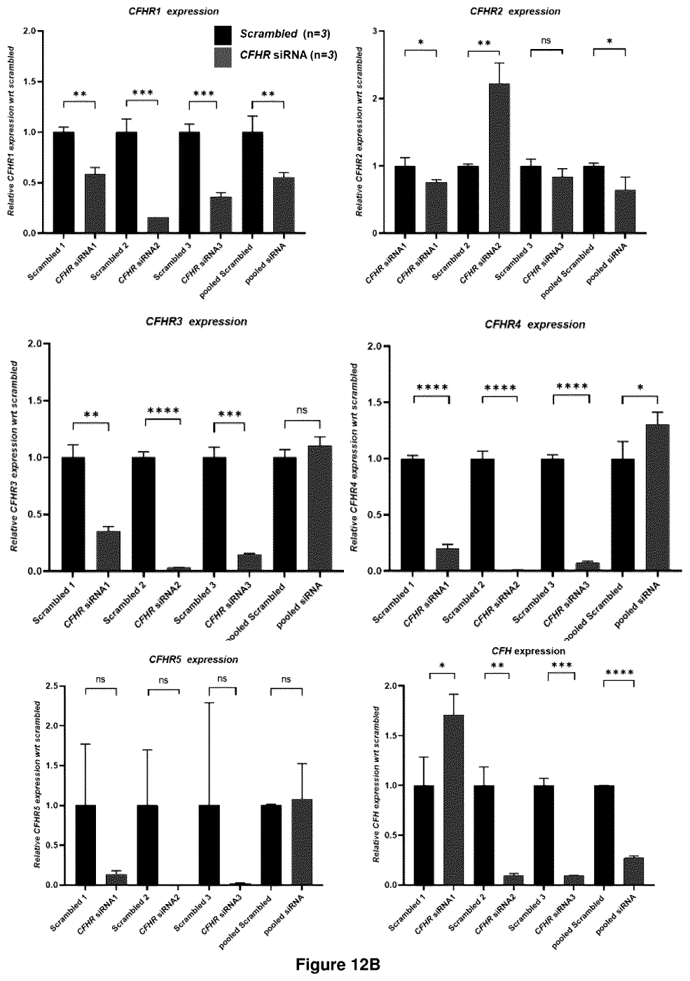

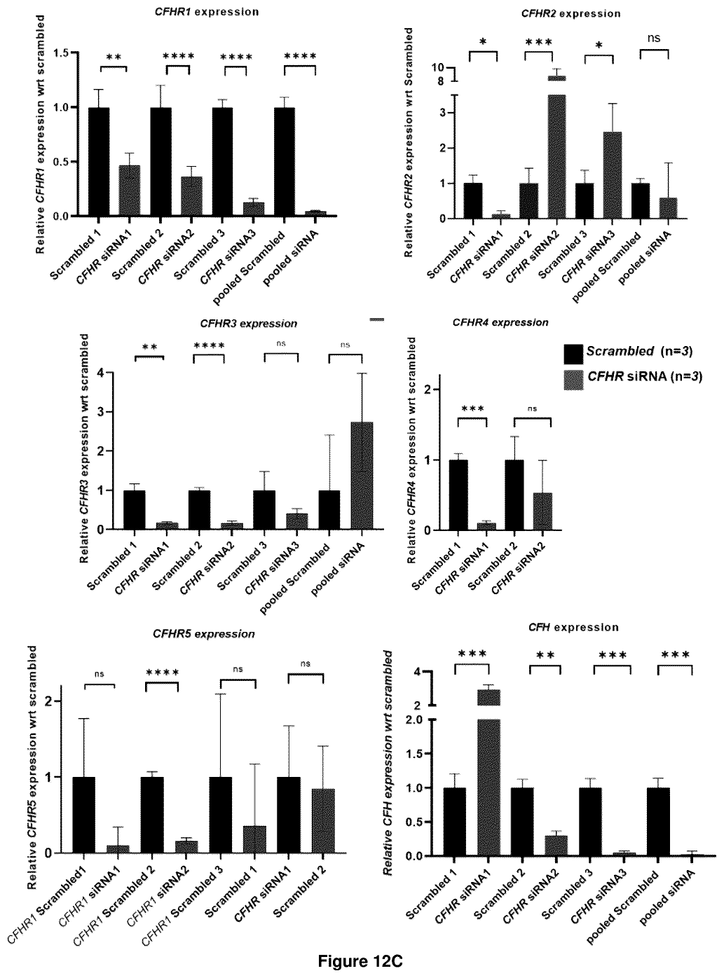

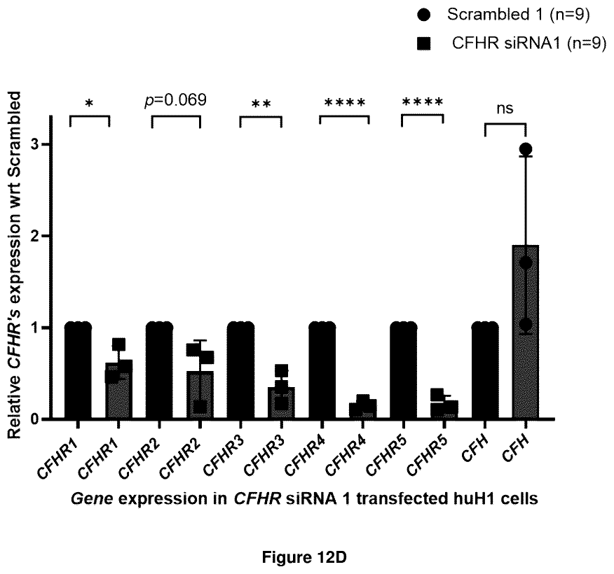

FIGS. 12A to 12C show that all three siRNAs had a significant knockdown effect on the expression of CFHR1. CFHR siRNA1 also reduced expression of CFHR2. Interestingly, all three siRNAs were also found to reduce gene expression of CFHR3, CFHR4 and CFHR5. This effect was not expected from the degree of sequence complementarity of these siRNAs with said targets.

FIG. 12D shows the combined results from three separate transfections of CFHR siRNA1 on the expression of CFH family genes. Whilst it is able to knockdown expression of each of CFHR1-CFHR5, siRNA1 was found not to reduce the expression of CFH, which is advantageous for the treatment of complement-related disorders and makes this siRNA an ideal candidate for therapeutic use.

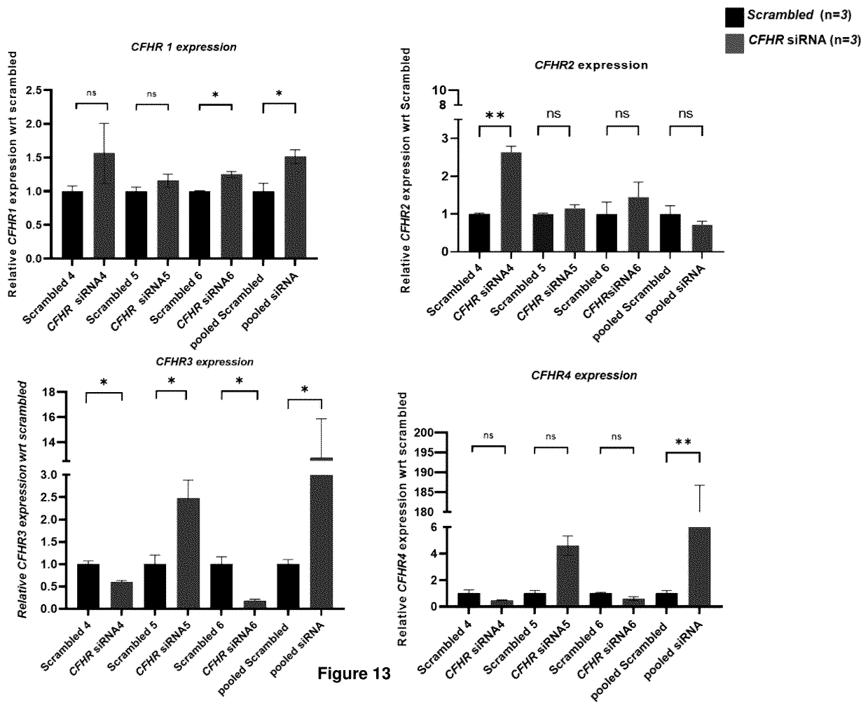

FIG. 13 shows the effect of CFHR siRNA4, siRNA5 and siRNA6 on expression of CFH family genes. siRNA4 and siRNA6 were found to significantly reduce expression of CFHR3 and CFHR4. This was unexpected as only siRNA5 was designed to target both genes. Instead, siRNA5 was found to increase expression of CFHR3 and CFHR4. None of these siRNAs reduced expression of CFHR1 or CFHR2.

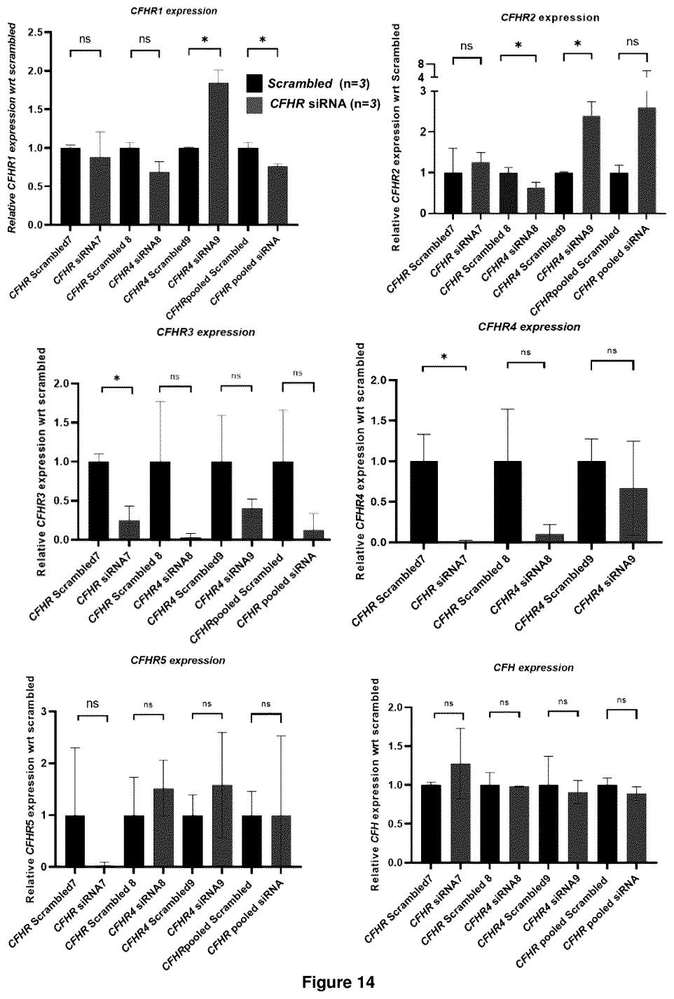

FIG. 14 shows the effect of CFHR siRNA7, siRNA8 and siRNA9 on the expression of CFH family genes. Only CFHR siRNA7 was found to significantly reduce expression of CFHR3 and CFHR4 genes. None of these siRNAs had any effect on the expression of CFHR1 or CFH, although siRNA8 was able to significantly reduce expression of CFHR2.

### Example 5: The Effect of siRNA Knockdown on FHR Proteins

siRNAs 1 to 20 in Example 4 are tested for their effect on the expression of FH family proteins.

siRNAs that reduce gene expression of CFH family genes are found to reduce expression of the corresponding encoded FH family proteins.

### Example 6: CRISPR/Cas Knockdown of FH Family Genes

GuideRNA sequences according to SEQ ID NO:224 to 227 are tested for their effect on the expression of FH family genes and/or proteins, e.g. using a CRISPR/Cas system.

A CRISPR/Cas system comprising a gRNA according to SEQ ID NO: 224 is found to reduce expression of CFHR1, CFHR2, CFHR3, CFHR4 and CFHR5.

A CRISPR/Cas system comprising a gRNA according to SEQ ID NO: 225, 226 or 227 is found to reduce expression of CFHR1, CFHR2, and CFHR5.

### Example 7: In Vivo Knockdown of FH Family Genes

Transgenic rodents are generated to express human FH family genes, e.g. one or more of CFHR1, CFHR2, CFHR3, CFHR4, CFHR5, FH and/or FHL-1. The rodents may comprise a humanised liver, in which the native liver has been ablated and then engrafted with human hepatocytes, which proliferate to restore the functioning liver.

siRNAs as described in Example 4 and CRISPR/Cas systems comprising gRNAs as in Example 6 are administered to the rodents and tested for their ability in vivo to reduce gene and/or protein expression of one or more of CFHR1, CFHR2, CFHR3, CFHR4, CFHR5, FH and/or FHL-1.

TABLE 11
NCBI BLAST analysis of percentage identity between siRNA target sequences SEQ ID NO: 158-177 and FH family proteins.

| siRNA target | | Percentage | | |
|---|---|---|---|---|
| (SEQ ID NO) | Target description | identity | E value | Accession |
| 158 | Homo sapiens complement factor H related 1 (CFHR1), transcript variant 6, mRNA | 100 | 6.00E−04 | NM_001379310.1 |
| 158 | Homo sapiens complement factor H related 1 (CFHR1), transcript variant 2, mRNA | 100 | 6.00E−04 | NM_001379306.1 |
| 158 | Homo sapiens complement factor H related 1 (CFHR1), transcript variant 7, mRNA | 100 | 6.00E−04 | NM_001379311.1 |
| 158 | Homo sapiens complement factor H related 1 (CFHR1), transcript variant 1, mRNA | 100 | 6.00E−04 | NM_002113.3 |
| 158 | Homo sapiens complement factor H related 1 (CFHR1), transcript variant 8, mRNA | 100 | 6.00E−04 | NM_001379312.1 |
| 158 | Homo sapiens complement factor H related 1 (CFHR1), transcript variant 3, mRNA | 100 | 6.00E−04 | NM_001379307.1 |
| 158 | Homo sapiens complement factor H related 1 (CFHR1), transcript variant 5, mRNA | 100 | 6.00E−04 | NM_001379309.1 |
| 158 | Homo sapiens complement factor H related 1 (CFHR1), transcript variant 4, mRNA | 100 | 6.00E−04 | NM_001379308.1 |
| 158 | Homo sapiens complement factor H related 2 (CFHR2), transcript variant 1, mRNA | 100 | 6.00E−04 | NM_005666.4 |
| 158 | Homo sapiens complement factor H related 2 (CFHR2), transcript variant 2, mRNA | 100 | 6.00E−04 | NM_001312672.1 |
| 159 | Homo sapiens complement factor H related 1 (CFHR1), transcript variant 6, mRNA | 100 | 6.00E−04 | NM_001379310.1 |
| 159 | Homo sapiens complement factor H related 1 (CFHR1), transcript variant 2, mRNA | 100 | 6.00E−04 | NM_001379306.1 |
| 159 | Homo sapiens complement factor H related 1 (CFHR1), transcript variant 7, mRNA | 100 | 6.00E−04 | NM_001379311.1 |
| 159 | Homo sapiens complement factor H related 1 (CFHR1), transcript variant 1, mRNA | 100 | 6.00E−04 | NM_002113.3 |
| 159 | Homo sapiens complement factor H related 1 (CFHR1), transcript variant 8, mRNA | 100 | 6.00E−04 | NM_001379312.1 |
| 159 | Homo sapiens complement factor H related 1 (CFHR1), transcript variant 3, mRNA | 100 | 6.00E−04 | NM_001379307.1 |
| 159 | Homo sapiens complement factor H related 1 (CFHR1), transcript variant 5, mRNA | 100 | 6.00E−04 | NM_001379309.1 |
| 159 | Homo sapiens complement factor H related 1 (CFHR1), transcript variant 4, mRNA | 100 | 6.00E−04 | NM_001379308.1 |
| 160 | Homo sapiens complement factor H related 1 (CFHR1), transcript variant 6, mRNA | 100 | 6.00E−04 | NM_001379310.1 |
| 160 | Homo sapiens complement factor H related 1 (CFHR1), transcript variant 2, mRNA | 100 | 6.00E−04 | NM_001379306.1 |
| 160 | Homo sapiens complement factor H related 1 (CFHR1), transcript variant 7, mRNA | 100 | 6.00E−04 | NM_001379311.1 |
| 160 | Homo sapiens complement factor H related 1 (CFHR1), transcript variant 1, mRNA | 100 | 6.00E−04 | NM_002113.3 |
| 160 | Homo sapiens complement factor H related 1 (CFHR1), transcript variant 8, mRNA | 100 | 6.00E−04 | NM_001379312.1 |
| 160 | Homo sapiens complement factor H related 1 (CFHR1), transcript variant 3, mRNA | 100 | 6.00E−04 | NM_001379307.1 |
| 160 | Homo sapiens complement factor H related 1 (CFHR1), transcript variant 5, mRNA | 100 | 6.00E−04 | NM_001379309.1 |
| 160 | Homo sapiens complement factor H related 1 (CFHR1), transcript variant 4, mRNA | 100 | 6.00E−04 | NM_001379308.1 |
| 161 | Homo sapiens complement factor H related 3 (CFHR3), transcript variant 1, mRNA | 100 | 6.00E−04 | NM_021023.6 |
| 161 | Homo sapiens complement factor H related 3 (CFHR3), transcript variant 2, mRNA | 100 | 6.00E−04 | NM_001166624.2 |
| 162 | Homo sapiens complement factor H related 3 (CFHR3), transcript variant 1, mRNA | 100 | 6.00E−04 | NM_021023.6 |
| 162 | Homo sapiens complement factor H related 4 (CFHR4), transcript variant 3, mRNA | 100 | 6.00E−04 | NM_006684.5 |
| 162 | Homo sapiens complement factor H related 4 (CFHR4), transcript variant 2, mRNA | 100 | 6.00E−04 | NM_001201551.2 |
| 162 | Homo sapiens complement factor H related 4 (CFHR4), transcript variant 1, mRNA | 100 | 6.00E−04 | NM_001201550.3 |
| 162 | Homo sapiens complement factor H related 3 (CFHR3), transcript variant 2, mRNA | 100 | 6.00E−04 | NM_001166624.2 |
| 163 | Homo sapiens complement factor H related 3 (CFHR3), transcript variant 1, mRNA | 100 | 6.00E−04 | NM_021023.6 |
| 163 | Homo sapiens complement factor H related 3 (CFHR3), transcript variant 2, mRNA | 100 | 6.00E−04 | NM_001166624.2 |
| 164 | Homo sapiens complement factor H related 4 (CFHR4), transcript variant 3, mRNA | 100 | 6.00E−04 | NM_006684.5 |
| 164 | Homo sapiens complement factor H related 4 (CFHR4), transcript variant 2, mRNA | 100 | 6.00E−04 | NM_001201551.2 |
| 164 | Homo sapiens complement factor H related 4 (CFHR4), transcript variant 1, mRNA | 100 | 6.00E−04 | NM_001201550.3 |
| 165 | Homo sapiens complement factor H related 4 (CFHR4), transcript variant 2, mRNA | 100 | 6.00E−04 | NM_001201551.2 |
| 165 | Homo sapiens complement factor H related 4 (CFHR4), transcript variant 1, mRNA | 100 | 6.00E−04 | NM_001201550.3 |
| 166 | Homo sapiens complement factor H related 4 (CFHR4), transcript variant 3, mRNA | 100 | 6.00E−04 | NM_006684.5 |
| 166 | Homo sapiens complement factor H related 4 (CFHR4), transcript variant 2, mRNA | 100 | 6.00E−04 | NM_001201551.2 |
| 166 | Homo sapiens complement factor H related 4 (CFHR4), transcript variant 1, mRNA | 100 | 6.00E−04 | NM_001201550.3 |
| 166 | Homo sapiens complement factor H related 3 (CFHR3), transcript variant X3, RNA | 100 | 6.00E−04 | XR_002958987.1 |
| 166 | Homo sapiens complement factor H related 3 (CFHR3), transcript variant X2, RNA | 100 | 6.00E−04 | XR_001736938.1 |
| 166 | Homo sapiens complement factor H related 3 (CFHR3), transcript variant X1, RNA | 100 | 6.00E−04 | XR_001736937.1 |
| 166 | Homo sapiens complement factor H related 3 (CFHR3), transcript variant X4, RNA | 100 | 6.00E−04 | XR_426757.2 |
| 167 | Homo sapiens complement factor H related 5 (CFHR5), mRNA | 100 | 6.00E−04 | NM_030787.4 |
| 168 | Homo sapiens complement factor H related 5 (CFHR5), mRNA | 100 | 6.00E−04 | NM_030787.4 |
| 169 | Homo sapiens complement factor H related 1 (CFHR1), transcript variant 6, mRNA | 100 | 6.00E−04 | NM_001379310.1 |
| 169 | Homo sapiens complement factor H related 1 (CFHR1), transcript variant 2, mRNA | 100 | 6.00E−04 | NM_001379306.1 |
| 169 | Homo sapiens complement factor H related 1 (CFHR1), transcript variant 7, mRNA | 100 | 6.00E−04 | NM_001379311.1 |
| 169 | Homo sapiens complement factor H related 1 (CFHR1), transcript variant 1, mRNA | 100 | 6.00E−04 | NM_002113.3 |
| 169 | Homo sapiens complement factor H related 1 (CFHR1), transcript variant 8, mRNA | 100 | 6.00E−04 | NM_001379312.1 |
| 169 | Homo sapiens complement factor H related 1 (CFHR1), transcript variant 3, mRNA | 100 | 6.00E−04 | NM_001379307.1 |
| 169 | Homo sapiens complement factor H related 1 (CFHR1), transcript variant 5, mRNA | 100 | 6.00E−04 | NM_001379309.1 |
| 169 | Homo sapiens complement factor H related 1 (CFHR1), transcript variant 4, mRNA | 100 | 6.00E−04 | NM_001379308.1 |
| 169 | Homo sapiens complement factor H (CFH), transcript variant 1, mRNA | 100 | 6.00E−04 | NM_000186.4 |
| 170 | Homo sapiens complement factor H related 1 (CFHR1), transcript variant 6, mRNA | 100 | 6.00E−04 | NM_001379310.1 |
| 170 | Homo sapiens complement factor H related 1 (CFHR1), transcript variant 2, mRNA | 100 | 6.00E−04 | NM_001379306.1 |
| 170 | Homo sapiens complement factor H related 1 (CFHR1), transcript variant 7, mRNA | 100 | 6.00E−04 | NM_001379311.1 |
| 170 | Homo sapiens complement factor H related 1 (CFHR1), transcript variant 1, mRNA | 100 | 6.00E−04 | NM_002113.3 |
| 170 | Homo sapiens complement factor H related 1 (CFHR1), transcript variant 8, mRNA | 100 | 6.00E−04 | NM_001379312.1 |
| 170 | Homo sapiens complement factor H related 1 (CFHR1), transcript variant 3, mRNA | 100 | 6.00E−04 | NM_001379307.1 |
| 170 | Homo sapiens complement factor H related 1 (CFHR1), transcript variant 5, mRNA | 100 | 6.00E−04 | NM_001379309.1 |
| 170 | Homo sapiens complement factor H related 1 (CFHR1), transcript variant 4, mRNA | 100 | 6.00E−04 | NM_001379308.1 |
| 170 | Homo sapiens complement factor H (CFH), transcript variant 1, mRNA | 100 | 6.00E−04 | NM_000186.4 |
| 171 | Homo sapiens complement factor H related 1 (CFHR1), transcript variant 6, mRNA | 100 | 6.00E−04 | NM_001379310.1 |
| 171 | Homo sapiens complement factor H related 1 (CFHR1), transcript variant 2, mRNA | 100 | 6.00E−04 | NM_001379306.1 |
| 171 | Homo sapiens complement factor H related 1 (CFHR1), transcript variant 7, mRNA | 100 | 6.00E−04 | NM_001379311.1 |
| 171 | Homo sapiens complement factor H related 1 (CFHR1), transcript variant 1, mRNA | 100 | 6.00E−04 | NM_002113.3 |
| 171 | Homo sapiens complement factor H related 1 (CFHR1), transcript variant 8, mRNA | 100 | 6.00E−04 | NM_001379312.1 |
| 171 | Homo sapiens complement factor H related 1 (CFHR1), transcript variant 3, mRNA | 100 | 6.00E−04 | NM_001379307.1 |
| 171 | Homo sapiens complement factor H related 1 (CFHR1), transcript variant 5, mRNA | 100 | 6.00E−04 | NM_001379309.1 |
| 171 | Homo sapiens complement factor H related 1 (CFHR1), transcript variant 4, mRNA | 100 | 6.00E−04 | NM_001379308.1 |
| 171 | Homo sapiens complement factor H (CFH), transcript variant 1, mRNA | 100 | 6.00E−04 | NM_000186.4 |
| 172 | Homo sapiens complement factor H related 2 (CFHR2), transcript variant 1, mRNA | 100 | 6.00E−04 | NM_001312672.1 |
| 172 | Homo sapiens complement factor H related 2 (CFHR2), transcript variant 2, mRNA | 100 | 6.00E−04 | NM_001312672.1 |
| 173 | Homo sapiens complement factor H related 2 (CFHR2), transcript variant 1, mRNA | 100 | 6.00E−04 | NM_005666.4 |
| 174 | Homo sapiens complement factor H related 3 (CFHR3), transcript variant 1, mRNA | 100 | 6.00E−04 | NM_021023.6 |
| 174 | Homo sapiens complement factor H related 3 (CFHR3), transcript variant 2, mRNA | 100 | 6.00E−04 | NM_001166624.2 |
| 175 | Homo sapiens complement factor H related 3 (CFHR3), transcript variant 1, mRNA | 100 | 6.00E−04 | NM_021023.6 |
| 175 | Homo sapiens complement factor H related 3 (CFHR3), transcript variant 2, mRNA | 100 | 6.00E−04 | NM_001166624.2 |
| 176 | Homo sapiens complement factor H related 3 (CFHR3), transcript variant 1, mRNA | 100 | 6.00E−04 | NM_021023.6 |
| 176 | Homo sapiens complement factor H related 3 (CFHR3), transcript variant 2, mRNA | 100 | 6.00E−04 | NM_001166624.2 |
| 177 | Homo sapiens complement factor H related 4 (CFHR4), transcript variant 3, mRNA | 100 | 6.00E−04 | NM_006684.5 |
| 177 | Homo sapiens complement factor H related 4 (CFHR4), transcript variant 2, mRNA | 100 | 6.00E−04 | NM_001201551.2 |
| 177 | Homo sapiens complement factor H related 4 (CFHR4), transcript variant 1, mRNA | 100 | 6.00E−04 | NM_001201550.3 |

1. An agent for reducing gene and/or protein expression of one or more Factor H family proteins.

2. The agent according to claim 1, wherein the one or more Factor H family proteins are Factor H-related proteins, optionally wherein the Factor H-related proteins are selected from FHR1, FHR2, FHR3, FHR4 and FHR5.

3. The agent according to claim 1, wherein the agent is an inhibitory nucleic acid.

4. The agent according to claim 3, wherein the inhibitory nucleic acid comprises or encodes antisense nucleic acid targeting a nucleotide sequence of RNA encoded by one or more genes encoding the one or more Factor H family proteins.

5. The agent according to claim 3, wherein the inhibitory nucleic acid comprises or encodes antisense nucleic acid targeting a nucleotide sequence comprising, or consisting of, SEQ ID NO:158, 159, 160, 161, 162, 163, 164, 165, 166, 167, 168, 169, 170, 171, 172, 173, 174, 175, 176 or 177.

6. The agent according to claim 3, wherein the inhibitory nucleic acid comprises or encodes antisense nucleic acid comprising or consisting of a sequence having at least 75% sequence identity to SEQ ID NO:178, 179, 180, 181, 182, 183, 184, 185, 186, 187, 188, 189, 190, 191, 192, 193, 194, 195, 196 or 197.

7. The agent according to claim 3, wherein the inhibitory nucleic acid is an siRNA, shRNA, miRNA or antisense oligonucleotide.

8. The agent according to claim 1, wherein the agent is a meganuclease, a zinc finger nuclease (ZFN), a transcription activator-like effector-based nuclease (TALEN), or a CRISPR-Cas system.

9. The agent according to claim 8, wherein the CRISPR-Cas system comprises a sequence having at least 75% sequence identity to SEQ ID NO: 224, 225, 226 or 227.

10. The agent according to claim 1, wherein the agent does not reduce gene and/or protein expression of FH and/or FHL-1.

11. A nucleic acid, optionally isolated, encoding the agent according to claim 1.

12. An expression vector, comprising the nucleic acid according to claim 11.

13. A composition comprising the agent according to claim 1, and a pharmaceutically acceptable carrier, diluent, excipient, or adjuvant.

14. A cell comprising the agent according to claim 1.

15. An in vitro or in vivo method for reducing gene and/or protein expression of one or more Factor H family proteins, comprising contacting a cell with the agent according to claim 1.

16.-19. (canceled)

20. A method of treating or preventing a complement-related disorder in a subject, comprising administering to the subject a therapeutically- or prophylactically-effective amount of the agent according to claim 1.

21.-23. (canceled)

24. The method of according to claim 20, wherein prior to administering the agent, the method comprises:
(a) determining the level of a complement protein selected from one or more of FHR1, FHR2, FHR3, FHR4 and/or FHR5, and optionally FHL-1, in a blood sample obtained from the subject; and
(b) determining whether the level of the complement protein is elevated as compared to the level of that complement protein in blood in a control subject that does not have a complement-related disorder.

25. The method according to claim 20, wherein the complement-related disorder is selected from: macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, kidney injury/damage/dysfunction, glomerular diseases, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, anti-neutrophilic cytoplasmic autoantibodies (ANCA) vasculitis, neurodegeneration/neurodegenerative disease, dementia, multiple sclerosis (MS), Lewy body disease, Amyotrophic lateral sclerosis (ALS), Huntington's disease, prion diseases, cancer, lung cancer, glioblastoma e.g. glioblastoma multiforme (GBM), stroke, insulin resistance, diabetes, an infectious disease, Parkinson's disease, and/or Alzheimer's disease.
